(19) United States
(12) Patent Application Publication
(10) Pub. No.: US 2006/0166983 A1
(43) Pub. Date: Jul. 27, 2006

(54) Indole derivatives as ppar modulators

(75) Inventors: Scott Eugene Conner, Elizabethtown, IN (US); James Allen Knobelsdorf, Noblesville, IN (US); Nathan Bryan Mantlo, Brownsburg, IN (US); Daniel Ray Mayhugh, Carmel, IN (US); Xiaodong Wang, Carmel, IN (US); Guoxin Zhu, Noblesville, IN (US); Jeffrey Michael Schkeryantz, Fishers, IN (US); Pierre-Yves Michellys, San Diego, CA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Eli Lilly and Company

(21) Appl. No.: 10/541,555

(22) PCT Filed: Dec. 31, 2003
(86) PCT No.: PCT/US03/41698

§ 371 Date: Dec. 23, 2005

Related U.S. Application Data
(60) Provisional application No. 60/438,541, filed on Jan. 6, 2003.

(51) Int. Cl. A61K 31/5377; A61K 31/454; A61K 31/4439; A61K 31/405; C07D 413/02; C07D 405/02; C07D 403/02

(52) U.S. Cl. 514/232.5; 514/323; 514/414; 546/201; 548/465; 544/153

(57) ABSTRACT

The present invention is directed to a method of treatment by modulating a peroxisome proliferator activated receptor by employing a compound of Structural Formula (I). The variables in (I) are defined herein. Also included are compounds, methods of making compounds, and pharmaceutical compositions. The compounds of the present invention are believed to be effective in treating and preventing Syndrome X, Type II diabetes, hyperglycemia, hyperlipidemia, obesity, coagaulopathy, hypertension, atherosclerosis, and other disorders related to Syndrome X and cardiovascular diseases.

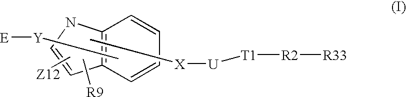

### RELATED APPLICATIONS

This application claims the benefit of U.S. Provisional Application No. 60/438,541, filed Jan. 6, 2003, the entire teachings of which are incorporated herein by reference.

### BACKGROUND OF THE INVENTION

Peroxisome Proliferator Activated Receptors (PPARs) are members of the nuclear hormone receptor super family, which are ligand-activated transcription factors regulating gene expression. Various subtypes of PPARs have been discovered. These include, for example, PPARA, NUC1, PPARγ and PPARδ.

PPARα, PPARγ and PPARδ receptors have been implicated in diabetes mellitus, cardiovascular disease, obesity, Syndrome X and gastrointestinal disease, such as, inflammatory bowel disease. Syndrome X is the combination of symptoms which include hyperinsulemia combined with hypertension, elevated body weight, elevated triglycerides and elevated LDL.

Current PPAR agonist treatment for Syndrome X relates to the use of thiazolidinediones (TZDs) or other insulin sensitivity enhancers (ISEs). A need exists for new pharmaceutical agents which affect treat or prevent cardiovascular disease, particularly that associated with Syndrome X, while preventing or minimizing weight gain, and

more preferably while improving insulin sensitivity. It may be especially desirable when the active pharmaceutical agent selectively modulates a PPAR receptor subtype to provide an especially desirable pharmacological profile. In some instances, it can be desirable when the active pharmacological agent selectively modulates more than one PPAR receptor subtype to provide a desired pharmacological profile.

### SUMMARY OF THE INVENTION

The present invention is directed to a method of treating a mammal in need of treatment for a disease, wherein the disease is treatable by modulating a peroxisome proliferator activated receptor, comprising the step of administering to the mammal in need thereof a therapeutically effective amount of a compound represented by Structural Formula I:

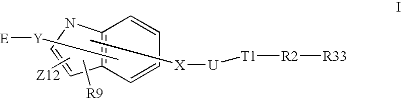

and stereoisomers, pharmaceutically acceptable salts, solvates and hydrates thereof, wherein:
(a) T1 is selected from the group consisting of 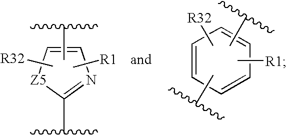
(b) R1 is selected from the group consisting of hydrogen, C₁-C₈ alkyl, C₁-C₈ alkenyl, aryl-C₀₋₄-alkyl, aryl-C₁₋₆-heteroalkyl, heteroaryl-C₀₋₄-alkyl, and C₃-C₆ cycloalkylaryl-C₀₋₂-alkyl, wherein C₁-C₈ alkyl, C₁-C₈ alkenyl, aryl-C₀₋₄-alkyl, aryl-C₁₋₆-heteroalkyl, heteroaryl-C₀₋₄-alkyl, and C₃-C₆ cycloalkylaryl-C₀₋₂-alkyl are each optionally substituted with from one to three substituents independently selected from R1′;
(c) R1′, R26, R27, R28, R31, Z14′, and Z15′ are each independently selected from the group consisting of hydrogen, hydroxy, cyano, nitro, halo, oxo, C₁-C₆ alkyl, C₁-C₆ alkyl-COOR12, C₁-C₆ alkoxy, C₁-C₆ haloalkyl, C₁-C₆ haloalkyloxy, C₃-C₇ cycloalkyl, optionally substituted aryloxy, optionally substituted aryl-C₀₋₄-alkyl, optionally substituted heteroaryl, optionally substituted heterocycloalkyl, C(O)R13, COOR14, OC(O)R15, OS(O)₂R16, N(R17)₂, NR18C(O)R19, NR20SO₂R21, SR22, S(O)R23, S(O)₂R24, and S(O)₂N(R25)₂; R12, R13, R14, R5, R16, R17, R8, R19, R20, R21, R22, R23, R24 and R25 are each independently selected from the group consisting of hydrogen, C₁-C₆ alkyl and aryl;
(d) R2 is selected from the group consisting of C₀-C₈ alkyl and C₁₋₆-heteroalkyl;
(e) X is selected from the group consisting of a bond, O, S, S(O)₂ and N;
(f) U is an aliphatic linker wherein one carbon atom of the aliphatic linker may be replaced with O, NH or S, and wherein such aliphatic linker is optionally substituted with R30;
(g) Y is selected from the group consisting of C, O, S, NH and a single bond;
(h) E is C(R3)(R4)A or A and wherein
   (i) A is selected from the group consisting of C₀-C₆ alkylcarboxyl, C₀-C₆ alkyltetrazole, C₁-C₆ alkylnitrile, C₀-C₆ alkylcarboxamide, C₀-C₆ alkylsulfonamide and C₀-C₆ alkylacylsulfonamide; wherein C₀-C₆ alkylsulfonamide, C₀-C₆ alkylacylsulfonamide and C₀-C₆ alkyltetrazole are each optionally substituted with from one to two groups independently selected from R⁷;
   (ii) each R⁷ is independently selected from the group consisting of hydrogen, C₁-C₆ haloalkyl, aryl-C₀-C₄ alkyl and C₁-C₆ alkyl, wherein such alkyl and arylalkyl are each optionally substituted with from one to two groups independently selected from R7′; each R7′ is independently selected from halo, C₁-C₆ alkyl, and haloC₁-C₆ alkyl;
   (iii) R3 is selected from the group consisting of hydrogen, C₁-C₅ alkyl, and C₁-C₅ alkoxy; and
   (iv) R4 is selected from the group consisting of hydrogen, C₁-C₅ alkyl, C₁-C₅ alkoxy, aryloxy, C₃-C₆ cycloalkyl, and aryl C₀-C₄ alkyl, and R3 and R4 are optionally combined to form a C₃-C₄ cycloalkyl, and wherein alkyl, alkoxy, cycloalkyl and aryl-alkyl are each optionally substituted with one to three each independently selected from R26;
(i) Z5 is S or O;
(j) Z12 is selected from the group consisting of hydrogen and -Z13C₀-C₃alkylZ14;
(k) Z13 is selected from the group consisting of a single bond, CO, CO₂, CONZ15, and SO₂;
(l) Z14 is selected from the group consisting of aryl and heteroaryl, wherein the aryl and heteroaryl is each optionally substituted with from one to three substituents independently selected from Z14′;
(m) Z15 is selected from the group consisting of hydrogen aryl and heteroaryl, wherein the aryl and heteroaryl is each optionally substituted with from one to three substituents independently selected from Z15′;
(n) R9 is selected from the group consisting of hydrogen, C₁-C₄ alkyl, C₁-C₄ alkylenyl, halo, aryl-C₀-C₄ alkyl, heteroaryl, C₁-C₆ allyl, and OR29, and wherein aryl-C₀-C₄ alkyl, heteroaryl are each optionally substituted with from one to three independently selected from R27; R29 is selected from the group consisting of hydrogen and C₁-C₄ alkyl;
(o) R10, R11 are each independently selected from the group consisting of hydrogen, hydroxy, cyano, nitro, halo, oxo, C₁-C₆ alkyl, C₀-C₆ alkyl-COOR12″, C₁-C₆ alkoxy, C₁-C₆ haloalkyl, C₁-C₆ haloalkyloxy, C₃-C₇ cycloalkyl, aryl-C₀₋₄-alkyl, aryl-C₁₋₆-heteroalkyl, heteroaryl-C₀₋₄-alkyl, C₃-C₆ cycloalkylaryl-C₀₋₂-alkyl, aryloxy, C(O)R13′, COOR14′, OC(O)R15′, OS(O)₂R16′, N(R17′)₂, NR18′C(O)R19′, NR20′SO₂R21′, SR22′, S(O)R23′, S(O)₂R24′, and S(O)₂N(R25′)₂; and wherein aryl-C₀₋₄-alkyl, aryl-C₁₋₆-heteroalkyl, heteroaryl-C₀₋₄-alkyl, and C₃-C₆ cycloalkylaryl-C₀₋₂-alkyl are each optionally substituted with from one to three independently selected from R28;
(p) R12′, R12″, R13′, R14′, R15′, R16′, R17′, R18′, R19′, R20′, R21′, R22′, R23′, R24′, and R25′ are each independently selected from the group consisting of hydrogen, C₁-C₆ alkyl and aryl;
(q) R30 is selected from the group consisting of C₁-C₆ alkyl, aryl-C₀₋₄-alkyl, aryl-C₁₋₆-heteroalkyl, heteroaryl-C₀₋₄-alkyl, and C₃-C₆ cycloalkylaryl-C₀₋₂-alkyl, and wherein C₁-C₆ alkyl, aryl-C₀₋₄-alkyl, aryl-C₁₋₆-heteroalkyl, heteroaryl-C₀₋₄-alkyl, and C₃-C₆ cycloalkylaryl-C₀₋₂-alkyl are each optionally substituted with from one to three substituents each independently selected from R31;
(r) R32 is selected from the group consisting of a bond, hydrogen, halo, C₁-C₆ alkyl, C₁-C₆ haloalkyl, and C₁-C₆ alkyloxo;
(s) R33 is selected from the group consisting of C₁-C₈ alkyl, C₁-C₈ alkoxy, phenyl, thiophene, pyridine, piperidine, 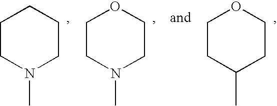
wherein the C₁-C₈ alkyl, C₁-C₈ alkoxy, phenyl, thiophene, pyridine, piperidine, 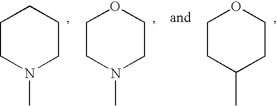
are each optionally substituted with R10 and R11.

A compound is represented by Structural Formula I′, and stereoisomers, pharmaceutically acceptable salts, solvates and hydrates thereof: 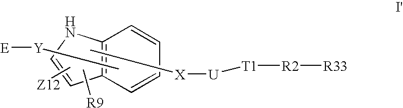
wherein the variables are as defined in Structural Formula I above, except as provided below.

When Y is C or a bond, at least one of R1, R2, R3, and R4 is C₁-C₄ alkyl.

R33 is selected from the group consisting of C₂-C₈ alkyl, C₁-C₈ alkoxy, phenyl, thiophene, pyridine, piperidine, 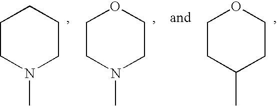
wherein the C₂-C₈ alkyl, C₁-C₈ alkoxy, phenyl, thiophene, pyridine, piperidine, 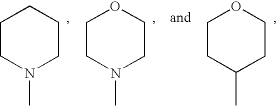
are each optionally substituted with R10 and R11.

Another compound is represented by Structural Formula I″, and stereoisomers, pharmaceutically acceptable salts, solvates and hydrates thereof: 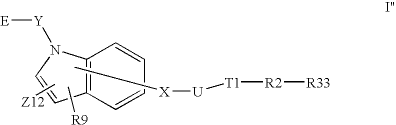
wherein the variables are as defined in Structural Formula I above, except that R33 is as defined for Structural Formula I′ in the preceding paragraph.

In one embodiment, the present invention also relates to pharmaceutical compositions comprising at least one compound of the present invention, or a pharmaceutically acceptable salt, solvate, hydrate, or stereioisomer thereof, and a pharmaceutically acceptable carrier.

In another embodiment, the present invention relates to a method of selectively modulating a PPAR delta receptor by contacting the receptor with at least one compound represented by Structural Formula I, or a pharmaceutically acceptable salt, solvate, hydrate, or stereioisomer thereof.

In another embodiment, the present invention relates to a method of modulating one or more of the PPAR alpha, beta, gamma, and/or delta receptors.

In a further embodiment, the present invention relates to a method of making a compound represented by Structural Formula I.

The compounds of the present invention are believed to be effective in treating and preventing Syndrome X, Type II diabetes, hyperglycemia, hyperlipidemia, obesity, coagaulopathy, hypertension, atherosclerosis, and other disorders related to Syndrome X and cardiovascular diseases. Further, compounds of this invention can be useful for lowering fibrinogen, increasing HDL levels, treating renal disease, controlling desirable weight, treating demyelinating diseases, treating certain viral infections, and treating liver disease. In addition, the compounds can be associated with fewer clinical side effects than compounds currently used to treat such conditions.

### DETAILED DESCRIPTION OF TH INVENTION

The terms used to describe the instant invention have the following meanings.

As used herein, the term “aliphatic linker” or “aliphatic group” is a non-aromatic, consisting solely of carbon and hydrogen and may optionally contain one or more units of saturation, e.g., double and/or triple bonds (also refer herein as “alkenyl” and “alkynyl”). An aliphatic or aliphatic group may be straight chained, branched (also refer herein as “alkyl”) or cyclic (also refer herein as “cycloalkyl). When straight chained or branched, an aliphatic group typically contains between about 1 and about 10 carbon atoms, more typically between about 1 and about 6 carbon atoms. When cyclic, an aliphatic typically contains between about 3 and about 10 carbon atoms, more typically between about 3 and about 7 carbon atoms. Aliphatics are preferably C₁-C₁₀ straight chained or branched alkyl groups (i.e. completely saturated aliphatic groups), more preferably C₁-C₆ straight chained or branched alkyl groups. Examples include, but are not limited to methyl, ethyl, propyl, n-propyl, iso-propyl, n-butyl, sec-butyl, and tert-butyl. Additional examples include, but are not limited to, cyclopropyl, cyclopentyl, cyclohexyl, cyclopentyl, cyclohexylyl and the like.

The term “alkyl,” unless otherwise indicated, refers to those alkyl groups of a designated number of carbon atoms of either a straight or branched saturated configuration. As used herein, “C₀ alkyl” means that there is no carbon and therefore represents a bond. Examples of “alkyl” include, but are not limited to, methyl, ethyl, n-propyl, isopropyl, n-butyl, isobutyl, sec-butyl and tert-butyl, pentyl, hexyl, isopentyl and the like. Alkyl as defined above may be optionally substituted with a designated number of substituents as set forth in the embodiment recited above. As used herein, the term “alkyloxo” means an alkyl group of the designated number of carbon atoms with a “═O” substituent.

The term “alkenyl” means hydrocarbon chain of a specified number of carbon atoms of either a straight or branched configuration and having at least one carbon-carbon double bond, which may occur at any point along the chain, such as ethenyl, propenyl, butenyl, pentenyl, vinyl, alkyl, 2-butenyl and the like. Alkenyl as defined above may be optionally substituted with designated number of substituents as set forth in the embodiment recited above.

The term “alkynyl” means hydrocarbon chain of a specified number of carbon atoms of either a straight or branched configuration and having at least one carbon-carbon triple bond, which may occur at any point along the chain. Example of alkynyl is acetylene. Alkynyl as defined above may be optionally substituted with designated number of substituents as set forth in the embodiment recited above.

The term “heteroalkyl” refers to a means hydrocarbon chain of a specified number of carbon atoms wherein at least one carbon is replaced by a heteroatom selected from the group consisting of O, N and S.

The term “cycloalkyl” refers to a saturated or partially saturated carbocycle containing one or more rings of from 3 to 12 carbon atoms, typically 3 to 7 carbon atoms. Examples of cycloalkyl includes, but are not limited to cyclopropyl, cyclobutyl, cyclopentyl, cyclohexyl and cycloheptyl, and the like. “Cycloalkyaryl” means that an aryl is fused with a cycloalkyl, and “Cycloalkylaryl-alkyl” means that the cycloalkylaryl is linked to the parent molecule through the alkyl. Cycloalkyl as defined above may be optionally substituted with a designated number of substituents as set forth in the embodiment recited above.

The term “halo” refers to fluoro, chloro, bromo and iodo.

The term “haloalkyl” is a C₁-C₆ alkyl group, which is substituted with one or more halo atoms selected from F, Br, Cl and I. An example of a haloalkyl group is trifluoromethyl (CF₃).

The term “alkoxy” and “alkyloxy” represents an alkyl group of indicated number of carbon atoms attached through an oxygen bridge, such as methoxy, ethoxy, propoxy, isopropoxy, butoxy, tert-butoxy, pentoxy, and the like. Alkoxy as defined above may be optionally substituted with a designated number of substituents as set forth in the embodiment recited above.

The term “haloalkyloxy” represents a C₁-C₆ haloalkyl group attached through an oxygen bridge, such as OCF₃. The “haloalkyloxy” as defined above may be optionally substituted with a designated number of substituents as set forth in the embodiment recited above.

The term “oxo” means a group of the formula: “═O”. The term “sulfo” means a group of the formula “═S”.

The term “aryl” includes carbocyclic aromatic ring systems (e.g. phenyl), fused polycyclic aromatic ring systems (e.g. naphthyl and anthracenyl) and aromatic ring systems fused to carbocyclic non-aromatic ring systems (e.g., 1,2,3,4-tetrahydronaphthyl). “Aryl” as defined above may be optionally substituted with a designated number of substituents as set forth in the embodiment recited above.

The term “arylalkyl” refers to an aryl alkyl group which is linked to the parent molecule through the alkyl group, which may be further optionally substituted with a designated number of substituents as set forth in the embodiment recited above. When arylalkyl is arylC₀alkyl, then the aryl group is bonded directly to the parent molecule. Likewise, arylheteroalkyl means an aryl group linked to the parent molecule through the heteroalkyl group.

The term “acyl” refers to alkylcarbonyl species.

The term “heteroaryl” group, as used herein, is an aromatic ring system having at least one heteroatom such as nitrogen, sulfur or oxygen and includes monocyclic, bicyclic or tricyclic aromatic ring of 5- to 14-carbon atoms containing one or more heteroatoms selected from the group consisting of O, N, and S. The “heteroaryl” as defined above may be optionally substituted with a designated number of substituents as set forth in the embodiment recited above. Examples of heteroaryl are, but are not limited to, furanyl, indolyl, thienyl (also referred to herein as “thiophenyl”) thiazolyl, imidazolyl, isoxazoyl, oxazoyl, pyrazoyl, pyrrolyl, pyrazinyl, pyridyl, pyrimidyl, pyrimidinyl and purinyl, cinnolinyl, benzofuranyl, benzothienyl, benzotriazolyl, benzoxazolyl, quinoline, isoxazolyl, isoquinoline and the like. The term “heteroarylalkyl” means that the heteroaryl group is linked to the parent molecule through the alkyl portion of the heteroarylalkyl.

The term “heterocycloalkyl” refers to a non-aromatic ring which contains one or more oxygen, nitrogen or sulfur and includes a monocyclic, bicyclic or tricyclic non-aromatic ring of 5 to 14 carbon atoms containing one or more heteroatoms selected from O, N or S. The “heterocycloalkyl” as defined above may be optionally substituted with a designated number of substituents as set forth in the embodiment recited above. Examples of heterocycloalkyl include, but are not limited to, morpholine, piperidine, piperazine, pyrrolidine, and thiomorpholine. As used herein, alkyl groups include straight chained and branched hydrocarbons, which are completely saturated.

As used herein, sulfonamide means the group —NHSO₂—, e.g., when A is sulfonamide, E-A is C(R3)(R4)-NHSO₂—. As used herein, acylsulfonamide means —C(O)NHSO₂—, e.g., when A is acylsulfonamide, E-A is C(R3)(R4)-C(O)NHSO₂—.

In Structural Formulas depicted herein, when more substitutents are indicated on a group than are chemically possible, e.g., when T1 is 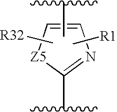
and Z5 is S or O, one skilled in the art will appreciate that excess substituents are intended in the alternative, e.g., that T1 above can be substituted with a substituent selected from R1 and R32.

A bond to the center of a cyclic group indicates that the bond is to any substitutable atom in the ring. For example, when T1 is the five membered ring shown above, R32 and R1 are bonded to any substitutable atom in the ring. When a bond passes through a ring and ends in the center of a second ring, the bond is to any substitutable ring atom in either ring. For example, in Structural Formula I, E-Y— is bonded to any substitutable ring atom in either the five or six membered ring. In Structural Formula II, shown below, E-Y— is bonded to any substitutable ring atom in the five membered ring only.

As used herein, the phrase “selectively modulate” means a compound whose EC50 for the stated PPAR receptor is at least ten fold lower than its EC50 for the other PPAR receptor subtypes.

When one of the compounds of the invention has more than one chiral substituent it may exist in diastereoisomeric forms. The diastereoisomeric pairs may be separated by methods known to those skilled in the art, for example chromatography or crystallization and the individual enantiomers within each pair may be separated using methods familiar to the skilled artisan. The present invention includes each diastereoisomer of compounds of the invention and mixtures thereof.

The compounds of the invention may contain one or more chiral centers, and exist in different optically active forms. When a compound of the invention contains one chiral center, the compounds exist in two enantiomeric forms and the present invention includes both enantiomers and mixtures of enantiomers, such as racemic mixtures. The enantiomers may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

Certain of the compounds of the invention may exist in different stable conformational forms which may be separable. Torsional asymmetry due to restricted rotation about an asymmetric single bond, for example because of steric hindrance or ring strain, may permit separation of different conformers. The present invention includes each conformational isomer of compounds of Structural Formula I and mixtures thereof.

Certain compounds of the invention may exist in zwitterionic form and the present invention includes each zwitterionic form of compounds of Structural Formula I and mixtures thereof.

“Pharmaceutically-acceptable salt” refers to salts of the compounds of the invention which are considered to be acceptable for clinical and/or veterinary use. Typical pharmaceutically-acceptable salts include those salts prepared by reaction of the compounds of the present invention with a mineral or organic acid or an organic or inorganic base. Such salts are known as acid additiona salts and base addition salts, respectively. It will be recognized that the particular counterion forming a part of any salt of this invention is not of a critical nature, so long as the salt as a whole is pharmaceutically-acceptable and as long as the counterion does not contribute undesired qualities to the salt as a whole. These salts may be prepared by methods known to the skilled artisan.

The term, “active ingredient” means the compounds of the invention as well as the sterioisomers, salts, solvates, and hydrates,

The term “pharmaceutically acceptable” means that the carrier, diluent, excipients and salt are pharmaceutically compatible with the other ingredients of the composition. Pharmaceutical compositions of the present invention are prepared by procedures known in the art using well known and readily available ingredients.

“Preventing” refers to reducing the likelihood that the recipient will incur or develop any of the pathological conditions described herein. The term “preventing” is particularly applicable to a patient that is susceptible to the particular patholical condition.

“Treating” refers to mediating a disease or condition and preventing, or mitigating, its further progression or ameliorate the symptoms associated with the disease or condition.

“Pharmaceutically-effective amount” means that amount of active ingredient, that will elicit the biological or medical response of a tissue, system, or mammal. Such an amount can be administered prophylactically to a patient thought to be susceptible to development of a disease or condition. Such amount when administered prophylactically to a patient can also be effective to prevent or lessen the severity of the mediated condition. Such an amount is intended to include an amount which is sufficient to modulate a selected PPAR receptor or to prevent or mediate a disease or condition. Generally, the effective amount of a Compound of Formula I will be between 0.02 through 5000 mg per day. Preferably the effective amount is between 1 through 1,500 mg per day. Preferably the dosage is from 1 through 1,000 mg per day.

The desired dose may be presented in a single dose or as divisded doses administered at appropriate intervals.

A “mammal” is an individual animal that is a member of the taxonomic class Mammalia. The class Mammalia includes humans, monkeys, chimpanzees, gorillas, cattle, swine, horses, sheep, dogs, cats, mice, and rats.

Administration to a human is most preferred. The compounds and compositions of the present invention are useful for the treatment and/or prophylaxis of cardiovascular disease, for raising serum HDL cholesterol levels, for lowering serum triglyceride levels and for lower serum LDL cholesterol levels. Elevated triglyceride and LDL levels, and low HDL levels, are risk factors for the development of heart disease, stroke, and circulatory system disorders and diseases.

The compounds and compositions of the present invention are also useful for treating and/or preventing obesity.

Further, these compounds and compositions are useful for the treatment and/or prophylaxis of non-insulin dependent diabetes mellitus (NIDDM) with reduced or no body weight gains by the patients. Furthermore, the compounds and compositions of the present invention are useful to treat or prevent acute or transient disorders in insulin sensitivity, such as sometimes occur following surgery, trauma, myocardial infarction, and the like. The physician of ordinary skill will know how to identify humans who will benefit from administration of the compounds and compositions of the present invention.

The present invention further provides a method for the treatment and/or prophylaxis of hyperglycemia in a human or non-human mammal which comprises administering an effective amount of active ingredient, as defined herein, to a hyperglycemic human or non-human mammal in need thereof.

The invention also relates to the use of a compound of Formula I as described above, for the manufacture of a medicament for treating a PPAR receptor mediated condition.

A therapeutically effective amount of a compound of the invention can be used for the preparation of a medicament useful for treating Syndrome X, diabetes, treating obesity, lowering tryglyceride levels, lowering serum LDL levels, raising the plasma level of high density lipoprotein, and for treating, preventing or reducing the risk of developing atherosclerosis, and for preventing or reducing the risk of having a first or subsequent atherosclerotic disease event in mammals, particularly in humans. In general, a therapeutically effective amount of a compound of the present invention typically reduces serum triglyceride levels of a patient by about 20% or more, and increases serum HDL levels in a patient. Preferably, HDL levels will be increased by about 30% or more. In adition, a therapeutically effective amount of a compound, used to prevent or treat NIDDM, typically reduces serum glucose levels, or more specifically HbAlc, of a patient by about 0.7% or more.

When used herein Syndrome X includes pre-diabetic insulin resistance syndrome and the resulting complications thereof, insulin resistance, non-insulin dependent diabetes, dyslipidemia, hyperglycemia obesity, coagulopathy, hypertension and other complications associated with diabetes. The methods and treatments mentioned herein include the above and encompass the treatment and/or prophylaxis of any one of or any combination of the following: pre-diabetic insulin resistance syndrome, the resulting complications thereof, insulin resistance, Type II or non-insulin dependent diabetes, dyslipidemia, hyperglycemia, obesity and the complications associated with diabetes including cardiovascular disease, especially atherosclerosis.

The compositions are formulated and administered in the same general manner as detailed herein. The compounds of the instant invention may be used effectively alone or in combination with one or more additional active agents depending on the desired target therapy. Combination therapy includes administration of a single pharmaceutical dosage composition which contains a compound of the invention, a stereoisomer, salt, solvate and/or hydrate thereof (“Active Igredient”) and one or more additional active agents, as well as administration of a compound of Active Ingredient and each active agent in its own separate pharmaceutical dosage formulation. For example, an Active Ingredient and an insulin secretogogue such as biguanides, thiazolidinediones, sulfonylureas, insulin, or α-glucosidose inhibitors can be administered to the patient together in a single oral dosage composition such as a tablet or capsule, or each agent administered in separate oral dosage formulations. Where separate dosage formulations are used, an Active Ingredient and one or more additional active agents can be administered at essentially the same time, i.e., concurrently, or at separately staggered times, i.e., sequentially; combination therapy is understood to include all these regimens.

An example of combination treatment or prevention of atherosclerosis may be wherein an Active Ingredient is administered in combination with one or more of the following active agents: antihyperlipidemic agents; plasma HDL-raising agents; antihypercholesterolemic agents, fibrates, vitamins, aspirin, and the like. As noted above, the Active Ingredient can be administered in combination with more than one additional active agent.

Another example of combination therapy can be seen in treating diabetes and related disorders wherein the Active Ingredient can be effectively used in combination with, for example, sulfonylureas, biguanides, thiazolidinediones, α-glucosidase inhibitors, other insulin secretogogues, insulin as well as the active agents discussed above for treating atherosclerosis.

The Active Ingredients of the present invention, have valuable pharmacological properties and can be used in pharmaceutical compositions containing a therapeutically effective amount of Active Ingredient of the present invention, in combination with one or more pharmaceutically acceptable excipients. Excipients are inert substances such as, without limitation carriers, diluents, fillers, flavoring agents, sweeteners, lubricants, solubilizers, suspending agents, wetting agents, binders, disintegrating agents, encapsulating material and other conventional adjuvants. Proper formulation is dependent upon the route of administration chosen. Pharmaceutical compositions typically contain from about 1 to about 99 weight percent of the Active Ingredient of the present invention.

Preferably, the pharmaceutical formulation is in unit dosage form. A “unit dosage form” is a physically discrete unit containing a unit dose, suitable for administration in human subjects or other mammals. For example, a unit dosage form can be a capsule or tablet, or a number of capsules or tablets. A “unit dose” is a predetermined quantity of the Active Ingredient of the present invention, calculated to produce the desired therapeutic effect, in association with one or more pharmaceutically-acceptable excipients. The quantity of active ingredient in a unit dose may be varied or adjusted from about 0.1 to about 1500 milligrams or more according to the particular treatment involved. It may be preferred that the unit dosage is from about 1 mg to about 1000 mg.

The dosage regimen utilizing the compounds of the present invention is selected by one of ordinary skill in the medical or veterinary arts, in view of a variety of factors, including, without limitation, the species, age, weight, sex, and medical condition of the recipient, the severity of the condition to be treated, the route of administration, the level of metabolic and excretory function of the recipient, the dosage form employed, the particular compound and salt thereof employed, and the like.

Advantageously, compositions containing a compound of the invention or the salts thereof may be provided in dosage unit form, preferably each dosage unit containing from about 1 to about 500 mg be administered although it will, of course, readily be understood that the amount of the compound or compounds of of the invention actually to be administered will be determined by a physician, in the light of all the relevant circumstances.

Preferably, the compounds of the present invention are administered in a single daily dose, or the total daily dose may be administered in divided doses, two, three, or more times per day. Where delivery is via transdermal forms, of course, administration is continuous.

Suitable routes of administration of pharmaceutical compositions of the present invention include, for example, oral, eyedrop, rectal, transmucosal, topical, or intestinal administration; parenteral delivery (bolus or infusion), including intramuscular, subcutaneous, intramedullary injections, as well as intrathecal, direct intraven-tricular, intravenous, intraperitoneal, intranasal, or intraocular injections. The compounds of the invention can also be administered in a targeted drug delivery system, such as, for example, in a liposome coated with endothelial cell-specific antibody.

Solid form formulations include powders, tablets and capsules.

Sterile liquid formulations include suspensions, emulsions, syrups, and elixirs.

Pharmaceutical compositions of the present invention can be manufactured in a manner that is itself known, e.g., by means of conventional mixing, dissolving, granulating, dragee-making, levigating, emulsifying, encapsulating, entrapping, lyophilizing processes, and/or coupled with soluble polymers as targeted drug carriers.

The following pharmaceutical formulations 1 and 2 are illustrative only and are not intended to limit the scope of the invention in any way.

### Formulation 1

Hard gelatin capsules are prepared using the following ingredients:

| | Quantity | |
|---|---|---|
| | (mg/capsule) | |
| Active Ingredient | 250 | |
| Starch, dried | 200 | |
| Magnesium stearate | 10 | |
| Total | 460 | mg |

### Formulation 2

A tablet is prepared using the ingredients below:

| | Quantity | |
|---|---|---|
| | (mg/tablet) | |
| Active Ingredient | 250 | |
| Cellulose, microcrystalline | 400 | |
| Silicon dioxide, fumed | 10 | |
| Stearic acid | 5 | |
| Total | 665 | mg |

The components are blended and compressed to form tablets each weighing 665 mg

In yet another embodiment of the compounds of the present invention, the compound is radiolabelled, such as with carbon-14, or tritiated. Said radiolabelled or tritiated compounds are useful as reference standards for in vitro assays to identify new selective PPAR receptor agonists.

The compounds of the present invention can be useful for modulating insulin secretion and as research tools. Certain compounds and conditions within the scope of this invention are preferred. The following conditions, invention embodiments, and compound characteristics listed in tabular form are preferred and may be independently combined to produce a variety of preferred compounds and process conditions. The following list of embodiments of this invention is not intended to limit the scope of this invention in any way.

In further respective embodiments, the variables in Structural Formulas I, I′, and I″ are as defined above, except that A is selected from the group consisting of C₀-C₆ alkylcarboxyl, C₀-C₆ alkyltetrazole, C₁-C₆ alkylnitrile, C₀-C₆ alkylsulfonamide and C₀-C₆ alkylacylsulfonamide; wherein C₀-C₆ alkylsulfonamide, C₀-C₆ alkylacylsulfonamide and C₀-C₆ alkyltetrazole are each optionally substituted with from one to two groups independently selected from R⁷.

In another embodiment, the compounds represented by Structural Formulas I, I′, and I″ are further represented by Structural Formulas II, II′, and II″, respectively:

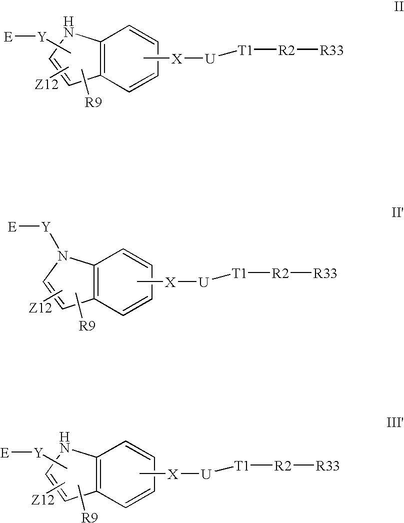

The variables are as defined for Structural Formulas I, I′, and I″, except as provided below:
R1′, R26, R27, R28, R31, Z14′, and Z15′ are each independently selected from the group consisting of hydrogen, hydroxy, cyano, nitro, halo, oxo, C₁-C₆ alkyl, C₁-C₆ alkyl-COOR12, C₁-C₆ alkoxy, C₁-C₆ haloalkyl, C₁-C₆ haloalkyloxy, C₃-C₇ cycloalkyl, aryloxy, aryl-C₀₋₄-alkyl, heteroaryl, heterocycloalkyl, C(O)R13, COOR14, OC(O)R15, OS(O)₂R16, N(R17)₂, NR18C(O)R19, NR20SO₂R21, SR22, S(O)R23, S(O)R24, and S(O)₂N(R25)₂; R12, R13, R14, R15, R16, R17, R18, R19, R20, R21, R22, R23, R24 and R25 are each independently selected from the group consisting of hydrogen, C₁-C₆ alkyl and aryl;
A is selected from the group consisting of carboxyl, tetrazole, C₁-C₆ alkylnitrile, carboxamide, sulfonamide and acylsulfonamide; wherein sulfonamide, acylsulfonamide and tetrazole are each optionally substituted with from one to two groups independently selected from R⁷; and
R33 is selected from the group consisting of phenyl, thiophene, pyridine, piperidine, 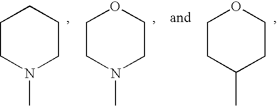
wherein the phenyl, thiophene, pyridine, piperidine, 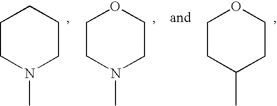
are each optionally substituted with R10 and R11.

In other embodiments, the compounds represented by Structural Formulas I and II, e.g., those employed in the disclosed method, are further represented by one of Structural Formulas III-VI:

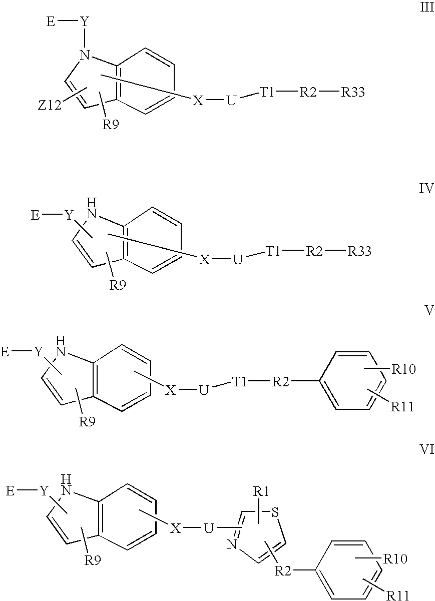

The variables in Structural Formulas III-VI are as defined for Structural Formulas I and II.

In other embodiments, the compounds represented by Structural Formulas I′ and II′ are further represented by one of Structural Formulas III′-VI′:

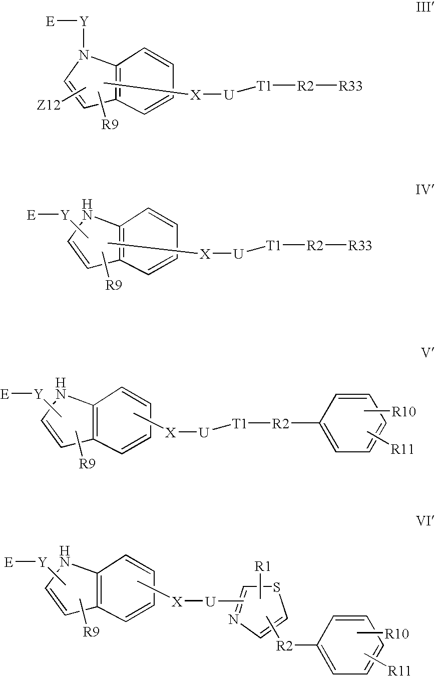

The variables in Structural Formulas III′-VI′ are as described for Structural Formulas I′ and II′.

In other embodiments, the compounds represented by Structural Formulas I″ and II″ are further represented by one of Structural Formulas IV″-VI″:

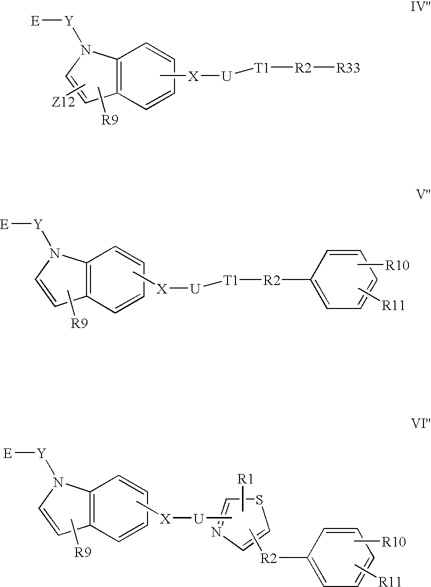

The variables in Structural FOrmulas III″-VI″ are as described for Structural Formulas I″ and II″.

In other embodiments, the compounds represented by Structural Formulas V, V′ and V″ are further represented by one of Structural Formulas VII, VII′ and VII″, respectively:

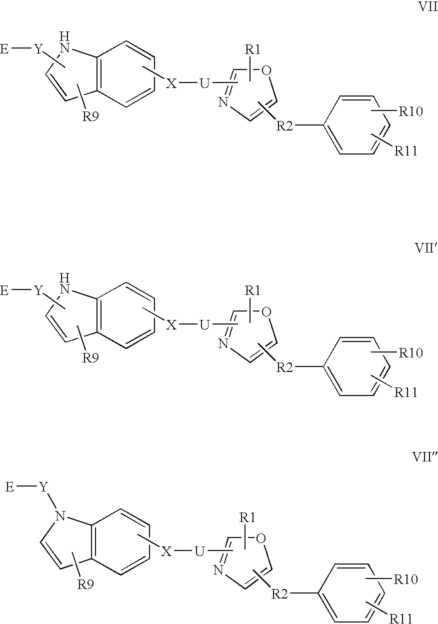

The variables for Structural Formulas VII, VII′ and VII″ are as described for Structural Formulas V, V′ and V″.

In other embodiments, the compounds represented by Structural Formula V, V′ and V″ are further represented by one of Structural Formulas VIII, VIII′ and VIII″, respectively:

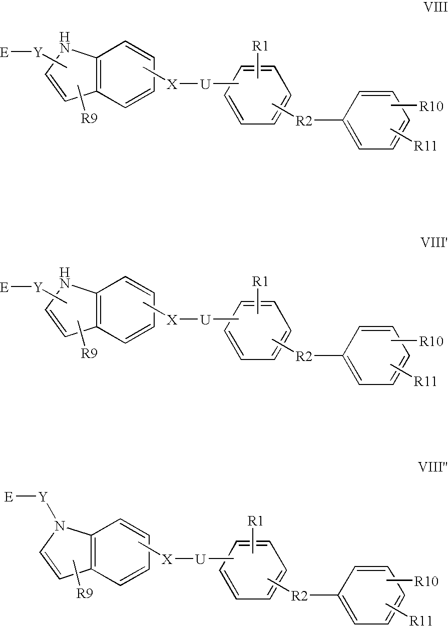

The variables in Structural Formulas VIII, VIII′ and VIII″ are as described for Structural Formulas V, V′ and V″, respectively.

In each of Structural Formulas I-VIII, III′-VIII′, and IV″-VIII″, in one embodiment, X is a bond; in another embodiment, X is —S—; and in another embodiment, X is —O—.

In other embodiments when X is —O—, E is —COOH, C₁-C₆ alkylcarboxyl, or C(R3)(R4)-C₁-C₆ alkyl-COOH. More preferably, X is —O—, E is —COOH, C₁-C₆ alkylcarboxyl, or C(R3)(R4)-C₁-C₆ alkyl-COOH and R10 and R11 are each independently selected from the group consisting of hydrogen, halo, oxo, C₁-C₆ alkyl, C₁-C₆ alkyl-COOR12″, C₁-C₆ alkoxy, C₁-C₆ haloalkyl, and C₁-C₆ haloalkyloxy. In one preferred embodiment, R10 is selected from the group consisting of C₃-C₇ cycloalkyl, aryl-C₀₋₄-alkyl, aryl-C₁₋₆-heteroalkyl, heteroaryl-C₀₋₄-alkyl, C₃-C₆ cycloalkylaryl-C₀₋₂-alkyl, and aryloxy; and even more preferably, R10 is CF₃. In another embodiment, X, E, R10 and R11 have the values just described and R9 is selected from the group consisting of hydrogen and C₁-C₃ alkyl; more preferably, R1, R3, and R4 are each independently selected from the group consisting of hydrogen and C₁-C₂ alkyl; still more preferably, X, E, R9, R10, R11 have the values just described R2 is a bond, and in a preferred embodiment, U is saturated C₁-C₃ alkyl and is optionally substituted with C₁-C₃ alkyl.

In one embodiment, a compound represented by any of Structural Formulas I-VIII, I′-VIII′, or I″-VIII″ is in the S conformation; in another embodiment, the compound is in the R conformation; in yet another embodiment, the compound is radiolabeled.

In embodiments of the methdod, employing any compound represented by Structural Formulas I-VIII, I′-VIII′, or I″-VIII″, the disease is selected from the group consisting of diabetes mellitus, Syndrome X, and atherosclerosis; in one preferred embodiment, the disease is diabetes mellitus; in another preferred embodiment, the disease is Syndrome X.

As used herein, Structural Formulas are related in groups according to roman numerals, e.g., Structural Formulas II-VIII further define compounds of the method represented by Structural Formula I; Structural Formulas II′-VIII′ further define compounds represented by Structural Formula I′; and Structural Formulas III″-VIII″ further define compounds represented by Structural Formula I″.

Some preferred values for the variables used in the Structural Formulas disclosed herein are:
(a) T1 is 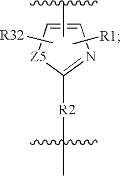
(b) T1 is 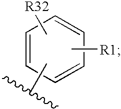
(c) R3 is methyl;
(d) R4 is hydrogen;
(e) R3 and R4 are each hydrogen;
(f) R3 and R4 are each methyl;
(g) A is carboxyl;
(h) X is —O;
(i) X is —S—;
(j) U is CH;
(k) U is CH₂CH;
(l) R9 is methyl;
(m) R9 is hydrogen;
(n) R9 is C₁-C₃ alkyl;
(o) R9 is hydrogen;
(p) Y and X are substituted para to one another through the phenyl ring of the headpiece;
(q) R9 is selected from the group consisting of hydrogen, alkyl, alkenyl, halo, arylakyl, heteroaryl, allyl and OR29;
(r) R9 is substituted arylalkyl or heteroaryl;
(s) R10 is CF₃;
(t) R10 is haloalkyl;
(u) R10 is haloalkyloxy;
(v) R11 is hydrogen
(w) R10 and R11 are each hydrogen;
(x) R11 is haloalkyl;
(y) R1 is C₁-C₄ alkyl;
(z) R32 is hydrogen;
(aa) R2 is a bond;
(bb) R2 is C₁-C₂ alkyl;
(cc) Y is O;
(dd) Y is S;
(ee) Y is C;
(ff) E is C(R3)(R4)A;
(gg) R3 is hydrogen;
(hh) R3 is C₁-C₂ alkyl;
(ii) R4 is C₁-C₂ alkyl;
(jj) R3 and R4 are each hydrogen;
(kk) R3 and R4 are each methyl;
(ll) A is COOH.

In other embodiments, the compound represented by Structural Formula I, e.g., the compound of the method, is selected from:
{5-[5-Methyl-2-(4-trifluoromethyl-phenyl)-oxazol-4-ylmethoxy]-indol-1-yl}-acetic acid;
[5-(5-Methyl-2-phenyl-oxazol-4-ylmethoxy)-indol-1-yl]-acetic acid;
{5-[2-(4-Fluoro-phenyl)-5-methyl-oxazol-4-ylmethoxy]-indol-1-yl}-acetic acid;
{5-[2-(4-Benzyloxy-phenyl)-5-methyl-oxazol-4-ylmethoxy]-indol-1-yl}-acetic acid;
2-Methyl-2-(5-{2-[2-(4-trifluoromethyl-phenyl)-oxazol-4-ylmethoxy]-ethoxy}-indol-1-yl)-propionic acid;
{5-[4-Ethyl-2-(4-trifluoromethyl-phenyl)-thiazol-5-ylmethoxy]-indol-1-yl}-acetic acid;
2-Methyl-2-(5-{2-[4-methyl-2-(4-trifluoromethyl-phenyl)-thiazol-5-ylmethoxy]-ethoxy}-indol-1-yl)-propionic acid;
{5-[2-(3,5-Bis-trifluoromethyl-phenyl)-4-methyl-thiazol-5-ylmethoxy]-indol-1-yl}-acetic acid;
{5-[4-Methyl-2-(4-trifluoromethyl-phenyl)-oxazol-5-ylmethoxy]-indol-1-yl}-acetic acid;
2-Methyl-2-{5-[4-methyl-2-(4-trifluoromethyl-phenyl)-oxazol-5-ylmethoxy]-indol-1-yl}-propionic acid;
Racemic 2-{5-[4-Methyl-2-(4-trifluoromethyl-phenyl)-oxazol-5-ylmethoxy]-indol-1-yl}-propionic acid;
{5-[2-(4-Bromo-phenyl)-4-methyl-thiazol-5-ylmethoxy]-indol-1-yl}-acetic acid;
{5-[4-Butyl-2-(4-trifluoromethyl-phenyl)-thiazol-5-ylmethoxy]-indol-1-yl}-acetic acid;
2-{5-[4-Butyl-2-(4-trifluoromethyl-phenyl)-thiazol-5-ylmethoxy]-indol-1-yl}-propionic acid;
{5-[4-Phenethyl-2-(4-trifluoromethyl-phenyl)-thiazol-5-ylmethoxy]-indol-1-yl}-acetic acid;
2-{5-[4-(2-Chloro-6-fluoro-phenoxymethyl)-2-(4-trifluoromethyl-phenyl)-thiazol-5-ylmethoxy]-indol-1-yl}-propionic acid;
{5-[4-Phenoxymethyl-2-(4-trifluoromethyl-phenyl)-thiazol-5-ylmethoxy]-indol-1-yl}-acetic acid;
Racemic 2-Methyl-2-{5-[4-phenoxymethyl-2-(4-trifluoromethyl-phenyl)-thiazol-5-ylmethoxy]-indol-1-yl}-propionic acid;
2-Methyl-2-{5-[4-phenoxymethyl-2-(4-trifluoromethyl-phenyl)-thiazol-5-ylmethoxy]-indol-1-yl}-propionic acid;
3-{5-[4-Propyl-2-(4-trifluoromethyl-phenyl)-thiazol-5-ylmethoxy]-indol-1-yl}-propionic acid;
5-{5-[4-Methyl-2-(4-trifluoromethyl-phenyl)-thiazol-5-ylmethoxy]-indol-1-yl}-pentanoic acid;
5-{5-[4-Propyl-2-(4-trifluoromethyl-phenyl)-thiazol-5-ylmethoxy]-indol-1-yl}-pentanoic acid;
{5-[4-Isopropyl-2-(4-trifluoromethyl-phenyl)-thiazol-5-ylmethoxy]-indol-1-yl}-acetic acid;
{5-[4-(2-Chloro-6-fluoro-phenoxymethyl)-2-(4-trifluoromethyl-phenyl)-thiazol-5-ylmethoxy]-indol-1-yl}-acetic acid;
2-{5-[4-(2-Chloro-6-fluoro-phenoxymethyl)-2-(4-trifluoromethyl-phenyl)-thiazol-5-ylmethoxy]-indol-1-yl}-2-methyl-propionic acid;
5-{5-[5-Methyl-2-(4-trifluoromethyl-phenyl)-oxazol-4-ylmethoxy]-indol-1-yl}-pentanoic acid;
5-{5-[2-(4-Bromo-phenyl)-5-methyl-oxazol-4-ylmethoxy]-indol-1-yl}-pentanoic acid;
5-{5-[4-Phenethyl-2-(4-trifluoromethyl-phenyl)-thiazol-5-ylmethoxy]-indol-1-yl}-pentanoic acid;
4-[1-(4-Carboxy-butyl)-1H-indol-5-yloxymethyl]-2-(4-trifluoromethylphenyl)-thiazole-5-carboxylic acid;
3-{5-[2-(4-Bromo-phenyl)-5-methyl-oxazol-4-ylmethoxy]-indol-1-yl}-propionic acid;
3-{5-[4-Phenethyl-2-(4-trifluoromethyl-phenyl)-thiazol-5-ylmethoxy]-indol-1-yl}-propionic acid;
3-{5-[4-Methyl-2-(4-trifluoromethyl-phenyl)-thiazol-5-ylmethoxy]-indol-1-yl}-propionic acid;
4-[1-(2-Carboxy-ethyl)-1H-indol-5-yloxymethyl]-2-(4-trifluoromethyl-phenyl)-thiazole-5-carboxylic acid;
{5-[4-Isopropyl-2-(4-trifluoromethyl-phenyl)-thiazol-5-ylmethylsulfanyl]-indol-1-yl}-acetic acid;
{5-[4-Ethyl-2-(4-trifluoromethyl-phenyl)-oxazol-5-ylmethoxy]-indol-1-yl}-acetic acid;
(5-{2-[5-Methyl-2-(4-trifluoromethyl-phenyl)-oxazol-4-yl]-ethoxy}-indol-1-yl)-acetic acid;
{5-[2-(5-Methyl-2-pyridin-4-yl-thiazol-4-yl)-ethoxy]-indol-1-yl}-acetic acid;
{5-[2-(5-Methyl-2-morpholin-4-yl-thiazol-4-yl)-ethoxy]-indol-1-yl}-acetic acid;
(5-{2-[5-Methyl-2-(4-trifluoromethyl-phenyl)-oxazol-4-yl]-ethoxy}-indol-1-yl)-acetic acid;
(5-{2-[5-Methyl-2-(tetrahydro-pyran-4-yl)-oxazol-4-yl]-ethoxy}-indol-1-yl)-acetic acid;
{5-[2-(2-Butoxy-5-methyl-oxazol-4-yl)-ethoxy]-indol-1-yl}-acetic acid;
{5-[2-(5-Methyl-2-pyridin-3-yl-thiazol-4-yl)-ethoxy]-indol-1-yl}-acetic acid;
{5-[2-(5-Methyl-2-pyridin-2-yl-thiazol-4-yl)-ethoxy]-indol-1-yl}-acetic acid;
(5-{2-[2-(5-Bromo-thiophen-2-yl)-5-methyl-oxazol-4-yl]-ethoxy}-indol-1-yl)-acetic acid;
{5-[3-(4-Butyl-phenoxy)-propoxy]-indol-1-yl}-acetic acid;
(5-{2-[2-(3-Bromo-phenyl)-5-methyl-oxazol-4-yl]-ethoxy}-indol-1-yl)-acetic acid;
(5-{2-[5-Ethyl-2-(4-trifluoromethyl-phenyl)-thiazol-4-yl]-ethoxy}-indol-1-yl)-acetic acid;
2-(5-{2-[5-Ethyl-2-(4-trifluoromethyl-phenyl)-thiazol-4-yl]-ethoxy}-indol-1-yl)-propionic acid;
(5-{2-[2-(2-Chloro-phenyl)-5-ethyl-thiazol-4-yl]-ethoxy}-indol-1-yl)-acetic acid;
2-(5-{2-[2-(2-Chloro-phenyl)-5-ethyl-thiazol-4-yl]-ethoxy}-indol-1-yl)-propionic acid;
(5-{2-[5-Propyl-2-(4-trifluoromethyl-phenyl)-thiazol-4-yl]-ethoxy}-indol-1-yl)-acetic acid;
2-(5-{2-[5-Propyl-2-(4-trifluoromethyl-phenyl)-thiazol-4-yl]-ethoxy}-indol-1-yl)-propionic acid;
2-(5-{2-[2-(2-Chloro-phenyl)-5-ethyl-thiazol-4-yl]-ethoxy}-indol-1-yl)-2-methyl-propionic acid;
Racemic 2-(5-{2-[5-Ethyl-2-(4-trifluoromethyl-phenyl)-oxazol-4-yl]-ethoxy}-indol-1-yl)-propionic acid;
(5-{2-[5-Ethyl-2-(4-trifluoromethyl-phenyl)-oxazol-4-yl]-ethoxy}-indol-1-yl)-acetic acid;
2-(5-{2-[5-Ethyl-2-(4-trifluoromethyl-phenyl)-oxazol-4-yl]-ethoxy}-indol-1-yl)-2-methyl-propionic acid;
3-(5-{2-[2-(2-Chloro-phenyl)-5-ethyl-thiazol-4-yl]ethoxy}-indol-1-yl)-propionic acid;
3-(5-{2-[5-Ethyl-2-(4-trifluoromethyl-phenyl)-oxazol-4-yl]-ethoxy}-indol-1-yl)-propionic acid;
2-Methyl-2-(5-{2-[5-propyl-2-(4-trifluoromethyl-phenyl)-thiazol-4-yl]-ethoxy}-indol-1-yl)-propionic acid;
Racemic-(5-{1-[4-Methyl-2-(4-trifluoromethyl-phenyl)-thiazol-5-yl]-ethoxy}-indol-1-yl)-acetic acid;
Racemic-(5-{1-[4-Methyl-2-(4-trifluoromethyl-phenyl)-thiazol-5-yl]-ethoxy}-indol-1-yl)-acetic acid;
Racemic-(5-{1-[4-Isopropyl-2-(4-trifluoromethyl-phenyl)-thiazol-5-yl]-ethoxy}-indol-1-yl)-acetic acid;
Racemic-(5-{2-[5-Ethyl-2-(4-trifluoromethyl-phenyl)-thiazol-4-yl]-1-methyl-ethoxy}-indol-1-yl)-acetic acid;
Racemic-(5-{2-[5-Methyl-2-(4-trifluoromethyl-phenyl)-thiazol-4-yl]-propoxy}-indol-1-yl)-acetic acid;
(5-{2-[5-Methyl-2-(4-trifluoromethyl-phenyl)-oxazol-4-yl]-propoxy}-indol-1-yl)-acetic acid;
(S)-(5-{2-[5-Methyl-2-(4-trifluoromethyl-phenyl)-oxazol-4-yl]-propoxy}-indol-1-yl)-acetic acid;
(R)-(5-{2-[5-Methyl-2-(4-trifluoromethyl-phenyl)-oxazol-4-yl]-propoxy}-indol-1-yl)-acetic acid;
Racemic-(5-{1-[4-Propyl-2-(4-trifluoromethyl-phenyl)-thiazol-5-yl]-ethoxy}-indol-1-yl)-acetic acid;
Racemic-(6-{1-[4-Isopropyl-2-(4-trifluoromethyl-phenyl)-thiazol-5-yl]-ethoxy}-1H-indol-3-yl)-acetic acid;
Racemic-(6-{2-[5-Methyl-2-(4-trifluoromethyl-phenyl)-thiazol-4-yl]-propoxy}-1H-indol-3-yl)-acetic acid;
Racemic-(1-Methyl-6-{2-[5-methyl-2-(4-trifluoromethyl-phenyl)-thiazol-4-yl]-propoxy}-1H-indol-3-yl)-acetic acid;
(S)-(6-{2-[5-Methyl-2-(4-trifluoromethyl-phenyl)-thiazol-4-yl]-propoxy}-1H-indol-3-yl)-acetic acid;
(R)-(6-{2-[5-Methyl-2-(4-trifluoromethyl-phenyl)-thiazol-4-yl]-propoxy}-1H-indol-3-yl)-acetic acid;
Racemic-(6-Hydroxy-5-{1-[4-isopropyl-2-(4-trifluoromethyl-phenyl)-thiazol-5-yl]-ethyl}-1H-indol-3-yl)-acetic acid;
(6-{2-[4-Isopropyl-2-(4-trifluoromethyl-phenyl)-thiazol-5-yl]-propoxy}-1-methyl-1H-indol-3-yl)-acetic acid;
(1-Methyl-6-{2-[4-methyl-2-(4-trifluoromethyl-phenyl)-thiazol-5-yl]-propoxy}-1H-indol-3-yl)-acetic acid;
(6-{2-[5-Ethyl-2-(4-trifluoromethyl-phenyl)-thiazol-4-yl]-propoxy}-1-methyl-1H-indol-3-yl)-acetic acid;
(R)-(6-{2-[4-Isopropyl-2-(4-trifluoromethyl-phenyl)-thiazol-5-yl]-propoxy}-1-methyl-1H-indol-3-yl)-acetic acid;
(S)-(6-{2-[4-Isopropyl-2-(4-trifluoromethyl-phenyl)-thiazol-5-yl]-propoxy}-1-methyl-1H-indol-3-yl)-acetic acid;
Racemic-(1-Methyl-6-{2-[4-methyl-2-(4-trifluoromethyl-phenyl)-thiazol-5-yl]-propoxy}-1H-indol-3-yl)-acetic acid;
Racemic-(1-Ethyl-6-{2-[4-isopropyl-2-(4-trifluoromethyl-phenyl)-thiazol-5-yl]-propoxy}-1H-indol-3-yl)-acetic acid;
Racemic-(6-{2-[4-Isopropyl-2-(4-trifluoromethyl-phenyl)-thiazol-5-yl]-propoxy}-1-propyl-1H-indol-3-yl)-acetic acid;
Racemic-(5-{1-[4-Ethyl-2-(4-trifluoromethyl-phenyl)-oxazol-5-yl]-ethoxy}-indol-1-yl)-acetic acid;
{5-[4-Propyl-2-(4-trifluoromethyl-phenyl)-thiazol-5-ylmethoxy]-indol-1-yl}-acetic acid;
Racemic-2-{5-[4-Propyl-2-(4-trifluoromethyl-phenyl)-thiazol-5-ylmethoxy]-indol-1-yl}-propionic acid;
2-Methyl-2-{5-[4-propyl-2-(4-trifluoromethyl-phenyl)-thiazol-5-ylmethoxy]-indol-1-yl}-propionic acid;
Racemic-2-{5-[4-Phenethyl-2-(4-trifluoromethyl-phenyl)-thiazol-5-ylmethoxy]-indol-1-yl}-propionic acid;
2-Methyl-2-{5-[4-phenethyl-2-(4-trifluoromethyl-phenyl)-thiazol-5-ylmethoxy]-indol-1-yl}-propionic acid;
{5-[4-Phenethyl-2-(4-trifluoromethyl-phenyl)-thiazol-5-ylmethoxy]-indol-1-yl}-acetic acid;
{5-[4-Phenyl-2-(4-trifluoromethyl-phenyl)-thiazol-5-ylmethoxy]-indol-1-yl}-acetic acid;
{5-[4-tert-Butyl-2-(4-trifluoromethyl-phenyl)-thiazol-5-ylmethoxy]-indol-1-yl}-acetic acid;
{5-[4-[2-(2-Chloro-6-fluoro-phenyl)-ethyl]-2-(4-trifluoromethyl-phenyl)-thiazol-5-ylmethoxy]-indol-1-yl}-acetic acid;
2-Methyl-2-[5-(5-methyl-2-phenyl-oxazol-4-ylmethoxy)-indol-1-yl]-propionic acid;
2-{5-[2-(4-Trifluoromethyl-phenyl)-5-methyl-oxazol-4-ylmethoxy]-indol-1-yl}-2-methyl-propionic acid;
2-{5-[2-(4-Fluoro-phenyl)-5-methyl-oxazol-4-ylmethoxy]-indol-1-yl}-2-methyl-propionic acid;
2-{5-[2-(4-Bromo-phenyl)-5-methyl-oxazol-4-ylmethoxy]-indol-1-yl}-2-methyl-propionic acid;
2-Methyl-2-(5-{2-[5-methyl-2-(4-trifluoromethyl-phenyl)-oxazol-4-yl]-ethoxy}-indol-1-yl)-propionic acid;
2-(5-{2-[2-(4-Bromo-phenyl)-5-methyl-oxazol-4-yl]-ethoxy}-indol-1-yl)-2-methyl-propionic acid;
2-(5-{2-[2-(5-Bromo-thiophen-2-yl)-5-methyl-oxazol-4-yl]-ethoxy}-indol-1-yl)-2-methyl-propionic acid;
2-Methyl-2-{5-[2-(5-methyl-2-phenyl-thiazol-4-yl)-ethoxy]-indol-1-yl}-propionic acid;
{5-[4-Isopropyl-2-(4-trifluoromethyl-phenyl)-thiazol-5-ylmethoxy]-2-methyl-indol-1-yl}-acetic acid;
2-{5-[2-(3,5-Bis-trifluoromethyl-phenyl)-4-methyl-thiazol-5-ylrnethoxy]-indol-1-yl}-2-methyl-propionic acid;
{4-[4-Propyl-2-(4-trifluoromethyl-phenyl)-thiazol-5-ylmethoxy]-indol-1-yl}-acetic acid;
Racemic-(5-{1-[4-[2-(2-Chloro-6-fluoro-phenyl)-ethyl]-2-(4-trifluoromethyl-phenyl)-thiazol-5-yl]-ethoxy}-indol-1-yl)-acetic acid;
Racemic-(5-{1-[4-Methyl-2-(4-trifluoromethyl-phenyl)-thiazol-5-yl]-2-phenyl-ethoxy}-indol-1-yl)-acetic acid;
Racemic-(5-{1-[4-Phenethyl-2-(4-trifluoromethyl-phenyl)-thiazol-5-yl]-ethoxy}-indol-1-yl)-acetic acid;
2-{5-[4-Ethyl-2-(4-trifluoromethyl-phenyl)-thiazol-5-ylmethoxy]-indol-1-yl}-2-methyl-propionic acid;
Racemic(6-{1-[4-Isopropyl-2-(4-trifluoromethyl-phenyl)-thiazol-5-yl]-ethoxy}-1H-indol-3-yl)-acetic acid;
Racemic(6-{1-[4-methyl-2-(4-trifluoromethyl-phenyl)-thiazol-5-yl]-ethoxy}-1H-indol-3-yl)-acetic acid;
(R)-(5-{1-[4-[2-(2-Chloro-6-fluoro-phenyl)-ethyl]-2-(4-trifluoromethyl-phenyl)-thiazol-5-yl]-ethoxy}-indol-1-yl)-acetic acid;
(S)-(5-{1-[4-[2-(2-Chloro-6-fluoro-phenyl)-ethyl]-2-(4-trifluoromethyl-phenyl)-thiazol-5-yl]-ethoxy}-indol-1-yl)-acetic acid;
(S)-(5-{1-[4-[2-phenylethyl]-2-(4-trifluoromethyl-phenyl)-thiazol-5-yl]-ethoxy}-indol-1-yl)-acetic acid;
(R)-(5-{1-[4-[2-phenylethyl]-2-(4-trifluoromethyl-phenyl)-thiazol-5-yl]-ethoxy}-indol-1-yl)-acetic acid;
(R)-(6-{1-[4-Isopropyl-2-(4-trifluoromethyl-phenyl)-thiazol-5-yl]-ethoxy}-1H-indol-3-yl)-acetic acid;
(s)-(6-{1-[4-Isopropyl-2-(4-trifluoromethyl-phenyl)-thiazol-5-yl]-ethoxy}-1H-indol-3-yl)-acetic acid;
(S)-(5-{1-[4-Propyl-2-(4-trifluoromethyl-phenyl)-thiazol-5-yl]-ethoxy}-indol-1-yl)-acetic acid;
(R)-(5-{1-[4-Propyl-2-(4-trifluoromethyl-phenyl)-thiazol-5-yl]-ethoxy}-indol-1-yl)-acetic acid;
(R)-(5-{1-[4-Isopropyl-2-(4-trifluoromethyl-phenyl)-thiazol-5-yl]-ethoxy}-indol-1-yl)-acetic acid;
(R)-(5-{1-[4-Methyl-2-(4-trifluoromethyl-phenyl)-thiazol-5-yl]-ethoxy}-indol-1-yl)-acetic acid;
(S)-(5-{1-[4-Methyl-2-(4-trifluoromethyl-phenyl)-thiazol-5-yl]-ethoxy)-indol-1-yl)-acetic acid;
(S)-(5-{2-[5-Ethyl-2-(4-trifluoromethyl-phenyl)-thiazol-4-yl]-1-methyl-ethoxy}-indol-1-yl)-acetic acid;
(R)-(5-{2-[5-Ethyl-2-(4-trifluoromethyl-phenyl)-thiazol-4-yl]-1-methyl-ethoxy}-indol-1-yl)-acetic acid;
(R)-(5-{2-[5-Methyl-2-(4-trifluoromethyl-phenyl)-thiazol-4-yl]-propoxy}-1H-indol-3-yl)-acetic acid;
(S)-(5-{2-[5-Methyl-2-(4-trifluoromethyl-phenyl)-thiazol-4-yl]-propoxy}-1H-indol-3-yl)-acetic acid;
(5-{2-[5-Methyl-2-(4-trifluoromethyl-phenyl)-thiazol-4-yl]-ethoxy}-1H-indol-3-yl)-acetic acid;
(R)-(1-Methyl-5-{2-[5-methyl-2-(4-trifluoromethyl-phenyl)-thiazol-4-yl]-propoxy}-1H-indol-3-yl)-acetic acid;
(S)-(1-Methyl-5-{2-[5-Methyl-2-(4-trifluoromethyl-phenyl)-thiazol-4-yl]-propoxy}-1H-indol-3-yl)-acetic acid;
(1-Methyl-5-{2-[5-methyl-2-(4-trifluoromethyl-phenyl)-thiazol-4-yl]-ethoxy}-1H-indol-3-yl)-acetic acid;
5-{2-[4-Isopropyl-2-(4-trifluoromethyl-phenyl)-oxazol-5-yl]-propoxy}-1H-indole-2-carboxylic acid;
5-{2-[4-Isopropyl-2-(4-trifluoromethyl-phenyl)-oxazol-5-yl]-propoxy}-1-methyl-1H-indole-2-carboxylic acid;
N-(2-{5-[5-Methyl-2-(4-trifluoromethyl-phenyl)-oxazol-4-ylmethoxy]-indol-1-yl}-acetyl)-methanesulfonamide;
N-(2-{5-[5-Methyl-2-(4-trifluoromethyl-phenyl)-oxazol-4-ylmethoxy]-indol-1-yl}-acetyl)-benzenesulfonamide;
N-[2-(5-{2-[5-Ethyl-2-(4-trifluoromethyl-phenyl)-thiazol-4-yl]-ethoxy}-indol-1-yl)-acetyl]-methanesulfonamide;
N-[2-(5-{2-[5-Ethyl-2-(4-trifluoromethyl-phenyl)-thiazol-4-yl]-ethoxy}-indol-1-yl)-acetyl]-benzenesulfonamide; and
{1-[4-Isopropyl-2-(4-trifluoromethyl-phenyl)-thiazol-5-ylmethyl]-1H-indol-5-yloxy}-acetic acid.

In other embodiments, the compound represented by Structural Formula I′ is selected from:
Racemic-(6-{1-[4-Isopropyl-2-(4-trifluoromethyl-phenyl)-thiazol-5-yl]-ethoxy}-1H-indol-3-yl)-acetic acid;
Racemic-(6-{2-[5-Methyl-2-(4-trifluoromethyl-phenyl)-thiazol-4-yl]-propoxy}-1H-indol-3-yl)-acetic acid;
Racemic-(1-Methyl-6-{2-[5-methyl-2-(4-trifluoromethyl-phenyl)-thiazol-4-yl]-propoxy}-1H-indol-3-yl)-acetic acid;
(S)-(6-{2-[5-Methyl-2-(4-trifluoromethyl-phenyl)-thiazol-4-yl]-propoxy}-1H-indol-3-yl)-acetic acid;
(R)-(6-{2-[5-Methyl-2-(4-trifluoromethyl-phenyl)-thiazol-4-yl]-propoxy}-1H-indol-3-yl)-acetic acid;
Racemic-(6-Hydroxy-5-{1-[4-isopropyl-2-(4-trifluoromethyl-phenyl)-thiazol-5-yl]-ethyl}-1H-indol-3-yl)-acetic acid;
(6-{2-[(4-Isopropyl-2-(4-trifluoromethyl-phenyl)-thiazol-5-yl]-propoxy}-1-methyl-1H-indol-3-yl)-acetic acid;
(1-Methyl-6-{2-[4-methyl-2-(4 trifluoromethyl-phenyl)-thiazol-5-yl]-propoxy}-1H-indol-3-yl)-acetic acid;
(6-{2-[5-Ethyl-2-(4-trifluoromethyl-phenyl)-thiazol-4-yl]-propoxy}-1-methyl-1H-indol-3-yl)-acetic acid;
(R)-(6-{2-[4-Isopropyl-2-(4-trifluoromethyl-phenyl)-thiazol-5-yl]-propoxy}-1-methyl-1H-indol-3-yl)-acetic acid;
(S)-(6-{2-[4-Isopropyl-2-(4-trifluoromethyl-phenyl)-thiazol-5-yl]-propoxy}-1-methyl-1H-indol-3-yl)-acetic acid;
Racemic-(1-Methyl-6-{(2-[4-methyl-2-(4-trifluoromethyl-phenyl)-thiazol-5-yl]-propoxy}-1H-indol-3-yl)-acetic acid;
Racemic-(1-Ethyl-6-{2-[4-isopropyl-2-(4-trifluoromethyl-phenyl)-thiazol-5-yl]-propoxy}-1H-indol-3-yl)-acetic acid;
Racemic-(6-{2-[(4-Isopropyl-2-(4-trifluoromethyl-phenyl)-thiazol-5-yl]-propoxy}-1-propyl-1H-indol-3-yl)-acetic acid;
{1-[4-Isopropyl-2-(4-trifluoromethyl-phenyl)-thiazol-5-ylmethyl]-1H-indol-5-yloxy}-acetic acid;
Racemic(6-{1-[4-Isopropyl-2-(4-trifluoromethyl-phenyl)-thiazol-5-yl]-ethoxy}-1H-indol-3-yl)-acetic acid;
Racemic(6-{1-[4-methyl-2-(4-trifluoromethyl-phenyl)-thiazol-5-yl]-ethoxy}-1H-indol-3-yl)-acetic acid;
(R)-(6-{1-[4-Isopropyl-2-(4-trifluoromethyl-phenyl)-thiazol-5-yl]-ethoxy}-1H-indol-3-yl)-acetic acid;
(S)-(6-{-[4-Isopropyl-2-(4-trifluoromethyl-phenyl)-thiazol-5-yl]-ethoxy}-1H-indol-3-yl)-acetic acid;
(R)-(5-{(2-[5-Methyl-2-(4-trifluoromethyl-phenyl)-thiazol-4-yl]-propoxy}-1H-indol-3-yl)-acetic acid;
(S)-(5-{2-[5-Methyl-2-(4-trifluoromethyl-phenyl)-thiazol-4-yl]-propoxy}-1H-indol-3-yl)-acetic acid;
(5-{2-[5-Methyl-2-(4-trifluoromethyl-phenyl)-thiazol-4-yl]-ethoxy}-1H-indol-3-yl)-acetic acid;
(R)-(1-Methyl-5-{2-[5-methyl-2-(4-trifluoromethyl-phenyl)-thiazol-4-yl]-propoxy}-1H-indol-3-yl)-acetic acid;
(S)-(1-Methyl-5-{2-[5-Methyl-2-(4-trifluoromethyl-phenyl)-thiazol-4-yl]-propoxy}-1H-indol-3-yl)-acetic acid;
(1-Methyl-5-{2-[5-methyl-2-(4-trifluoromethyl-phenyl)-thiazol-4-yl]-ethoxy}-1H-indol-3-yl)-acetic acid;
5-{2-[4-Isopropyl-2-(4-trifluoromethyl-phenyl)-oxazol-5-yl]-propoxy}-1H-indole-2-carboxylic acid; and
5-{2-[4-Isopropyl-2-(4-trifluoromethyl-phenyl)-oxazol-5-yl]-propoxy}-1-methyl-1H-indole-2-carboxylic acid.

In other embodiments, the compound represented by Structural Formula I′ is selected from:
{5-[5-Methyl-2-(4-trifluoromethyl-phenyl)-oxazol-4-ylmethoxy]-indol-1-yl}-acetic acid;
[5-(5-Methyl-2-phenyl-oxazol-4-ylmethoxy)-indol-1-yl]-acetic acid;
{5-[2-(4-Fluoro-phenyl)-5-methyl-oxazol-4-ylmethoxy]-indol-1-yl}-acetic acid;
{5-[2-(4-Benzyloxy-phenyl)-5-methyl-oxazol-4-ylmethoxy]-indol-1-yl}-acetic acid;
2-Methyl-2-(5-{2-[2-(4-trifluoromethyl-phenyl)-oxazol-4-ylmethoxy]-ethoxy}-indol-1-yl)-propionic acid;
{5-[4-Ethyl-2-(4-trifluoromethyl-phenyl)-thiazol-5-ylmethoxy]-indol-1-yl}-acetic acid;
2-Methyl-2-(5-{2-[4-methyl-2-(4-trifluoromethyl-phenyl)-thiazol-5-ylmethoxy]-ethoxy}-indol-1-yl)-propionic acid;
{5-[2-(3,5-Bis-trifluoromethyl-phenyl)-4-methyl-thiazol-5-ylmethoxy]-indol-1-yl}-acetic acid;
(5-[4-Methyl-2-(4-trifluoromethyl-phenyl)-oxazol-5-ylmethoxy]-indol-1-yl}-acetic acid;
2-Methyl-2-{5-[4-methyl-2-(4-trifluoromethyl-phenyl)-oxazol-5-ylmethoxy]-indol-1-yl}-propionic acid;
Racemic 2-{5-[4-Methyl-2-(4-trifluoromethyl-phenyl)-oxazol-5-ylmethoxy]-indol-1-yl}-propionic acid;
{5-[2-(4-Bromo-phenyl)-4-methyl-thiazol-5-ylmethoxy]-indol-1-yl}-acetic acid;
{5-[4-Butyl-2-(4-trifluoromethyl-phenyl)-thiazol-5-ylmethoxy]-indol-1-yl}-acetic acid;
2-{5-[4-Butyl-2-(4-trifluoromethyl-phenyl)-thiazol-5-ylmethoxy]-indol-1-yl}-propionic acid;
{5-[4-Phenethyl-2-(4-trifluoromethyl-phenyl)-thiazol-5-ylmethoxy]-indol-1-yl}-acetic acid;
2-{5-[4-(2-Chloro-6-fluoro-phenoxymethyl)-2-(4-trifluoromethyl-phenyl)-thiazol-5-ylmethoxy]-indol-1-yl}-propionic acid;
Racemic 2-Methyl-2-{5-[4-phenoxymethyl-2-(4-trifluoromethyl-phenyl)-thiazol-5-ylmethoxy]-indol-1-yl}-propionic acid;
2-Methyl-2-{5-[4-phenoxymethyl-2-(4-trifluoromethyl-phenyl)-thiazol-5-ylmethoxy]-indol-1-yl}-propionic acid;
3-{5-[4-Propyl-2-(4-trifluoromethyl-phenyl)-thiazol-5-ylmethoxy]-indol-1-yl}-propionic acid;
5-{5-[4-Methyl-2-(4-trifluoromethyl-phenyl)-thiazol-5-ylmethoxy]-indol-1-yl}-pentanoic acid;
5-{5-[4-Propyl-2-(4-trifluoromethyl-phenyl)-thiazol-5-ylmethoxy]-indol-1-yl}-pentanoic acid;
{5-[4-Isopropyl-2-(4-trifluoromethyl-phenyl)-thiazol-5-ylmethoxy]-indol-1-yl}-acetic acid;
2-{5-[4-(2-Chloro-6-fluoro-phenoxymethyl)-2-(4-trifluoromethyl-phenyl)-thiazol-5-ylmethoxy]-indol-1-yl}-2-methyl-propionic acid;
5-{5-[5-Methyl-2-(4-trifluoromethyl-phenyl)-oxazol-4-ylmethoxy]-indol-1-yl}-pentanoic acid;
5-{5-[2-(4-Bromo-phenyl)-5-methyl-oxazol-4-ylmethoxy]-indol-1-yl}-pentanoic acid;
3-{5-[2-(4-Bromo-phenyl)-5-methyl-oxazol-4-ylmethoxy]-indol-1-yl}-propionic acid;
3-{5-[4-Methyl-2-(4-trifluoromethyl-phenyl)-thiazol-5-ylmethoxy]-indol-1-yl}-propionic acid;
{5-[4-Isopropyl-2-(4-trifluoromethyl-phenyl)-triazol-5-ylmethylsulfanyl]-indol-1-yl}-acetic acid;
{5-[4-Ethyl-2-(4-trifluoromethyl-phenyl)-oxazol-5-ylmethoxy]-indol-1-yl}-acetic acid;
(5-{2-[5-Methyl-2-(4-trifluoromethyl-phenyl)-oxazol-4-yl]-ethoxy}-indol-1-yl)-acetic acid;
{5-[2-(5-Methyl-2-pyridin-4-yl-thiazol-4-yl)-ethoxy]-indol-1-yl}-acetic acid;
{5-[2-(5-Methyl-2-morpholin-4-yl-thiazol-4-yl)-ethoxy]-indol-1-yl}-acetic acid;
(5-{2-[5-Methyl-2-(4-trifluoromethyl-phenyl)-oxazol-4-yl]-ethoxy}-indol-1-yl)-acetic acid;
(5-{2-[5-Methyl-2-(tetrahydro-pyran-4-yl)-oxazol-4-yl]-ethoxy}-indol-1-yl)-acetic acid;
{5-[2-(2-Butoxy-5-methyl-oxazol-4-yl)-ethoxy]-indol-1-yl}-acetic acid;
{5-[2-(5-Methyl-2-pyridin-3-yl-thiazol-4-yl)-ethoxy]-indol-1-yl}-acetic acid;
{5-[2-(5-Methyl-2-pyridin-2-yl-thiazol-4-yl)-ethoxy]-indol-1-yl}-acetic acid;
(5-{2-[2-(5-Bromo-thiophen-2-yl)-5-methyl-oxazol-4-yl]-ethoxy}-indol-1-yl)-acetic acid;
(5-{2-[2-(3-Bromo-phenyl)-5-methyl-oxazol-4-yl]-ethoxy}-indol-1-yl)-acetic acid;
(5-{2-[5-Ethyl-2-(4-trifluoromethyl-phenyl)-thiazol-4-yl]-ethoxy}-indol-1-yl)-acetic acid;
2-(5-{2-[5-Ethyl-2-(4-trifluoromethyl-phenyl)-thiazol-4-yl]-ethoxy}-indol-1-yl)-propionic acid;
(5-{2-[2-(2-Chloro-phenyl)-5-ethyl-thiazol-4-yl]-ethoxy}-indol-1-yl)-acetic acid;
2-(5-{2-[2-(2-Chloro-phenyl)-5-ethyl-thiazol-4-yl]-ethoxy}-indol-1-yl)-propionic acid;
(5-{2-[5-Propyl-2-(4-trifluoromethyl-phenyl)-thiazol-4-yl]-ethoxy}-indol-1-yl)-acetic acid;
2-(5-{2-[5-Propyl-2-(4-trifluoromethyl-phenyl)-thiazol-4-yl]-ethoxy}-indol-1-yl)-propionic acid;
2-(5-{2-[2-(2-Chloro-phenyl)-5-ethyl-thiazol-4-yl]-ethoxy}-indol-1-yl)-2-methyl-propionic acid;
Racemic 2-(5-{2-[5-Ethyl-2-(4-trifluoromethyl-phenyl)-oxazol-4-yl]-ethoxy}-indol-1-yl)-propionic acid;
(5-{2-[5-Ethyl-2-(4-trifluoromethyl-phenyl)-oxazol-4-yl]-ethoxy}-indol-1-yl)-acetic acid;
2-(5-{2-[5-Ethyl-2-(4-trifluoromethyl-phenyl)-oxazol-4-yl]-ethoxy}-indol-1-yl)-2-methyl-propionic acid; 3-(5-{2-[2-(2-Chloro-phenyl)-5-ethyl-thiazol-4-yl]-ethoxy}-indol-1-yl)-propionic acid;
3-(5-{2-[5-Ethyl-2-(4-trifluoromethyl-phenyl)-oxazol-4-yl]-ethoxy}-indol-1-yl)-propionic acid;
2-Methyl-2-(5-{2-[5-propyl-2-(4-trifluoromethyl-phenyl)-thiazol-4-yl]-ethoxy}-indol-1-yl)-propionic acid;
Racemic-(5-{1-[4-Methyl-2-(4-trifluoromethyl-phenyl)-thiazol-5-yl]-ethoxy}-indol-1-yl)-acetic acid;
Racemic-(5-{1-[4-Methyl-2-(4-trifluoromethyl-phenyl)-thiazol-5-yl]-ethoxy}-indol-1-yl)-acetic acid;
Racemic-(5-{1-[4-Isopropyl-2-(4-trifluoromethyl-phenyl)-thiazol-5-yl]-ethoxy}-indol-1-yl)-acetic acid;
Racemic-(5-{2-[5-Ethyl-2-(4-trifluoromethyl-phenyl)-thiazol-4-yl]-1-methyl-ethoxy}-indol-1-yl)-acetic acid;
Racemic-(5-{2-[5-Methyl-2-(4-trifluoromethyl-phenyl)-thiazol-4-yl]-propoxy}-indol-1-yl)-acetic acid;
(5-{2-[5-Methyl-2-(4-trifluoromethyl-phenyl)-oxazol-4-yl]-propoxy}-indol-1-yl)-acetic acid;
(S)-(5-{2-[5-Methyl-2-(4-trifluoromethyl-phenyl)-oxazol-4-yl]-propoxy}-indol-1-yl)-acetic acid;
(R)-(5-{2-[5-Methyl-2-(4-trifluoromethyl-phenyl)-oxazol-4-yl]-propoxy}-indol-1-yl)-acetic acid;
Racemic-(5-{1-[4-Propyl-2-(4-trifluoromethyl-phenyl)-thiazol-5-yl]-ethoxy}-indol-1-yl)-acetic acid;
Racemic-(5-{1-[4-Ethyl-2-(4-trifluoromethyl-phenyl)-oxazol-5-yl]-ethoxy}-indol-1-yl)-acetic acid;
{5-[4-Propyl-2-(4-trifluoromethyl-phenyl)-thiazol-5-ylmethoxy]-indol-1-yl}-acetic acid;
Racemic-2-{5-[4-Propyl-2-(4-trifluoromethyl-phenyl)-thiazol-5-ylmethoxy]-indol-1-yl}-propionic acid;
2-Methyl-2-{5-[4-propyl-2-(4-trifluoromethyl-phenyl)-thiazol-5-ylmethoxy]-indol-1-yl}-propionic acid;
Racemic-2-{5-[4-Phenethyl-2-(4-trifluoromethyl-phenyl)-thiazol-5-ylmethoxy]-indol-1-yl}-propionic acid;
2-Methyl-2-{5-[4-phenethyl-2-(4-trifluoromethyl-phenyl)-thiazol-5-ylmethoxy]-indol-1-yl}-propionic acid;
{5-[4-tert-Butyl-2-(4-trifluoromethyl-phenyl)-thiazol-5-ylmethoxy]-indol-1-yl}-acetic acid;
2-Methyl-2-[5-(5-methyl-2-phenyl-oxazol-4-ylmethoxy)-indol-1-yl]-propionic acid;
2-{5-[2-(4-Trifluoromethyl-phenyl)-5-methyl-oxazol-4-ylmethoxy]-indol-1-yl}-2-methyl-propionic acid;
2-{5-[2-(4-Fluoro-phenyl)-5-methyl-oxazol-4-ylmethoxy]-indol-1-yl}-2-methyl-propionic acid;
2-{5-[2-(4-Bromo-phenyl)-5-methyl-oxazol-4-ylmethoxy]-indol-1-yl}-2-methyl-propionic acid;
2-Methyl-2-(5-{2-[5-methyl-2-(4-trifluoromethyl-phenyl)-oxazol-4-yl]-ethoxy}-indol-1-yl)-propionic acid;
2-(5-{2-[2-(4-Bromo-phenyl)-5-methyl-oxazol-4-yl]-ethoxy}-indol-1-yl)-2-methyl-propionic acid;
2-(5-{2-[2-(5-Bromo-thiophen-2-yl)-5-methyl-oxazol-4-yl]-ethoxy}-indol-1-yl)-2-methyl-propionic acid;
2-Methyl-2-{5-[2-(5-methyl-2-phenyl-thiazol-4-yl)-ethoxy]-indol-1-yl}-propionic acid;
{5-[4-Isopropyl-2-(4-trifluoromethyl-phenyl)-thiazol-5-ylmethoxy]-2-methyl-indol-1-yl}-acetic acid;
2-{5-[2-(3,5-Bis-trifluoromethyl-phenyl)-4-methyl-thiazol-5-ylmethoxy]-indol-1-yl}-2-methyl-propionic acid;
{4-[4-Propyl-2-(4-trifluoromethyl-phenyl)-thiazol-5-ylmethoxy]-indol-1-yl}-acetic acid;
Racemic-(5-{1-[4-[2-(2-Chloro-6-fluoro-phenyl)-ethyl]-2-(4-trifluoromethyl-phenyl)-thiazol-5-yl]-ethoxy}-indol-1-yl)-acetic acid;
Racemic-(5-{1-[4-Methyl-2-(4-trifluoromethyl-phenyl)-thiazol-5-yl]-2-phenyl-ethoxy}-indol-1-yl)-acetic acid;
Racemic-(5-{1-[4-Phenethyl-2-(4-trifluoromethyl-phenyl)-thiazol-5-yl]-ethoxy)-indol-1-yl)-acetic acid;
2-{5-[4-Ethyl-2-(4-trifluoromethyl-phenyl)-thiazol-5-ylmethoxy]-indol-1-yl}-2-methyl-propionic acid;
(R)-(5-{1-[4-[2-(2-Chloro-6-fluoro-phenyl)-ethyl]-2-(4-trifluoromethyl-phenyl)-thiazol-5-yl]-ethoxy}-indol-1-yl)-acetic acid;
(S)-(5-{1-[4-[2-(2-Chloro-6-fluoro-phenyl)-ethyl]-2-(4-trifluoromethyl-phenyl)-thiazol-5-yl]-ethoxy)-indol-1-yl)-acetic acid;
(S)-(5-{1-[4-[2-phenylethyl]-2-(4-trifluoromethyl-phenyl)-thiazol-5-yl]-ethoxy}-indol-1-yl)-acetic acid;
(R)-(5-{1-[4-[2-phenylethyl]-2-(4-trifluoromethyl-phenyl)-thiazol-5-yl]-ethoxy)-indol-1-yl)-acetic acid;
(S)-(5-{1-[4-Propyl-2-(4-trifluoromethyl-phenyl)-thiazol-5-yl]-ethoxy}-indol-1-yl)-acetic acid;
(R)-(5-{1-[4-Propyl-2-(4-trifluoromethyl-phenyl)-thiazol-5-yl]-ethoxy}-indol-1-yl)-acetic acid;
(R)-(5-{1-[4-Isopropyl-2-(4-trifluoromethyl-phenyl)-thiazol-5-yl]-ethoxy}-indol-1-yl)-acetic acid;
(R)-(5-{1-[4-Methyl-2-(4-trifluoromethyl-phenyl)-thiazol-5-yl]-ethoxy}-indol-1-yl)-acetic acid;
(S)-(5-{1-[4-Methyl-2-(4-trifluoromethyl-phenyl)-thiazol-5-yl]-ethoxy}-indol-1-yl)-acetic acid;
(S)-(5-{2-[5-Ethyl-2-(4-trifluoromethyl-phenyl)-thiazol-4-yl]-1-methyl-ethoxy}-indol-1-yl)-acetic acid;
(R)-(5-{2-[5-Ethyl-2-(4-trifluoromethyl-phenyl)-thiazol-4-yl]-1-methyl-ethoxy}-indol-1-yl)-acetic acid;
N-(2-{5-[5-Methyl-2-(4-trifluoromethyl-phenyl)-oxazol-4-ylmethoxy]-indol-1-yl}-acetyl)-methanesulfonamide;
N-(2-{5-[5-Methyl-2-(4-trifluoromethyl-phenyl)-oxazol-4-ylmethoxy]-indol-1-yl}-acetyl)-benzenesulfonamide; and
N-[2-(5-{2-[5-Ethyl-2-(4-trifluoromethyl-phenyl)-thiazol-4-yl]-ethoxy}-indol-1-yl)-acetyl]-methanesulfonamide.

In other embodiments, the compound represented by Structural Formula I″ is selected from:
{5-[5-Methyl-2-(4-trifluoromethyl-phenyl)-oxazol-4-ylmethoxy]-indol-1-yl}-acetic acid;
[5-(5-Methyl-2-phenyl-oxazol-4-ylmethoxy)-indol-1-yl]-acetic acid;
{5-[2-(4-Fluoro-phenyl)-5-methyl-oxazol-4-ylmethoxy]-indol-1-yl}-acetic acid;
{5-[2-(4-Benzyloxy-phenyl)-5-methyl-oxazol-4-ylmethoxy]-indol-1-yl}-acetic acid;
2-Methyl-2-(5-{2-[2-(4-trifluoromethyl-phenyl)-oxazol-4-ylmethoxy]-ethoxy}-indol-1-yl)-propionic acid;
{5-[4-Ethyl-2-(4-trifluoromethyl-phenyl)-thiazol-5-ylmethoxy]-indol-1-yl}-acetic acid;
2-Methyl-2-(5-{2-[4-methyl-2-(4-trifluoromethyl-phenyl)-thiazol-5-ylmethoxy]-ethoxy}-indol-1-yl)-propionic acid;
{5-[2-(3,5-Bis-trifluoromethyl-phenyl)-4-methyl-thiazol-5-ylmethoxy]-indol-1-yl}-acetic acid;
{5-[4-Methyl-2-(4-trifluoromethyl-phenyl)-oxazol-5-ylmethoxy]-indol-1-yl}-acetic acid;
2-Methyl-2-{5-[4-methyl-2-(4-trifluoromethyl-phenyl)-oxazol-5-ylmethoxy]-indol-1-yl}-propionic acid;
Racemic 2-{5-[4-Methyl-2-(4-trifluoromethyl-phenyl)-oxazol-5-ylmethoxy]-indol-1-yl}-propionic acid;
{5-[2-(4-Bromo-phenyl)-4-methyl-thiazol-5-ylmethoxy]-indol-1-yl}-acetic acid;
{5-[4-Butyl-2-(4-trifluoromethyl-phenyl)-thiazol-5-ylmethoxy]-indol-1-yl}-acetic acid;
2-{5-[4-Butyl-2-(4-trifluoromethyl-phenyl)-thiazol-5-ylmethoxy]-indol-1-yl}-propionic acid;
{5-[4-Phenethyl-2-(4-trifluoromethyl-phenyl)-thiazol-5-ylmethoxy]-indol-1-yl}-acetic acid;
2-{5-[4-(2-Chloro-6-fluoro-phenoxymethyl)-2-(4-trifluoromethyl-phenyl)-thiazol-5-ylmethoxy]-indol-1-yl}-propionic acid;
{5-[4-Phenoxymethyl-2-(4-trifluoromethyl-phenyl)-thiazol-5-ylmethoxy]-indol-1-yl}-acetic acid;
Racemic 2-Methyl-2-{5-[4-phenoxymethyl-2-(4-trifluoromethyl-phenyl)-thiazol-5-ylmethoxy]-indol-1-yl}-propionic acid;
2-Methyl-2-{5-[4-phenoxymethyl-2-(4-trifluoromethyl-phenyl)-thiazol-5-ylmethoxy]-indol-1-yl}-propionic acid;
3-{5-[4-Propyl-2-(4-trifluoromethyl-phenyl)-thiazol-5-ylmethoxy]-indol-1-yl}-propionic acid;
5-{5-[4-Methyl-2-(4-trifluoromethyl-phenyl)-thiazol-5-ylmethoxy]-indol-1-yl}-pentanoic acid;
5-{5-[4-Propyl-2-(4-trifluoromethyl-phenyl)-thiazol-5-ylmethoxy]-indol-1-yl}-pentanoic acid;
(5-[4-Isopropyl-2-(4-trifluoromethyl-phenyl)-thiazol-5-ylmethoxy]-indol-1-yl}-acetic acid;
(5-[4-(2-Chloro-6-fluoro-phenoxymethyl)-2-(4-trifluoromethyl-phenyl)-thiazol-5-ylmethoxy]-indol-1-yl}-acetic acid;
2-{5-[4-(2-Chloro-6-fluoro-phenoxymethyl)-2-(4-trifluoromethyl-phenyl)-thiazol-5-ylmethoxy]-indol-1-yl}-2-methyl-propionic acid;
5-{5-[5-Methyl-2-(4-trifluoromethyl-phenyl)-oxazol-4-ylmethoxy]-indol-1-yl}-pentanoic acid;
5-{5-[2-(4-Bromo-phenyl)-5-methyl-oxazol-4-ylmethoxy]-indol-1-yl}-pentanoic acid;
5-{5-[4-Phenethyl-2-(4-trifluoromethyl-phenyl)-thiazol-5-ylmethoxy]-indol-1-yl}-pentanoic acid;
4-[1-(4-Carboxy-butyl)-1H-indol-5-yloxymethyl]-2-(4-trifluoromethyl-phenyl)-thiazole-5-carboxylic acid;
3-{5-[2-(4-Bromo-phenyl)-5-methyl-oxazol-4-ylmethoxy]-indol-1-yl}-propionic acid;
3-{5-[4-Phenethyl-2-(4-trifluoromethyl-phenyl)-thiazol-5-ylmethoxy]-indol-1-yl}-propionic acid;
3-{5-[4-Methyl-2-(4-trifluoromethyl-phenyl)-thiazol-5-ylmethoxy]-indol-1-yl}-propionic acid;
4-[1-(2-Carboxy-ethyl)-1H-indol-5-yloxymethyl]-2-(4-trifluoromethyl-phenyl)-thiazole-5-carboxylic acid;
{5-[4-Isopropyl-2-(4-trifluoromethyl-phenyl)-thiazol-5-ylmethylsulfanyl]-indol-1-yl}-acetic acid;
{5-[4-Ethyl-2-(4-trifluoromethyl-phenyl)-oxazol-5-ylmethoxy]-indol-1-yl}-acetic acid;
(5-{2-[5-Methyl-2-(4-trifluoromethyl-phenyl)-oxazol-4-yl]-ethoxy}-indol-1-yl)-acetic acid;
{5-[2-(5-Methyl-2-pyridin-4-yl-thiazol-4-yl)-ethoxy]-indol-1-yl}-acetic acid;
{5-[2-(5-Methyl-2-morpholin-4-yl-thiazol-4-yl)-ethoxy]-indol-1-yl}-acetic acid;
(5-{2-[5-Methyl-2-(4-trifluoromethyl-phenyl)-oxazol-4-yl]-ethoxy}-indol-1-yl)-acetic acid;
(5-{2-[5-Methyl-2-(tetrahydro-pyran-4-yl)-oxazol-4-yl]-ethoxy}-indol-1-yl)-acetic acid;
{5-[2-(2-Butoxy-5-methyl-oxazol-4-yl)-ethoxy]-indol-1-yl}-acetic acid;
{5-[2-(5-Methyl-2-pyridin-3-yl-thiazol-4-yl)-ethoxy]-indol-1-yl}-acetic acid;
{5-[2-(5-Methyl-2-pyridin-2-yl-thiazol-4-yl)-ethoxy]-indol-1-yl}-acetic acid;
(5-{2-[2-(5-Bromo-thiophen-2-yl)-5-methyl-oxazol-4-yl]-ethoxy}-indol-1-yl)-acetic acid;
{5-[3-(4-Butyl-phenoxy)-propoxy]-indol-1-yl}-acetic acid;
(5-{2-[2-(3-Bromo-phenyl)-5-methyl-oxazol-4-yl]-ethoxy}-indol-1-yl)-acetic acid;
(5-{2-[5-Ethyl-2-(4-trifluoromethyl-phenyl)-thiazol-4-yl]-ethoxy}-indol-1-yl)-acetic acid;
2-(5-{2-[5-Ethyl-2-(4-trifluoromethyl-phenyl)-thiazol-4-yl]-ethoxy}-indol 1-yl)-propionic acid;
(5-{2-[2-(2-Chloro-phenyl)-5-ethyl-thiazol-4-yl]-ethoxy}-indol-1-yl)-acetic acid;
2-(5-{2-[2-(2-Chloro-phenyl)-5-ethyl-thiazol-4-yl]-ethoxy}-indol-1-yl)-propionic acid;
(5-{2-[5-Propyl-2-(4-trifluoromethyl-phenyl)-thiazol-4-yl]-ethoxy}-indol-1-yl)-acetic acid;
2-(5-{2-[5-Propyl-2-(4-trifluoromethyl-phenyl)-thiazol-4-yl]-ethoxy}-indol-1-yl)-propionic acid;
2-(5-{2-[2-(2-Chloro-phenyl)-5-ethyl-thiazol-4-yl]-ethoxy}-indol-1-yl)-2-methyl-propionic acid;
Racemic 2-(5-{2-[5-Ethyl-2-(4-trifluoromethyl-phenyl)-oxazol-4-yl]-ethoxy}-indol-1-yl)-propionic acid;
(5-{2-[5-Ethyl-2-(4-trifluoromethyl-phenyl)-oxazol-4-yl]-ethoxy}-indol-1-yl)-acetic acid;
2-(5-{2-[5-Ethyl-2-(4-trifluoromethyl-phenyl)-oxazol-4-yl]-ethoxy}-indol-1-yl)-2-methyl-propionic acid;
3-(5-{2-[2-(2-Chloro-phenyl)-5-ethyl-thiazol-4-yl]-ethoxy}-indol-1-yl)-propionic acid;
3-(5-{2-[5-Ethyl-2-(4-trifluoromethyl-phenyl)-oxazol-4-yl]-ethoxy}-indol-1-yl)-propionic acid;
2-Methyl-2-(5-{2-[5-propyl-2-(4-trifluoromethyl-phenyl)-thiazol-4-yl]-ethoxy}-indol-1-yl)-propionic acid;
Racemic-(5-{1-[4-Methyl-2-(4-trifluoromethyl-phenyl)-thiazol-5-yl]-ethoxy}-indol-1-yl)-acetic acid; Racemic-(5-{1-[4-Methyl-2-(4-trifluoromethyl-phenyl)-thiazol-5-yl]-ethoxy}-indol-1-yl)-acetic acid;
Racemic-(5-{1-[4-Isopropyl-2-(4-trifluoromethyl-phenyl)-thiazol-5-yl]-ethoxy}-indol-1-yl)-acetic acid;
Racemic-(5-{2-[5-Ethyl-2-(4-trifluoromethyl-phenyl)-thiazol-4-yl]-1-methyl-ethoxy}-indol-1-yl)-acetic acid;
Racemic-(5-{2-[5-Methyl-2-(4-trifluoromethyl-phenyl)-thiazol-4-yl]-propoxy}-indol-1-yl)-acetic acid;
(5-{2-[5-Methyl-2-(4-trifluoromethyl-phenyl)-oxazol-4-yl]-propoxy}-indol-1-yl)-acetic acid;
(S)-(5-{2-[5-Methyl-2-(4-trifluoromethyl-phenyl)-oxazol-4-yl]-propoxy}-indol-1-yl)-acetic acid;
(R)-(5-{2-[5-Methyl-2-(4-trifluoromethyl-phenyl)-oxazol-4-yl]-propoxy}-indol-1-yl)-acetic acid;
Racemic-(5-{1-[4-Propyl-2-(4-trifluoromethyl-phenyl)-thiazol-5-yl]-ethoxy}-indol-1-yl)-acetic acid;
Racemic-(5-{1-[4-Ethyl-2-(4-trifluoromethyl-phenyl)-oxazol-5-yl]-ethoxy}-indol-1-yl)-acetic acid;
{5-[4-Propyl-2-(4-trifluoromethyl-phenyl)-thiazol-5-ylmethoxy)-indol-1-yl}-acetic acid;
Racemic-2-{5-[4-Propyl-2-(4-trifluoromethyl-phenyl)-thiazol-5-ylmethoxy]-indol-1-yl)-propionic acid;
2-Methyl-2-{5-[4-propyl-2-(4-trifluoromethyl-phenyl)-thiazol-5-ylmethoxy]-indol-1-yl}-propionic acid;
Racemic-2-{5-[4-Phenethyl-2-(4-trifluoromethyl-phenyl)-thiazol-5-ylmethoxy]-indol-1-yl}-propionic acid;
2-Methyl-2-{5-[4-phenethyl-2-(4-trifluoromethyl-phenyl)-thiazol-5-ylmethoxy]-indol-1-yl}-propionic acid;
{5-[4-Phenethyl-2-(4-trifluoromethyl-phenyl)-thiazol-5-ylmethoxy]-indol-1-yl}-acetic acid;
(5-[4-Phenyl-2-(4-trifluoromethyl-phenyl)-thiazol-5-ylmethoxy)-indol-1-yl}-acetic acid;
{5-[4-tert-Butyl-2-(4-trifluoromethyl-phenyl)-thiazol-5-ylmethoxy]-indol-1-yl}-acetic acid;
{5-[4-[2-(2-Chloro-6-fluoro-phenyl)-ethyl]-2-(4-trifluoromethyl-phenyl)-thiazol-5-ylmethoxy]-indol-1-yl}-acetic acid;
2-Methyl-2-[5-(5-methyl-2-phenyl-oxazol-4-ylmethoxy)-indol-1-yl]-propionic acid;
2-{5-[2-(4-Trifluoromethyl-phenyl)-5-methyl-oxazol-4-ylmethoxy]-indol 1-yl}-2-methyl-propionic acid;
2-{5-[2-(4-Fluoro-phenyl)-5-methyl-oxazol-4-ylmethoxy]-indol-1-yl}-2-methyl-propionic acid;
2-{5-[2-(4-Bromo-phenyl)-5-methyl-oxazol-4-ylmethoxy]-indol-1-yl}-2-methyl-propionic acid;
2-Methyl-2-(5-{2-[5-methyl-2-(4-trifluoromethyl-phenyl)-oxazol-4-yl]-ethoxy}-indol-1-yl)-propionic acid;
2-(5-{2-[2-(4-Bromo-phenyl)-5-methyl-oxazol-4-yl]-ethoxy}-indol-1-yl)-2-methyl-propionic acid;
2-(5-{2-[2-(5-Bromo-thiophen-2-yl)-5-methyl-oxazol-4-yl]-ethoxy}-indol-1-yl)-2-methyl-propionic acid;
2-Methyl-2-{5-[2-(5-methyl-2-phenyl-thiazol-4-yl)-ethoxy]-indol-1-yl}-propionic acid;
{5-[4-Isopropyl-2-(4-trifluoromethyl-phenyl)-thiazol-5-ylmethoxy]-2-methyl-indol-1-yl}-acetic acid;
2-{5-[2-(3,5-Bis-trifluoromethyl-phenyl)-4-methyl-thiazol-5-ylmethoxy]-indol-1-yl}-2-methyl-propionic acid;
{4-[4-Propyl-2-(4-trifluoromethyl-phenyl)-thiazol-5-ylmethoxy]-indol-1-yl}-acetic acid;
Racemic-(5-{1-[4-[2-(2-Chloro-6-fluoro-phenyl)-ethyl]-2-(4-trifluoromethyl-phenyl)-thiazol-5-yl]-ethoxy}-indol-1-yl)-acetic acid;
Racemic-(5-{1-[4-Methyl-2-(4-trifluoromethyl-phenyl)-thiazol-5-yl]-2-phenyl-ethoxy}-indol-1-yl)-acetic acid;
Racemic-(5-{1-[4-Phenethyl-2-(4-trifluoromethyl-phenyl)-thiazol-5-yl]-ethoxy}-indol-1-yl)-acetic acid;
2-{5-[4-Ethyl-2-(4-trifluoromethyl-phenyl)-thiazol-5-ylmethoxy]-indol-1-yl}-2-methyl-propionic acid;
(R)-(5-{1-[4-[2-(2-Chloro-6-fluoro-phenyl)-ethyl]-2-(4-trifluoromethyl-phenyl)-thiazol-5-yl]-ethoxy}-indol-1-yl)-acetic acid;
(S)-(5-{1-[4-[2-(2-Chloro-6-fluoro-phenyl)-ethyl]-2-(4-trifluoromethyl-phenyl)-thiazol-5-yl]-ethoxy}-indol-1-yl)-acetic acid;
(S)-(5-{1-[4-[2-phenylethyl]-2-(4-trifluoromethyl-phenyl)-thiazol-5-yl]-ethoxy}-indol-1-yl)-acetic acid;
(R)-(5-{1 [4-[2-phenylethyl]-2-(4-trifluoromethyl-phenyl)-thiazol-5-yl]-ethoxy}-indol-1-yl)-acetic acid;
(S)-(5-{1-[4-Propyl-2-(4-trifluoromethyl-phenyl)-thiazol-5-yl]-ethoxy}-indol-1-yl)-acetic acid;
(R)-(5-{1-[4-Propyl-2-(4-trifluoromethyl-phenyl)-thiazol-5-yl]-ethoxy}-indol-1-yl)-acetic acid;
(R)-(5-{1-[4-Isopropyl-2-(4-trifluoromethyl-phenyl)-thiazol-5-yl]-ethoxy}-indol-1-yl)-acetic acid;
(R)-(5-{1-[4-Methyl-2-(4-trifluoromethyl-phenyl)-thiazol-5-yl]-ethoxy}-indol-1-yl)-acetic acid;
(S)-(5-{1-[4-Methyl-2-(4-trifluoromethyl-phenyl)-thiazol-5-yl]-ethoxy}-indol-1-yl)-acetic acid;
(S)-(5-{2-[5-Ethyl-2-(4-trifluoromethyl-phenyl)-thiazol-4-yl]-1-methyl-ethoxy}-indol-1-yl)-acetic acid;
(R)-(5-{2-[5-Ethyl-2-(4-trifluoromethyl-phenyl)-thiazol-4-yl]-1-methyl-ethoxy}-indol-1-yl)-acetic acid;
N-(2-{5-[5-Methyl-2-(4-trifluoromethyl-phenyl)-oxazol-4-ylmethoxy]-indol-1-yl}-acetyl)-methanesulfonamide;
N-(2-{5-[5-Methyl-2-(4-trifluoromethyl-phenyl)-oxazol-4-ylmethoxy]-indol-1-yl}-acetyl)-benzenesulfonamide; and
N-[2-(5-{2-[5-Ethyl-2-(4-trifluoromethyl-phenyl)-thiazol-4-yl]-ethoxy}-indol-1-yl)-acetyl]-methanesulfonamide.

### Synthesis

Compounds of the present invention have been formed as specifically described in the examples. Further, many compounds are prepared as more generally using a) alkylation of phenol/thiophenol with an alkylating agent, b) a Mitsunobu protocol (O. Mitsunobu, 1981 Synthesis, p1); c) and other methods known to the skilled artisan. Alternative synthesis methods may also be effective and known to the skilled artisan.

For example, an intermediate like A is alkylated with an alkylating agent B in the presence of a base (e.g. K2CO3, Cs2CO3 etc.). Hydrolysis in the presence of aqueous NaOH or LiOH gave the acid product.

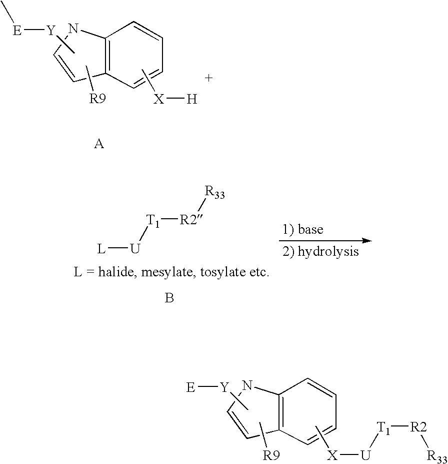

Alternatively, an intermediate like A is coupled with an alcohol C under Mitsunobu reaction condition (DEAD/PPh3, ADDP/Pbu3 etc.). Hydrolysis in the presence of aqueous NaOH or LiOH gave the acid product: 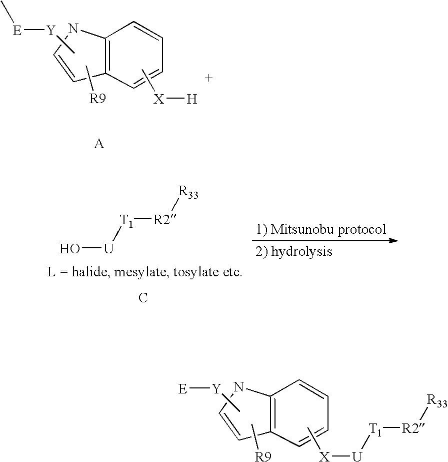
Intermediates A can be made by one of the following methods.
Hydroxy-indol-1-yl carboxylic acid analogs:
Hydroxy-indol-1-yl carboxylic acid analogs: 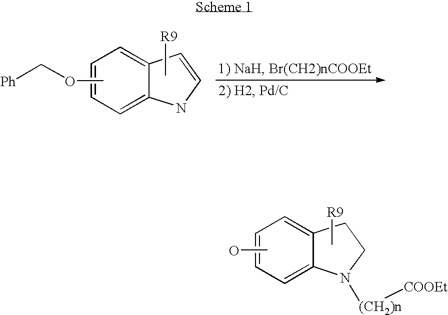
Mercaptyl-indol-1-yl carboxylic acid analogs: 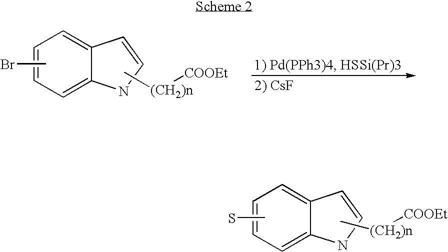
Hydroxy-indol-3-yl acetic acid analogs: 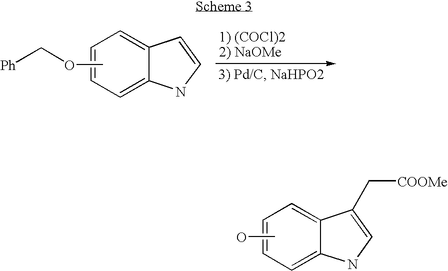
Thiophenol analogs: 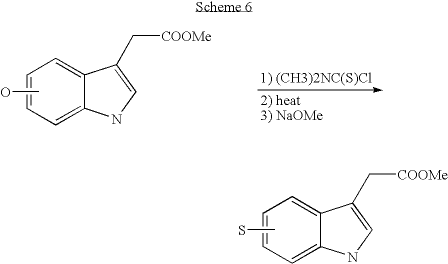
   Intermediates B, and C can be made in one of the following methods.
5-Hydroxymethyl thiazole: 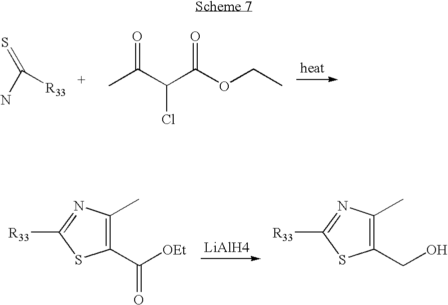
4-Hydroxyethyl triazole: 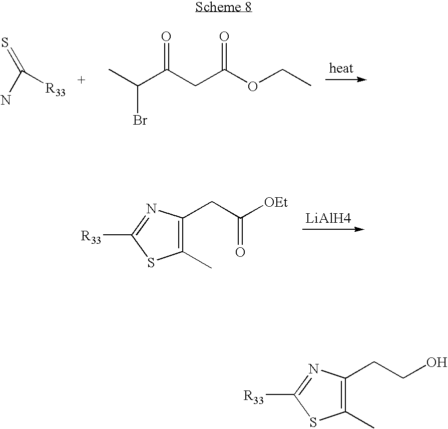
5-Hydroxyethyl oxazole: 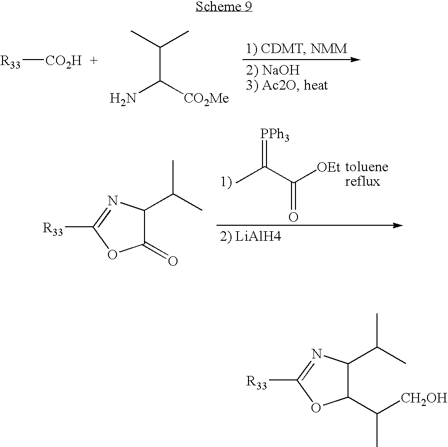 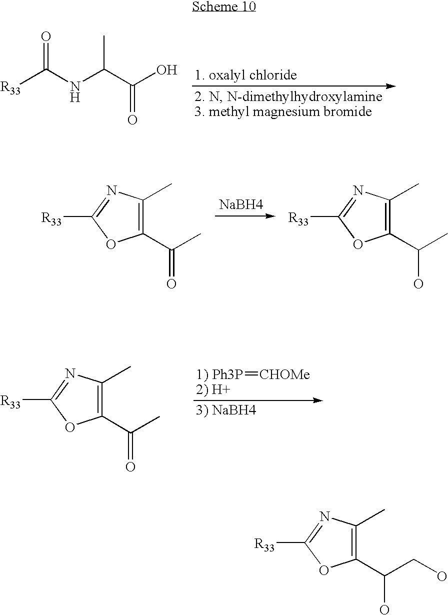
5-Hydroxymethyl oxazole: 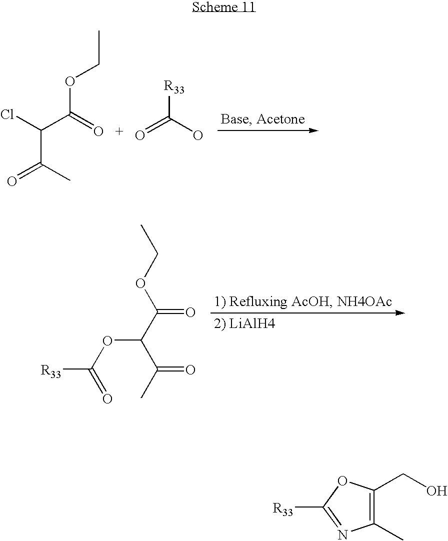
4-Hydroxylethyl oxazole:

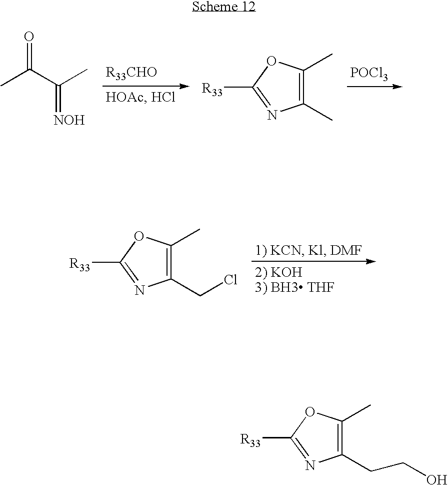

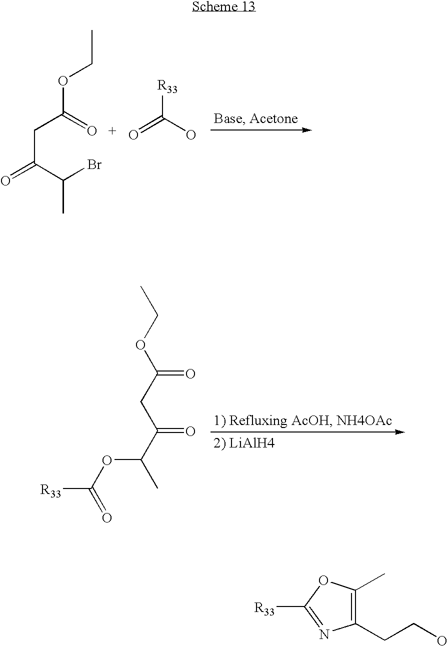

### EXEMPLIFICATION

The Examples provided herein are illustrative of the invention claimed herein and are not intended to limit the scope of the claimed invention in any way.

### Instrumental Analysis

Infrared spectra are recorded on a Perkin Elmer 781 spectrometer. ¹H NMR spectra are recorded on a Varian 400 MHz spectrometer at ambient temperature. Data are reported as follows: chemical shift in ppm from internal standard tetramethylsilane on the 6 scale, multiplicity (b=broad, s=singlet, d=doublet, t=triplet, q=quartet, qn=quintet and m=multiplet), integration, coupling constant (Hz) and assignment. ¹³C NMR are recorded on a Varian 400 MHz spectrometer at ambient temperature. Chemical shifts are reported in ppm from tetramethylsilane on the δ scale, with the solvent resonance employed as the internal standard (CDCl₃ at 77.0 ppm and DMSO-d₆ at 39.5 ppm). High resolution mass spectra are obtained on VG ZAB 3F or VG 70 SE spectrometers. Analytical thin layer chromatography is performed on EM Reagent 0.25 mm silica gel 60-F plates. Visualization is accomplished with UV light.

### Preparation 1

### (5-Hydroxy-indol-1-yl)-acetic acid ethyl ester

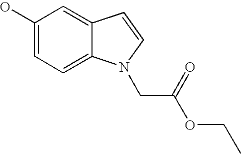

### Step A

5-benzyloxyindole (10.0 g, 44.79 mmol) is dissolved into anhydrous DMF (100 mL) and cooled to 0° C. in an ice water bath. Sodium hydride (2.6 g, 67.18 mmol) is dissolved into anhydrous DMF (100 mL), then slowly added to the indole solution using an addition funnel. The reaction is allowed to stir at 0° C. for 1 h., then the ice bath is removed and the solution is allowed to warm slowly to room temperature. The solution is then cooled back down to 0° C. and ethyl bromoacetate (11.2 g, 67.18 mmol) is then added in one portion. The reaction is allowed to stir at 0° C. for 1 h., then the ice bath is removed and the solution is allowed to warm slowly to room temperature. Upon completion, the reaction is quenched carefully using water, then diluted with EtOAc (300 mL). Brine (100 mL) is added and the two layers are seperated in a seperatory funnel. The organic layer is rinsed with water (2×75 mL) and then dried over anhydrous magnesium sulfate. The organic layer is then concentrated and purified using flash column chromatography (5% EtOAc/Hexanes) to yield 11.86 g (86%) of (5-Benzyloxy-indol-1-yl)-acetic acid ethyl ester.

### Step B

(5-Benzyloxy-indol-1-yl)-acetic acid ethyl ester (3.49 g, 11.31 mmol) is dissolved in EtOH (50 mL) and glacial acetic acid is added (2.0 mL). Palladium on carbon (20% by wt., 0.700 g) is then added to the homogenous solution, and a hydrogen filled balloon is connected to the round bottom flask. A vaccuum is created within the flask until the ethanol began to bubble, and the hydrogen allowed to enter the flask; this procedure is repeated three times, then the reaction is left to stir at room temperature overnight. Upon completion, the reaction is diluted with DCM (200 mL), and water (100 mL) is added. The mixture is filtered through a celite plug and the two phases are seperated. The organic layer is washed with brine (2×75 mL), then dried over anhydrous magnesium sulfate, and concentrated to yield the title compound (2.42 g) in 98% yield. The residual acetic acid is removed by flash column chromatography.

¹H NMR (DMSO-d₆, 400 MHz): δ 1.18 (t, J=7.34 Hz, 3H), 4.12 (q, J=6.85 Hz, 2H), 4.99 (s, 2H), 5.73 (s, 1H), 6.23 (d, J=2.94 Hz, 1H), 6.60 (dd, J₁=1.96 Hz, J₂=8.80 Hz, 1H), 6.84 (d, J=2.45 Hz, 1H), 7.12 (d, J=8.80 Hz, 1H), 7.19 (d, J=2.94 Hz, 2H); MS (ES, m/z):C₁₂H₁₃NO₃: 220.21 (M⁺+1), 218.7 (M⁺−1).

The following compounds are prepared in a manner substantially similar to that used to prepare the compound of preparation 1.

### Preparation 2

### 3-(5-Hydroxy-indol-1-yl)-propionic acid ethyl ester

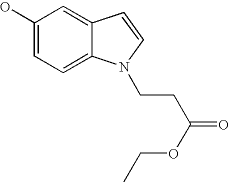

### Preparation 3

### 4-(5-Hydroxy-indol-1-yl)-butyric acid ethyl ester

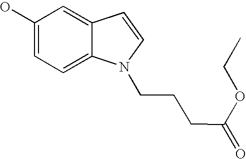

### Preparation 4

### 5-(5-Hydroxy-indol-1-yl)-pentanoic acid ethyl ester

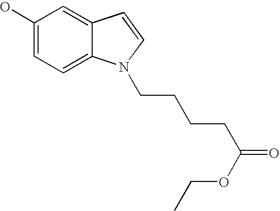

### Preparation 5

### 2-(5-Hydroxy-indol-1-yl)-propionic acid ethyl ester

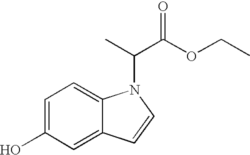

### Step A

### 2-(5-Benzyloxy-indol-1-yl)-propionic acid ethyl ester

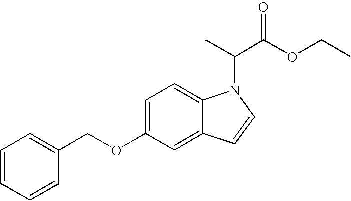

To a mixture of NaH (60%, 4.92 g, 0.205 mol) in DMF (60 mL) is added 5-benzoxyindole at 0˜5° C., then stirred 30 min. ethyl 2-bromopropionate is added dropwise, the mixture is allowed to warm to room temperature and heated at 70° C. overnight, cooled to room temperature, diluted with ethyl acetate, washed with water and brine, dried over sodium sulfate. Concentration and column chromatography on silica gel eluted with hexanes and ethyl acetate yields the title compound (29 g).

### Step B

### 2-(5-Hydroxy-indol-1-yl)-propionic acid ethyl ester

A mixture of 2-(5-Benzyloxy-indol-1-yl)-propionic acid ethyl ester (16 g) and Pd/C (5%, 1.93 g) in ethanol (190 mL) is stirred under 60 PSI of hydrogen overnight. Filtration and concentration yields the title compound.

### Preparation 6

### 2-(5-Hydroxy-indol-1-yl)-2-methyl-propionic acid ethyl ester

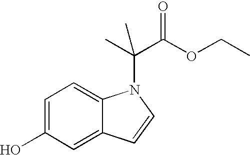

### Step A

### 2-(5-Benzyloxy-indol-1-yl)-2-methyl-propionic acid ethyl ester

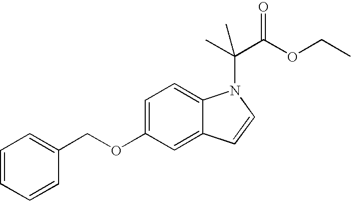

To a solution of 2-(5-benzyloxy-indol-1-yl)-propionic acid ethyl ester (20 g, 61.5 mmol) in THF (180 mL) is added LDA (2.0 M toluene, 37 mL) dropwise at −78° C. After the addition of LDA, the mixture is stirred for 30 min, then methyl iodide (8.77 g, 122.6 mmol) is added. The reaction mixture is allowed to warm to room temperature, after stirred for 2 hrs, quenched by water, extracted with ethyl acetate, dried over sodium sulfate. Concentration yields the title compound.

### Step B

### 2-(5-Hydroxy-indol-1-yl)-2-methyl-propionic acid ethyl ester

A mixture of 2-(5-benzyloxy-indol-1-yl)-2-methyl-propionic acid ethyl ester (15.6 g) and Pd/C (5%, 1.93 g) in ethanol (190 mL) is stirred under 60 PSI of hydrogen overnight. Filtration and concentration yields the title compound (11 g).

### Preparation 7

### (5-Hydroxy-2-methyl-indol-1-yl)-acetic acid ethyl ester

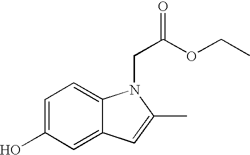

### Step A

### (5-Methoxy-2-methyl-indol-1-yl)-acetic acid ethyl ester

To a solution of 2-methyl-5-methoxylindole (5.10 g, 31.6 mmol) in DMF (200 mL) is added sodium hydride (60%, 1.9 g, 47.4 mmol) at 0˜5° C., stirred for 30 min, ethyl 2-bromoacetate (8.35 g, 50 mmol) is added. After 2 hr at room temperature, the reaction is quenched by water, extracted with ether. Combined organic layers are washed with water and brine, dried over sodium sulfate. Concentration yields the crude title product, which is used for next step without further purification.

### Step B

### (5-hydroxy-2-methyl-indol-1-yl)-acetic acid ethyl ester

To a solution of (5-methoxy-2-methyl-indol-1-yl)-acetic acid ethyl ester (0.87 g, 3.51 mmol) in methylene chloride (25 mL) is added BBr3 (1.0 mL, 10.5 mmol) at −20° C. After stirred at −20° C. for 2 hrs, the reaction mixture is poured into ice, extracted with methylene chloride, dried over sodium sulfate. Concentration yields the crude title compound, which is used for next step without further purification.

### Preparation 8

### {5-[3-(Toluene-4-sulfonyloxy)-propoxy]-indol-1-yl}-acetic acid ethyl ester

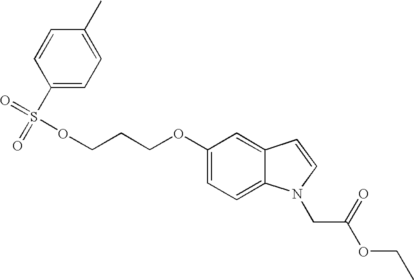

[5-(3-Hydroxy-propoxy)-indol-1-yl]-acetic acid ethyl ester (2.0 g, 6.55 mmol) is dissolved into anhydrous dichlormethane (DCM) (35 mL), then dimethylamino pridine (300 mg, 1.965 mmol), tosic anhydyhride (4.3 g, 13.1 mmol), and pyridine (2.3 mL, 23 mmol) are added. The reaction allowed to stir at room temperature under nitrogen. Upon completion, the reaction is then diluted with DCM (100 mL) and saturated sodium bicarbonate solution (50 mL) is added and the two layers are seperated in a seperatory funnel. The organic layer is rinsed with water (2×75 mL) and brine (2×50 mL), then dried over anhydrous magnesium sulfate. The organic layer is then concentrated and purified using flash column chromatography (5% EtOAc/Hexanes) to yield 1.92 g (64%) of the title compound.

The following compounds are prepared in a similar manner:

### Preparation 9

### 2-Methyl-2-{5-[3-toluene-4-sulfonyloxy)-propoxy]-indol-1-yl}-propionic acid ethyl ester

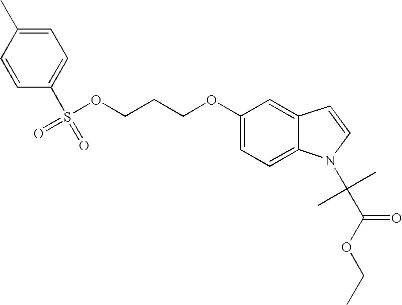

### Preparation 10

### {5-[2-(Toluene-4-sulfonyloxy)-ethoxy]-indol-1-yl}-acetic acid ethyl ester

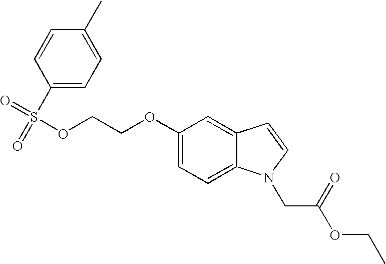

### Preparation 11

### (5-Mercapto-indol-1-yl)-acetic acid ethyl ester

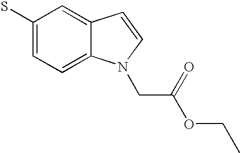

### Step A

(5-Bromo-indol-1-yl)-acetic acid ethyl ester (2.0 g, 7.09 mmol) is dissolved into anhydrous tetrahydrofuran (THF) (10 mL) and the reaction vessel purged with nitrogen a few times. Tetrakis triphenylphosphine palladium (175 mg, 0.15 mmol) is then added to the indole solution in one portion, purge again. Meanwhile, triisopropylsilylthiol (1.67 mL, 7.8 mmol) is dissolved in anhydrous THF (20 mL) and potassium hydride (0.483 mg, 7.8 mmol) is then slowly added. This mixture is heated to 50° C. for 4 h. After this solution has cooled to room temperature, it is transferred to the indole solution via kannula. This solution is the heated to 70° C. until the reaction is complete. Upon completion, the reaction is quenched carefully using water, then diluted with EtOAc (300 mL). Brine (100 mL) is added and the two layers are separated in a separatory funnel. The organic layer is rinsed with water (2×75 mL) and then dried over anhydrous magnesium sulfate. The organic layer is then concentrated and purified using flash column chromatography (5% EtOAc/Hexanes) to yield 1.3 g (50%) of (5-Triisopropylsilanylsulfanyl-indol-1-yl)-acetic acid ethyl ester.

### Step B

(5-Triisopropylsilanylsulfanyl-indol-1-yl)-acetic acid ethyl ester (60 mg, 0.1621 mmol) is dissolved in n-methylpyrrolidinone (NMP) (5 mL) and cesium fluoride (0.243 mmol) is added. The reaction is allowed to stir at room temperature until complete. This solution may be used in the coupling step (next) without further purification.

### Preparation 12

### 6-Hydroxy-1H-indol-3-yl)-acetic acid methyl ester

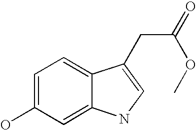

### Step A

### (6-Benzyloxy-1H-indol-3-yl)-oxo-acetic acid methyl ester

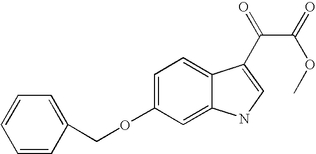

To a solution of 6-benzoxyindole (1.05 g, 4.7 mmol) in ether (8 mL) is added oxayl chloride (0.45 mL) at 0˜5° C., stirred for 2 hrs. The reaction mixture is cooled to −78° C., sodium methoxide (25% w/w in methanol, 2.4 mL) is added, warmed up to room temperature, quenched by water. Solid product is collected by filtration, washed by water and dried under vacuum.

### Step B

### (6-Hydroxy-1H-indol-3-yl)-acetic acid methyl ester

A mixture of (6-benzyloxy-1H-indol-3-yl)-oxo-acetic acid methyl ester (1.45 g, 4.7 mmol) and Pd/C (10%, 0.9 g) in dioxane (38 mL) is degassed and filled with nitrogen for three times, then a solution of NaH2PO2 (6 g) in water (5 mL) is added dropwise at 100° C. The reaction mixture is heated overnight, filtered through celite and concentrated. The residue is taken into ethyl acetate, washed with water and brine, dried over sodium sulfate. Concentration and column chromatography on silica gel yields the title compound (600 mg).

The following compounds are made in a similar manner:

### Preparation 13

### (6-Hydroxy-1-methyl-1H-indol-3-yl)-acetic acid methyl ester

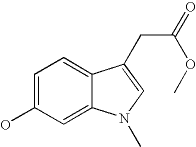

### Preparation 14

### (5-Hydroxy-1H-indol-3-yl)-acetic acid methyl ester

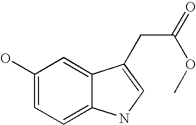

### Preparation 15

### (5-Hydroxy-1-methyl-1H-indol-3-yl)-acetic acid methyl ester

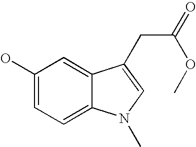

### Preparation 16

### (1-Ethyl-6-hydroxy-1H-indol-3-yl)-acetic acid ethyl ester

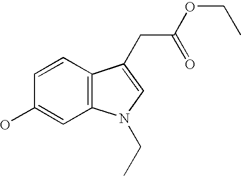

### Step A

To a solution of (6-Benzyloxy-1H-indol-3-yl)-oxo-acetic acid ethyl ester (3.0 g, 9.7 mmol) in anhydrous dimethyl formamide (50 mL) at 0° C. under nitrogen is added sodium hydride (0.600 g, 14.5 mmol) in small portions. The reaction is allowed to warm to room temperature slowly and monitored by TLC. Upon complete conversion, the reaction is cooled back down to 0° C. and ethyl bromide (1.5 mL, 20 mmol) is slowly added to the slurry. The reaction is allowed to warm slowly to room temperature and monitored by TLC. After complete consumption of the starting material, the reaction is quenched with water, then diluted with ethyl acetate, and the two phases are separated. The organic layer is washed, dried, filtered and concentrated. The crude (6-Benzyloxy-1-ethyl-1H-indol-3-yl)-oxo-acetic acid ethyl ester (0.843 g, 2.40 mmol), 25% yield, is further purified using flash column chromatography.

### Step B

(6-Benzyloxy-1-ethyl-1H-indol-3-yl)-oxo-acetic acid ethyl ester (0.843 g, 2.40 mmol) is dissolved in anhydrous dioxane (10 mL) then purged and back filled with nitrogen a few times. Palladium on carbon (10%) (0.200 g, 20% by wt.) is added and the reaction followed by heating to reflux. Slow addition of a saturated solution of sodium hypophosphite is initiated and the reaction is monitored by TLC. After the starting material is completely consumed, the reaction is allowed to cool to room temperature, diluted with dichloromethane and celite added. The mixture is filtered through a plug of celite and the two phases are separated. The organic layer is washed with water and brine, dried over sodium sulfate, then concentrated. The residue is further purified using flash column chromatography. The (1-Ethyl-6-hydroxy-1H-indol-3-yl)-acetic acid ethyl ester (0.564 g, 2.28 mmol) is formed in 95% yield.

The following compound is made in a similar manner:

### Preparation 17

### (1-propyl-6-hydroxy-1H-indol-3-yl)-acetic acid ethyl ester

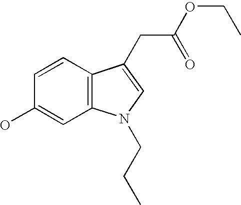

### Preparation 26

### 3-Oxo-5-phenyl-pentanoic acid ethyl ester

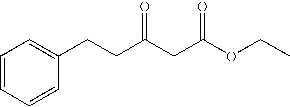

Ethyl acetoacetate (2.32 g, 20 mmol) is added to a pre-cold solution of LDA (2.0 M, 20 mL, 40 mmol) in THF (100 mL) at 0° C. After addition, the mixture is stirred for 30 min, then benzyl bromide (3.42 g, 20 mmol) is added dropwise. After stirred at 0° C. for 30 min, the reaction is quenched by 5 N HCl, extracted with ethyl ether. The combined organic layers are washed with water and brine until it is neutral. Concentration and column chromatography yields 1.6 g of the title compounds.

The following compounds are made in a similar manner:

### Preparation 27

### 5-(2-Chloro-6-fluoro-phenyl)-3-oxo-pentanoic acid ethyl ester

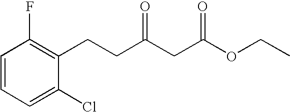

### Preparation 28

### 3-Oxo-hept-6-enoic acid ethyl ester

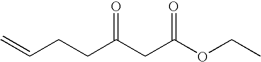

### Preparation 29

### 2-Chloro-3-Oxo-5-phenyl-pentanoic acid ethyl ester

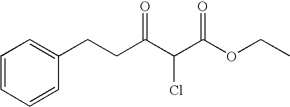

To a solution of 3-oxo-5-phenyl-pentanoic acid ethyl ester (1.6 g, 7.76 mmol) in methylene chloride (18 mL) is added sulfuryl chloride (1.15 g, 8.53 mmol) dropwise. After stirred at room temperature for 6 hours, the reaction mixture is poured into water, extracted with methylene chloride, washed wished water and brine, dried over sodium sulfate. Concentration yields the crude title compounds, which is used for the next step without further purification.

The following compounds are made in a similar manner:

### Preparation 30

### 5-(2-Chloro-6-fluoro-phenyl)-2-chloro-3-oxo-pentanoic acid ethyl ester

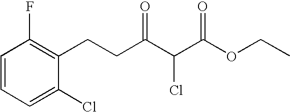

### Preparation 31

### 2-Chloro-3-oxo-hept-6-enoic acid ethyl ester

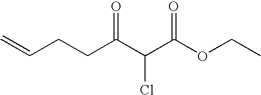

### Preparation 32

### 2-Chloro-3-oxo-heptanoic acid ethyl ester

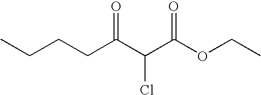

### Preparation 33

### 2-Chloro-3-oxo-hexanoic acid ethyl ester

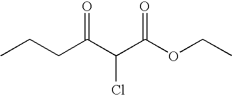

### Preparation 34

### 2-Chloro-4-methyl-3-oxo-pentanoic acid ethyl ester

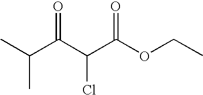

### Preparation 35

### 2-Chloro-4,4-dimethyl-3-oxo-pentanoic acid ethyl ester

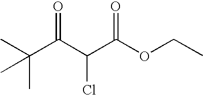

### Preparation 36

### 2-Chloro-3-oxo-3-phenyl-propionic acid ethyl ester

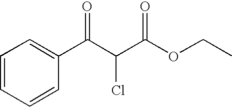

### Preparation 37

### 2-(4-Bromo-phenyl)₄-methyl-thiazole-5-carboxylic acid ethyl ester

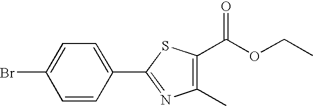

4-Bromo-thiobenzamide (5 g) in toluene is heated at reflux for 1 h in a flask equipped with a Dean-Stark trap. The dry 4-bromo-thioamide (3.4 g, 15 mmol) and ethyl 2-chloroacetoacetate (2.71 g, 16.4 mmol) are heated in ethanol (1000 mL) for overnight. The cooled reaction is concentrated and purified by short path chromatrography. The fractions that contained pure product are concentrated to yield 1.5 g (30.6%) ester as a solid.

Th following thiazoles are made in a similar manner:

### Preparation 38

### 4-Ethyl-2-(4-trifluoromethyl-phenyl)-thiazole-5-carboxylic acid ethyl ester

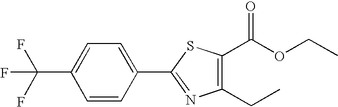

### Preparation 39

### 4-Isoproyl-2-(4-trifluoromethyl-phenyl)-thiazole-5-carboxylic acid ethyl ester

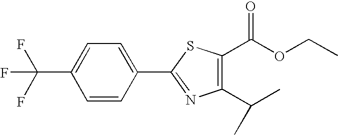

### Preparation 40

### 4-Propyl-2-(4-trifluoromethyl-phenyl)-thiazole-5-carboxylic acid ethyl ester

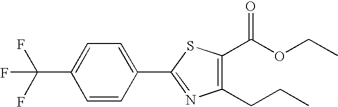

### Preparation 41

### 4-Butyl-2-(4-trifluoromethyl-phenyl)-thiazole-5-carboxylic acid ethyl ester

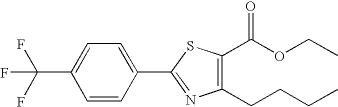

### Preparation 42

### 4-But-3-enyl-2-(4-trifluoromethyl-phenyl)-thiazole-5-carboxylic acid ethyl ester

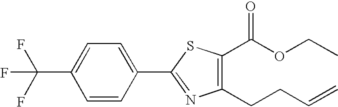

### Preparation 43

### 4-tert-Butyl-2-(4-trifluoromethyl-phenyl)-thiazole-5-carboxylic acid ethyl ester

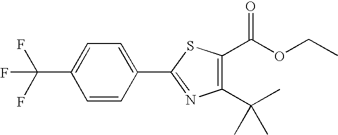

### Preparation 44

### 4-Phenethyl-2-(4-trifluoromethyl-phenyl)-thiazole-5-carboxylic acid ethyl ester

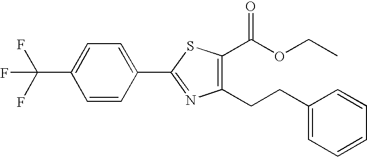

### Preparation 45

### 4-[2-(2-Chloro-6-fluoro-phenyl)-ethyl]-2-(4-trifluoromethyl-phenyl)-thiazole-5-carboxylic acid ethyl ester

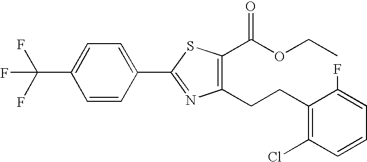

### Preparation 46

### 4-Phenyl-2-(4-trifluoromethyl-phenyl)-thiazole-5-carboxylic acid ethyl ester

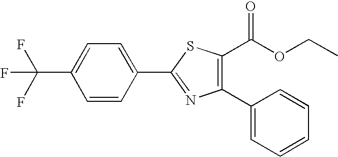

### Preparation 47

### 4-Phenoxymethyl-2-(4-trifluoromethyl-phenyl)-thiazole-5-carboxylic acid ethyl ester

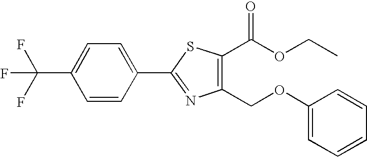

### Step A

### 4-Bromomethyl-2-(4-trifluoromethyl-phenyl)-thiazole-5-carboxylic acid ethyl ester

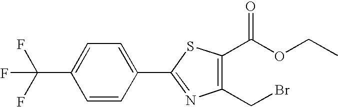

4-Methyl-2-(4-trifluoromethyl-phenyl)-thiazole-5-carboxylic acid ethyl ester (1.6 g, 5.00 mmol) is dissolved into chloroform (50 mL) then N-bromosuccinimide (1.0 g, 5.5 mmol) and 2,2′-azobisisobutyronitrile (0.412 g, 2.5 mmol) are added and the reaction is heated to reflux. The reaction is monitored by TLC until no starting material remained. The reaction is allowed to cool to room temperature, then diluted with more chloroform (100 mL). Water (50 mL) is added and the two phases are separated. The organic layer is washed with brine, then dried over anhydrous sodium sulfate. The material is then concentrated and further purified using flash column chromatography to yield 1.97 g or 99% yield.

### Step B

### 4-Phenoxymethyl-2-(4-trifluoromethyl-phenyl)-thiazole-5-carboxylic acid ethyl ester

Phenol (0.518 g, 5.5 mmol) is combined with anhydrous acetonitrile (20 mL) and cesium carbonate (2.3 g, 10 mmol) and allowed to stir at room temperature under nitrogen. To the reaction is added 4-bromomethyl-2-(4-trifluoromethyl-phenyl)-thiazole-5-carboxylic acid ethyl ester (1.97 g, 5.00 mmol). The reaction is monitored by TLC until all of the bromide is consumed. The reaction is diluted with ethyl ether (100 mL), then o.1N NaOH (50 mL) is added. The two phases are separated, then the organic layer is washed with water (50 mL) and brine (50 mL). The organic layer is dried over anhydrous sodium sulfate, then concentrated. The material is further purified using flash chromatography to yield 1.75 g or 86% yield of the product.

The following compounds are made in a similar manner:

### Preparation 48

### 4-(3,5-Bis-trifluoromethyl-phenoxymethyl)-2-(4-trifluoromethyl-phenyl)-thiazole-5-carboxylic acid ethyl ester

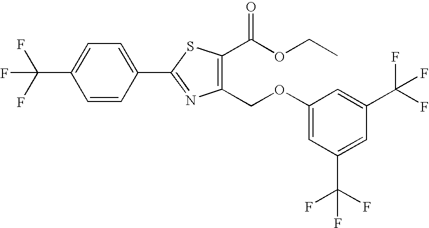

### Preparation 49

### 4-(2-Chloro-6-fluoro-phenoxymethyl)-2-(4-trifluoromethyl-phenyl)-thiazole-5-carboxylic acid ethyl ester

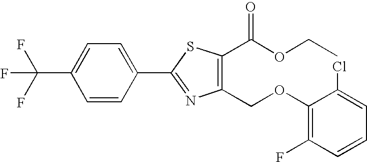

### Preparation 50

### 4-(4-Bromo-phenylsulfanylmethyl)-2-(4-trifluoromethyl-phenyl)-thiazole-5-carboxylic acid ethyl ester

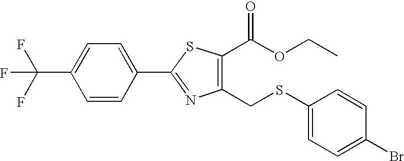

### Preparation 51

### 4-Phenylsulfanylmethyl-2-(4-trifluoromethyl-phenyl)-thiazole-5-carboxylic acid ethyl ester

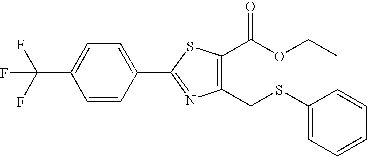

### Preparation 52

### [4-Methyl-2-(4-trifluoromethyl-phenyl)-thiazol-5-yl]-methanol

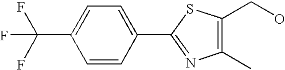

A THF (60 mL) solution of 4-methyl-2-(4-trifluoromethyl-phenyl)-thiazole-5-carboxylic acid ethyl ester (14.9 g, 47.3 mmol) is cooled to 0° C. and a 1M LiAlH₄ (47.3 mL, 47.3 mmol) is added slowly. The reaction is warmed to room temperature slowly, after stirring at room temperature for 2 h, tlc (15% EtOAc/hexane) showed that all the starting ester had been consumed. The reaction is cooled and carefully quenched with 2.4 mL water, 2.4 mL 5N NaOH and 7 mL water. The light tan solid is filter through celite and dried to give 7.70 g crude product. Recrystallization from methanol yields pure alcohol.

The following compounds are made in a similar manner:

### Preparation 53

### [4-Methyl-2-(4-bromo-phenyl)-thiazol-5-yl]-methanol

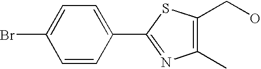

### Preparation 54

### [4-Ethyl-2-(4-trifluoromethyl-phenyl)-thiazol-5-yl]-methanol

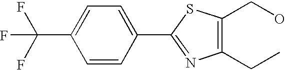

### Preparation 55

### [4-Isopropyl-2-(4-trifluoromethyl-phenyl)-thiazol-5-yl]-methanol

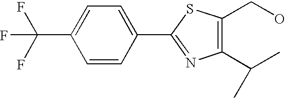

### Preparation 56

### [4-Propyl-2-(4-trifluoromethyl-phenyl)-thiazol-5-yl]-methanol

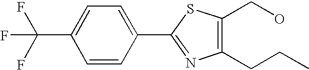

### Preparation 57

### [4-Butyl-2-(4-trifluoromethyl-phenyl)-thiazol-5-yl]-methanol

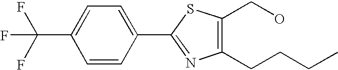

### Preparation 58

### [4-But-3-enyl-2-(4-trifluoromethyl-phenyl)-thiazol-5-yl]-methanol

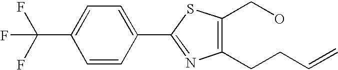

### Preparation 59

### [4-tert-Butyl-2-(4-trifluoromethyl-phenyl)-thiazol-5-yl]-methanol

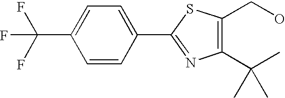

### Preparation 60

### 4-Phenethyl-2-(4-trifluoromethyl-phenyl)-thiazol-5-yl]-methanol

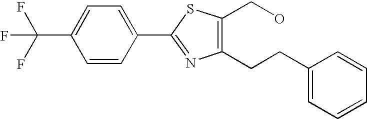

### Preparation 61

### 4-[2-(2-Chloro-6-fluoro-phenyl)-ethyl]-2-(4-trifluoromethyl-phenyl)-thiazol-5-yl]-methanol

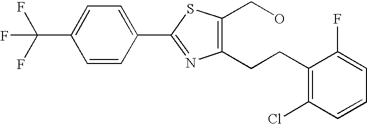

### Preparation 62

### [4-Phenyl-2-(4-trifluoromethyl-phenyl)-thiazol-5-yl]-methanol

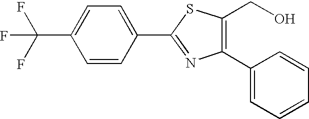

### Preparation 63

### [4-Phenoxymethyl-2-(4-trifluoromethyl-phenyl)-thiazol-5-yl]-methanol

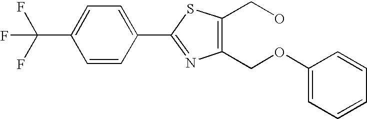

### Preparation 64

### [4-(2-Chloro-6-fluoro-phenoxymethyl)-2-(4-trifluoromethyl-phenyl)-thiazol-5-yl]-methanol

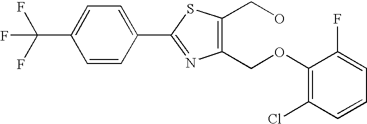

### Preparation 65

### [4-(3,5-Bis-trifluoromethyl-phenoxymethyl)-2-(4-trifluoromethyl-phenyl)-thiazol-5-yl]-methanol

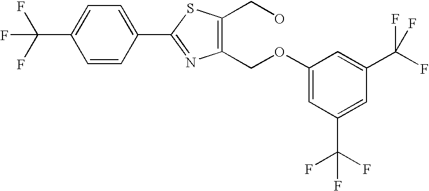

### Preparation 66

### [4-Phenylsulfanylmethyl-2-(4-trifluoromethyl-phenyl)-thiazol-5-yl]-methanol

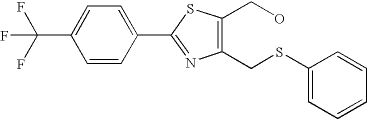

### Preparation 67

### [4-(4-Bromo-phenylsulfanylmethyl)-2-(4-trifluoromethyl-phenyl)-thiazol-5-yl]-methanol

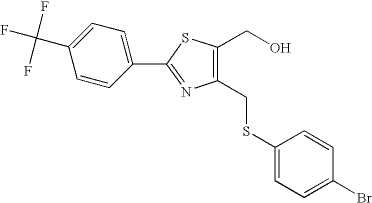

### Preparation 68

### 5-Chloromethyl-4-methyl-2-(4-trifluoromethyl-phenyl)-thiazole

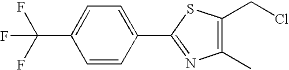

A solution of [4-methyl-2-(4-trifluoromethyl-phenyl)-thiazol-5-yl]-methanol (1.03 g, 3.75 mmol) and triethyl amine (1.05 mL, 7.5 mmol) in methylene chloride (15 mL) is cooled to 0° C., then MeSO2Cl is added dropwise. After 2 hrs, TLC indicated that the reaction is not complete, 10 mol % more of triethyl amine and MeSO₂Cl are added. After additional 2 hrs, the reaction mixture is diluted with methylene chloride and washed with sodium bicarbonate, water and brine, dried over sodium sulfate. Concentration yields the crude title compound, which is used for the next step without further purification.

The following compounds are made in a similar manner:

### Preparation 69

### 5-Chloromethyl-4-methyl-2-(4-bromophenyl)-thiazole

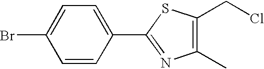

### Preparation 70

### 5-Chloromethyl-4-isopropyl-2-(4-trifluoromethyl-phenyl)-thiazole

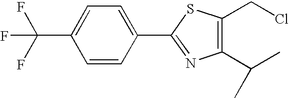

### Preparation 71

### 5-Chloromethyl-4-propyl-2-(4-trifluoromethyl-phenyl)-thiazole

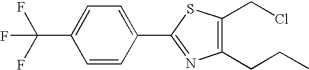

### Preparation 72

### 5-Chloromethyl-4-butyl-2-(4-trifluoromethyl-phenyl)-thiazole

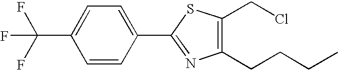

### Preparation 73

### 4-But-3-enyl-5-chloromethyl-2-(4-trifluoromethyl-phenyl)-thiazole

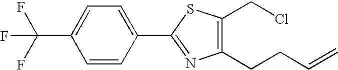

### Preparation 74

### 5-Chloromethyl-4-tert-butyl-2-(4-trifluoromethyl-phenyl)-thiazole

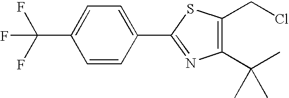

### Preparation 75

### 5-Chloromethyl-4-phenethyl-2-(4-trifluoromethyl-phenyl)-thiazole

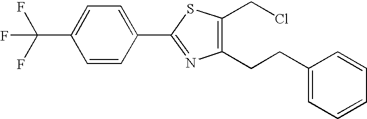

### Preparation 76

### 5-Chloromethyl-4-[2-(2-Chloro-6-fluoro-phenyl)-ethyl]-2-(4-trifluoromethyl-phenyl)-thiazole

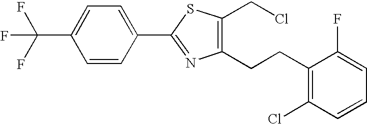

### Preparation 77

### 5-Chloromethyl-4-phenoxymethyl-2-(4-trifluoromethyl phenyl)-thiazole

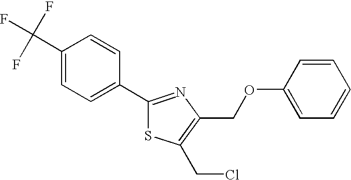

### Preparation 78

### 4-(2-Chloro-6-fluoro-phenoxymethyl)-5-chloromethyl-2-(4-trifluoromethyl-phenyl)-thiazole

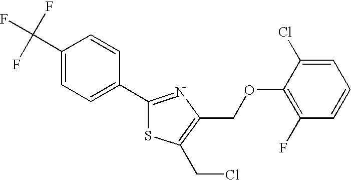

### Preparation 79

### 4-(3,5-Bis-trifluoromethyl-phenoxymethyl)-5-chloromethyl-2-(4-trifluoromethyl-phenyl)-thiazole

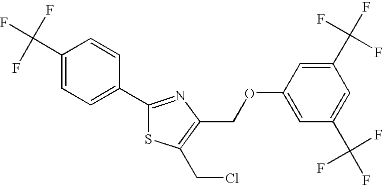

### Preparation 80

### 5-Chloromethyl-4-phenylsulfanylmethyl-2-(4-trifluoromethyl-phenyl)-thiazole

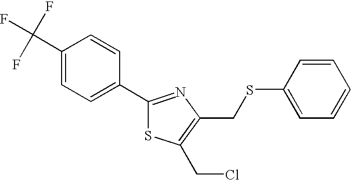

### Preparation 81

### 4-(4-Bromo-phenylsulfanylmethyl)-5-chloromethyl-2-(4-trifluoromethyl-phenyl)-thiazole

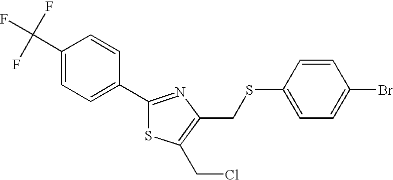

### Preparation 82

### 5-Chloromethyl-4-ethyl-2-(4-trifluoromethyl-phenyl)-thiazole

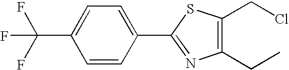

### Preparation 83

### 2-(3,5-Bis-trifluoromethyl-phenyl)-5-chloromethyl-4-methyl-thiazole

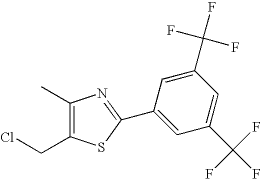

### Step A

To a solution of 2-Bromo-4-methyl-thiazole-5-carboxylic acid methyl ester (0.850 g, 3.39 mmol) in toluene:ethanol (1:1) (30 mL) at room temperature under nitrogen is added 3,5-bistrifluormethylbenzene boronic acid (1.0 g, 3.74 mmol). The reaction is purged of air and flushed with nitrogen a few times, followed by addition of tetrakis triphenylphosphine palladium (0.200 g, 0.17 mmol) and sodium carbonate (2.7 mL, 2.5M soln., 6.8 mmol). The reaction is purged again, then heated to reflux under nitrogen and monitored by TLC. After complete consumption of the starting material, the reaction is allowed to cool to room temperature, then diluted with ethyl acetate, celite added, filtered, and the two phases are separated. The organic layer is washed, dried, filtered and concentrated. The crude 2-(3,5-Bis-trifluoromethyl-phenyl)-4-methyl-thiazole-5-carboxylic acid methyl ester (0.545 g, 1.42 mmol), 42% yield, is further purified using flash column chromatography.

### Step B

2-(3,5-Bis-trifluoromethyl-phenyl)-4-methyl-thiazole-5-carboxylic acid methyl ester (0.545 g, 1.42 mmol), is dissolved in anhydrous tetrahydrofuran (6 mL) and cooled to 0° C. with stirring under nitrogen. Lithium aluminumhydride, 1.0M in THF, (1.40 mL, 1.40 mmol) is added and the reaction is monitored by TLC. After the starting material is completely consumed, the reaction is quenched with water, base, more water, and celite added, followed by dilution with ether. The mixture is filtered through a plug of celite and the two phases are separated. The organic layer is washed with water and brine, dried over sodium sulfate, then concentrated. The residue is further purified using flash column chromatography. The [2-(3,5-Bis-trifluoromethyl-phenyl)-4-methyl-thiazol-5-yl]-methanol (0.460 g, 1.35 mmol) is formed in 95% yield.

### Step C

[2-(3,5-Bis-trifluoromethyl-phenyl)-4-methyl-thiazol-5-yl]-methanol (0.460 g, 1.35 mmol), is dissolved in anhydrous dichloromethane (6 mL) and cooled to 0° C. with stirring under nitrogen. Triethyl amine (0.350 mL, 2.60 mmol) and methane sulfonyl chloride (0.200 mL, 2.0 mmol) are added and the reaction is monitored by TLC. After the starting material is completely consumed, the reaction is diluted with dichloromethane and extracted against saturated sodium bicarbonate solution. The two phases are separated and the organic layer is washed with water and brine, dried over sodium sulfate, then concentrated. The residue is further purified using flash column chromatography. The 2-(3,5-Bis-trifluoromethyl-phenyl)-5-chloromethyl-4-methyl-thiazole is formed quantitatively and used without further purification.

### Preparation 84

### 4-Ethyl-2-(4-trifluoromethyl-phenyl)-oxazole-5-carboxylic acid methyl ester

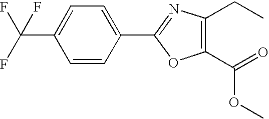

To a solution of 4-trifluoromethyl benzoic acid (0.100 g, 0.239 mmole) in methanol (2.0 mL), is added sodium hydroxide (0.093 g, 0.287 mmole) and stirred at room temperature for 2 hours. The mixture is concentrated to dryness in vacuo to give sodium 4-trifluoromethyl-benzoate as a white solid. It is then mixed with NH₄OAc (8.32 g, 107.9 mmole) in glacial acetic acid (500 mL) and heated at 100° C. for 16 hours. After removed the solvents on rota-vapor, the residue is partitioned between ethyl acetate (300 mL) and saturated sodium bicarbonate (300 mL). Extracted the aqueous layer with ethyl acetate (300 mL) one more time. The combined organic is wash with brine (3×500 mL), dried over Na₂SO₄, filtered and concentrated. The crude product is purified by chromatography on silica gel column, gradient elute with 0 to 10% ethyl acetate in hexane and concentrated to provide the titled compound as a white solid. Mass [EI+] 300 (M⁺+H).

### Preparation 85

### [4-Ethyl-2-(4-trifluoromethyl-phenyl)-oxazol-5-yl]-methanol

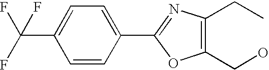

To a solution of 4-Ethyl-2-(4-trifluoromethyl-phenyl)-oxazole-5-carboxylic acid methyl ester (4.63 g, 15.47 mmole) in THF (100 mL), is added LiBH₄ in one portion at 0° C. The reaction is warmed up to room temperature and stirred for an hour. Additional LiBH₄ is added and the reaction is heated at 60° C. for 30 minutes. The excess amount of LiBH₄ is destroyed using 6N HCl (50 mL) dropwise at 0° C. The mixture is partitioned between ethyl acetate (300 mL) and brine (300 mL). The organic layer is washed with brine (3×300 mL), dried over Na₂SO₄, filtered and concentrated. The crude product is purified by flash chromatography, eluting with 60% ethyl acetate in hexane and concentrated to provide the titled compound as a white solid. Mass [EI+] 272 (M+H)⁺.

### Preparation 56

### [4-Methyl-2-(4-trifluoromethyl-phenyl)-oxazol-5-yl]-methanol

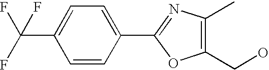

### Step 1

### 2-(4-tert-Butyl-benzoylamino)-propionic acid methyl ester

D,L Alanine methyl ester (18.5 g, 132 mmol), triethylamine (42 mL, 300 mmol) and dichloromethane (300 mL) are stirred in an ice/water bath. 4-(Trifluromethyl)benzoyl chloride (25 g, 120 mmol) is added dropwise and the resulting mixture is allowed to stir for 20 hr at room temperature. 500 mL water and 100 mL 1M hydrochloric acid are successively added. The organic layer is separated, washed with 250 mL each of saturated sodium hydrogen carbonate, water, and brine, dried over anhydrous magnesium sulfate, filtered, and concentrated to 100 mL volume. The mixture is diluted with 200 mL hexanes, cooled to 0° C. for 1 hr, and the white solid filtered and dried under vacuum to afford 2-(4-tert-Butyl-benzoylamino)-propionic acid methyl ester, 26.5 g, 80%. MS (ES): 276 (M⁺+1).

### Step 2

### 2-(4-tert-Butyl-benzoylamino)-propionic acid

A mixture of 2-(4-tert-Butyl-benzoylamino)-propionic acid methyl ester (26.3 g, 95.6 mmol), 200 mL 1M sodium hydroxide, and 100 mL tetrahydrofuran is stirred at room temperature 20 hr. The resulting clear solution is cooled on an ice/water bath and the pH is adjusted to 2 with concentrated hydrochloric acid. The product is extracted with three 250 mL portions of ethyl acetate. The combined extracts are washed with 100 mL each of water and brine, dried over anhydrous magnesium sulfate, filtered, and concentrated to afford 2-(4-tert-Butyl-benzoylamino)-propionic acid as a white solid, 24.6 g, 95%. MS M⁺+1 260.

### Step 3

### [4-Methyl-2-(4-trifluoromethyl-phenyl)-oxazol-5-yl]-methanol

To a solution of 2-(4-Trifluoromethyl-benzoylamino)-propionic acid (33.4 g, 128 mmol) is added oxalyl chloride (111 mL, 1.27 Mol) and 1 drop of DMF and the solution stirred overnight. The volatiles are removed in vacuo and toluene (20 mL) is added. The toluene is then removed in vacuo. To the resultant crude oil is dissolve in 50 mL methylene chloride, cooled to 0° C. and triethylamine (27 mL, 192 mmol) is added followed by methanol (50 mL). After 3 hrs the volatiles are removed in vacuo and the crude oil is purified by flash column chromatography (20%-50% ethyl acetate/hexanes) to provide 12.6 g (35%) of 4-Methyl-2-(4-trifluoromethyl-phenyl)-oxazole-5-carboxylic acid methyl ester. This ester (2.0 g, 7.0 mmol) is reduced to the alcohol by dissolution in THF (50 mL) and adding 4 eq. LiBH₄ (0.610 g, 28.0 mmol) to provide 1.8 g (100%) [4-Methyl-2-(4-trifluoromethyl-phenyl)-oxazol-5-yl]-methanol. MS M++1 258.

### Preparation 87

### 2-[5-Ethyl-2-(4-trifluoromethyl-phenyl)-thiazol-4-yl]-ethanol

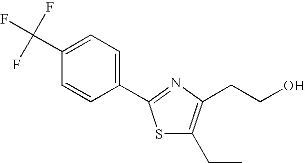

### Preparation 88

### 2-[5-Propyl-2-(4-trifluoromethyl-phenyl)-thiazol-4-yl]-ethanol

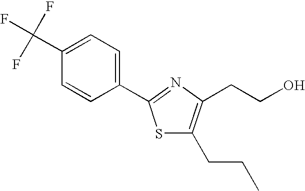

### Preparation 89

### Toluene-4-sulfonic acid 2-[5-propyl-2-(4-trifluoromethyl-phenyl)-thiazol-4-yl]-ethyl ester

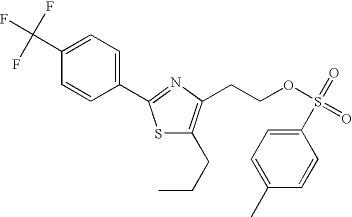

### Step A

3-Oxo-heptanoic acid methyl ester (25 g, 0.157 Mol) is dissolved into anhydrous dichloromethane (DCM) (200 mL) and then cooled to 0° C.-5° C. while stirring. A solution of bromine (25.25 g, 0.160 Mol) in DCM (50 mL) is added dropwise over 2 h. to the solution of the beta keto-ester. After the addition, the mixture is allowed to stir 0.5 h. at 0° C., then the ice bath is removed and the mixture is allowed to stir at room temperature for 18 h. TLC will show complete consumtion of starting material, then ice water (200 g) is added with stirring. The organic layer is collected and washed with cold water (2×) and brine. The filtered solution is dried over anhydrous sodium sulfate, then concentrated to a clear liquid. The crude 4-Bromo-3-oxo-heptanoic acid methyl ester (31.5 g, 0.135 Mol), 86% yield, is used without further purification.

### Step B

4-Bromo-3-oxo-heptanoic acid methyl ester (6.0 g, 25.0 mmol) is dissolved into denatured ethanol (100 mL) and para-trifluoromethyl thiobenzamide (5.0 g, 24.4 mmol) is added in one portion. The reaction is purged of air and flushed with nitrogen then heated to reflux. The reaction is monitored by TLC and HPLC and when complete, the reaction is allowed to cool to room temperature. The solvent is removed and the reaction is diluted with ethyl acetate (200 mL), followed by washes with saturated sodium bicarbonate solution, water, and brine. The ethyl acetate solution is dried over anhydrous sodium sulfate, then concentrated and further purified using flash column chromatography (10% EtOAc/Hexanes) to yield pure [[5-Propyl-2-(4-trifluoromethyl-phenyl)-thiazol-4-yl]-acetic acid methyl ester (8.66 g, 24.2 mmol) or 98% yield.

### Step C

[[5-Propyl-2-(4-trifluoromethyl-phenyl)-thiazol-4-yl]-acetic acid methyl ester (8.66 g, 24.2 mmol) is dissolved into anhydrous tetrahydrofuran (THF) (100 mL) and then cooled to 0° C. with stirring. Lithium aluminum hydride (24.2 mL, 1M in THF, 24.2 mmol) is slowly added by syringe and the reaction is monitored by TLC. Upon complete conversion, the reaction is carefully quenched using water, base, and water. Celite is added to the reaction, followed by diethyl ether and the mixture is then filtered through a celite plug. The two phases are then separated and the organic layer is washed using water and brine. The organic layer is the dried over anhydrous sodium sulfate and concentrated. The pure 2-[5-Propyl-2-(4-trifluoromethyl-phenyl)-thiazol-4-yl]-ethanol (5.739 g, 18.2 mmol) is obtained in 75% yield after flash column chromatography.

### Step D

The 2-[5-Propyl-2-(4-trifluoromethyl-phenyl)-thiazol-4-yl]-ethanol (5.739 g, 18.2 mmol) is dissolved into anhydrous dichloromethane (DCM) (100 mL) and dimethylamino pyridine (0.670 g, 5.46 mmol), tosic anhydride (11.9 g, 36.4 mmol), and pyridine (5 mL, 64 mmol) are added at room temperature. The reaction is monitored by TLC, and upon complete consumption of the starting alcohol, the reaction is diluted with DCM and extracted against saturated sodium bicarbonate solution. The organic layer is washed with water and brine, then dried over anhydrous sodium sulfate and concentrated. The pure Toluene-4-sulfonic acid 2-[5-propyl-2-(4-trifluoromethyl-phenyl)-thiazol-4-yl]-ethyl ester (4.46 g, 9.5 mmol) is obtained after flash column chromatography.

The following compounds are prepared in a similar manner:

### Preparation 90

### Toluene-4-sulfonic acid 2-[5-ethyl-2-(4-trifluoromethyl-phenyl)-thiazol-4-yl]-ethyl ester

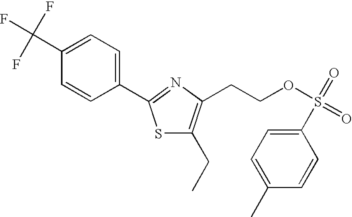

### Preparation 91

### Toluene-4-sulfonic acid 2-[2-(2-chloro-phenyl)-5-ethyl-thiazol-4-yl]-ethyl ester

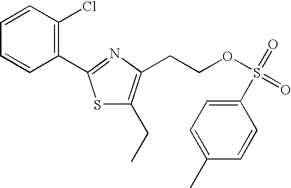

### Preparation 92

### 4-Isopropyl-2-(4-trifluoromethyl-phenyl)-thiazole-5-carboxylic acid

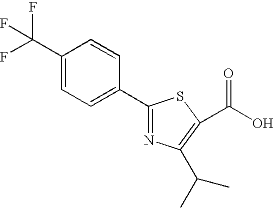

To a solution of 4-isopropyl-2-(4-trifluoromethyl-phenyl)-thiazole-5-carboxylic acid ethyl ester (0.62 g, 1.80 mmol) in THF (10 mL) is added 5M NaOH (3.5 mL, 17.50 mmol). The mixture is heated at 70° C. for 12 h. Upon cooling to RT, the mixture is acidified with 5M HCl and extracted with EtOAc. The organics are washed with water and brine, and dried with MgSO₄. After concentration, the title compound is obtained (0.46 g, 81%). The material is used without further purification.

The following compounds are made in a similar manner:

### Preparation 93

### [5-Ethyl-2-(4-trifluoromethyl-phenyl)-thiazol-4-yl]-acetic acid

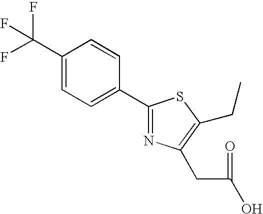

### Preparation of 94

### 1-[4-Methyl-2-(4-trifluoromethyl-phenyl)-thiazol-5-yl]-ethanol

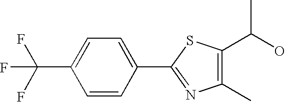

### Step A

### 4-Methyl-2-(4-trifluoromethyl-phenyl)-thiazole-5-carbaldehyde

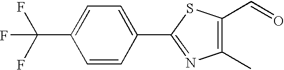

A mixture of [4-methyl-2-(4-trifluoromethyl-phenyl)-thiazol-5-yl]-methanol (5.0 g, 18.3 mmol) and MnO2 (2.4 g, 27.5 mmol) in chloroform (110 mL) are heated to reflux for 48 hrs, cooled to room temperature, filtered through celite. Concentration yields 5 gram of the title compound.

### Step B

### 1-[4-Methyl-2-(4-trifluoromethyl-phenyl)-thiazol-5-yl]-ethanol

To a solution of 4-methyl-2-(4-trifluoromethyl-phenyl)-thiazole-5-carbaldehyde (1.5 g, 5.53 mmole) in THF (50 mL) at 0° C., is added a solution of methyl magnesium bromide in diethyl ether (3.0M, 2.0 mL, 6.0 mmole) dropwise. The reaction is stirred for 5 minutes and then warmed up to room temperature for 2 hrs. The reaction is quenched with NH₄Cl_{(aq)} (10 mL), partitioned between ethyl ether (50 mL) and water (50 mL). Extracted the aqueous layer with ethyl ether (2×50 mL). The combined organic is washed with brine (3×100 mL), dried over Na₂SO₄, filtered and concentrated. Column chromatography on silica gel yields 1.35 gram of the title compound.

The following compounds are made in a similar manner:

### Preparation of 95

### 1-[4-isopropyl-2-(4-trifluoromethyl-phenyl)-thiazol-5-yl]-ethanol

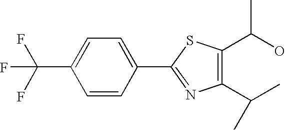

To a solution of 4-methyl-2-(4-trifluoromethyl-phenyl)-thiazole-5-carbaldehyde (0.5 g, 1.84 mmole) in THF (20 mL) at 0° C., is added a solution of benzyl magnesium chloride in tetrahedronfuran (2.0M, 1.0 mL, 2 mmole) dropwise. The reaction is stirred for 5 minutes and then warmed up to room temperature for 2 hrs. The reaction is quenched with NH₄Cl_{(aq)}, partitioned between ethyl ether and water. Extracted the aqueous layer with ethyl ether. The combined organic is washed with brine, dried over Na₂SO₄, filtered and concentrated. Column chromatography on silica gel yields the title compound.

### Preparation of 96

### 1-[4-Phenethyl-2-(4-trifluoromethyl-phenyl)-thiazol-5-yl]-ethanol

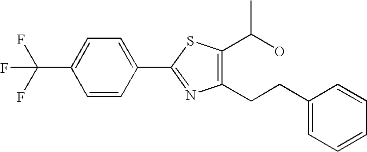

### Preparation of 97

### 1-[4-[2-(2-Chloro-6-fluoro-phenyl)-ethyl]-2-(4-trifluoromethyl-phenyl)-thiazol-5-yl]-ethanol

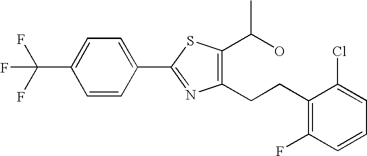

### Preparation of 98

### 1-[4-Phenylsulfanylmethyl-2-(4-trifluoromethyl-phenyl)-thiazol-5-yl]-ethanol

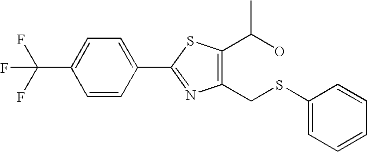

### Preparation of 99

### 1-[4-(2-Chloro-6-fluoro-phenoxymethyl)-2-(4-trifluoromethyl-phenyl)-thiazol-5-yl]-ethanol

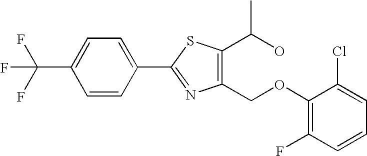

### Preparation of 100

### 1-[4-(3,5-Bis-trifluoromethyl-phenoxymethyl)-2-(4-trifluoromethyl-phenyl)-thiazol-5-yl]ethanol

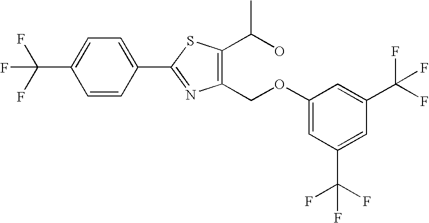

### Preparation 101

### 1-[4-Phenyl-2-(4-trifluoromethyl-phenyl)-thiazol-5-yl]-ethanol

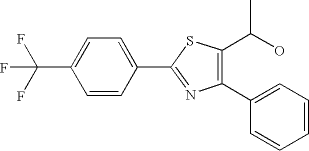

### Preparation 102

### 1-[4-Methyl-2-(4-trifluoromethyl-phenyl)-thiazol-5-yl]-2-phenyl-ethanol

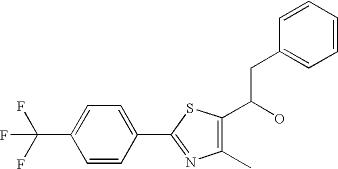

To a solution of 4-methyl-2-(4-trifluoromethyl-phenyl)-thiazole-5-carbaldehyde (0.5 g, 1.84 mmole) in THF (20 mL) at 0° C., is added a solution of benzyl magnesium chloride in tetrahedronfuran (2.0M, 1.0 mL, 2 mmole) dropwise. The reaction is stirred for 5 minutes and then warmed up to room temperature for 2 hrs. The reaction is quenched with NH₄Cl_{(aq)}, partitioned between ethyl ether and water. Extracted the aqueous layer with ethyl ether. The combined organic is washed with brine, dried over Na₂SO₄, filtered and concentrated. Column chromatography on silica gel yields the title compound.

### Preparation 103

### 1-[2-(4-Bromo-phenyl)₄-isopropyl-oxazol-5-yl]-ethanol

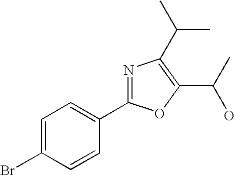

### Step A

### 2-(4-Bromo-phenyl)-4-isopropyl-oxazole-5-carboxylic acid ethyl ester

A solution of 4-bromo-benzoic acid (34.0 g, 0.169 mol) in DMF (450 mL) is treated at ambient temperature portionwise with NaH (6.4 g, 0.16 mol, 60% oil dispersion). The suspension is heated to 90° C. and 2-choro-4-methyl-3-oxo-pentanoic acid ethyl ester (27.7 g, 0.144 mol) is added neat. The remaining chloride is washed into the reaction flask using DMF (25 mL). The reaction mixture is stirred for 18 h, cooled, and treated with water (600 mL). The mixture is extracted with EtOAc (750 mL). The organic layer is washed with brine (2×250 mL), dried (Na₂SO₄), and concentrated to a foam (56 g). This diester is dissolved in acetic acid (500 mL), treated at ambient temperature with ammonium acetate (80 g, 1.0 mol), and heated at 120° C. for 20 h. The reaction mixture is cooled, concentrated, and partitioned between EtOAc (500 mL) and saturated NaHCO₃ solution (3×125 mL). The organic layer is dried (Na₂SO₄), and concentrated. The crude product is purified by silica gel flash chromatography using hexanes:ethyl acetate (6:1) to give the title compound (26.6 g, 55%).

### Step B

### [2-(4-Bromo-phenyl)₄-isopropyl-oxazol-5-yl]-methanol

A solution of 2-(4-bromo-phenyl)₄-isopropyl-oxazole-5-carboxylic acid ethyl ester (20.6 g, 60.9 mmol) in THF (300 mL) is cooled in an ice-water bath and treated portionwise with LiAlH₄ (2.8 g, 73 mmol). The reaction is complete after 1.5 h. Ice chips (˜10 g) are added to quench the excess hydride reagent, and anhydrous Na₂SO₄ (˜50 g) is added. The thick suspension is stirred 30 min, filtered through celite, and washed with THF (600 mL). The filtrate is dried (Na₂SO₄) and concentrated. The crude product is purified by silica gel flash chromatography using hexanes:ethyl acetate (3:1) to give a white solid (17.9 g, 99%).

### Step C

### 2-(4-Bromo-phenyl)-4-isopropyl-oxazole-5-carbaldehyde

A solution of [2-(4-bromo-phenyl)-4-isopropyl-oxazol-5-yl]-methanol (17.9 g, 60.4 mmol) in CH₂Cl₂ (450 mL) is treated at ambient temperature with acetic acid 1,1-diacetoxy-3-oxo-1□⁵-ioda-2-oxa-indan-1-yl ester (39 g, 92 mmol, Dess Martin reagent). The suspension is stirred 1 h and is partitioned between 10% aqueous Na₂S₂O₃ solution (250 mL) and CH₂Cl₂ (150 mL). The organic layer is washed with saturated NaHCO₃ (2×250 mL), and the combined aqueous layers are back-extracted with Et₂O (300 mL). The combined organic layers are dried (Na₂SO₄) and concentrated. The crude product is purified by silica gel flash chromatography using hexanes:ethyl acetate (6:1) to give an offwhite solid (14.4 g, 81%).

### Step D

### 1-[2-(4-Bromo-phenyl)-4-isopropyl-oxazol-5-yl]-ethanol

A solution of 2-(4-bromo-phenyl)₄-isopropyl-oxazole-5-carbaldehyde (14.4 g, 84.9 mmol) in THF (300 mL) is cooled to −78° C. and treated dropwise with methyl magnesium bromide (25 mL, 75 mmol, 3M Et₂O). After 1 h, more methyl magnesium bromide (12 mL, 36 mmol) is added. The reaction mixture is stirred 1.5 h, and saturated NH₄Cl solution (10 ml) is added dropwise. The mixture is partitioned between saturated NH₄Cl solution (10 ml), 1N HCl (25 mL), and Et₂O (300 mL). The organic layer is washed with brine (150 mL), dried (Na₂SO₄), and concentrated. The crude product is purified by silica gel flash chromatography using hexanes:ethyl acetate (9:1 to 5:1) to give an offwhite solid (9.5 g, 63%).

### Preparation 104

### 1-[2-(-bromo-phenyl)₄-isopropyl-oxazol-5-yl]-ethanol

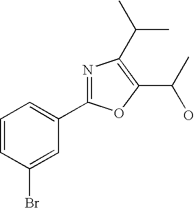

By the sequence above as preparation 78, 4-bromo-benzoic acid is converted to 1-[2-(4-bromo-phenyl)-4-isopropyl-oxazol-5-yl]-ethanol.
2-(3-Bromo-phenyl)-4-isopropyl-oxazole-5-carboxylic acid ethyl ester: 135 mmol scale, 35%
[2-(3-Bromo-phenyl)-4-isopropyl-oxazol-5-yl]-methanol: 45 mmol scale, 100%
2-(3-Bromo-phenyl)-4-isopropyl-oxazole-5-carbaldehyde: 45 mmol, 69%
1-[2-(3-Bromo-phenyl)-4-isopropyl-oxazol-5-yl]-ethanol: 29 mmol scale, 100%

### Preparation 105

### 1-[4-Methyl-2-(4-trifluoromethyl-phenyl)-oxazol-5-yl]-ethanol

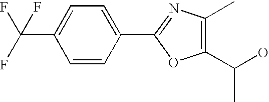

### Step 1

### 2-(4-tert-Butyl-benzoylamino)-propionic acid methyl ester

D,L Alanine methyl ester (18.5 g, 132 mmol), triethylamine (42 mL, 300 mmol) and dichloromethane (300 mL) are stirred in an ice/water bath. 4-(Trifluromethyl)benzoyl chloride (25 g, 120 mmol) is added dropwise and the resulting mixture is allowed to stir for 20 hr at room temperature. 500 mL water and 100 mL 1M hydrochloric acid are successively added. The organic layer is separated, washed with 250 mL each of saturated sodium hydrogen carbonate, water, and brine, dried over anhydrous magnesium sulfate, filtered, and concentrated to 100 mL volume. The mixture is diluted with 200 mL hexanes, cooled to 0° C. for 1 hr, and the white solid filtered and dried under vacuum to afford 2-(4-tert-Butyl-benzoylamino)-propionic acid methyl ester, 26.5 g, 80%. MS (ES): 276 (M⁺+1).

### Step 2

### 2-(4-tert-Butyl-benzoylamino)-propionic acid

A mixture of 2-(4-tert-Butyl-benzoylamino)-propionic acid methyl ester (26.3 g, 95.6 mmol), 200 mL 1M sodium hydroxide, and 100 mL tetrahydrofuran is stirred at room temperature 20 hr. The resulting clear solution is cooled on an ice/water bath and the pH is adjusted to 2 with concentrated hydrochloric acid. The product is extracted with three 250 mL portions of ethyl acetate. The combined extracts are washed with 100 mL each of water and brine, dried over anhydrous magnesium sulfate, filtered, and concentrated to afford 2-(4-tert-Butyl-benzoylamino)-propionic acid as a white solid, 24.6 g, 95%. MS M⁺+1 260.

### Step 3

### [4-Methyl-2-(4-trifluoromethyl-phenyl)-oxazol-5-yl]-methanol

To a solution of 2-(4-Trifluoromethyl-benzoylamino)-propionic acid (33.4 g, 128 mmol) is added oxalyl chloride (111 mL, 1.27 Mol) and 1 drop of DMF and the solution stirred overnight. The volatiles are removed in vacuo and toluene (20 mL) is added. The toluene is then removed in vacuo. To the resultant crude oil is dissolve in 50 mL methylene chloride, cooled to 0° C. and triethylamine (27 mL, 192 mmol) is added followed by methanol (50 mL). After 3 hrs the volatiles are removed in vacuo and the crude oil is purified by flash column chromatography (20%-50% ethyl acetate/hexanes) to provide 12.6 g (35%) of 4-Methyl-2-(4-trifluoromethyl-phenyl)-oxazole-5-carboxylic acid methyl ester. This ester (2.0 g, 7.0 mmol) is reduced to the alcohol by dissolution in THF (50 mL) and adding 4 eq. LiBH₄ (0.610 g, 28.0 mmol) to provide 1.8 g (100%) [4-Methyl-2-(4-trifluoromethyl-phenyl)-oxazol-5-yl]-methanol. MS M⁺+1 258.

### Step 4

### 4-Methyl-2-(4-trifluoromethyl-phenyl)-oxazole-5-carbaldehyde

[4-Methyl-2-(4-trifluoromethyl-phenyl)-oxazol-5-yl]-methanol (2.42 g, 9.41 mmol) and 100 mL dichloromethane are stirred at room temperature. Dess-Martin periodinane (8.0 g, 18.8 mmol) is added and the resulting mixture is stirred 4 hr at room temperature. The mixture is diluted with 100 mL saturated sodium hydrogen carbonate. The organic layer is separated, washed with 50 mL each of water and brine, dried over anhydrous magnesium sulfate, filtered, and concentrated. The crude product is purified by silica gel chromatography eluting with a mixture of 8:2 hexanes:ethyl acetate affording 4-methyl-2-(4-trifluoromethyl-phenyl)-oxazole-5-carbaldehyde as a white solid, 2.12 g, 89%. MS (M⁺+1) 256.

### Step 5

### 1-[4-Methyl-2-(4-trifluoromethyl-phenyl)-oxazol-5-yl]-ethanol

A solution of 4-methyl-2-(4-trifluoromethyl-phenyl)-oxazole-5-carbaldehyde (1.32 g, 5.16 mmol) and 50 mL tetrahydrofuran is stirred at 0° C. Methyl magnesium bromide (2.2 mL, 6.71 mmol, 3M) is added dropwise and the resulting mixture is allowed to stir at room temperature 30 min. The reaction is not complete, so an additional amound of methyl magnesium bromide (1 mL, 3 mmol) is added and the reaction stirred an additional 1 hr at room temperature. The mixture is cooled on an ice/water bath and aqueous ammonium chloride (10 mL) is added. The product is extracted with three 75 mL portions of ethyl acetate, the combined extracts are dried over anhydrous magnesium sulfate, filtered and concentrated. The crude product is purified by silica gel chromatography eluting with a mixture of 1:1 hexanes:ethyl acetate to afford 1-[4-Methyl-2-(4-trifluoromethyl-phenyl)-oxazol-5-yl]-ethanol as an ivory solid, 1.12 g, 80%. MS (M⁺+1) 272.

### Preparation 106

### 2-Methyl-1-[4-methyl-2-(4-trifluoromethyl-phenyl)-oxazol-5-yl]-propan-1-ol

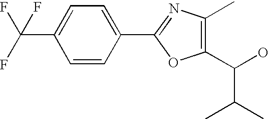

This compound is prepared in analogous fashion using preparation 105. Steps 1-4 are identical as previously described. Step 5 is performed using isoproyl magnesium bromide to afford 2-methyl-1-[4-methyl-2-(4-trifluoromethyl-phenyl)-oxazol-5-yl]-propan-1-ol.

### Preparation 107

### 1-[4-Isopropyl-2-(4-trifluoromethyl-phenyl)-oxazol-5-yl]-ethanol

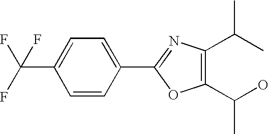

### Step 1

### 2-chloro-4-methyl-3-oxo-pentanoic acid ethyl ester

Ethyl isobutyryl acetate (12.0 g, 75.85 mmol) is stirred at 0° C. in dichloromethane (75 mL). Sulfuryl chloride (6.5 mL, 80 mmol) is added dropwise and the resulting mixture is allowed to stir 20 hr at room temperature. The reaction mixture is cooled to 0° C. and aqueous saturated sodium hydrogen carbonate (200 mL) is added cautiously. The layers are separated, the aqueous layer is washed with dichloromethane (100 mL), the combined organic layers are washed with water and brine (100 mL each), dried over anhydrous magnesium sulfate, filtered, and concentrated to constant weight to give 2-chloro-4-methyl-3-oxo-pentanoic acid ethyl ester as a colorless oil, 14.6 g, 100%. MS (M⁺+1) 193.

### Step 2

4-Isopropyl-2-(4-trifluoromethyl-phenyl)-oxazole-5-carboxylic acid ethyl ester Sodium Hydride, 60% mineral oil (1.9 g, 46.3 mmol) and dimethylformamide (50 mL) are stirred at room tmperature and 4-(trifluoromethyl)benzoic acid (8.0 g, 42.1 mmol) is added. To the resulting slurry is added 2-chloro-4-methyl-3-oxo-pentanoic acid ethyl ester (8.5 g, 44.2 mmol) and the resulting mixture is heated to 90° C. for 3 hr. The reaction mixture is cooled, diluted with water (100 mL), and product is extracted with ethyl acetate (100 mL). The organic layer is washed with water (three 100 mL portions) and brine (100 mL), dried over anhydrous magnesium sulfate, filtered, and concentrated to constant weight to give 4-trifluoromethyl-benzoic acid 1-ethoxycarbonyl-3-methyl-2-oxo-butyl ester as a colorless oil, 14.6 g, 100%. The resulting oil is stirred in a mixture of acetic acid (100 mL) and ammonium acetate (9.75 g, 126.5 mmol) at reflux 1 hr, then 20 hr at room temperature. The solvent is removed in vacuo and the residue is partioned between aqueous saturated sodium hydrogen carbonate (100 mL) and ethyl acetate (100 mL. The layers are separated, the aqueous layer is washed with ethyl acetate (100 mL). The organic extracts are combined, washed with water and brine (100 mL each) dried over anhydrous magnesium sulfate, filtered, and concentrated. The residue is purified over silica eluting with 9:1 hexanes:ethyl acetate to afford 4-Isopropyl-2-(4-trifluoromethyl-phenyl)-oxazole-5-carboxylic acid ethyl ester as a white solid, 8.1 g, 60%. MS (M⁺+1) 328.

### Step 3

### [4-Isopropyl-2-(4-trifluoromethyl-phenyl)-oxazol-5-yl]-methanol

4-Isopropyl-2-(4-trifluoromethyl-phenyl)-oxazole-5-carboxylic acid ethyl ester (0.53 g, 1.62 mmol) is stirred in tetrahydrofuran (25 mL) at 0° C. Lithium aluminum hydride (0.122 g, 3.23 mmol) is added and the mixture is stirred 18 hr at room temperature. The mixture is diluted carefully with 1M aqueous hydrochloric acid (10 mL), and the product is extracted with ethyl acetate (three 75 mL portions). The extracts are combined, dried over anhydrous magnesium sulfate, filtered, and concentrated to afford [4-Isopropyl-2-(4-trifluoromethyl-phenyl)-oxazol-5-yl]-methanol as a white solid, 0.46 g, 100%. MS (M⁺+1) 286

### Step 4

### 4-Isopropyl-2-(4-trifluoromethyl-phenyl)-oxazole-5-carbaldehyde

[4-Isopropyl-2-(4-trifluoromethyl-phenyl)-oxazol-5-yl]-methanol (0.46 g, 1.612 mmol), Dess-Martin periodinane (1.36 g, 3.22 mmol) and dichloromethane (25 mL) are stirred 1 hr at room temperature. The mixture is diluted with aqueous saturated sodium hydrogen carbonate (100 mL) and dichloromethane (100 mL). The layers are separated, the aqueous layer is washed with dichloromethane (100 mL). The organic washes are combined, washed with brine (50 mL), dried over anhydrous magnesium sulfate, filtered, and concentrated in vacuo. The product is purified over silica eluting with 3:1 hexanes:ethyl acetate to afford 4-Isopropyl-2-(4-trifluoromethyl-phenyl)-oxazole-5-carbaldehyde as a white solid, 0.41 g, 90%. %. MS (M⁺+1) 284.

### Step 5

### 1-[4-Isopropyl-2-(4-trifluoromethyl-phenyl)-oxazol-5-yl]-ethanol

Followed a similar procedure in step 5 of preparation 105.

### Preparation 108

### 1-[4-tert-Butyl-2-(4-trifluoromethyl-phenyl)-oxazol-5-yl]-ethanol

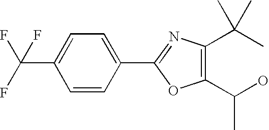

### Preparation 109

### 1-[4-Methyl-2-(4-trifluoromethyl-phenyl)-oxazol-5-yl]-propan-1-ol

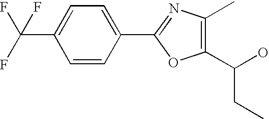

### Step 1

### 4-methyl-2-(4-trifluoromethyl-phenyl)-oxazole-5-carboxylic acid methoxy-methyl-amide

To a solution of 2-(4-Trifluoromethyl-benzoylamino)-propionic acid (5.0 g, 19.14 mmol) is added oxalyl chloride (16.7 mL, 191.4 mmol) and 2 drops of DMF and the solution stirred overnight. The volatiles are removed in vacuo and toluene (20 mL) is added. The toluene is then removed in vacuo. To the resultant crude oil is dissolve in dichloromethane (100 mL), cooled to 0° C. and triethylamine (13.4 mL, 96 mmol) is added followed by N,O-dimethyl hydroxylamine hydrochloride (9.4 g, 96 mmol). After 1 hr the mixture is partioned between 1M aqueous hydrochloric acid and ethyl acetate. The organic layer is dried over anhydrous magnesium sulfate, filtered, concentrated in vacuo. The product is purified over silica eluting with 8:2 hexanes:ethyl acetate to afford 4-methyl-2-(4-trifluoromethyl-phenyl)-oxazole-5-carboxylic acid methoxy-methyl-amide as a white crystalline solid, 2.4 g, 40%. MS (M⁺+1) 315.

### Step 2

### 1-[4-methyl-2-(4-trifluoromethyl-phenyl)-oxazol-5-yl]-propan-1-one

4-methyl-2-(4-trifluoromethyl-phenyl)-oxazole-5-carboxylic acid methoxy-methyl-amide (1.0 g, 3.18 mmol) is stirred in tetrahydrofuran (15 mL) at −78° C. Ethyl magnesium bromide, 3M/ether (2.1 mL, 4.14 mmol) is added and the mixture is warmed to room temperature. The mixture is diluted with aqueous saturated ammonium chloride and washed with ethyl acetate (three 50 mL portions). The combined washes are dried over anhydrous magnesium sulfate, filtered and concentrated in vacuo. The residue is purified over silica eluting with 7:3 hexanes:ethyl acetate to afford 1-[4-methyl-2-(4-trifluoromethyl-phenyl)-oxazol-5-yl]-propan-1-one as an ivory solid, 0.70 g, 78%. MS (M⁺+1) 284.

### Step 3

### 1-[4-methyl-2-(4-trifluoromethyl-phenyl)-oxazol-5-yl]-propan-1-ol

1-[4-methyl-2-(4-trifluoromethyl-phenyl)-oxazol-5-yl]-propan-1-one (0.68 g, 2.4 mmol) and tetrahydrofuran (5 mL) are stirred at 0° C. Lithium borohydride (0.14 g, 6.36 mmol) is added and the mixture is stirred 10 min at 0° C., and 30 min at room temperature. The mixture is diluted with 1M aqueous hydrochloric acid and washed with ethyl acetate (three 50 mL portions). The organic washes are dried over anhydrous magnesium sulfate, filtered, and concentrated in vacuo. The residue is purified over silica eluting with 8:2 hexanes:ethyl acetate to 1:1 hexanes ethyl acetate to afford 1-[4-methyl-2-(4-trifluoromethyl-phenyl)-oxazol-5-yl]-propan-1-ol as ivory solid, 0.69 g, 100%. MS (M⁺+1) 286.

### Preparation 110

### 1-[4-Methyl-2-(4-trifluoromethyl-phenyl)-oxazol-5-yl]-but-3-en-1-ol

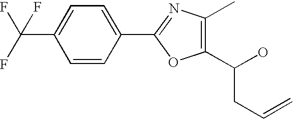

### Preparation 111

### 1-[4-Methyl-2-(4-trifluoromethyl-phenyl)-oxazol-5-yl]-pentan-1-ol

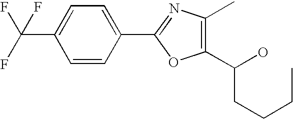

### Preparation 112

### 1-[4-Isopropyl-2-(4-trifluoromethoxy-phenyl)-oxazol-5-yl]-ethanol

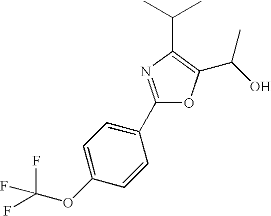

### Preparation 113

### 1-[2-(4-Bromo-phenyl)-4-isopropyl-oxazol-5-yl]-ethanol

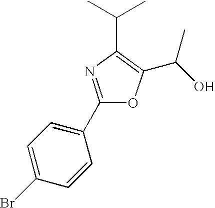

### Preparation 114

### 2-[4-Isopropyl-2-(4-trifluoromethyl-phenyl)-oxazol-5-yl]-propan-1-ol

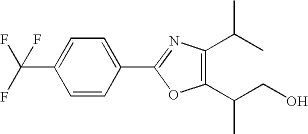

### Step 1

### 4-Isopropyl-2-(4-trifluoromethyl-phenyl)-4H-oxazol-5-one

3-Methyl-2-(4-trifluoromethyl-benzoylamino)-butyric acid methyl ester (4.75 g, 16.42 mmol) is dissolved in acetic anhydride (25 mL) and heated to 95° C. for 3 hr. The mixture is concentrated in vacuo and the residue is partitioned between aqueous saturated sodium hydrogen carbonate (100 mL) and ethyl acetate (100 mL) the layers are separated, the organic phase is washed with water and brine (100 mL each), dried over anhydrous magnesium sulfate, filtered, concentrated in vacuo, and purified over silica gel eluting with 9:1 hexanes:ethyl acetate to afford 4-Isopropyl-2-(4-trifluoromethyl-phenyl)-4H-oxazol-5-one as a colorless oil which solidifies to a white crystalline solid on standing, 4.14 g, 93%. MS (M⁺+1) 272.

### Step 2

### 2-[4-Isopropyl-2-(4-trifluoromethyl-phenyl)-oxazol-5-yl]-propionic acid ethyl ester

4-Isopropyl-2-(4-trifluoromethyl-phenyl)-4H-oxazol-5-one (1.0 g, 3.69 mmol) and (carbethoxyethylidine)triphenyl-phosphorane (2.67 g, 7.37 mmol) are stirred in toluene (20 mL) at reflux 3 hr. The mixture is concentrated in vacuo and the residue is purified over silica eluting with 9:1 hexanes:ethyl acetate affording 2-[4-Isopropyl-2-(4-trifluoromethyl-phenyl)-oxazol-5-yl]-propionic acid ethyl ester as a pale orange oil, 1.11 g, 85%. MS (M⁺+1) 356.

### Step 3

### 2-[4-Isopropyl-2-(4-trifluoromethyl-phenyl)-oxazol-5-yl]-propan-1-ol

2-[4-Isopropyl-2-(4-trifluoromethyl-phenyl)-oxazol-5-yl]-propionic acid ethyl ester (1.11 g, 3.12 mmol) and tetrahydrofuran (50 mL) are cooled to 0° C. Lithium aluminum hydride (0.24 g, 6.25 mmol) is added and the resulting mixture is stirred 20 hr at room temperature. The mixture is cooled to 0° C. and 1M aqueous hydrochloric acid (50 mL) is carefully added. The mixture is then diluted with ethyl acetate (100 mL) and the layers are separated. The aqueous layer is washed with ethyl acetate (100 mL) and the organic washes are combined, washed with water and brine (50 mL each), dried over anhydrous magnesium sulfate, filtered and concentrated to constant weight to give 2-[4-Isopropyl-2-(4-trifluoromethyl-phenyl)-oxazol-5-yl]-propan-1-ol as a pale orange oil, 1.03 g, 100%. MS (M⁺+1) 314. The following compound is made in similar manner:

### Preparation 115

### 2-[4-Isopropyl-2-(4-trifluoromethoxy-phenyl)-oxazol-5-yl]-propan-1-ol

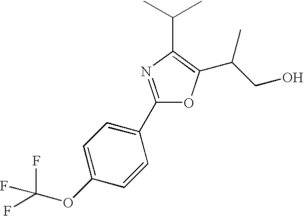

### Preparation 116

### 2-[4-Isopropyl-2-(4-phenoxy-phenyl)-oxazol-5-yl]-propan-1-ol

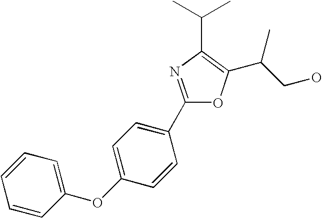

### Preparation 117

### 2-[2-(4-Bromo-phenyl)-4-isopropyl-oxazol-5-yl]-propan-1-ol

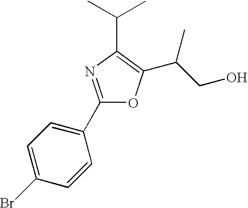

### Preparation 118

### 2-[5-Methyl-2-(4-trifluoromethyl-phenyl)-thiazol-4-yl]-propan-1-ol and 2-Methyl-2-[5-methyl-2-(4-trifluoromethyl-phenyl)-thiazol-4-yl]-propan-1-ol

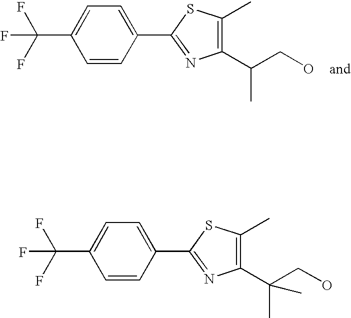

### Step A

### 4-Bromo-2-methyl-3-oxo-pentanoic acid methyl ester and 4-Bromo-2,2-dimethyl-3-oxo-pentanoic acid methyl ester

A solution of bromine (18.4 g, 115 mmol) in chloroform (30 mL) is added to a mixture of 2-methyl-3-oxo-pentanoic acid methyl ester and 2,2-dimethyl-3-oxo-pentanoic acid methyl ester (16.5 g) in chloroform (120 mL) at 0-5° C. dropwise. After the addition of bromine, the mixture is allowed to warm up to room temperature slowly and stirred overnight. The reaction is then quenched by ice water, the layers are separated. The organic layer is washed with cold water and brine, dried over sodium sulfate. Concentration yields the title compounds, which is used for next step without further purification.

### Step B

### 2-[5-Methyl-2-(4-trifluoromethyl-phenyl)-thiazol-4-yl]-propionic acid methyl ester and 2-Methyl-2-[5-methyl-2-(4-trifluoromethyl-phenyl)-thiazol-4-yl]-propionic acid methyl ester

A mixture of 4-Trifluoromethyl-thiobenzamide (7.70 g, 37.5 mmol) and the crude product from step A (9.0 g, 40 mmol) in ethanol (500 mL) is heated to reflux for 4 days. Solvent is evaporated and the residue is purified by chromatography on silica gel yielding the title compounds (11 g).

### Step C

### 2-[5-Methyl-2-(4-trifluoromethyl-phenyl)-thiazol-4-yl]-propan-1-ol and 2-Methyl-2-[5-methyl-2-(4-trifluoromethyl-phenyl)-thiazol-4-yl]-propan-1-ol

To a solution of 2-[5-Methyl-2-(4-trifluoromethyl-phenyl)-thiazol-4-yl]-propionic acid methyl ester and 2-Methyl-2-[5-methyl-2-(4-trifluoromethyl-phenyl)-thiazol-4-yl]-propionic acid methyl ester (10.6 g) in THF (50 mL) is added a solution of lithium aluminum hydride in THF (1.0 M, 33 mL) at 0° C. After 2 hrs, the reaction is quenched by water and sodium hydroxide, filtered, concentrated. Chromatography on silica gel yields 2-[5-Methyl-2-(4-trifluoromethyl-phenyl)-thiazol-4-yl]-propan-1-ol (4.3 g) and 2-Methyl-2-[5-methyl-2-(4-trifluoromethyl-phenyl)-thiazol-4-yl]-propan-1-ol (2.6 g).

### Preparation 119

### 2-[5-Methyl-2-(4-trifluoromethyl-phenyl)-oxazol-4-yl]-propan-1-ol and 2-Methyl-2-[5-methyl-2-(4-trifluoromethyl-phenyl)-oxazol-4-yl]-propan-1-ol

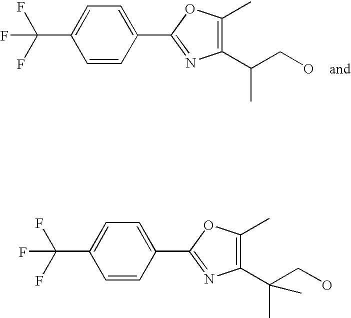

### Step A

### 4-Bromo-2-methyl-3-oxo-pentanoic acid methyl ester and 4-Bromo-2,2-dimethyl-3-oxo-pentanoic acid methyl ester

A solution of bromine (18.4 g, 115 mmol) in chloroform (20 mL) is added to a mixture of 2-methyl-3-oxo-pentanoic acid methyl ester and 2,2-dimethyl-3-oxo-pentanoic acid methyl ester (ca. 115 mmol) in chloroform (120 mL) at 0-5° C. dropwise. After the addition of bromine, the mixture is allowed to warm up to room temperature slowly and stirred overnight. The reaction is then quenched by ice water, the layers are separated. The organic layer is washed with cold water and brine, dried over sodium sulfate. Concentration yields the title compounds, which is used for next step without further purification.

### Step B

### 2-[5-Methyl-2-(4-trifluoromethyl-phenyl)-oxazol-4-yl]-propionic acid methyl ester and 2-Methyl-2-[5-methyl-2-(4-trifluoromethyl-phenyl)-oxazol-4-yl]-propionic acid methyl ester

To a solution of 4-Trifluoromethyl-benzoic acid (7.6 g, 40 mmol) in methanol (100 mL) is added sodium hydroxide (1.6 g, 40 mmol), stirred for 30 min, methanol is evaporated. The residue is taken into DMF (50 mL) and the crude product from step A (10 g) is added. The mixture is stirred overnight, diluted with ethyl acetate, washed with water and brine, dried over sodium sulfate, and concentrated. The residue is taken into ethanol (150 mL) and treated with ammonium acetate (6.17 g) and heated at 70° C. for 12 hrs. Ethanol is evaporated, the residue is mixed with ammonium acetate (12.3 g) in glacial acid (750 mL) and heated at 100° C. for 2 days. Solvent is evaporated and the residue is taken into ethyl acetate, washed with water and brine, dried. Chromatography on silica gel yields 2-[5-Methyl-2-(4-trifluoromethyl-phenyl)-oxazol-4-yl]-propionic acid methyl ester (3.40 g) and 2-Methyl-2-[5-methyl-2-(4-trifluoromethyl-phenyl)-oxazol-4-yl]-propionic acid methyl ester (2.80 g).

### Step C

### 2-[5-Methyl-2-(4-trifluoromethyl-phenyl)-oxazol-4-yl]-propan-1-ol

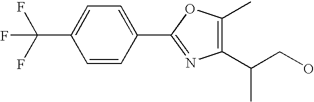

To a solution of 2-[5-methyl-2-(4-trifluoromethyl-phenyl)-ozazol-4-yl]-propionic acid methyl ester (3.4 g) from step B in THF (20 mL) is added a solution of lithium aluminum hydride in THF (1.0 M, 14 mL) at 0° C. After 2 hrs, the reaction is quenched by water and sodium hydroxide, filtered, concentrated. Chromatography on silica gel yields 2-[5-Methyl-2-(4-trifluoromethyl-phenyl)-oxazol-4-yl]-propan-1-ol (0.88 g).

### Step D

### and 2-Methyl-2-[5-methyl-2-(4-trifluoromethyl-phenyl)-oxazol-4-yl]-propan-1-ol

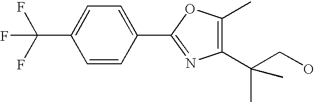

To a solution of 2-methyl-2-[5-methyl-2-(4-trifluoromethyl-phenyl)-oxiazol-4-yl]-propionic acid methyl ester (2.8 g) from step B in THF (14 mL) is added a solution of lithium aluminum hydride in THF (1.0 M, 13 mL) at 0° C. After 2 hrs, the reaction is quenched by water and sodium hydroxide, filtered, concentrated. Chromatography on silica gel yields 2-Methyl-2-[5-methyl-2-(4-trifluoromethyl-phenyl)-oxazol-4-yl]-propan-1-ol (2.3 g).

### Preparation 120

### 2-Methyl-2-[5-ethyl-2-(4-trifluoromethyl-phenyl)-thiazol-4-yl]-propan-1-ol

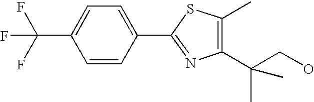

### Step A

### 4-Bromo-2,2-methyl-3-oxo-hexanoic acid methyl ester

A solution of bromine (24 g, 150 mmol) in chloroform (30 mL) is added to 2,2-dimethyl-3-oxo-hexanoic acid methyl ester (25.9 g, 150 mmol) in chloroform (126 mL) at 0-5° C. dropwise. After the addition of bromine, the mixture is allowed to warm up to room temperature slowly and stirred overnight. The reaction is then quenched by ice water, the layers are separated. The organic layer is washed with cold water and brine, dried over sodium sulfate. Concentration yields the title compounds (36.9 g), which is used for next step without further purification.

### Step B

### 2-Methyl-2-[5-ethyl-2-(4-trifluoromethyl-phenyl)-thiazol-4-yl]-propionic acid methyl ester

A mixture of 4-Trifluoromethyl-thiobenzamide (12.3 g, 60 mmol) and the crude product from step A (16.6 g, 66 mmol) in ethanol (600 mL) is heated to reflux for 3 days. Solvent is evaporated and the residue is purified by chromatography on silica gel yielding the title compounds (14.5 g).

### Step C

### 2-Methyl-2-[5-ethyl-2-(4-trifluoromethyl-phenyl)-thiazol-4-yl]-propan-1-ol

To a solution of 2-Methyl-2-[5-ethyl-2-(4-trifluoromethyl-phenyl)-thiazol-4-yl]-propionic acid methyl ester (14.5 g, 40.6 mmol) in THF (100 mL) is added a solution of lithium aluminum hydride in THF (1.0 M, 41 mL) at 0° C. After 2 hrs, the reaction is quenched by water and sodium hydroxide, filtered, concentrated. Chromatography on silica gel yields 2-Methyl-2-[5-ethyl-2-(4-trifluoromethyl-phenyl)-thiazol-4-yl]-propan-1-ol (12.3 g).

### Preparation 121

### 2-[4-Methyl-2-(4-trifluoromethyl-phenyl)-thiazol-5-yl]-propan-1-ol

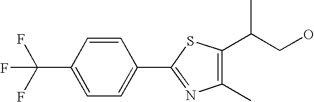

### Step A

### 1-[4-Methyl-2-(4-trifluoromethyl-phenyl)-thiazol-5-yl]-ethanone

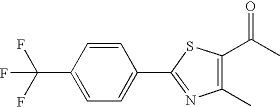

A mixture of 1-[4-Methyl-2-(4-trifluoromethyl-phenyl)-thiazol-5-yl]-ethanol (1.0 g, 3.48 mmol) and MnO2 (0.45 g, 5.22 mmol) in chloroform (30 mL) is heated to reflux, after 24 hrs, additional MnO2 (300 mg) is added and refluxed for another 9 hrs, the reaction mixture is filtered through celite. Concentration of filtrate yields the title compound (1.0 g).

### Step B

### 5-(2-Methoxy-1-methyl-vinyl)₄-methyl-2-(4-trifluoromethyl-phenyl)-thiazole

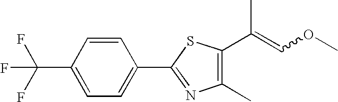

To a solution of (methoxymethyl)triphenyl phosphonium chloride (15.5 g, 45.2 mmole) in toluene (330 mL) is added potassium t-butoxide (5.07 g, 45.2 mmole) in one portion and stirred for 30 minutes, then a solution of 1-[4-Methyl-2-(4-trifluoromethyl-phenyl)-thiazol-5-yl]-ethanone (8.6 g, 30.1 mmole) in toluene (20 mL) is added. The reaction is stirred for 4 hours, quenched by NH4Cl aqueous solution, extracted with ethyl acetate and then concentrated on rota vapor. The residue is purified on a silica gel column, eluting with 0-10% ethyl acetate in hexane and concentrated to provide the title compound (7.0 g).

### Step C

### 2-[4-Methyl-2-(4-trifluoromethyl-phenyl)-thiazol-5-yl]-propionaldehyde

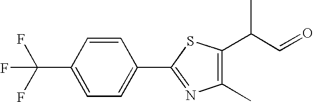

5-(2-Methoxy-1-methyl-vinyl)-4-methyl-2-(4-trifluoromethyl-phenyl)-thiazole (7.0 g, 22.3 mmol) in THF (200 mL) is treated with concentrated HCl aqueous solution (7 mL) at 50° C. for 2 hours. The reaction mixture is diluted with ethyl acetate, washed with sodium bicarbonate aqueous solution, dried over sodium sulfate. Concentration and column chromatography on silica gel provided the title compound (3.5 g).

### Step D

### 2-[4-Methyl-2-(4-trifluoromethyl-phenyl)-thiazol-5-yl]-propan-1-ol

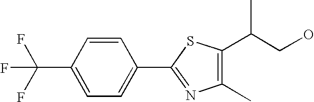

To a solution of 2-[4-methyl-2-(4-trifluoromethyl-phenyl)-thiazol-5-yl]-propionaldehyde (2.0 g, 6.68 mmol) in ethanol (30 mL) is added to NaBH₄ (0.25 g, 6.6 mmol) in portions at 0° C. The reaction is kept at 0° C. for 15 minutes and warmed up to room temperature for 2 hours. The reaction is quenched using water, extracted with ethyl acetate, dried over sodium sulfate. Concentration and column chromatography on silica gel eluted with hexanes and ethyl acetate yields the title compound (2.0 g).

### Preparation 122

### 2-[5-Ethyl-2-(4-trifluoromethyl-phenyl)-thiazol-4-yl]-propan-1-ol

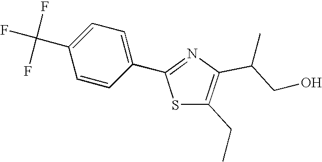

### Step A

3-Oxo-hexanoic acid ethyl ester (29.5 g, 0.1865 Mol) is dissolved into anhydrous dichloromethane (DCM) (400 mL) and then cooled to 0° C.-5° C. while stirring. A solution of bromine (30.4 g, 0.190 Mol) in DCM (80 mL) is added dropwise over 2 h. to the solution of the beta keto-ester. After the addition, the mixture is allowed to stir 0.5 h. at 0° C., then the ice bath is removed and the mixture is allowed to stir at room temperature for 18 h. TLC will show complete consumtion of starting material, then ice water (200 g) is added with stirring. The organic layer is collected and washed with cold water (2×) and brine. The filtered solution is dried over anhydrous sodium sulfate, then concentrated to a clear liquid. The crude 4-Bromo-3-oxo-hexanoic acid ethyl ester (40.2 g, 0.1695 Mol), 91% yield, is used without further purification.

### Step B

4-Bromo-3-oxo-hexanoic acid ethyl ester (4.68 g, 20.98 mmol) is dissolved into denatured ethanol (100 mL) and para-trifluoromethyl thiobenzamide (4.31 g, 21 mmol) is added in one portion. The reaction is purged of air and flushed with nitrogen then heated to reflux. The reaction is monitored by TLC and HPLC and when complete, the reaction is allowed to cool to room temperature. The solvent is removed and the reaction is diluted with ethyl acetate (200 mL), followed by washes with saturated sodium bicarbonate solution, water, and brine. The ethyl acetate solution is dried over anhydrous sodium sulfate, then concentrated and further purified using flash column chromatography (10% EtOAc/Hexanes) to yield pure [5-Ethyl-2-(4-trifluoromethyl-phenyl)-thiazol-4-yl]-acetic acid ethyl ester (5.09 g, 14.82 mmol) or 71% yield.

### Step C

[5-Ethyl-2-(4-trifluoromethyl-phenyl)-thiazol-4-yl]-acetic acid ethyl ester (2.02 g, 6.13 mmol) is dissolved into anhydrous tetrahydrofuran (THF) (10 mL) and lithium diisopropylamide (LDA) is slowly added at room temperature. This solution is allowed to stir at room temperature and monitored by TLC. After complete conversion, methyl iodide (582 mg, 4.00 mmol) is added slowly and the reaction is followed by TLC. After 18 h., the reaction is not complete, but is quenched with saturated ammonium chloride solution and diluted with diethyl ether. The two phases are separated and the organic layer is washed with water and brine, dried over anhydrous sodium sufate, then concentrated and purified using flash column chromatography (10% EtOAc/Hexanes). The pure 2-[5-Ethyl-2-(4-trifluoromethylphenyl)-thiazol-4-yl]-propionic acid ethyl ester (1.30 g, 3.64 mmol) is obtained in 59% yield.

### Step D

2-[5-Ethyl-2-(4-trifluoromethyl-phenyl)-thiazol-4-yl]-propionic acid ethyl ester (1.05 g, 3.06 mmol) is dissolved into anhydrous tetrahydrofuran (THF) (10 mL) and then cooled to 0° C. with stirring. Lithium aluminum hydride (3.10 mL, 1M in THF, 3.10 mmol) is slowly added by syringe and the reaction is monitored by TLC. Upon complete conversion, the reaction is carefully quenched using water, base, and water. Celite is added to the reaction, followed by diethyl ether and the mixture is then filtered through a celite plug. The two phases are then separated and the organic layer is washed using water and brine. The organic layer is the dried over anhydrous sodium sulfate and concentrated. The pure 2-[5-Ethyl-2-(4-trifluoromethyl-phenyl)-thiazol-4-yl]-propan-1-ol (0.930 g, 2.95 mmol) is obtained in 95% yield after flash column chromatography.

### Preparation 123

### 2-[4-Isopropyl-2-(4-trifluoromethyl-phenyl)-thiazol-5-yl]-propan-1-ol

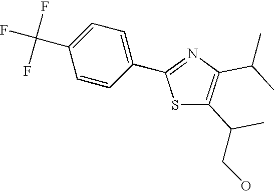

### Step A

4-Isopropyl-2-(4-trifluoromethyl-phenyl)-thiazole-5-carboxylic acid methyl ester (14 g, 40.1 mmol) is dissolved into anhydrous tetrahydrofuran (THF) (200 mL) and then cooled to −30° C. while stirring under nitrogen. N-methyl, N-methoxy amine hydrochloride (0.881 g, 9.04 mmol) is then added to the solution in one portion. Isopropyl magnesium chloride (8.73 mL, 2M soln. in THF, 17.46 mmol) is slowly added to the cooled suspension over 1 h. TLC will show complete consumtion of starting material, then 30% solution of ammonium chloride is added with stirring. The reaction is diluted with diethyl ether and extracted. The organic layer is collected and washed with cold water (2×) and brine. The solution is then dried over anhydrous sodium sulfate, filtered, and concentrated. The 4-Isopropyl-2-(4-trifluoromethyl-phenyl)-thiazole-5-carboxylic acid methoxy-methyl-amide (0.705 g, 1.97 mmol) is obtained in pure form after flash column chromatography.

### Step B

4-Isopropyl-2-(4-trifluoromethyl-phenyl)-thiazole-5-carboxylic acid methoxy-methyl-amide (7.56 g, 21.09 mmol) is suspended in anhydrous tetrahydrofuran (100 mL), and cooled to 0° C. with stirring under nitrogen. Methyl magnesium bromide (28 mL, 3.0M in diethyl ether, 84.36 mmol) is slowly added to the reaction over 1 h. The reaction is allowed to warm slowly to room temperature and monitored by TLC. Upon complete consumption of starting material, the reaction is carefully neutralized with 1N hydrochloric acid, extracted with diethyl ether, washed, dried, and concentrated. The 1-[4-Isopropyl-2-(4-trifluoromethyl-phenyl)-thiazol-5-yl]-ethanone (5.4 g, 17.23 mmol) 82% yield, is used without further purification.

### Step C

(Methoxymethyl)triphenylphosphinium chloride (8.86 g, 25.84 mmol) is suspended in anhydrous toluene (75 mL) and potassium tert-butoxide (2.90 g, 25.84 mmol) is carefully added. The solution is allowed to cool and stir at room temperature for 1 h. 1-[4-Isopropyl-2-(4-trifluoromethyl-phenyl)-thiazol-5-yl]-ethanone (5.4 g, 17.23 mmol) is then dissolved into anhydrous toluene (25 mL) and added to the reaction mixture by syringe. The reaction is allowed to stir at room temperature for several hours and is monitored by TLC. Upon complete consumption of starting material, the reaction is carefully quenched with saturated ammonium chloride solution, extracted with diethyl ether, washed, dried, and concentrated. The 4-Isopropyl-5-(2-methoxy-1-methyl-vinyl)-2-(4-trifluoromethyl-phenyl)-thiazole is used in the next step without further purification.

### Step D

4-Isopropyl-5-(2-methoxy-1-methyl-vinyl)-2-(4-trifluoromethyl-phenyl)-thiazole is dissolved into anhydrous tetrahydrofuran (100 mL) and concentrated hydrochloric acid (5 mL) is added with stirring under nitrogen. The reaction is heated to 50° C. and monitored by TLC. Upon complete consumption of starting material, the reaction is carefully neutralized with sodium hydroxide, extracted with diethyl ether, washed, dried, and concentrated. The 2-[4-Isopropyl-2-(4-trifluoromethyl-phenyl)-thiazol-5-yl]-propionaldehyde (4.6 g, 14.05 mmol), 82% two steps, is obtained in pure form after flash column chromatography.

### Step E

2-[4-Isopropyl-2-(4-trifluoromethyl-phenyl)-thiazol-5-yl]-propionaldehyde (4.05 g, 12.5 mmol) is dissolved into denatured ethanol (60 mL) at room temperature then cooled to 0° C. in an ice bath. Sodium borohydride (0.467 g, 12.5 mmol) is then carefully added in small portions. The reaction is allowed to warm slowly to room temperature and is monitored by TLC. Upon complete consumption of starting material, the reaction is carefully quenched with water and diluted with ethyl acetate. The ethanol is removed and the residue is extracted with ethyl acetate, washed, dried, and concentrated. The 2-[4-Isopropyl-2-(4-trifluoromethyl-phenyl)-thiazol-5-yl]-propan-1-ol (4.0 g, 12.14 mmol), 97%, is obtained in pure form after flash column chromatography.

### Preparation 124

### Toluene-4-sulfonic acid 2-[5-ethyl-2-(4-trifluoromethyl-phenyl)-oxazol-4-yl]-ethyl ester

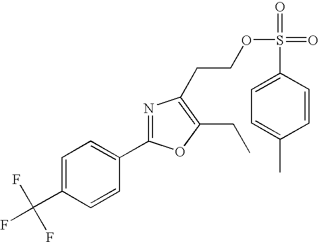

### Step A

To a solution of □,□,□-trifluoromethyl-para-toluic acid (5.0 g, 26.3 mmol) in anhydrous acetone (100 mL) at 0° C. under nitrogen is added 4-bromo-3-oxo-hexanoic acid ethyl ester (6.4 g, 27 mmol) and triethyl amine (3.5 mL, 27 mmol). The mixture is allowed to stir 0.5 h. at 0° C., then the ice bath is removed and the mixture allowed to stir at room temperature for 18 h. The reaction is monitored by TLC and HPLC until complete consumtion of starting material, then ice water added with stirring and the mixture is extracted. The organic layer is collected and washed with brine, then dried over anhydrous sodium sulfate. The crude 4-Trifluoromethyl-benzoic acid 3-ethoxycarbonyl-1-ethyl-2-oxo-propyl ester is used in the next step without further purification.

### Step B

4-Trifluoromethyl-benzoic acid 3-ethoxycarbonyl-1-ethyl-2-oxo-propyl ester (25 mmol) is dissolved in acetic acid (100 mL) and dry ammonium acetate (10 g, 100 mmol) is added, then the reaction is heated under nitrogen to reflux. The reaction is monitored by TLC and HPLC but complete consumption of the starting material is never observed, and then allowed to cool. The cooled reaction is concentrated and diluted with 250 mL ethyl acetate. The residue is washed with 100 mL saturated sodium bicarbonate followed by water and brine. The organic layer is dried over anhydrous sodium sulfate, then concentrated and purified by column chromatrography. The fractions that contained pure product are concentrated to yield [5-Ethyl-2-(4-trifluoromethyl-phenyl)-oxazol-4-yl]-acetic acid ethyl ester (4.0 g, 12.22 mmol) or 50% yield.

### Step C

[5-Ethyl-2-(4-trifluoromethyl-phenyl)-oxazol-4-yl]-acetic acid ethyl ester (4.0 g, 12.22 mmol) in anhydrous tetrahydrofuran (100 mL) is cooled to 0° C. and a 1M LiAlH₄ (12.2 mL, 12.2 mmol) solution is added slowly. The reaction is monitored by TLC until complete consumption of the starting material. The reaction is then carefully quenched with 2.4 mL water, 2.4 mL 5N NaOH and 7 mL water. The light tan solid is filter through celite and dried to give crude 2-[5-Ethyl-2-(4-trifluoromethyl-phenyl)-oxazol-4-yl]-ethanol (2.74 g, 9.60 mmol) or 79% yield.

### Step D

To a solution of 2-[5-Ethyl-2-(4-trifluoromethyl-phenyl)-oxazol-4-yl]-ethanol 2.74 g, 9.60 mmol) in nhydrous dichloromethane (50 mL) is added dimethylamino pyridine (0.500 g, 4.00 mmol), tosic anhydride (8.4 g, 24 mmol), and pyridine (3.4 mL, 42 mmol) at room temperature. The reaction is monitored by TLC, and upon complete consumption of the starting alcohol, the reaction is diluted with DCM and extracted against saturated sodium bicarbonate solution. The organic layer is washed with water and brine, then dried over anhydrous sodium sulfate and concentrated. The pure toluene-4-sulfonic acid 2-[5-ethyl-2-(4-trifluoromethyl-phenyl)-oxazol-4-yl]-ethyl ester (3.0 g, 6.82 mmol) is obtained after flash column chromatography.

### Preparation 125

### Toluene-4-sulfonic acid 2-methyl-2-[5-methyl-2-(4-trifluoromethyl-phenyl)-thiazol-4-yl]-propyl ester

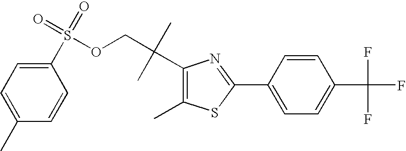

### Preparation 126

### Toluene-4-sulfonic acid 2-methyl-2-[5-methyl-2-(4-trifluoromethyl-phenyl)-oxazol-4-yl]-propyl ester

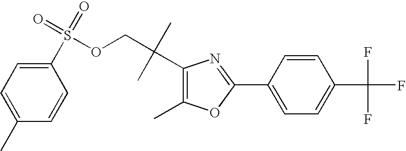

### Preparation 127

### 1-[5-Ethyl-2-(4-trifluoromethyl-phenyl)-thiazol-4-yl]-propan-2-ol

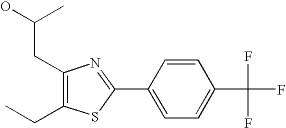

### Step A

To a solution of 2-[5-Ethyl-2-(4-trifluoromethyl-phenyl)-thiazol-4-yl]-ethanol (1.25 g, 4.16 mmol) in anhydrous dichloromethane (25 mL) at 0° C. under nitrogen is slowly added Dess-Martin periodinane (2.6 g, 6.24 mmol). The reaction is allowed to warm slowly to room temperature and monitored by TLC. After complete consumption of the starting material, the reaction is diluted with dichloromethane and the two phases are seperated. The organic layer is washed, dried, filtered and concentrated. The crude [5-Ethyl-2-(4-trifluoromethyl-phenyl)-thiazol-4-yl]-acetaldehyde (0.253 g, 0.840 mmol), 21% yield, is further purified using flash column chromatography.

### Step B

[5-Ethyl-2-(4-trifluoromethyl-phenyl)-thiazol-4-yl]-acetaldehyde (0.253 g, 0.840 mmol), is dissolved in anhydrous tetrahydrofuran (5 mL) and cooled to 0° C. with stirring under nitrogen. Methylmagnesium bromide, 3.0M in ether, (0.300 mL, 1.00 mmol) is added and the ice bath removed. After slowly warming to room temperature, the reaction is monitored by TLC. After the starting material is completely consumed, the reaction is quenched with saturated ammonium chloride solution and diluted with ether. The two phases are separated and the organic washed with water and brine, dried over sodium sulfate, then concentrated. The residue is further purified using flash column chromatography. The 1-[5-Ethyl-2-(4-trifluoromethyl-phenyl)-thiazol-4-yl]-propan-2-ol (0.222 g, 0.7049 mmol) is formed in 70% yield.

### Preparation 128

### C-[5-Methyl-2-(4-trifluoromethyl-phenyl)-oxazol-4-yl]-methylamine

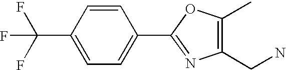

### Step A

### 4-Azidomethyl-5-methyl-2-(4-trifluoromethyl-phenyl)-oxazole

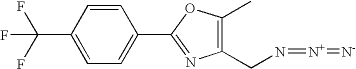

To a solution of 4-chloromethyl-5-methyl-2-(4-trifluoromethyl-phenyl)-oxazole (2.4 g, 8.71 mmol) in methanol (13 mL) is added sodium azide (1.13 g, 17.4 mmol) in water (10 mL). The mixture is heated to reflux for 3 hrs, cooled to room temperature, majority of the methanol is evaporated, the residue is extracted with ethyl acetate, dried, concentrated and column chromatography on silica gel yields the title compound (2.10 g).

### Step B

### C-[5-Methyl-2-(4-trifluoromethyl-phenyl)-oxazol-4-yl]-methylamine

A mixture of 4-azidomethyl-5-methyl-2-(4-trifluoromethyl-phenyl)-oxazole (1.70 g) and PtO2 (0.106 g) in ethyl acetate (50 mL) at room temperature under 60 psi of hydrogen for 5 hrs, the reaction mixture is filtered through celite and filtrate is concentrated giving the title compound (1.3 g, 84.2% yield).

### Preparation 129

### 2R-[5-Methyl-2-(4-trifluoromethyl-phenyl)-thiazol-4-yl]-propan-1-ol

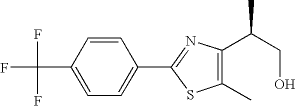

The racemic alcohol 2-[5-methyl-2-(4-trifluoromethyl-phenyl)-thiazol-4-yl]-propan-1-ol is resolved on a Chiralpak AD column (4.6×250 mm). Eluted with ethanol in heptane and concentrated the fractions to provide pure enantiomers.

### Preparation 130

### 2S-[5-Methyl-2-(4-trifluoromethyl-phenyl)-oxazol-4-yl]-propan-1-ol

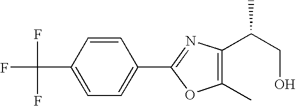

This compound is obtained in Preparation 129.

### Preparation 131

### (5-Hydroxy-indol-1-yl)-acetic acid ethyl ester

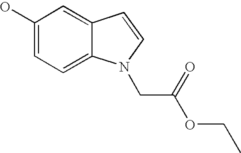

### Step A

5-benzyloxyindole (10.0 g, 44.79 mmol) is dissolved into anhydrous DMF (100 mL) and cooled to 0° C. in an ice water bath. Sodium hydride (2.6 g, 67.18 mmol) is dissolved into anhydrous DMF (100 mL), then slowly added to the indole solution using an addition funnel. The reaction is allowed to stir at 0° C. for 1 h., then the ice bath is removed and the solution is allowed to warm slowly to room temperature. The solution is then cooled back down to 0° C. and ethyl bromoacetate (11.2 g, 67.18 mmol) is then added in one portion. The reaction is allowed to stir at 0° C. for 1 h, then the ice bath is removed and the solution is allowed to warm slowly to room temperature. Upon completion, the reaction is quenched carefully using water, then diluted with EtOAc (300 mL). Brine (100 mL) is added and the two layers are seperated in a seperatory funnel. The organic layer is rinsed with water (2×75 mL) and then dried over anhydrous magnesium sulfate. The organic layer is then concentrated and purified using flash column chromatography (5% EtOAc/Hexanes) to yield 11.86 g (86%) of (5-Benzyloxy-indol-1-yl)-acetic acid ethyl ester.

### Step B

(5-Benzyloxy-indol-1-yl)-acetic acid ethyl ester (3.49 g, 11.31 mmol) is dissolved in EtOH (50 mL) and glacial acetic acid is added (2.0 mL). Palladium on carbon (20% by wt., 0.700 g) is then added to the homogenous solution, and a hydrogen filled balloon is connected to the round bottom flask. A vaccuum is created within the flask until the ethanol began to bubble, and the hydrogen allowed to enter the flask; this procedure is repeated three times, then the reaction is left to stir at room temperature overnight. Upon completion, the reaction is diluted with DCM (200 mL), and water (100 mL) is added. The mixture is filtered through a celite plug and the two phases are separated. The organic layer is washed with brine (2×75 mL), then dried over anhydrous magnesium sulfate, and concentrated to yield the title compound (2.42 g) in 98% yield. The residual acetic acid is removed by flash column chromatography.

¹H NMR (DMSO-d₆, 400 MHz): δ 1.18 (t, J=7.34 Hz, 3H), 4.12 (q, J=6.85 Hz, 2H), 4.99 (s, 2H), 5.73 (s, 1H), 6.23 (d, J=2.94 Hz, 1H), 6.60 (dd, J=1.96 Hz, J₂=8.80 Hz, 1H), 6.84 (d, J=2.45 Hz, 1H), 7.12 (d, J=8.80 Hz, 1H), 7.19 (d, J=2.94 Hz, 2H); MS (ES, M/z):C₁₂H₁₃NO₃: 220.21 (M⁺+1), 218.7 (M⁺−1).

The following compounds are prepared in a manner substantially similar to that used to prepare the compound of preparation 21.

### Preparation 132

### 3-(5-Hydroxy-indol-1-yl)-propionic acid ethyl ester

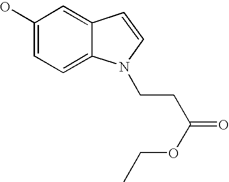

### Preparation 133

### 4-(5-Hydroxy-indol-1-yl)-butyric acid ethyl ester

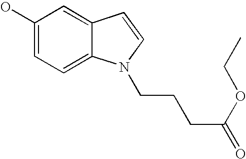

### Preparation 134

### 5-(5-Hydroxy-indol-1-yl)-pentanoic acid ethyl ester

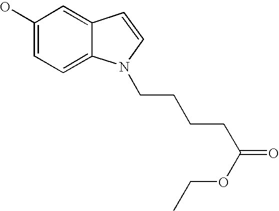

### Preparation 135

### 2-(5-Hydroxy-indol-1-yl)-propionic acid ethyl ester

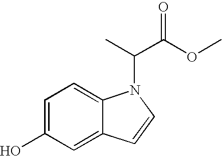

### Step A

### 2-(5-Benzyloxy-indol-1-yl)-propionic acid ethyl ester

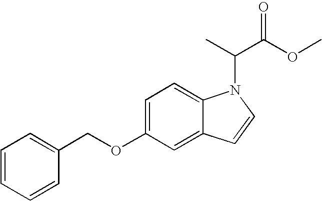

To a mixture of NaH (60%, 4.92 g, 0.205 mol) in DMF (60 mL) is added 5-benzoxyindole at 0˜5° C., then stirred 30 min. ethyl 2-bromopropionate is added dropwise, the mixture is allowed to warm to room temperature and heated at 70° C. overnight, cooled to room temperature, diluted with ethyl acetate, washed with water and brine, dried over sodium sulfate. Concentration and column chromatography on silica gel eluted with hexanes and ethyl acetate yields the title compound (29 g).

### Step B

### 2-(5-Hydroxy-indol-1-yl)-propionic acid ethyl ester

A mixture of 2-(5-Benzyloxy-indol-1-yl)-propionic acid ethyl ester (16 g) and Pd/C (5%, 1.93 g) in ethanol (190 mL) is stirred under 60 PSI of hydrogen overnight. Filtration and concentration yields the title compound.

### Preparation 136

### 2-(5-Hydroxy-indol-1-yl)-2-methyl-propionic acid ethyl ester

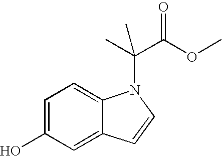

### Step A

### 2-(5-Benzyloxy-indol-1-yl)-2-methyl-propionic acid ethyl ester

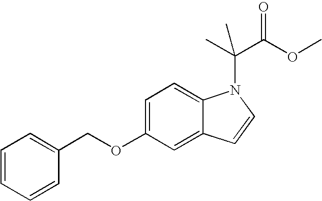

To a solution of 2-(5-benzyloxy-indol-1-yl)-propionic acid ethyl ester (20 g, 61.5 mmol) in THF (180 mL) is added LDA (2.0 M toluene, 37 mL) dropwise at −78° C. After the addition of LDA, the mixture is stirred for 30 min, then methyl iodide (8.77 g, 122.6 mmol) is added. The reaction mixture is allowed to warm to room temperature, after stirred for 2 hrs, quenched by water, extracted with ethyl acetate, dried over sodium sulfate. Concentration yields the title compound.

### Step B

### 2-(5-Hydroxy-indol-1-yl)-2-methyl-propionic acid ethyl ester

A mixture of 2-(5-benzyloxy-indol-1-yl)-2-methyl-propionic acid ethyl ester (15.6 g) and Pd/C (5%, 1.93 g) in ethanol (190 mL) is stirred under 60 PSI of hydrogen overnight. Filtration and concentration yields the title compound (11 g).

### Preparation 137

### (5-Hydroxy-2-methyl-indol-1-yl)-acetic acid ethyl ester

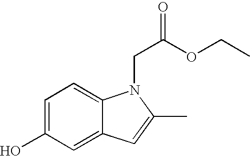

### Step A

### (5-Methoxy-2-methyl-indol-1-yl)-acetic acid ethyl ester

To a solution of 2-methyl-5-methoxylindole (5.10 g, 31.6 mmol) in DMF (200 mL) is added sodium hydride (60%, 1.9 g, 47.4 mmol) at 0˜5° C., stirred for 30 min, ethyl 2-bromoacetate (8.35 g, 50 mmol) is added. After 2 hr at room temperature, the reaction is quenched by water, extracted with ether. Combined organic layers are washed with water and brine, dried over sodium sulfate. Concentration yields the crude title product, which is used for next step without further purification.

### Step B

### (5-hydroxy-2-methyl-indol-1-yl)-acetic acid ethyl ester

To a solution of (5-methoxy-2-methyl-indol-1-yl)-acetic acid ethyl ester (0.87 g, 3.51 mmol) in methylene chloride (25 mL) is added BBr3 (1.0 mL, 10.5 mmol) at −20° C. After stirred at −20° C. for 2 hrs, the reaction mixture is poured into ice, extracted with methylene chloride, dried over sodium sulfate. Concentration yields the crude title compound, which is used for next step without further purification.

### Preparation 138

### {5-[3-(Toluene-4-sulfonyloxy)-propoxy]-indol-1-yl}-acetic acid ethyl ester

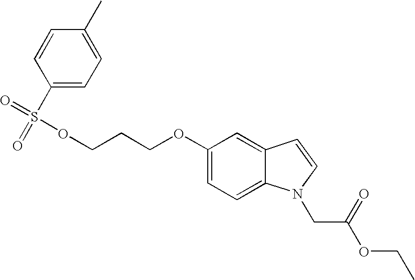

[5-(3-Hydroxy-propoxy)-indol-1-yl]-acetic acid ethyl ester (2.0 g, 6.55 mmol) is dissolved into anhydrous dichlormethane (O)CM) (35 mL), then dimethylamino pridine (300 mg, 1.965 mmol), tosic anhydyhride (4.3 g, 13.1 mmol), and pyridine (2.3 mL, 23 mmol) are added. The reaction allowed to stir at room temperature under nitrogen. Upon completion, the reaction is then diluted with DCM (100 mL) and saturated sodium bicarbonate solution (50 mL) is added and the two layers are seperated in a seperatory funnel. The organic layer is rinsed with water (2×75 mL) and brine (2×50 mL), then dried over anhydrous magnesium sulfate. The organic layer is then concentrated and purified using flash column chromatography (5% EtOAc/Hexanes) to yield 1.92 g (64%) of the title compound.

The following compounds are prepared in a similar manner:

### Preparation 139

### 2-Methyl-2-{5-[3-(toluene-4-sulfonyloxy)-propoxy]-indol-1-yl}-propionic acid ethyl ester

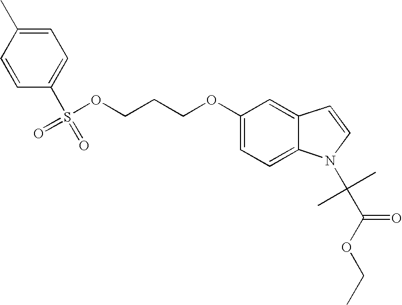

### Preparation 140

### (5-[2-(Toluene-4-sulfonyloxy)-ethoxy]-indol-1-yl}-acetic acid ethyl ester

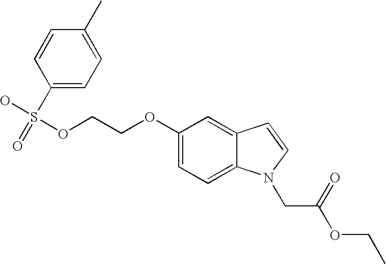

### Preparation 141

### (5-Mercapto-indol-1-yl)-acetic acid ethyl ester

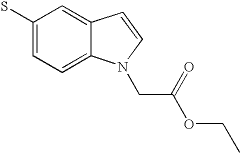

### Step A

(5-Bromo-indol-1-yl)-acetic acid ethyl ester (2.0 g, 7.09 mmol) is dissolved into anhydrous tetrahydrofuran (THF) (10 mL) and the reaction vessel purged with nitrogen a few times. Tetrakis triphenylphosphine palladium (175 mg, 0.15 mmol) is then added to the indole solution in one portion, purge again. Meanwhile, triisopropylsilylthiol (1.67 mL, 7.8 mmol) is dissolved in anhydrous THF (20 mL) and potassium hydride (0.483 mg, 7.8 mmol) is then slowly added. This mixture is heated to 50° C. for 4 h. After this solution has cooled to room temperature, it is transferred to the indole solution via kannula. This solution is the heated to 70° C. until the reaction is complete. Upon completion, the reaction is quenched carefully using water, then diluted with EtOAc (300 mL). Brine (100 mL) is added and the two layers are separated in a separatory funnel. The organic layer is rinsed with water (2×75 mL) and then dried over anhydrous magnesium sulfate. The organic layer is then concentrated and purified using flash column chromatography (5% EtOAc/Hexanes) to yield 1.3 g (50%) of (5-Triisopropylsilanylsulfanyl-indol-1-yl)-acetic acid ethyl ester.

### Step B

(5-Triisopropylsilanylsulfanyl-indol-1-yl)-acetic acid ethyl ester (60 mg, 0.1621 mmol) is dissolved in n-methylpyrrolidinone (NMP) (5 mL) and cesium fluoride (0.243 mmol) is added. The reaction is allowed to stir at room temperature until complete. This solution may be used in the coupling step (next) without further purification.

### Preparation 142

### (6-Hydroxy-1H-indol-3-yl)-acetic acid methyl ester

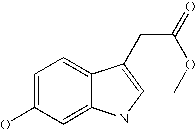

### Step A

### (6-Benzyloxy-1H-indol-3-yl)-oxo-acetic acid methyl ester

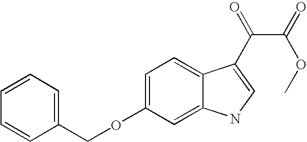

To a solution of 6-benzoxyindole (1.05 g, 4.7 mmol) in ether (8 mL) is added oxayl chloride (0.45 mL) at 0˜5° C., stirred for 2 hrs. The reaction mixture is cooled to −78° C., sodium methoxide (25% w/w in methanol, 2.4 mL) is added, warmed up to room temperature, quenched by water. Solid product is collected by filtration, washed by water and dried under vacuum.

### Step B

### (6-Hydroxy-1H-indol-3-yl)-acetic acid methyl ester

A mixture of (6-benzyloxy-1H-indol-3-yl)-oxo-acetic acid methyl ester (1.45 g, 4.7 mmol) and Pd/C (10%, 0.9 g) in dioxane (38 mL) is degassed and filled with nitrogen for three times, then a solution of NaH2PO2 (6 g) in water (5 mL) is added dropwise at 100° C. The reaction mixture is heated overnight, filtered through celite and concentrated. The residue is taken into ethyl acetate, washed with water and brine, dried over sodium sulfate. Concentration and column chromatography on silica gel yields the title compound (600 mg).

The following compounds are made in a similar manner:

### Preparation 143

### (6-Hydroxy-1-methyl-1H-indol-3-yl)-acetic acid methyl ester

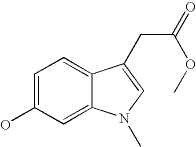

### Preparation 144

### (5-Hydroxy-1H-indol-3-yl)-acetic acid methyl ester

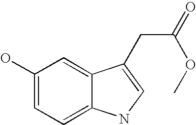

### Preparation 145

### (5-Hydroxy-1-methyl-1H-indol-3-yl)-acetic acid methyl ester

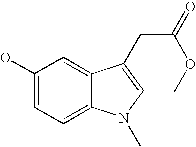

### Preparation 146

### (1-Ethyl-6-hydroxy-1H-indol-3-yl)-acetic acid ethyl ester

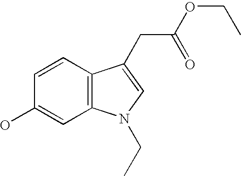

### Step A

To a solution of (6-Benzyloxy-1H-indol-3-yl)-oxo-acetic acid ethyl ester (3.0 g, 9.7 mmol) in anhydrous dimethyl formamide (50 mL) at 0° C. under nitrogen is added sodium hydride (0.600 g, 14.5 mmol) in small portions. The reaction is allowed to warm to room temperature slowly and monitored by TLC. Upon complete conversion, the reaction is cooled back down to 0° C. and ethyl bromide (1.5 mL, 20 mmol) is slowly added to the slurry. The reaction is allowed to warm slowly to room temperature and monitored by TLC. After complete consumption of the starting material, the reaction is quenched with water, then diluted with ethyl acetate, and the two phases are separated. The organic layer is washed, dried, filtered and concentrated. The crude (6-Benzyloxy-1-ethyl-1H-indol-3-yl)-oxo-acetic acid ethyl ester (0.843 g, 2.40 mmol), 25% yield, is further purified using flash column chromatography.

### Step B

(6-Benzyloxy-1-ethyl-1H-indol-3-yl)-oxo-acetic acid ethyl ester (0.843 g, 2.40 mmol) is dissolved in anhydrous dioxane (10 mL) then purged and back filled with nitrogen a few times. Palladium on carbon (10%) (0.200 g, 20% by wt.) is added and the reaction followed by heating to reflux. Slow addition of a saturated solution of sodium hypophosphite is initiated and the reaction is monitored by TLC. After the starting material is completely consumed, the reaction is allowed to cool to room temperature, diluted with dichloromethane and celite added. The mixture is filtered through a plug of celite and the two phases are separated. The organic layer is washed with water and brine, dried over sodium sulfate, then concentrated. The residue is further purified using flash column chromatography. The (1-Ethyl-6-hydroxy-1H-indol-3-yl)-acetic acid ethyl ester (0.564 g, 2.28 mmol) is formed in 95% yield.

The following compound is made in a similar manner:

### Preparation 147

### (1-propyl-6-hydroxy-1H-indol-3-yl)-acetic acid ethyl ester

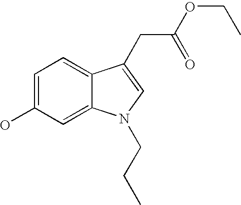

### EXAMPLE 1

### {5-[5-Methyl-2-(4-trifluoromethyl-phenyl)-oxazol-4-ylmethoxy]-indol-1-yl}-acetic acid

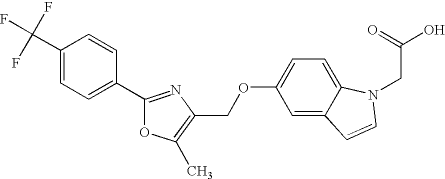

### Step 1

(5-Hydroxy-indol-1-yl)-acetic acid ethyl ester (109 mg, 0.500 mmol) is dissolved into anhydrous acetonitrile (ACN) (2 mL). Cesium carbonate (326 mg, 1.00 mmol) is added to the reaction, followed by the addition of 4-Chloromethyl-5-methyl-2-(4-trifluoromethyl-phenyl)-oxazole (135 mg, 0.490 mmol). The reaction is allowed to stir under nitrogen at room temperature and monitored by TLC and HPLC. Upon complete consumption of the chloride, the reaction is diluted with diethyl ether and quenched with 0.1N NaOH. The two phases are separated, then the organic layer washed with water and brine. The organic phase is dried over anhydrous sodium sulfate and concentrated under vacuum. The residue is further purified using either EtOAc/Hexanes (1:9) or Acetone/Hexanes (1:9) gradients on silica gel chromatography to yield {5-[5-Methyl-2-(4-trifluoromethyl-phenyl)-oxazol-4-ylmethoxy]-indol-1-yl}-acetic acid ethyl ester (157 mg, 0.343 mmol) or 70%.

### Step 2

{5-[5-Methyl-2-(4-trifluoromethyl-phenyl)-oxazol-4-ylmethoxy]-indol-1-yl}-acetic acid ethyl ester (157 mg, 0.343 mmol) is dissolved in tetrahydrofuran (1 mL) and 5N NaOH (1 mL) is added. The mixture is heated to reflux until the conversion is complete. Upon complete conversion, the reaction is cooled to room temperature and 5N HCl (1 mL) is added. The mixture is diluted with diethyl ether and extracted with 1N HCl. The organic layer is washed with water and brine, then dried over anhydrous sodium sulfate. Concentration of the solvent reveals the pure {5-[5-Methyl-2-(4-trifluoromethyl-phenyl)-oxazol-4-ylmethoxy]-indol-1-yl}-acetic acid in near quantitative yield (140 mg, 0.326 mmol) MS (ES): 431.16 (M⁺+1).

The following compounds are made in a substantially similar manner:

### EXAMPLE 2

### [5-(5-Methyl-2-phenyl-oxazol-4-ylmethoxy)-indol-1-yl]-acetic acid

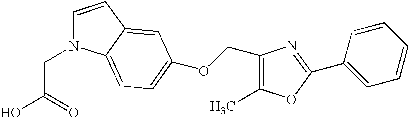

MS (ES): 363.22 (M⁺+1).

### EXAMPLE 4

### {5-[2-(4-Fluoro-phenyl)-5-methyl-oxazol-4-ylmethoxy]-indol-1-yl}-acetic acid

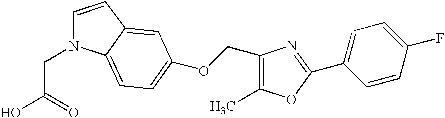

MS (ES): 381.17 (M⁺+1).

### EXAMPLE 5

### {5-[2-(4-Benzyloxy-phenyl)-5-methyl-oxazol-4-ylmethoxy]-indol-1-yl}-acetic acid

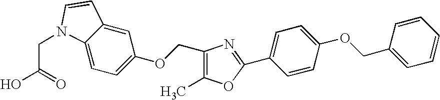

MS (ES): 469.17 (M⁺+1).

### EXAMPLE 6

### 2-Methyl-2-(5-{2-[2-(4-trifluoromethyl-phenyl)-oxazol-4-ylmethoxy]-ethoxy}-indol-1-yl)-propionic acid

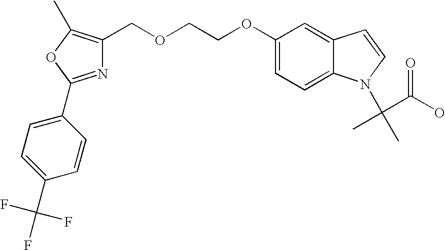

MS (ES): 503.03 (M⁺+1)

### EXAMPLE 7

### {5-[4-Ethyl-2-(4-trifluoromethyl-phenyl)-thiazol-5-ylmethoxy]-indol-1-yl}-acetic acid

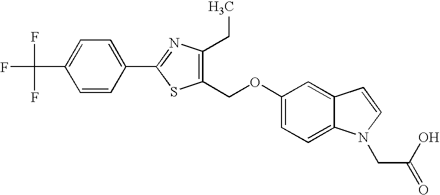

MS (ES): 461.0 (M⁺+1)

### EXAMPLE 8

### 2-Methyl-2-(5-{2-[4-methyl-2-(4-trifluoromethyl-phenyl)-thiazol-5-ylmethoxy]-ethoxy}-indol-1-yl)-propionic acid

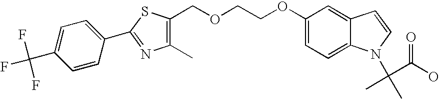

MS (ES): 519.62 (M⁺+1).

### EXAMPLE 9

### {5-[2-(3,5-Bis-trifluoromethyl-phenyl)₄-methyl-thiazol-5-ylmethoxy]-indol-1-yl}-acetic acid

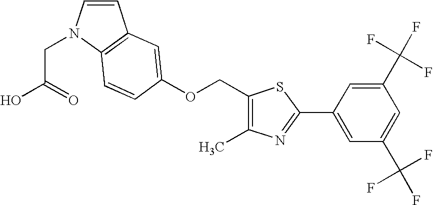

MS (ES): 514.93 (M⁺+1).

### EXAMPLE 10

### {5-[4-Methyl-2-(4-trifluoromethyl-phenyl)-oxazol-5-ylmethoxy]-indol-1-yl}-acetic acid

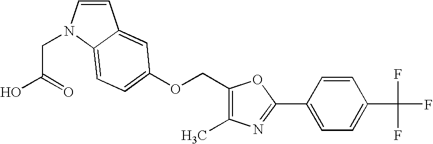

MS (ES): 431.03 (M⁺+1).

### EXAMPLE 11

### 2-Methyl-2-{5-[4-methyl-2-(4-trifluoromethyl-phenyl)-oxazol-5-ylmethoxy]-indol-1-yl}-propionic acid

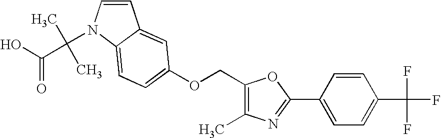

MS (ES): 459.03 (M⁺+1).

### EXAMPLE 12

### Racemic 2-{5-[4-Methyl-2-(4-trifluoromethyl-phenyl)-oxazol-5-ylmethoxy]-indol-1-yl}-propionic acid

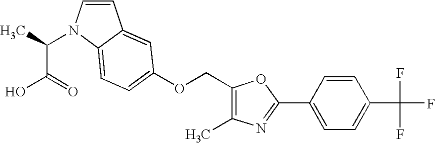

MS (ES): 445.04 (M⁺+1).

### EXAMPLE 13

### {5-[2-(4-Bromo-phenyl)-4-methyl-thiazol-5-ylmethoxy]-indol-1-yl}-acetic acid

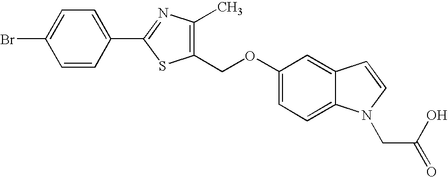

MS (ES): 454.89 (M⁺−1).

### EXAMPLE 14

### {5-[4-Butyl-2-(4-trifluoromethyl-phenyl)-thiazol-5-ylmethoxy]-indol-1-yl}-acetic acid

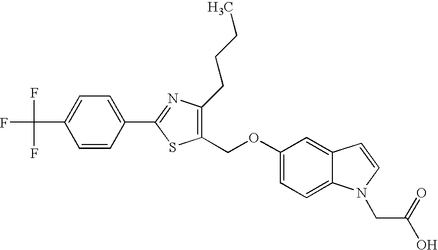

MS (ES): 488.99 (M⁺+1).

### EXAMPLE 15

### 2-{5-[4-Butyl-2-(4-trifluoromethyl-phenyl)-thiazol-5-ylmethoxy]-indol-1-yl}-propionic acid

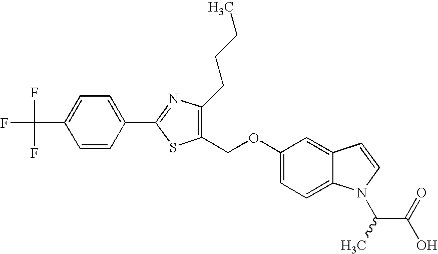

MS (ES): 503.00 (M⁺+1).

### EXAMPLE 16

### {5-[4-Phenethyl-2-(4-trifluoromethyl-phenyl)-thiazol-5-ylmethoxy]-indol-1-yl}-acetic acid

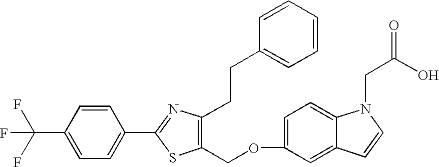

MS (ES): 537.0 (M⁺+H).

### EXAMPLE 17

### 2-{5-[4-(2-Chloro-6-fluoro-phenoxymethyl)-2-(4-trifluoromethyl-phenyl)-thiazol-5-ylmethoxy]-indol-1-yl}-propionic acid

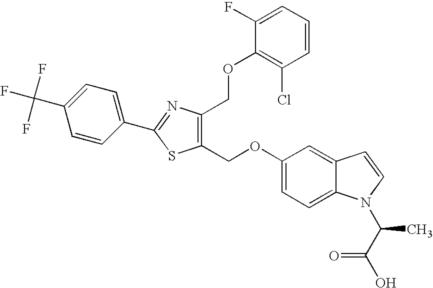

MS (ES): 604.85 (M⁺+H).

### EXAMPLE 18

### {5-[4-Phenoxymethyl-2-(4-trifluoromethyl-phenyl)-thiazol-5-ylmethoxy]-indol-1-yl}-acetic acid

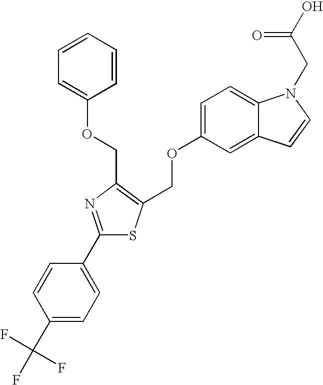

MS (ES): 538.92 (M⁺+H).

### EXAMPLE 19

### Racemic 2-Methyl-2-{5-[4-phenoxymethyl-2-(4-trifluoromethyl-phenyl)-thiazol-5-ylmethoxy]-indol-1-yl}-propionic acid

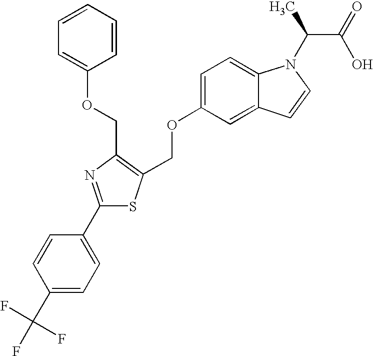

MS (ES): 552.93 (M⁺+H).

### EXAMPLE 20

### 2-Methyl-2-{5-[4-phenoxymethyl-2-(4-trifluoromethyl-phenyl)-thiazol-5-ylmethoxy]-indol-1-yl}-propionic acid

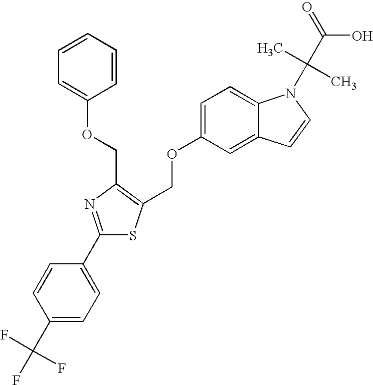

MS (ES): 565.15 (M⁺−H).

### EXAMPLE 21

### 3-{5-[4-Propyl-2-(4-trifluoromethyl-phenyl)-thiazol-5-ylmethoxy]-indol-1-yl}-propionic acid

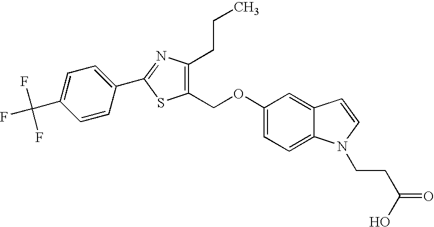

MS (ES): 487.53 (M⁺−H).

### EXAMPLE 22

### 5-{5-[4-Methyl-2-(4-trifluoromethyl-phenyl)-thiazol-5-ylmethoxy]-indol-1-yl}-pentanoic acid

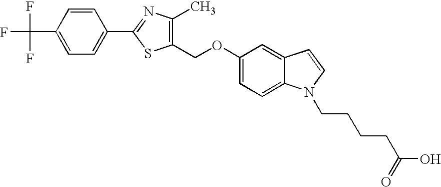

MS (ES): 488.99 (M⁺+H).

### EXAMPLE 23

### 5-{5-[4-Propyl-2-(4-trifluoromethyl-phenyl)-thiazol-5-ylmethoxy]-indol-1-yl}-pentanoic acid

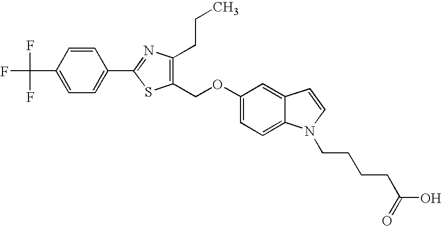

MS (ES): 515.1 (M⁺−H).

### EXAMPLE 24

### {5-[4-Isopropyl-2-(4-trifluoromethyl-phenyl)-thiazol-5-ylmethoxy]-indol-1-yl}-acetic acid

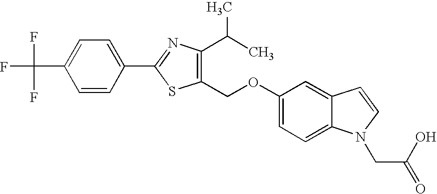

MS (ES): 473.01 (M⁺−H).

### EXAMPLE 25

### {5-[4-(2-Chloro-6-fluoro-phenoxymethyl)-2-(4-trifluoromethyl-phenyl)-thiazol-5-ylmethoxy]-indol-1-yl}-acetic acid

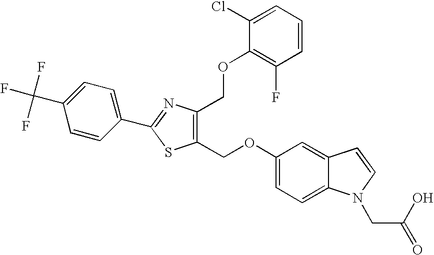

MS (ES): 588.11 (M⁺−H).

### EXAMPLE 26

### 2-{5-[4-(2-Chloro-6-fluoro-phenoxymethyl)-2-(4-trifluoromethyl-phenyl)-thiazol-5-ylmethoxy]-indol-1-yl}-2-methyl-propionic acid

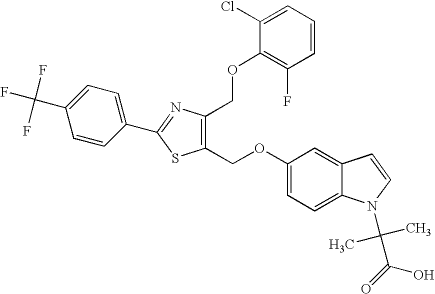

MS (ES): 617.15 (M⁺−H).

### EXAMPLE 27

### 5-{5-[5-Methyl-2-(4-trifluoromethyl-phenyl)-oxazol-4-ylmethoxy]-indol-1-yl}-pentanoic acid

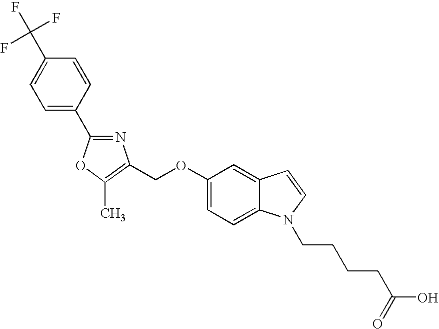

MS (ES): 471.18 (M⁺−H).

### EXAMPLE 28

### 5-{5-[2-(4-Bromo-phenyl)-5-methyl-oxazol-4-ylmethoxy]-indol-1-yl}-pentanoic acid

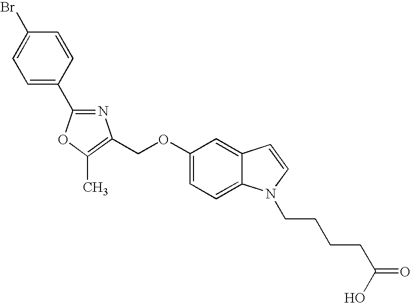

MS (ES): 483.12 (M⁺−H).

### EXAMPLE 29

### 5-{5-[4-Phenethyl-2-(4-trifluoromethyl-phenyl)-thiazol-5-ylmethoxy]-indol-1-yl}-pentanoic acid

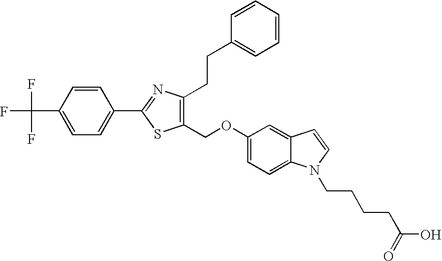

MS (ES): 579.65 (M⁺+H).

### EXAMPLE 30

### 4-[1-(4-Carboxy-butyl)-1H-indol-5-yloxymethyl]-2-(4-trifluoromethyl-phenyl)-thiazole-5-carboxylic acid

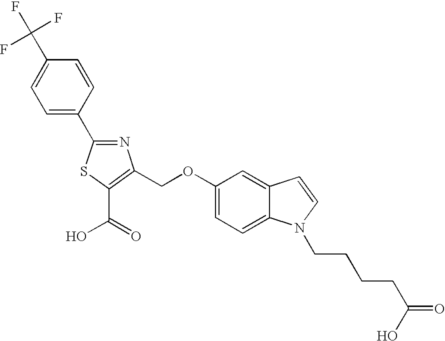

MS (ES): 517.1 (M⁺−H).

### EXAMPLE 31

### 3-{5-[2-(4-Bromo-phenyl)-5-methyl-oxazol-4-ylmethoxy]-indol-1-yl}-propionic acid

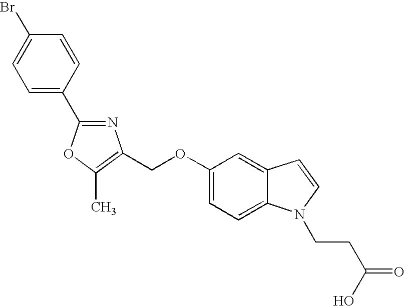

MS (ES): 456.30 (M⁺+H).

### EXAMPLE 32

### 3-{5-[4-Phenethyl-2-(4-trifluoromethyl-phenyl)-thiazol-5-ylmethoxy]-indol-1-yl}-propionic acid

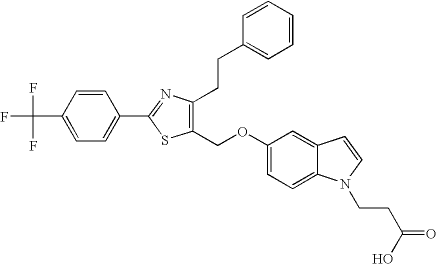

MS (ES): 549.1 (M⁺−H).

### EXAMPLE 33

### 3-{5-[4-Methyl-2-(4-trifluoromethyl-phenyl)-thiazol-5-ylmethoxy]-indol-1-yl}-propionic acid

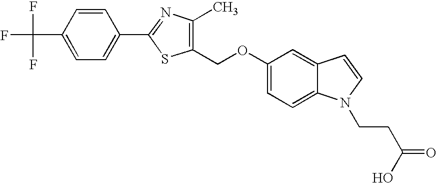

MS (ES): 459.1 (M⁺−H).

### EXAMPLE 34

### 4-[1-(2-Carboxy-ethyl)-1H-indol-5-yloxymethyl]-2-(4-trifluoromethyl-phenyl)-thiazole-5-carboxylic acid

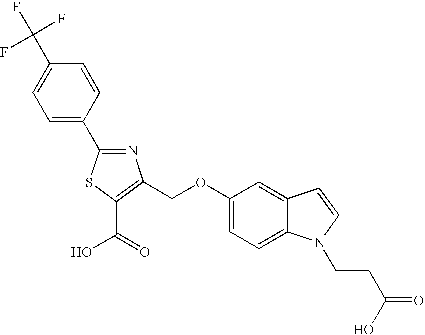

MS (ES): 487.07 (M⁺−H).

### EXAMPLE 35

### {5-[4-Isopropyl-2-(4-trifluoromethyl-phenyl)-thiazol-5-ylmethylsulfanyl]-indol-1-yl}-acetic acid

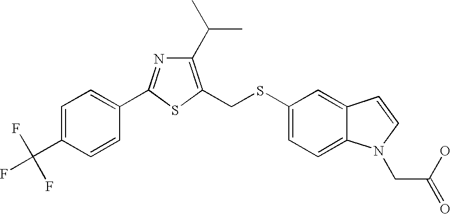

MS (ES): 491.15 (M⁺+H).

### EXAMPLE 36

### {5-[4-Ethyl-2-(4-trifluoromethyl-phenyl)-oxazol-5-ylmethoxy]-indol-1-yl}-acetic acid

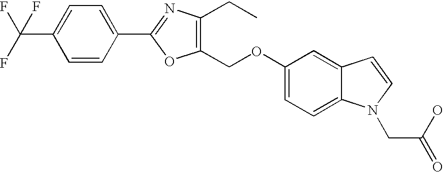

MS [EI+] 445 (M+H)⁺, 443 (M+H)⁻.

### EXAMPLE 37

### (5-{2-[5-Methyl-2-(4-trifluoromethyl-phenyl)-oxazol-4-yl]-ethoxy}-indol-1-yl)-acetic acid

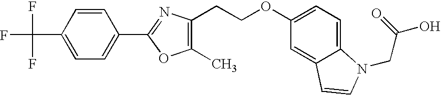

### Step 1

(5-Hydroxy-indol-1-yl)-acetic acid ethyl ester (109 mg, 0.500 mmol) is dissolved into anhydrous acetonitrile (ACN) (2 mL). Toluene-4-sulfonic acid 2-[5-methyl-2-(4-trifluoromethyl-phenyl)-oxazol-4-yl]-ethyl ester (208 mg, 0.490 mmol) is added to the reaction, followed by the addition of cesium carbonate (326 mg, 1.00 mmol). The reaction is allowed to stir under nitrogen at room temperature and monitored by TLC and HPLC. Upon complete consumption of the tosylate, the reaction is diluted with diethyl ether and quenched with 0.1N NaOH. The two phases are separated, then the organic layer washed with water and brine. The organic phase is dried over anhydrous sodium sulfate and concentrated under vacuum. The residue is further purified using either EtOAc/Hexanes (1:9) or Acetone/Hexanes (1:9) gradients on silica gel chromatography to yield (5-{2-[5-Methyl-2-(4-trifluoromethyl-phenyl)-oxazol-4-yl]-ethoxy}-indol-1-yl)-acetic acid ethyl ester (162 mg, 0.343 mmol) or 70%.

### Step 2

(5-{2-[5-Methyl-2-(4-trifluoromethyl-phenyl)-oxazol-4-yl]-ethoxy}-indol-1-yl)-acetic acid ethyl ester (330 mg, 0.343 mmol) is dissolved in tetrahydrofuran (1 mL) and 5N NaOH (1 mL) is added. The mixture is heated to reflux until the conversion is complete. Upon complete conversion, the reaction is cooled to room temperature and 5N HCl (1 mL) is added. The mixture is diluted with diethyl ether and extracted with 1N HCl. The organic layer is washed with water and brine, then dried over anhydrous sodium sulfate. Concentration of the solvent reveals the pure (5-{2-[5-Methyl-2-(4-trifluoromethyl-phenyl)-oxazol-4-yl]-ethoxy}-indol-1-yl)-acetic acid in near quantitative yield (145 mg, 0.326 mmol).

The following compounds are made in a substantially similar manner:

### EXAMPLE 38

### {5-[2-(5-Methyl-2-pyridin-4-yl-thiazol-4-yl)-ethoxy]-indol-1-yl}-acetic acid

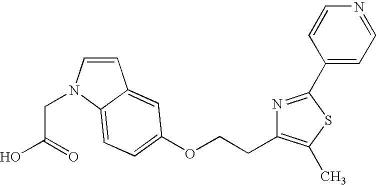

MS (ES): 394.20 (M⁺+1).

### EXAMPLE 39

### {5-[2-(5-Methyl-2-morpholin-4-yl-thiazol-4-yl)-ethoxy]-indol-1-yl}-acetic acid

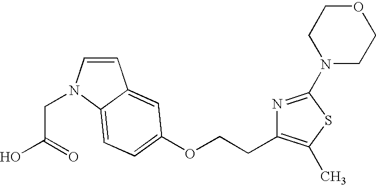

MS (ES): 400.0 (M⁺−H).

### EXAMPLE 40

### (5-{2-[5-Methyl-2-(4-trifluoromethyl-phenyl)-oxazol-4-yl]-ethoxy}-indol-1-yl)-acetic acid

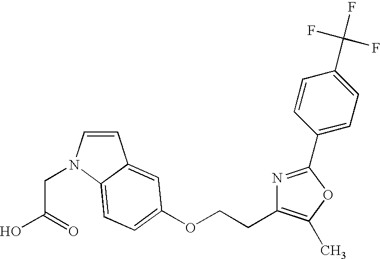

MS (ES): 443.1 (M⁺−H).

### EXAMPLE 41

### (5-{2-[5-Methyl-2-(tetrahydro-pyran-4-yl)-oxazol-4-yl]-ethoxy}-indol-1-yl)-acetic acid

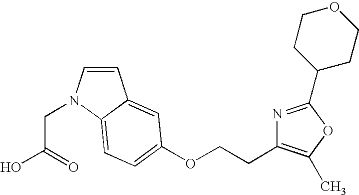

MS (ES): 385.0 (M⁺+1).

### EXAMPLE 42

### {5-[2-(2-Butoxy-5-methyl-oxazol-4-yl)-ethoxy]-indol-1-yl}-acetic acid

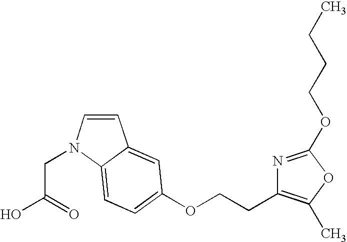

MS (ES): 373.21 (M⁺+1).

### EXAMPLE 43

### {5-[2-(5-Methyl-2-pyridin-3-yl-thiazol-4-yl)-ethoxy]-indol-1-yl}-acetic acid

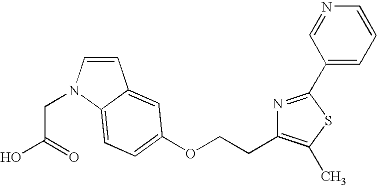

MS (ES): 394.1 (M⁺+1).

### EXAMPLE 44

### {5-[2-(5-Methyl-2-pyridin-2-yl-thiazol-4-yl)-ethoxy]-indol-1-yl}-acetic acid

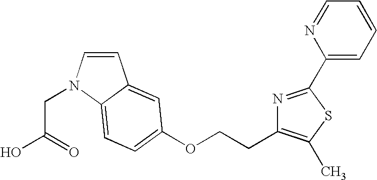

MS (ES): 394.1 (M⁺+1).

### EXAMPLE 45

### (5-{2-[2-(5-Bromo-thiophen-2-yl)-5-methyl-oxazol-4-yl]-ethoxy}-indol-1-yl)-acetic acid

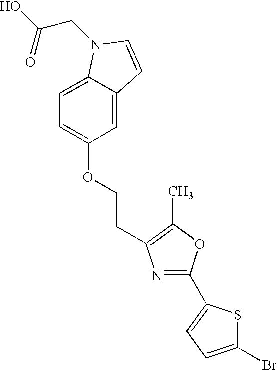

MS (ES): 463.0 (M⁺+1).

### EXAMPLE 46

### {5-[3-(4-Butyl-phenoxy)-propoxy]-indol-1-yl}-acetic acid

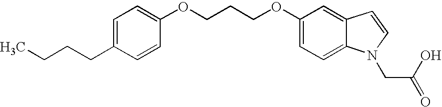

MS (ES): 382.2 (M⁺+1).

### EXAMPLE 47

### (5-{2-[2-(3-Bromo-phenyl)-5-methyl-oxazol-4-yl]-ethoxy}-indol-1-yl)-acetic acid

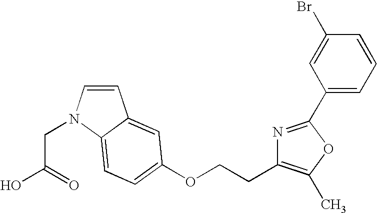

MS (ES): 454.99 (M⁺+1).

### EXAMPLE 48

### (5-{2-[5-Ethyl-2-(4-trifluoromethyl-phenyl)-thiazol-4-yl]-ethoxy}-indol-1-yl)-acetic acid

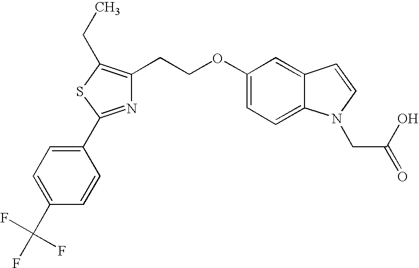

MS (ES): 473.03 (M⁺−1).

### EXAMPLE 49

### 2-(5-{2-[5-Ethyl-2-(4-trifluoromethyl-phenyl)-thiazol-4-yl]-ethoxy}-indol-1-yl)-propionic acid

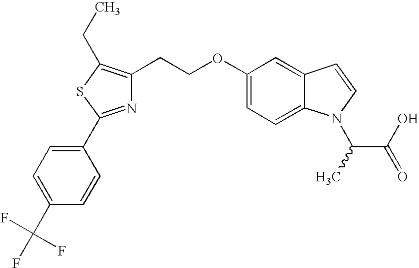

MS (ES): 488.99 (M⁺+1).

### EXAMPLE 50

### (5-{2-[2-(2-Chloro-phenyl)-5-ethyl-thiazol-4-yl]-ethoxy}-indol-1-yl)-acetic acid

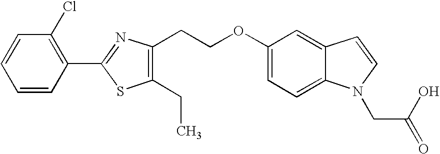

MS (ES): 441.00 (M⁺+H).

### EXAMPLE 51

### 2-(5-{2-[2-(2-Chloro-phenyl)-5-ethyl-thiazol-4-yl]-ethoxy}-indol-1-yl)-propionic acid

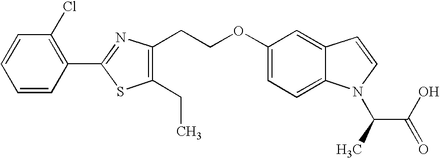

MS (ES): 455.02 (M⁺+H).

### EXAMPLE 52

### (5-{2-[5-Propyl-2-(4-trifluoromethyl-phenyl)-thiazol-4-yl]-ethoxy}-indol-1-yl)-acetic acid

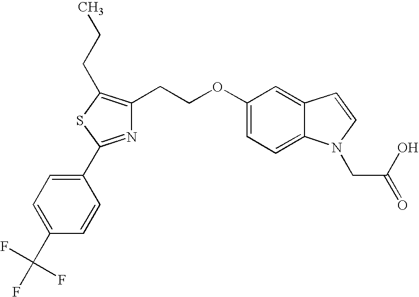

MS (ES): 489.02 (M⁺+H).

### EXAMPLE 53

### 2-(5-{2-[5-Propyl-2-(4-trifluoromethyl-phenyl)-thiazol-4-yl]-ethoxy}-indol-1-yl)-propionic acid

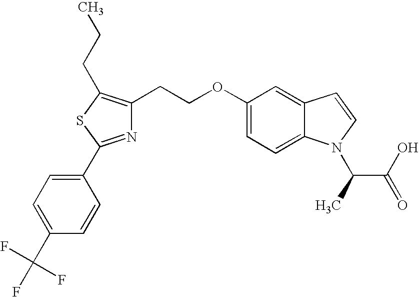

MS (ES): 503.03 (M⁺+H).

### EXAMPLE 54

### 2-(5-{2-[2-(2-Chloro-phenyl)-5-ethyl-thiazol-4-yl]-ethoxy}-indol-1-yl)-2-methyl-propionic acid

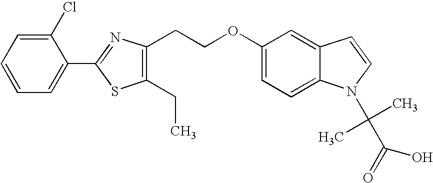

MS (ES): 470.8 (M⁺+H).

### EXAMPLE 55

### Racemic 2-(5-{2-[5-Ethyl-2-(4-trifluoromethyl-phenyl)-oxazol-4-yl]-ethoxy}-indol-1-yl)-propionic acid

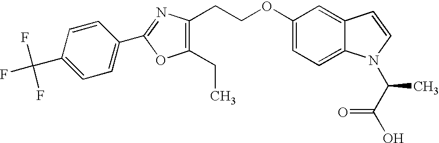

MS (ES): 473.0 (M⁺+H).

### EXAMPLE 56

### (5-{2-[5-Ethyl-2-(4-trifluoromethyl-phenyl)-oxazol-4-yl]-ethoxy}-indol-1-yl)-acetic acid

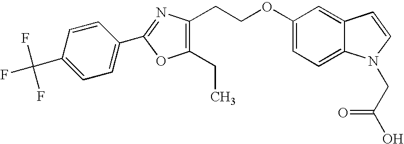

MS (ES): 459.0 (M⁺+H).

### EXAMPLE 57

### 2-(5-{2-[5-Ethyl-2-(4-trifluoromethyl-phenyl)-oxazol-4-yl]-ethoxy}-indol-1-yl)-2-methyl-propionic acid

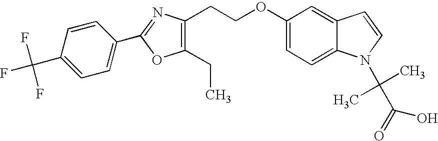

MS (ES): 487.53 (M⁺+H).

### EXAMPLE 58

### 3-(5-{2-[2-(2-Chloro-phenyl)-5-ethyl-thiazol-4-yl]-ethoxy}-indol-1-yl)-propionic acid

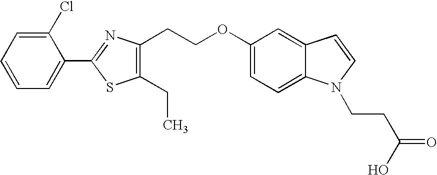

MS (ES): 455.22 (M⁺−H).

### EXAMPLE 59

### 3-(5-{2-[5-Ethyl-2-(4-trifluoromethyl-phenyl)-oxazol-4-yl]-ethoxy}-indol-1-yl)-propionic acid

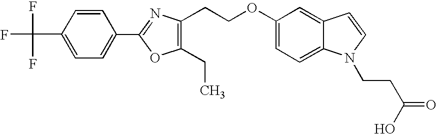

MS (ES): 471.15 (M⁺−H).

### EXAMPLE 60

### 2-Methyl-2-(5-{2-[5-propyl-2-(4-trifluoromethyl-phenyl)-thiazol-4-yl]-ethoxy}-indol-1-yl)-propionic acid

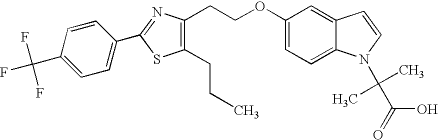

MS (ES): 515.03 (M⁺−H).

### EXAMPLE 61

### Racemic-(5-{1-[4-Methyl-2-(4-trifluoromethyl-phenyl)-thiazol-5-yl]-ethoxy}-indol-1-yl)-acetic acid

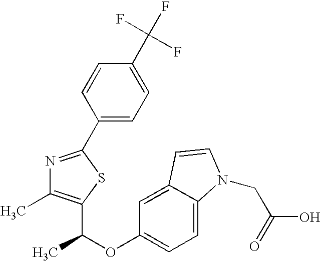

### Step 1

1-[4-Methyl-2-(4-trifluoromethyl-phenyl)-thiazol-S-yl]ethanol (373 mg, 1.3 mmol) is dissolved into anhydrous toluene (10 mL) and cooled in an ice bath to 0° C. with stirring under nitrogen. Tributyl phosphine (500 uL, 2.00 mmol) is added by syringe followed by 1-1′-azodicarbonyl-dipiperidine (505 mg, 2.00 mmol). Finally, 5-hydroxy-indol-1-yl)-acetic acid ethyl ester (438 mg, 2.00 mmol) is then added. The reaction is allowed to stir under nitrogen at 0° C. for 1 hour, then room temperature and monitored by TLC and HPLC. Upon completion, the reaction is diluted with hexanes and allowed to stir vigorously for 10 min. The resulting white precipitate is then filtered away and the solution is concentrated under vacuum. The residue is further purified using either EtOAc/Hexanes (1:9) or Acetone/Hexanes (1:9) gradients on silica gel chromatography to yield (5-{1-[4-Methyl-2-(4-trifluoromethyl-phenyl)-thiazol-5-yl]-ethoxy}-indol-1-yl)-acetic acid ethyl ester (254 mg, 0.520 mmol) or 40%.

### Step 2

5-{1-[4-Methyl-2-(4-trifluoromethyl-phenyl)-thiazol-5-yl]-ethoxy}-indol-1-yl)-acetic acid ethyl ester (254 mg, 0.520 mmol) is dissolved in tetrahydrofuran (1 mL) and 5N NaOH (1 mL) is added. The mixture is heated to reflux until the conversion is complete. Upon complete conversion, the reaction is cooled to room temperature and 5N HCl (1 mL) is added. The mixture is diluted with diethyl ether and extracted with 1N HCl. The organic layer is washed with water and brine, then dried over anhydrous sodium sulfate. Concentration of the solvent reveals the pure (5-{1-[4-Methyl-2-(4-trifluoromethyl-phenyl)-thiazol-5-yl]-ethoxy}-indol-1-yl)-acetic acid in near quantitative yield (227 mg, 0.494 mmol).

The following compounds are made in a substantially similar manner:

### EXAMPLE 62

### Racemic-(5-{1-[4-Methyl-2-(4-trifluoromethyl-phenyl)-thiazol-5-yl]-ethoxy}-indol-1-yl)-acetic acid

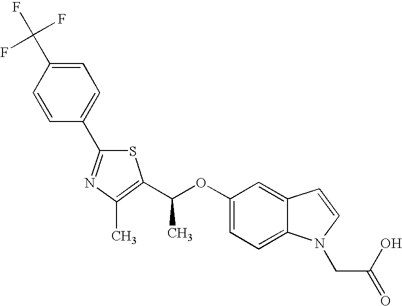

MS (ES): 461.2 (M⁺+H).

### EXAMPLE 63

### Racemic-(5-{1-[4-Isopropyl-2-(4-trifluoromethyl-phenyl)-thiazol-5-yl]-ethoxy}-indol-1-yl)-acetic acid

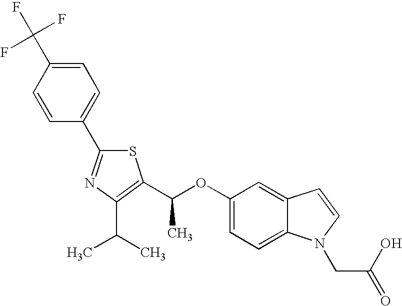

MS (ES): 489.54 (M⁺+H).

### EXAMPLE 64

### Racemic-(5-{2-[5-Ethyl-2-(4-trifluoromethyl-phenyl)-thiazol-4-yl]-1-methyl-ethoxy}-indol-1-yl)-acetic acid

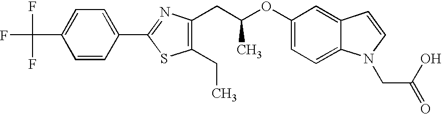

MS (ES): 489.2 (M⁺+H).

### EXAMPLE 65

### Racemic-(5-{2-[5-Methyl-2-(4-trifluoromethyl-phenyl)-thiazol-4-yl]-propoxy}-indol-1-yl)-acetic acid

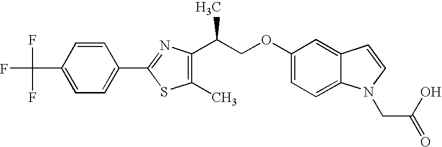

MS (ES): 475.22 (M⁺+H).

### EXAMPLE 66

### (5-{2-[5-Methyl-2-(4-trifluoromethyl-phenyl)-oxazol-4-yl]-propoxy}-indol-1-yl)-acetic acid

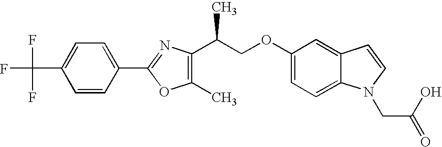

MS (ES): 459.21 (M⁺+H).

### EXAMPLE 67

### (S)-(5-{2-[5-Methyl-2-(4-trifluoromethyl-phenyl)-oxazol-4-yl]-propoxy}-indol-1-yl)-acetic acid

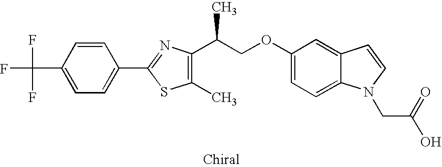

MS (ES): 459.21 (M⁺+H).

### EXAMPLE 68

### (R)-(5-{2-[5-Methyl-2-(4-trifluoromethyl-phenyl)-oxazol-4-yl]-propoxy}-indol-1-yl)-acetic acid

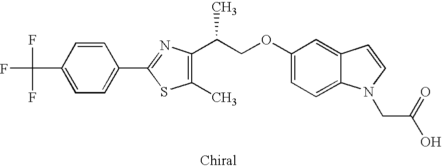

MS (ES): 459.21 (M⁺+H).

### EXAMPLE 69

### Racemic-(5-{1-[4-Propyl-2-(4-trifluoromethyl-phenyl)-thiazol-5-yl]-ethoxy}-indol-1-yl)-acetic acid

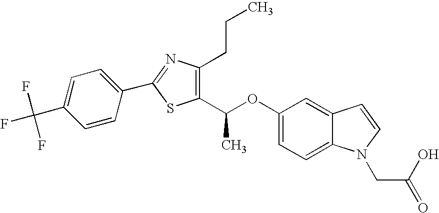

MS (ES): 489.56 (M⁺+H).

### EXAMPLE 70

### Racemic-(6-{1-[4-Isopropyl-2-(4-trifluoromethyl-phenyl)-thiazol-5-yl]ethoxy}-1H-indol-3-yl)-acetic acid

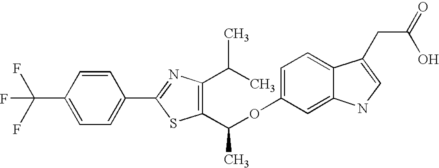

MS (ES): 489.2 (M⁺+H).

### EXAMPLE 71

### Racemic-(6-{2-[5-Methyl-2-(4-trifluoromethyl-phenyl)-thiazol-4-yl]-propoxy}-1H-indol-3-yl)-acetic acid

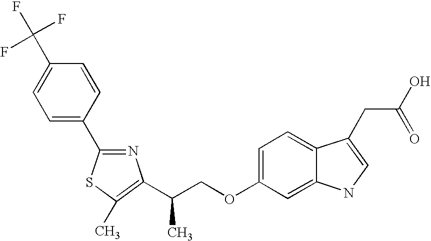

MS (ES): 475.20 (M⁺+H).

### EXAMPLE 72

### Racemic-(1-Methyl-6-{2-[5-methyl-2-(4-trifluoromethyl-phenyl)-thiazol-4-yl]-propoxy}-1H-indol-3-yl)-acetic acid

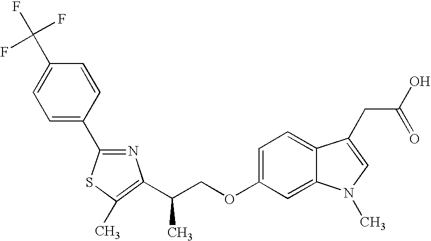

MS (ES): 489.2 (M⁺+H).

### EXAMPLE 73

### (S)-(6-{2-[5-Methyl-2-(4-trifluoromethyl-phenyl)-thiazol-4-yl]-propoxy}-1H-indol-3-yl)-acetic acid

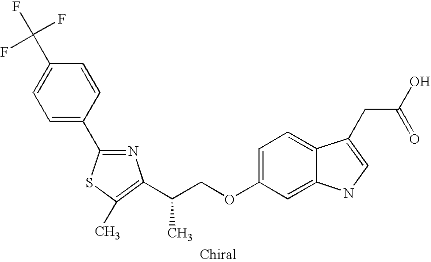

MS (ES): 475.20 (M⁺+H).

### EXAMPLE 74

### (R)-(6-{2-[5-Methyl-2-(4-trifluoromethyl-phenyl)-thiazol-4-yl]-propoxy}-1H-indol-3-yl)-acetic acid

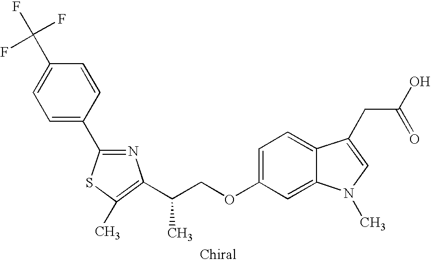

MS (ES): 489.2 (M⁺+H).

### EXAMPLE 75

### Racemic-(6-Hydroxy-5-{1-[4-isopropyl-2-(4-trifluoromethyl-phenyl)-thiazol-5-yl]-ethyl}-1H-indol-3-yl)-acetic acid

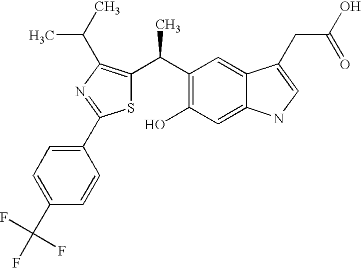

MS (ES): 489.09 (M⁺+H).

### EXAMPLE 76

### (6-{2-[4-Isopropyl-2-(4-trifluoromethyl-phenyl)-thiazol-5-yl]-propoxy}-1-methyl-1H-indol-3-yl)-acetic acid

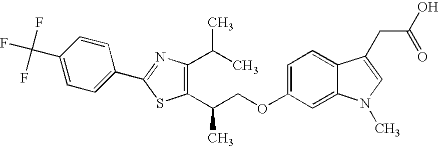

MS (ES): 517.1 (M⁺+H).

### EXAMPLE 77

### (1-Methyl-6-{2-[4-methyl-2-(4-trifluoromethyl-phenyl)-thiazol-5-yl]-propoxy}-1H-indol-3-yl)-acetic acid

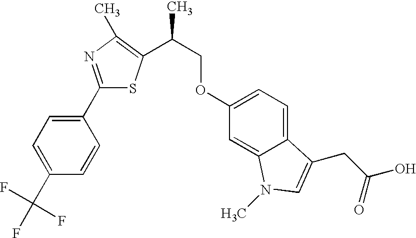

MS (ES): 489.06 (M⁺+H).

### EXAMPLE 78

### (6-{2-[5-Ethyl-2-(4-trifluoromethyl-phenyl)-thiazol-4-yl]-propoxy}-1-methyl-1H-indol-3-yl)-acetic acid

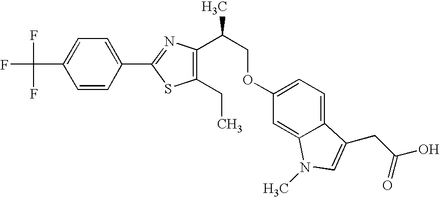

MS (ES): 503.1 (M⁺+H).

### EXAMPLE 79

### (R)-(6-{2-[4-Isopropyl-2-(4-trifluoromethyl-phenyl)-thiazol-5-yl]-propoxy}-1-methyl-1H-indol-3-yl)-acetic acid

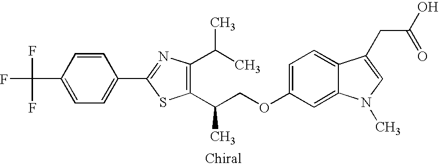

MS (ES): 517.12 (M++H).

### EXAMPLE 80

### (S)-(6-{2-[4-Isopropyl-2-(4-trifluoromethyl-phenyl)-thiazol-5-yl]-propoxy}-1-methyl-1H-indol-3-yl)-acetic acid

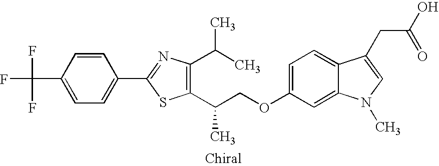

MS (ES): 517.12 (M⁺+H).

### EXAMPLE 81

### Racemic-(1-Methyl-6-{2-[4-methyl-2-(4-trifluoromethyl-phenyl)-thiazol-5-yl]-propoxy}-1H-indol-3-yl)-acetic acid

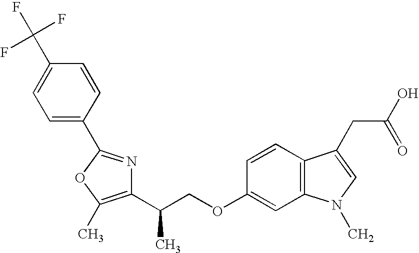

MS (ES): 473.12 (M⁺+H).

### EXAMPLE 82

### Racemic-(1-Ethyl-6-{2-[4-isopropyl-2-(4-trifluoromethyl-phenyl)-thiazol-5-yl]-propoxy}-1H-indol-3-yl)-acetic acid

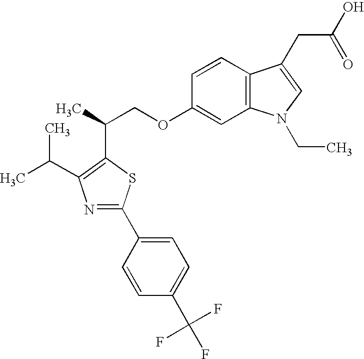

MS (ES): 531.2 (M⁺+H).

### EXAMPLE 83

### Racemic-(6-{2-[4-Isopropyl-2-(4-trifluoromethyl-phenyl)-thiazol-5-yl]-propoxy}-1-propyl-1H-indol-3-yl)-acetic acid

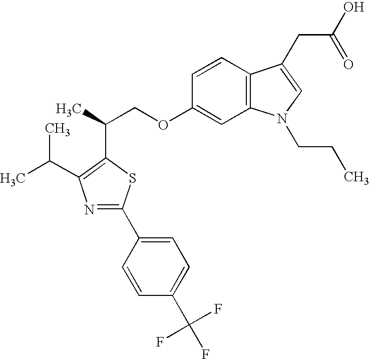

MS (ES): 545.65 (M⁺+H).

### EXAMPLE 84

### Racemic-(5-{1-[4-Ethyl-2-(4-trifluoromethyl-phenyl)-oxazol-5-yl]-ethoxy}-indol-1-yl)-acetic acid

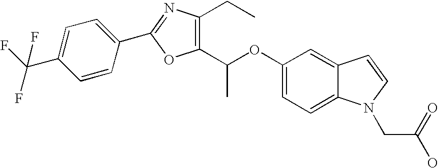

MS [EI+] 459 (M+H)⁺, 457 (M+H)⁻.

### EXAMPLE 85

### {5-[4-Propyl-2-(4-trifluoromethyl-phenyl)-thiazol-5-ylmethoxy]-indol-1-yl}-acetic acid

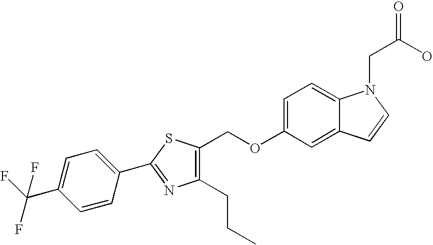

### Step A

### {5-[4-Propyl-2-(4-trifluoromethyl-phenyl)-thiazol-5-ylmethoxy]-indol-1-yl}-acetic acid ethyl ester

To a solution of 5-Chloromethyl-4-propyl-2-(4-trifluoromethyl-phenyl)-thiazole (160 mg, 0.5 mmol) and (5-Hydroxy-indol-1-yl)-acetic acid ethyl ester (110 mg, 0.5 mmol) in acetonitrile (3 mL) is added Cs2CO3 (325 mg, 1 mmol). The mixture is stirred at room temperature over night, quenched by water, extracted with ethyl acetate, dried over sodium sulfate. Concentration yields the crude product.

### Step B

### {5-[4-Propyl-2-(4-trifluoromethyl-phenyl)-thiazol-5-ylmethoxy]-indol-1-yl}-acetic acid

To a solution of {5-[4-Propyl-2-(4-trifluoromethyl-phenyl)-thiazol-5-ylmethoxy]-indol-1-yl}-acetic acid ethyl ester from step A in ethanol (2 mL) is added NaOH (5.0 M, 1 mL). After heated at 50° C. for 2 hrs, ethanol is evaporated. The residue is diluted with water, acidified with 5 N HCl, extracted with ether, dried over sodium sulfate. Concentration and purification by reversed phase HPLC (acetone/water/TFA as eluents) yields the title compound (120 mg). MS (ES): 473.0 (M⁺−1); the structure is also confirmed by ¹H NMR.

The following compounds are made in a similar manner, all structures are confirmed by MS and proton NMR:

### EXAMPLE 86

### Racemic-2-{5-[4-Propyl-2-(4-trifluoromethyl-phenyl)-thiazol-5-ylmethoxy]-indol-1-yl}-propionic acid

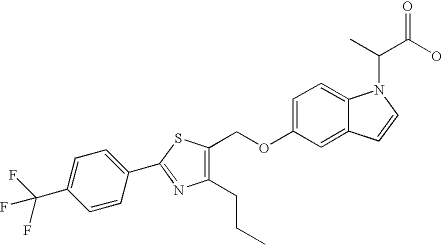

MS (ES): 489.0 (M⁺+1); the structure is also confirmed by ¹H NMR.

### EXAMPLE 87

### 2-Methyl-2-{5-[4-propyl-2-(4-trifluoromethyl-phenyl)-thiazol-5-ylmethoxy]-indol-1-yl}-propionic acid

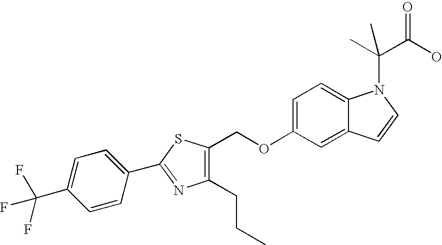

MS (ES): 503.0 (M⁺+1); the structure is also confirmed by ¹H NMR.

### EXAMPLE 88

### Racemic-2-{5-[4-Phenethyl-2-(4-trifluoromethyl-phenyl)-thiazol-5-ylmethoxy]-10 indol-1-yl}-propionic acid

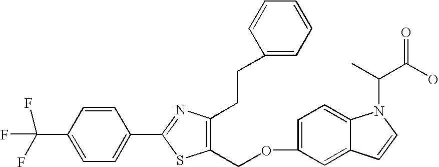

MS (ES): 551.0 (M⁺+1); the structure is also confirmed by ¹H NMR.

### EXAMPLE 89

### 2-Methyl-2-{5-[4-phenethyl-2-(4-trifluoromethyl-phenyl)-thiazol-5-ylmethoxy]-indol-1-yl}-propionic acid

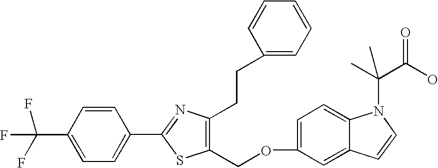

MS (ES): 565.0 (M⁺+1); the structure is also confirmed by ¹H NMR.

### EXAMPLE 90

### {5-[4-Phenethyl-2-(4-trifluoromethyl-phenyl)-thiazol-5-ylmethoxy]-indol-1-yl}-acetic acid

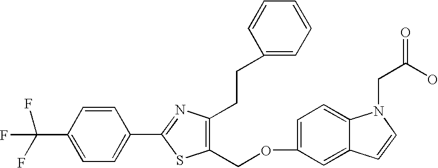

MS (ES): 537.0 (M⁺+1); the structure is also confirmed by ¹H NMR.

### EXAMPLE 91

### {5-[4-Phenyl-2-(4-trifluoromethyl-phenyl)-thiazol-5-ylmethoxy]-indol-1-yl}-acetic acid

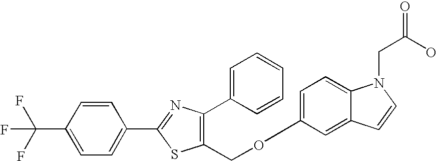

MS (ES): 509.1 (M⁺+1); the structure is also confirmed by ¹H NMR.

### EXAMPLE 92

### {5-[4-tert-Butyl-2-(4-trifluoromethyl-phenyl)-thiazol-5-ylmethoxy]-indol-1-yl}-15 acetic acid

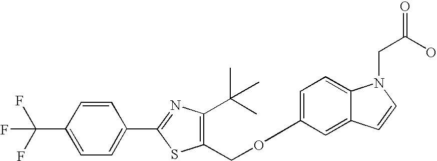

MS (ES): 489.2 (M⁺+1); the structure is also confirmed by ¹H NMR.

### EXAMPLE 93

### 20 {5-[4-[2-(2-Chloro-6-fluoro-phenyl)-ethyl]-2-(4-trifluoromethyl-phenyl)-thiazol-5-ylmethoxy]-indol-1-yl}-acetic acid

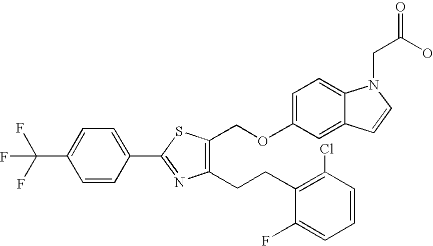

MS (ES): 589.1 (M⁺+1, ³⁵Cl), 591.2 (M⁺+1, ³⁷Cl); the structure is also confirmed by ¹HNMR.

### EXAMPLE 94

### 2-Methyl-2-[5-(5-methyl-2-phenyl-oxazol-4-ylmethoxy)-indol-1-yl]-propionic acid

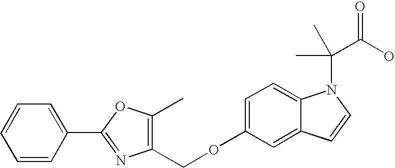

MS (ES): 391.2 (M⁺+1); the structure is also confirmed by ¹H NMR.

### EXAMPLE 95

### 2-{5-[2-(4-Trifluoromethyl-phenyl)-5-methyl-oxazol-4-ylmethoxy]-indol-1-yl}-2-methyl-propionic acid

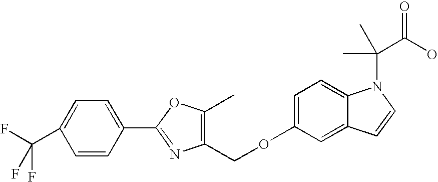

MS (ES): 459.11 (M⁺+1); the structure is also confirmed by ¹HNMR.

### EXAMPLE 96

### 2-{5-[2-(4-Fluoro-phenyl)-5-methyl-oxazol-4-ylmethoxy]-indol-1-yl}-2-methyl-propionic acid

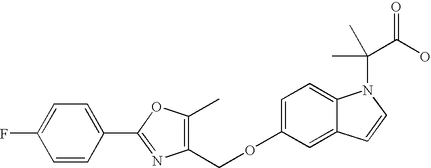

MS (ES): 409.13 (M⁺+1); the structure is also confirmed by ¹H NMR.

### EXAMPLE 97

### 2-{5-[2-(4-Bromo-phenyl)-5-methyl-oxazol-4-ylmethoxy]-indol-1-yl}-2-methyl-propionic acid

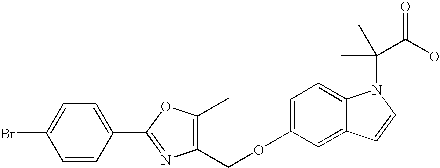

MS (ES): 485.0 (M⁺+1, ⁷⁹Br), 487.0 (M⁺+1, ⁸¹Br); the structure is also confirmed by ¹H NMR.

### EXAMPLE 98

### 2-Methyl-2-(5-{2-[5-methyl-2-(4-trifluoromethyl-phenyl)-oxazol-4-yl]-ethoxy}-indol-1-yl)-propionic acid

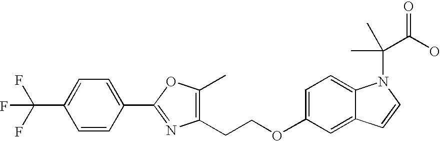

MS (ES): 473.12 (M⁺+1); the structure is also confirmed by ¹H NMR.

### EXAMPLE 99

### 2-(5-{2-[2-(4-Bromo-phenyl)-5-methyl-oxazol-4-yl]-ethoxy}-indol-1-yl)-2-methyl-propionic acid

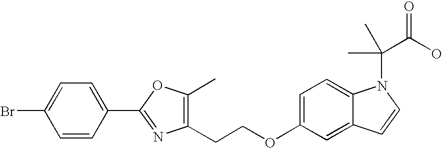

MS (ES): 483.0 (M⁺+1, ⁷⁹Br), 485.0 (M⁺+1, ⁸¹Br); the structure is also confirmed by ¹H NMR.

### EXAMPLE 100

### 2-(5-{2-[2-(5-Bromo-thiophen-2-yl)-5-methyl-oxazol-4-yl]-ethoxy}-indol-1-yl)-2-methyl-propionic acid

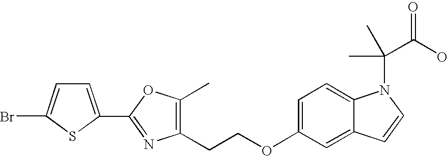

MS (ES): 489 (M⁺+1, ⁷⁹Br), 491.0 (M⁺+1, ⁸¹Br); the structure is also confirmed by ¹H NMR.

### EXAMPLE 101

### 2-Methyl-2-{5-[2-(5-methyl-2-phenyl-thiazol-4-yl)-ethoxy]-indol-1-yl}-propionic acid

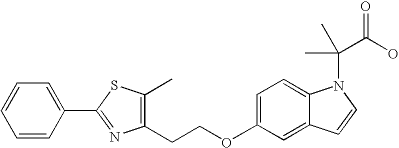

MS (ES): 421.15 (M⁺+1); the structure is also confirmed by ¹H NMR.

### EXAMPLE 102

### {5-[4-Isopropyl-2-(4-trifluoromethyl-phenyl)-thiazol-5-ylmethoxy]-2-methyl-indol-1-yl}-acetic acid

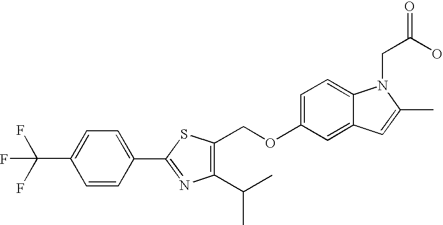

MS (ES): 489.2 (M⁺+1); the structure is also confirmed by ¹H NMR.

### EXAMPLE 103

### 2-{5-[2-(3,5-Bis-trifluoromethyl-phenyl)-4-methyl-thiazol-5-ylmethoxy]-indol-1-yl}-2-methyl-propionic acid

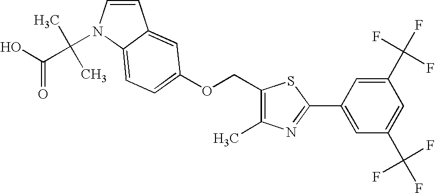

MS (ES): 542.90 (M⁺+1).

### EXAMPLE 104

### {4-[4-Propyl-2-(4-trifluoromethyl-phenyl)-thiazol-5-ylmethoxy]-indol-1-yl}-acetic acid

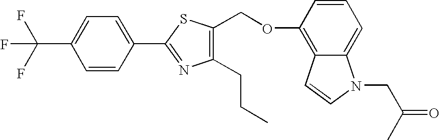

### Step A

### 4-[4-Propyl-2-(4-trifluoromethyl-phenyl)-thiazol-5-ylmethoxy]-1H-indole

To a solution of 5-Chloromethyl-4-propyl-2-(4-trifluoromethyl-phenyl)-thiazole (320 mg, 1 mmol) and 5-Hydroxy-indole (135 mg, 1 mmol) in DMF (5 mL) is added Cs2CO3 (650 mg, 2 mmol). The mixture is stirred at room temperature over night, quenched by water, extracted with ethyl acetate, dried over sodium sulfate. Concentration yields the crude product.

### Step B

### {5-[4-Propyl-2-(4-trifluoromethyl-phenyl)-thiazol-5-ylmethoxy]-indol-1-yl}-acetic acid

To a solution of 4-[4-Propyl-2-(4-trifluoromethyl-phenyl)-thiazol-5-ylmethoxy]-1H-indole from step A in DMF (3 mL) is added ethyl 2-bromacetate (3 mmol) and Cs2CO3 (980 mg, 3 mmol). After heated at 50° C. overnight, cooled to room temperature, treated with 5N NaOH (1.0 mL). The mixture is heated at 50° C. for 1 h, diluted with water, acidified with 5 N HCl, extracted with ether, dried over sodium sulfate. Concentration and purification by reversed phase HPLC (acetone/water/TFA as eluents) yields the title compound. MS (ES): 475.0 (M⁺+1); the structure is also confirmed by ¹H NMR.

### EXAMPLE 105

### {1-[4-Isopropyl-2-(4-trifluoromethyl-phenyl)-thiazol-5-ylmethyl]-1H-indol-5-yloxy}-acetic acid

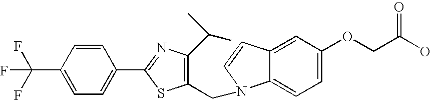

### Step A

### (1H-Indol-5-yloxy)-acetic acid ethyl ester

To a solution of 5-hydroxylindole (0.5 mg, 3.75 mmol) and ethyl 2-bromoacetate (0.42 mL) in acetonitrile (25 mL) is added Cs2CO3 (2.5 mg). The mixture is stirred at room temperature overnight, quenched by water, extracted with ethyl acetate, dried over sodium sulfate. Concentration yields the crude product.

### Step B

### {1-[4-Isopropyl-2-(4-trifluoromethyl-phenyl)-thiazol-5-ylmethyl]-1H-indol-5-yloxy}-acetic acid

To a solution of (1H-Indol-5-yloxy)-acetic acid ethyl ester from step A (110 mg, 0.5 mmol) in DMF (3 mL) is added 5-Cchloromethyl-4-isopropyl-2-(4-trifluoromethyl-phenyl)-thiazole (160 mg, 0.5 mmol) and Cs2CO3 (325 mg, 1 mmol), stirred overnight, treated with 1 N LiOH (2.0 mL). After stirred at room temperature for 2 hrs, diluted with water, acidified with 5 N HCl, extracted with ether, dried over sodium sulfate. Concentration and purification by reversed phase HPLC (acetone/water/TFA as eluents) yields the title compound. MS (ES): 475.2 (M⁺+1); the structure is also confirmed by ¹H NMR.

### EXAMPLE 106

### Racemic-(5-{1-[4-[2-(2-Chloro-6-fluoro-phenyl)-ethyl]-2-(4-trifluoromethyl-phenyl)-thiazol-5-yl]-ethoxy}-indol-1-yl)-acetic acid

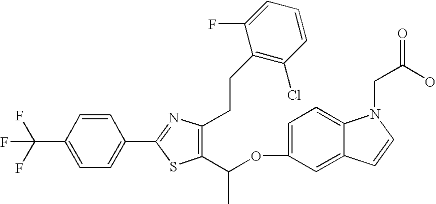

### Step A

### (5-{1-[4-[2-(2-Chloro-6-fluoro-phenyl)-ethyl]-2-(4-trifluoromethyl-phenyl)-thiazol-5-yl]-ethoxy}-indol-1-yl)-acetic acid ethyl ester

A solution of (5-hydroxy-indol-1-yl)-acetic acid ethyl ester (153 mg, 0.7 mmol) and 1-[4-[2-(2-chloro-6-fluoro-phenyl)-ethyl]-2-(4-trifluoromethyl-phenyl)-thiazol-5-yl]-ethanol (200 mg, 0.465 mmol) in toluene (3.0 mL) is degassed and filled with nitrogen for 3 times. Tributylphosphine (0.174 mL, 0.7 mmol) is added to the reaction mixture under nitrogen at 0° C., followed by addition of of 1,1′-(azodicarbonyl)-dipiperidine (177 mg, 0.7 mmol). The reaction mixture is allowed to warm to room temperature and stirred overnight, the mixture is loaded on silica gel column. Chromatography yields the title compound (130 mg).

### Step B

### (5-{1-[4-[2-(2-Chloro-6-fluoro-phenyl)-ethyl]-2-(4-trifluoromethyl-phenyl)-thiazol-5-yl]-ethoxy}-indol-1-yl)-acetic acid

(5-{-[4-[2-(2-Chloro-6-fluoro-phenyl)-ethyl]-2-(4-trifluoromethyl-phenyl)-thiazol-5-yl]-ethoxy}-indol-1-yl)-acetic acid ethyl ester (30 mg) is taken into THF (0.5 mL and treated with LiOH (1.0 N, 0.5 mL) for 2 hrs. The reaction mixture is acidified with 5 N HCl, extracted with ethyl ether, dried over sodium sulfate. Concentration yields the title compound. MS (ES): 603.2 (M⁺+1, ³⁵Cl), 605.2 (M⁺+1, ³⁷Cl), the structure is also confirmed by proton NMR.

The following compounds are made in a similar manner:

### EXAMPLE 107

### Racemic-(5-{1-[4-Methyl-2-(4-trifluoromethyl-phenyl)-thiazol-5-yl]-2-phenyl-ethoxy}-indol-1-yl)-acetic acid

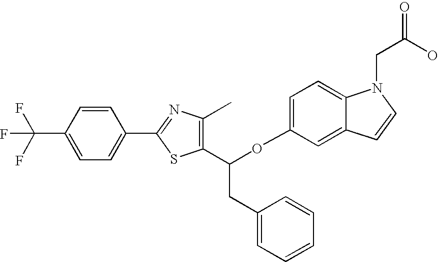

MS (ES): 537.2 (M⁺+1), the structure is also confirmed by proton NMR.

### EXAMPLE 108

### Racemic-(5-{1-[4-Phenethyl-2-(4-trifluoromethyl-phenyl)-thiazol-5-yl]-ethoxy}-indol-1-yl)-acetic acid

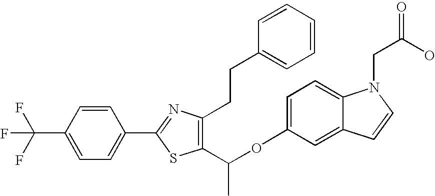

The structure is confirmed by proton NMR.

### EXAMPLE 109

### 2-{5-[4-Ethyl-2-(4-trifluoromethyl-phenyl)-thiazol-5-ylmethoxy]-indol-1-yl}-2-methyl-propionic acid

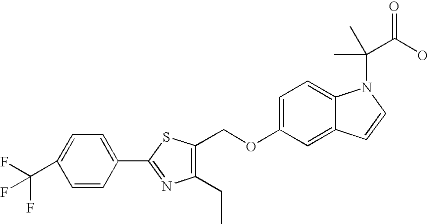

MS (ES): 488.99 (M⁺+1), the structure is also confirmed by proton NMR.

### EXAMPLE 110

### Racemic(6-{1-[4-Isopropyl-2-(4-trifluoromethyl-phenyl)-thiazol-5-yl]-ethoxy}-1H-indol-3-yl)-acetic acid

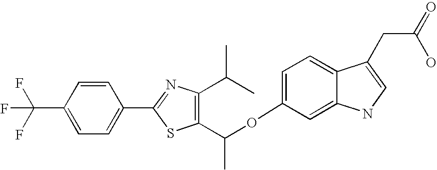

MS (ES): 489.2 (M⁺+1), the structure is also confirmed by proton NMR.

### EXAMPLE 111

### Racemic(6-{1-[4-methyl-2-(4-trifluoromethyl-phenyl)-thiazol-5-yl]-ethoxy}-1H-indol-3-yl)-acetic acid

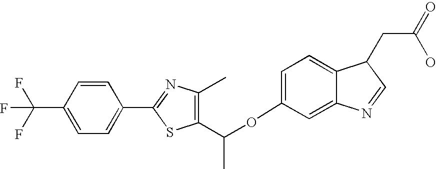

MS (ES): 461.2 (M⁺+1), the structure is also confirmed by proton NMR.

### EXAMPLE 112

### (R)-(5-{1-[4-[2-(2-Chloro-6-fluoro-phenyl)-ethyl]-2-(4-trifluoromethyl-phenyl)-thiazol-5-yl]ethoxy}-indol-1-yl)-acetic acid

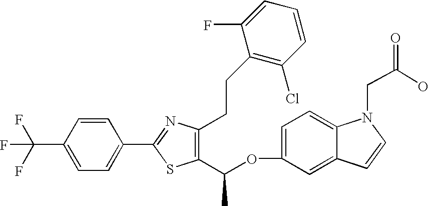

The racemic (5-{1-[4-[2-(2-chloro-6-fluoro-phenyl)-ethyl]-2-(4-trifluoromethyl-phenyl)-thiazol-5-yl]-ethoxy}-indol-1-yl)-acetic acid is resolved on a Chiralpak AD column (2.1×25 cm). Eluted with 20% isopropanol in heptane with 0.1% TFA and concentrated the fractions to provide a pure enantiomer.

### EXAMPLE 113

### (S)-(5-{1-[4-[2-(2-Chloro-6-fluoro-phenyl)-ethyl]-2-(4-trifluoromethyl-phenyl)-thiazol-5-yl]-ethoxy}-indol-1-yl)-acetic acid

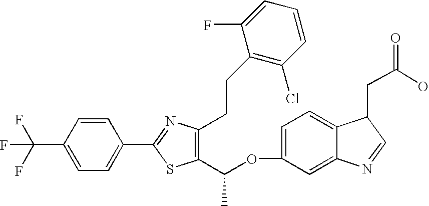

From the chiral separation in previous example also yields this enantiomer.

The following compounds are resolved by chiral HPLC column in a similar manner:

### EXAMPLE 114

### (S)-(5-{1-[4-[2-phenylethyl]-2-(4-trifluoromethyl-phenyl)-thiazol-5-yl]-ethoxy}-indol-1-yl)-acetic acid

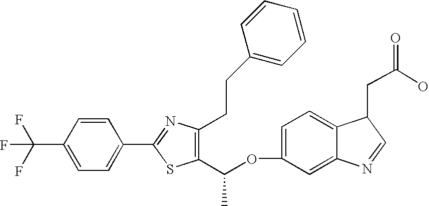

### EXAMPLE 115

### (R)-(5-{1-[4-[2-phenylethyl]-2-(4-trifluoromethyl-phenyl)-thiazol-5-yl]-ethoxy}-indol-1-yl)-acetic acid

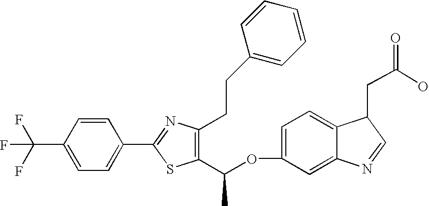

### EXAMPLE 116

### (R)-(6-{1-[4-Isopropyl-2-(4-trifluoromethyl-phenyl)-thiazol-5-yl]-ethoxy}-1H-indol-3-yl)-acetic acid

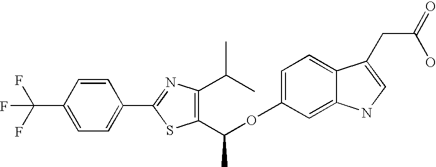

MS (ES): 489.2 (M⁺+1).

### EXAMPLE 117

### (S)-(6-{1-[4-Isopropyl-2-(4-trifluoromethyl-phenyl)-thiazol-5-yl]-ethoxy}-1H-indol-3-yl)-acetic acid

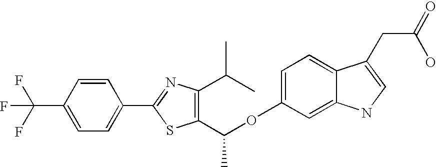

MS (ES): 489.2 (M⁺+1).

### EXAMPLE 118

### (s)-(5-{1-[4-Propyl-2-(4-trifluoromethyl-phenyl)-thiazol-5-yl]-ethoxy}-indol-1-yl)-acetic acid

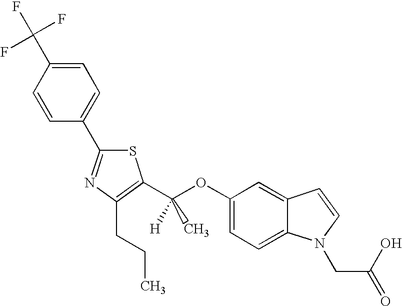

MS (ES): 489.2 (M⁺+H).

### EXAMPLE 119

### (R)-(5 {1-[4-Propyl-2-(4-trifluoromethyl-phenyl)-thiazol-5-yl]-ethoxy}-indol-1-yl)-acetic acid

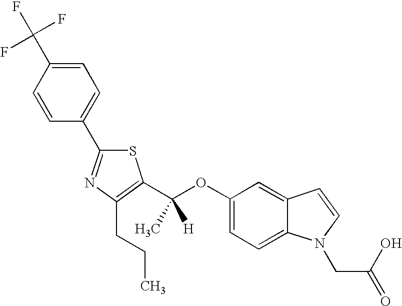

MS (ES): 489.2 (M⁺+H).

### EXAMPLE 120

### (R)-(5-{1-[4-Isopropyl-2-(4-trifluoromethyl-phenyl)-thiazol-5-yl]-ethoxy}-indol-1-yl)-acetic acid

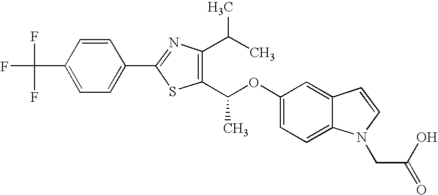

MS (ES): 489.2 (M⁺+H).

### EXAMPLE 121

### (R)-(5-{1-[4-Methyl-2-(4-trifluoromethyl-phenyl)-thiazol-5-yl]-ethoxy}-indol-1-yl)-acetic acid

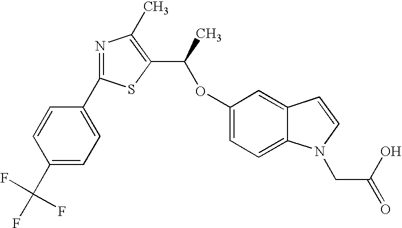

MS (ES): 461.1 (M⁺+H).

### EXAMPLE 122

### (S)-(5-{1-[4-Methyl-2-(4-trifluoromethyl-phenyl)-thiazol-5-yl]-ethoxy}-indol-1-yl)-acetic acid

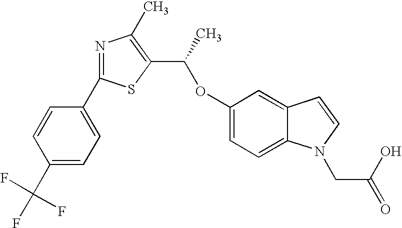

MS (ES): 461.10 (M⁺+H).

### EXAMPLE 123

### (s)-(5-{2-[5-Ethyl-2-(4-trifluoromethyl-phenyl)-thiazol-4-yl]-1-methyl-ethoxy}-indol-1-yl)-acetic acid

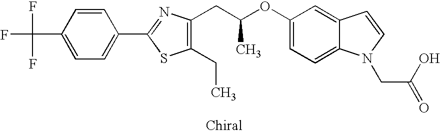

MS (ES): 489.2 (M⁺+H).

### EXAMPLE 124

### (R)-(5-{2-[5-Ethyl-2-(4-trifluoromethyl-phenyl)-thiazol-4-yl]-1-methyl-ethoxy}-indol-1-yl)-acetic acid

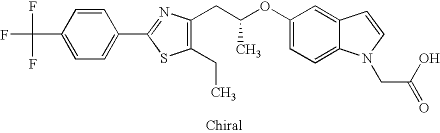

MS (ES): 489.2 (M⁺+H).

### EXAMPLE 140

### (R)-5-{2-[5-Methyl-2-(4-trifluoromethyl-phenyl)-thiazol-4-yl]-propoxy}-1H-indol-3-yl)-acetic acid

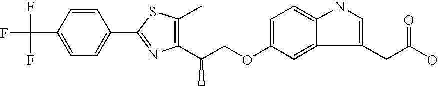

MS (ES):473.3 (M⁺−1).

### EXAMPLE 141

### (S)-(5-{2-[5-Methyl-2-(4-trifluoromethyl-phenyl)-thiazol-4-yl]-propoxy}-1H-indol-3-yl)-acetic acid

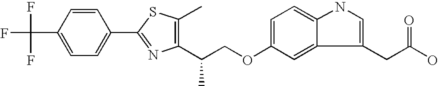

MS (ES):475.2 (M⁺+1).

### EXAMPLE 142

### (5-{2-[5-Methyl-2-(4-trifluoromethyl-phenyl)-thiazol-4-yl]-ethoxy}-1H-indol-3-yl)-acetic acid

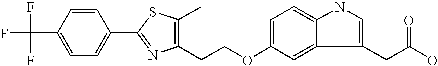

MS (ES):459.3 (M⁺−1).

### EXAMPLE 143

### (R)-(1-Methyl-5-{2-[5-methyl-2-(4-trifluoromethyl-phenyl)-thiazol-4-yl]-propoxy}-1H-indol-3-yl)-acetic acid

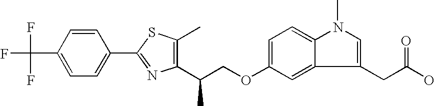

MS (ES):489.3 (M⁺−1).

### EXAMPLE 144

### (S)-(1-Methyl-5-{2-[5-Methyl-2-(4-trifluoromethyl-phenyl)-thiazol-4-yl]-propoxy}-1H-indol-3-yl)-acetic acid

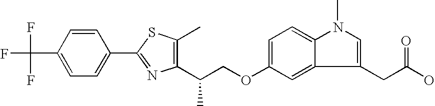

MS (ES):489.3 (M⁺+1).

### EXAMPLE 145

### (1-Methyl-5-{2-[5-methyl-2-(4-trifluoromethyl-phenyl)-thiazol-4-yl]-ethoxy}-1H-indol-3-yl)-acetic acid

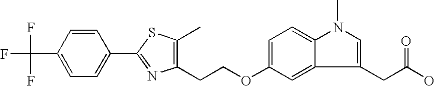

MS (ES):475.32 (M⁺+1).

### EXAMPLE 147

### 5-{2-[4-Isopropyl-2-(4-trifluoromethyl-phenyl)-oxazol-5-yl]-propoxy}-1H-indole-2-carboxylic acid

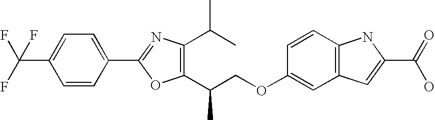

MS (ES): 473 (M⁺+1). The structure is confirmed by ¹H NMR spectroscopy.

### EXAMPLE 148

### 5-{2-[4-Isopropyl-2-(4-trifluoromethyl-phenyl)-oxazol-5-yl]-propoxy}-1-methyl-1H-indole-2-carboxylic acid

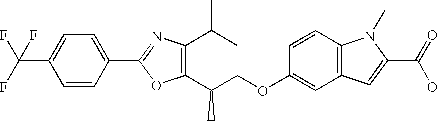

### Step A

### 5-{2-[4-Isopropyl-2-(4-trifluoromethyl-phenyl)-oxazol-5-yl]-propoxy}-1-methyl-1H-indole-2-carboxylic acid ethyl ester

5-{2-[4-Isopropyl-2-(4-trifluoromethyl-phenyl)-oxazol-5-yl]-propoxy}-1H-indole-2-carboxylic acid ethyl ester (0.117 g, 0.234 mmol) is stirred with sodium hydride, 60% mineral oil (0.011 g, 0.28 mmol) and NN-dimethyl formamide (12 mL). Iodomethane (16 uL, 0.26 mmol) is added and the mixture is stirred at 80 deg C. 2 hr, and room temperature 18 hr. The mixture is diluted with water (50 mL), and the product is extracted into ethyl acetate (2×50 mL). The combined extracts are dried over anhydrous magnesium sulfate, filtered, concentrated, and purified via silica gel chromatography eluting with 8:2 hexanes:ethyl acetate to 6:4 hexanes:ethyl acetate to afford the title compound as a tan solid, 0.062 g, 51%). MS M++1 515. The structure is confirmed by ¹H NMR spectroscopy.

### Step B

### 5-{2-[4-Isopropyl-2-(4-trifluoromethyl-phenyl)-oxazol-5-yl]-propoxy}-1-methyl-1H-indole-2-carboxylic acid

Hydrolysis of the ester from step A yields the title compound as a white solid. MS (ES) 487 (M⁺+1). The structure is confirmed by ¹H NMR spectroscopy.

### EXAMPLE 167

### N-(2-{5-[5-Methyl-2-(4-trifluoromethyl-phenyl)-oxazol-4-ylmethoxy]-indol-1-yl}-acetyl)-methanesulfonamide

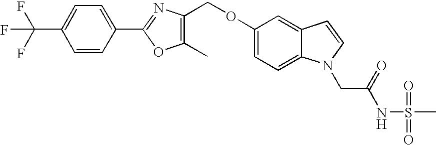

A solution of {5-[5-Methyl-2-(4-trifluoromethyl-phenyl)-oxazol-4-ylmethoxy]-indol-1-yl}-acetic acid (55 mg, 0.127 mmol), methanesulfonamide (12.1 mg, 0.127 mmol), 4-(dimethylamino)pyridine (16 mg, 0.127 mmol), and N,N-diisopropylethylamine (0.045 ml, 0.254 mmol) in dichloromethane (1 ml) is treated with 1-[3-(dimethylamino)propyl]-3-ethylcarbodiimide hydrochloride (39 mg, 0.203 mmol), and the resulting mixture is stirred at ambient temperature for 24 hours. Concentration of the reaction mixture yields a residue which is partitioned between 1N HCl (20 ml) and ethyl acetate (15 ml). The organic layer is washed with brine (25 ml), dried (Na₂SO₄), and concentrated to an oil which is purified by silica chromatography (20:1 hexanes:ethyl acetate to 5:1 hexanes:ethyl acetate) to provide the title compound as a colorless oil. MS: m/z (M+1) 508. The structure is also confirmed by proton NMR.

### EXAMPLE 168

### N-(2-{5-[5-Methyl-2-(4-trifluoromethyl-phenyl)-oxazol-4-ylmethoxy]-indol-1-yl}-acetyl)-benzenesulfonamide

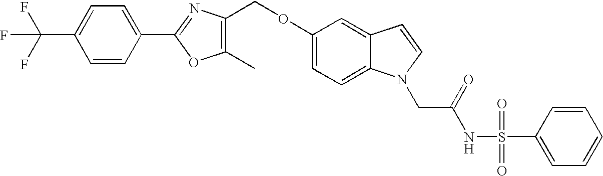

MS: m/z (M+1) 570. The structure is also confirmed by proton NMR.

### EXAMPLE 169

### N-[2-(5-{2-[5-Ethyl-2-(4-trifluoromethyl-phenyl)-thiazol-4-yl]-ethoxy}-indol-1-yl)-acetyl]-methanesulfonamide

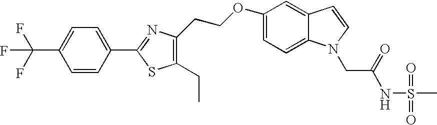

A solution of (5-{2-[5-Ethyl-2-(4-trifluoromethyl-phenyl)-thiazol-4-yl]-ethoxy}-indol-1-yl)-acetic acid (45 mg, 0.094 mmol), methanesulfonamide (9.0 mg, 0.094 mmol), 4-(dimethylamino)pyridine (12 mg, 0.094 mmol), and N,N-diisopropylethylamine (0.033 ml, 0.188 mmol) in dichloromethane (1 ml) is treated with 1-[3-(dimethylamino)propyl]-3-ethylcarbodiimide hydrochloride (29 mg, 0.15 mmol), and the resulting mixture is stirred at ambient temperature for 24 hours. Concentration of the reaction mixture yields a residue which is partitioned between 1N HCl (20 ml) and ethyl acetate (15 ml). The organic layer is washed with brine (25 ml), dried (Na₂SO₄), and concentrated to an oil which is purified by silica chromatography (20:1 hexanes:ethyl acetate to 5:1 hexanes:ethyl acetate) to provide the title compound as a colorless oil. MS: m/z (M+1) 552. The structure is also confirmed by proton NMR.

### EXAMPLE 170

### N-[2-(5-{2-[5-Ethyl-2-(4-trifluoromethyl-phenyl)-thiazol-4-yl]-ethoxy}-indol-1-yl)-acetyl]-benzenesulfonamide

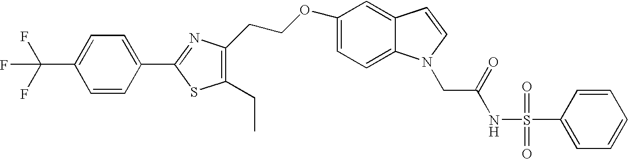

MS: m/z (M+1) 614. The structure is also confirmed by proton NMR.

### EXAMPLE 171

### Methyl 1-benzyl-2-(5-(3-(4-phenylphenoxy)propoxy)-1H-indol-1-yl)-carboxylate

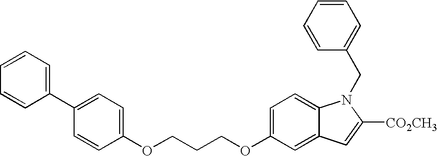

### EXAMPLE 172

### 1-benzyl-2-(5-(3-(4-phenylphenoxy)propoxy)-1H-indol-1-yl)-carboxylic acid

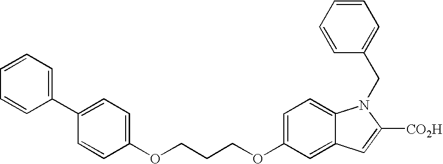

### EXAMPLE 173

### 2-(5-(3-(2-butanoyl-4-(thien-3-yl)-phenoxy)propoxy)-1H-indol-1-yl)-propanoic acid

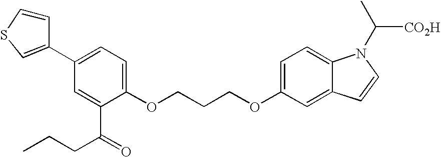

### EXAMPLE 174

### ethyl 2-(5-(3-(2-butanoyl-4-(thien-3-yl)-phenoxy)propoxy)-1H-indol-1-yl)-propanoate

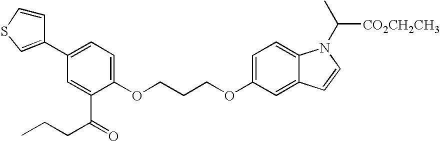

### EXAMPLE 175

### ethyl 2-(5-(3-(2-(prop-2-yl)-4-butanoylphenoxy)propoxy)-1H-indol-1-yl)-propanoate

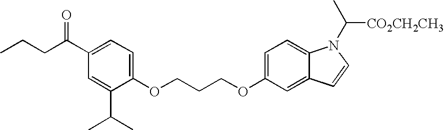

### EXAMPLE 176

### ethyl 2-(5-(3-(2-propyl-3-hydroxy-4-ethanoylphenoxy)propoxy)-1H-indol-1-yl)-propanoate

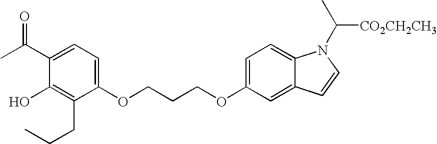

### EXAMPLE 177

### ethyl 2-(5-(3-(3-hydroxy-4-pentanoylphenoxy)propoxy)-1H-indol-1-yl)-propanoate

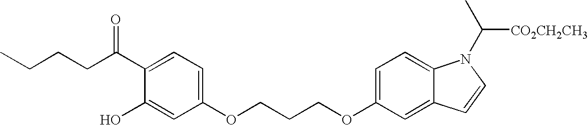

### EXAMPLE 178

### 2-(5-(3-(2-(benzofuran-2-yl)-4-phenylphenoxy)propoxy)-1H-indol-1-yl)-2-methylpropanoic acid

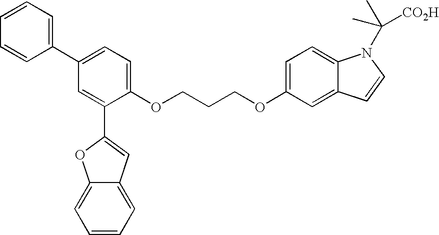

### EXAMPLE 179

### ethyl 2-(5-(3-(4-styrylphenoxy)propoxy)-1H-indol-1-yl)-2-methylpropanoate

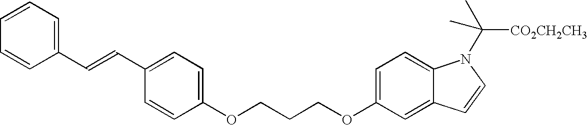

### Biological Assays

### Binding and Cotransfection Studies

The in vitro potency of compounds in modulating PPARα receptors are determined by the procedures detailed below. DNA-dependent binding (ABCD binding) is carried out using SPA technology with PPAR receptors. Tritium-labeled PPARα agonists are used as radioligands for generating displacement curves and IC₅₀ values with compounds of the invention. Cotransfection assays are carried out in CV-1 cells. The reporter plasmid contained an acylCoA oxidase (AOX) PPRE and TK promoter upstream of the luciferase reporter cDNA. Appropriate PPARs are constitutively expressed using plasmids containing the CMV promoter. For PPARα, interference by endogenous PPARγ in CV-1 cells is an issue. In order to eliminate such interference, a GAL4 chimeric system is used in which the DNA binding domain of the transfected PPAR is replaced by that of GAL4, and the GAL4 response element is utilized in place of the AOX PPRE. Cotransfection efficacy is determined relative to PPARα agonist reference molecules. Efficacies are determined by computer fit to a concentration-response curve, or in some cases at a single high concentration of agonist (10 μM).

These studies are carried out to evaluate the ability of compounds of the invention to bind to and/or activate various nuclear transcription factors, particularly huPPARα (“hu” indicates “human”). These studies provide in vitro data concerning efficacy and selectivity of compounds of the invention. Furthermore, binding and cotransfection data for compounds of the invention are compared with corresponding data for marketed compounds that act on huPPARα.

The binding and cotransfection efficacy values for compounds of the invention which are especially useful for modulating a PPAR receptor, are <100 nM and >50%, respectively.

### Evaluation of Triglyceride Reduction and HDL Cholesterol Elevation in HuapoAI Transgenic Mice

Compounds of the present invention are studied for effects upon HDL and triglyceride levels in human apoAI mice. For each compound tested, seven to eight week old male mice, transgenic for human apoAI (C57BL/6-tgn(apoal)1rub, Jackson Laboratory, Bar Harbor, Me.) are acclimated in individual cages for two weeks with standard chow diet (Purina 5001) and water provided ad libitum. After the acclimation, mice and chow are weighed and assigned to test groups (n=5) with randomization by body weight. Mice are dosed daily by oral gavage for 8 days using a 29 gauge, 1½ inch curved feeding needle (Popper & Sons). The vehicle for the controls, test compounds and the positive control (fenofibrate 100 mg/kg) is 1% carboxymethylcellulose (w/v) with 0.25% tween 80 (w/v). All mice are dosed daily between 6 and 8 a.m. with a dosing volume of 0.2 ml. Prior to termination, animals and diets are weighed and body weight change and food consumption are calculated. Three hours after last dose, mice are euthanized with CO2 and blood is removed (0.5-1.0 ml) by cardiac puncture. After sacrifice, the liver, heart, and epididymal fat pad are excised and weighed. Blood is permitted to clot and serum is separated from the blood by centrifugation.

Cholesterol and triglycerides are measured colorimetrically using commercially prepared reagents (for example, as available from Sigma #339-1000 and Roche #450061 for triglycerides and cholesterol, respectively). The procedures are modified from published work (McGowan M. W. et al., Clin Chem 29:538-542, 1983; Allain C. C. et al., Clin Chem 20:470-475, 1974. Commercially available standards for triglycerides and total cholesterol, respectively, commercial quality control plasma, and samples are measured in duplicate using 200 μl of reagent. An additional aliquot of sample, added to a well containing 200 μl water, provided a blank for each specimen. Plates are incubated at room temperature on a plate shaker and absorbance is read at 500 nm and 540 nm for total cholesterol and triglycerides, respectively. Values for the positive control are always within the expected range and the coefficient of variation for samples is below 10%. All samples from an experiment are assayed at the same time to minimize inter-assay variability.

Serum lipoproteins are separated and cholesterol quantitated by fast protein liquid chromatography (FPLC) coupled to an in line detection system. Samples are applied to a Superose 6 HR size exclusion column (Amersham Pharmacia Biotech) and eluted with phosphate buffered saline-EDTA at 0.5 ml/min. Cholesterol reagent (Roche Diagnostics Chol/HP 704036) at 0.16 ml/min mixed with the column effluent through a T-connection and the mixture passed through a 15 m×0.5 mm id knitted tubing reactor immersed in a 37 C water bath. The colored product produced in the presence of cholesterol is monitored in the flow strem at 505 nm and the analog voltage from the monitor is converted to a digital signal for collection and analysis. The change in voltage corresponding to change in cholesterol concentration is plotted vs time and the area under the curve corresponding to the elution of very low density lipoprotein (VLDL), low density lipoprotein (LDL) and high density lipoprotein (HDL) is calculated using Perkin Elmer Turbochrome software.

Triglyceride Serum Levels in Mice Dosed with a Compound of the Invention is Compared to Mice Receiving the Vehicle to identify compounds which could be particularly useful for lowering triglycerides. Generally, triglyceride decreases of greater than or equal to 30% (thirty percent) compared to control following a 30 mg/kg dose suggests a compound that can be especially useful for lowering triglyceride levels.

The percent increase of HDLc serum levels in mice receiving a compound of the invention is compared to mice receiving vehicle to identify compounds of the invention that could be particularly useful for elevating HDL levels. Generally, and increase of greater than or equal to 25% (twenty five percent) increase in HDLc level following a 30 mg/kg dose suggests a compound that can be especially useful for elevating HDLc levels.

It may be particularly desirable to select compounds of this invention that both lower triglyceride levels and increase HDLc levels. However, compounds that either lower triglyceride levels or increase HDLc levels may be desirable as well.

### Evaluation of Glucose Levels in db/db Mice

The effects upon plasma glucose associated with administering various dose levels of different compounds of the present invention and the PPAR gamma agonist rosiglitazone (BRL49653) or the PPAR alpha agonist fenofibrate, and the control, to male db/db mice, are studied.

Five week old male diabetic (db/db) mice [for example, C57BlKs/j-m+/+ Lepr(db), Jackson Laboratory, Bar Harbor, Me.] or lean littermates are housed 6 per cage with food and water available at all times. After an acclimation period of 2 weeks, animals are individually identified by ear notches, weighed, and bled via the tail vein for determination of initial glucose levels. Blood is collected (100 μl) from unfasted animals by wrapping each mouse in a towel, cutting the tip of the tail with a scalpel, and milking blood from the tail into a heparinized capillary tube. Sample is discharged into a heparinized microtainer with gel separator and retained on ice. Plasma is obtained after centrifugation at 4° C. and glucose measured immediately. Remaining plasma is frozen until the completion of the experiment, when glucose and triglycerides are assayed in all samples. Animals are grouped based on initial glucose levels and body weights. Beginning the following morning, mice are dosed daily by oral gavage for 7 days. Treatments are test compounds (30 mg/kg), a positive control agent (30 mg/kg) or vehicle [1% carboxymethylcellulose (w/v)/0.25% Tween80 (w/v); 0.3 ml/mouse]. On day 7, mice are weighed and bled (tail vein) 3 hours after dosing. Twenty-four hours after the 7^{th} dose (i.e., day 8), animals are bled again (tail vein). Samples obtained from conscious animals on days 0, 7 and 8 are assayed for glucose. After the 24-hour bleed, animals are weighed and dosed for the final time. Three hours after dosing on day 8, animals are anesthetized by inhalation of isoflurane and blood obtained via cardiac puncture (0.5-0.7 ml). Whole blood is transferred to serum separator tubes, chilled on ice and permitted to clot. Serum is obtained after centrifugation at 4° C. and frozen until analysis for compound levels. After sacrifice by cervical dislocation, the liver, heart and epididymal fat pads are excised and weighed.

Glucose is measured calorimetrically using commercially purchased reagents. According to the manufacturers, the procedures are modified from published work (McGowan, M. W., Artiss, J. D., Strandbergh, D. R. & Zak, B. Clin Chem, 20:470-5 (1974) and Keston, A. Specific calorimetric enzymatic analytical reagents for glucose. Abstract of papers 129th Meeting ACS, 31C (1956).); and depend on the release of a mole of hydrogen peroxide for each mole of analyte, coupled with a color reaction first described by Trinder (Trinder, P. Determination of glucose in blood using glucose oxidase with an alternative oxygen acceptor. Ann Clin Biochem, 6:24 (1969)). The absorbance of the dye produced is linearly related to the analyte in the sample. The assays are further modified in our laboratory for use in a 96 well format. The commercially available standard for glucose, commercially available quality control plasma, and samples (2 or 5 μl/well) are measured in duplicate using 200 μl of reagent. An additional aliquot of sample, pipetted to a third well and diluted in 200 μl water, provided a blank for each specimen. Plates are incubated at room temperature for 18 minutes for glucose on a plate shaker (DPC Micormix 5) and absorbance read at 500 nm on a plate reader. Sample absorbances are compared to a standard curve (100-800 for glucose). Values for the quality control sample are always within the expected range and the coefficient of variation for samples is below 10%. All samples from an experiment are assayed at the same time to minimize inter-assay variability.

### Evaluation of the Effects of Compounds of the Present Invention Upon A^{y} Mice Body Weight, Fat Mass, Glucose and Insulin Levels

Female A^{y} Mice

Female A^{y} mice are singly housed, maintained under standardized conditions (22° C., 12 h light:dark cycle), and provided free access to food and water throughout the duration of the study. At twenty weeks of age the mice are randomly assigned to vehicle control and treated groups based on body weight and body fat content as assessed by DEXA scanning (N-6). Mice are then dosed via oral gavage with either vehicle or a Compound of this invention (50 mg/kg) one hour after the initiation of the light cycle (for example, about 7 A.M.) for 18 days. Body weights are measured daily throughout the study. On day 14 mice are maintained in individual metabolic chambers for indirect calorimetry assessment of energy expenditure and fuel utilization. On day 18 mice are again subjected to DEXA scanning for post treatment measurement of body composition.

The results of p.o. dosing of compound for 18 days on body weight, fat mass, and lean mass are evaluated and suggest which compounds of this invention can be especially useful for maintaining desirable weight and/or promoting desired lean to fat mass.

Indirect calorimetry measurements revealing a significant reduction in respiratory quotient (RQ) in treated animals during the dark cycle [0.864+0.013 (Control) vs. 0.803+0.007 (Treated); p<0.001] is indicative of an increased utilization of fat during the animals' active (dark) cycle and can be used to selected especially desired compounds of this invention. Additionally, treated animals displaying significantly higher rates of energy expenditure than control animals suggest such compounds of this invention can be especially desired.

Male KK/A^{y} Mice

Male KK/A^{y} mice are singly housed, maintained under standardized conditions (22° C., 12 h light:dark cycle), and provided free access to food and water throughout the duration of the study. At twenty-two weeks of age the mice are randomly assigned to vehicle control and treated groups based on plasma glucose levels. Mice are then dosed via oral gavage with either vehicle or a Compound of this invention (30 mg/kg) one hour after the initiation of the light cycle (7 A.M.) for 14 days. Plasma glucose, triglyceride, and insulin levels are assessed on day 14.

The results of p.o. dosing of compound for 14 days on plasma glucose, triglycerides, and insulin are evaluated to identify compounds of this invention which may be especially desired.

Method to Elucidate the LDL-cholesterol Total-Cholesterol and Triglyceride Lowering Effect

Male Syrian hamsters (Harlan Sprague Dawley) weighing 80-120 g are placed on a high-fat cholesterol-rich diet for two to three weeks prior to use. Feed and water are provided ad libitum throughout the course of the experiment. Under these conditions, hamsters become hypercholesterolemic showing plasma cholesterol levels between 180-280 mg/dl. (Hamsters fed with normal chow have a total plasma cholesterol level between 100-150 mg/dl.) Hamsters with high plasma cholesterol (180 mg/dl and above) are randomized into treatment groups based on their total cholesterol level using the GroupOptimizeV211.xls program.

A Compound of this invention is dissolved in an aqueous vehicle (containing CMC with Tween 80) such that each hamster received once a day approx. 1 ml of the solution by garvage at doses 3 and 30 mg/kg body weight. Fenofibrate (Sigma Chemical, prepared as a suspension in the same vehicle) is given as a known alpha-agonist control at a dose of 200 mg/kg, and the blank control is vehicle alone. Dosing is performed daily in the early morning for 14 days.

Quantification of Plasma Lipids:

On the last day of the test, hamsters are bled (400 ul) from the suborbital sinus while under isoflurane anesthesia 2 h after dosing. Blood samples are collected into heparinized microfuge tubes chilled in ice bath. Plasma samples are separated from the blood cells by brief centrifugation. Total cholesterol and triglycerides are determined by means of enzymatic assays carried out automatically in the Monarch equipment (Instrumentation Laboratory) following the manufacturer's precedure. Plasma lipoproteins (VLDL, LDL and HDL) are resolved by injecting 25 ul of the pooled plasma samples into an FPLC system eluted with phosphate buffered saline at 0.5 ml/min through a Superose 6 HR 10/30 column (Pharmacia) maintained room temp. Detection and characterization of the isolated plasma lipids are accomplished by postcolumn incubation of the effluent with a Cholesterol/HP reagent (for example, Roche Lab System; infused at 0.12 ml/min) in a knitted reaction coil maintained at 37° C. The intensity of the color formed is proportional to the cholesterol concentration and is measured photometrically at 505 nm.

The effect of administration of a Compound of this invention for 14 days is studied for the percent reduction in LDL level with reference to the vehicle group. Especially desired compounds are markedly more potent than fenofibrate in LDL-lowering efficacy. Compounds of this invention that decrease LDL greater than or equal to 30% (thirty percent) compared to vehicle can be especially desired.

The total-cholesterol and triglyceride lowering effects of a Compound of this invention is also studied. The data for reduction in total cholesterol and triglyceride levels after treatment with a compound of this invention for 14 days is compared to the vehicle to suggest compounds that can be particularly desired. The known control fenofibrate did not show significant efficacy under the same experimental conditions.

Method to Elucidate the Fibrinogen-Lowering Effect of PPARModulators

Zucker Fatty Rat Model:

The life phase of the study on fibrinogen-lowering effect of compounds of this invention is part of the life phase procedures for the antidiabetic studies of the same compounds. On the last (14^{th}) day of the treatment period, with the animals placed under surgical anesthesia, ˜3 ml of blood is collected, by cardiac puncture, into a syringe containing citrate buffer. The blood sample is chilled and centrifuged at 4° C. to isolate the plasma that is stored at −70° C. prior to fibrinogen assay.

Quantification of Rat Plasma Fibrinogen:

Rat plasma fibrinogen levels are quantified by using a commercial assay system consists of a coagulation instrument following the manufacturer's protocol. In essence, 100 ul of plasma is sampled from each specimen and a 1/20 dilution is prepared with buffer. The diluted plasma is incubated at 37° C. for 240 seconds. Fifty microliters of clotting reagent thrombin solution (provided by the instrument's manufacturer in a standard concentration) is then added. The instrument monitors the clotting time, a function of fibrinogen concentration quantified with reference to standard samples.

Compounds that lower fibrinogen level greater than vehicle can be especially desired.

Cholesterol and triglyceride lowering effects of compounds of this invention are also studied in Zucker rats.

Method to Elucidate the Anti-Body Weight Gain and Anti-Appetite Effects of Compounds of this Invention

Fourteen-Day Study in Zucker Fatty Rat¹ or ZDF Rat² Models:

Male Zucker Fatty rats, non-diabetic (Charles River Laboratories, Wilmington, Mass.) or male ZDF rats (Genetic Models, Inc, Indianapolis, Ind.) of comparable age and weight are acclimated for 1 week prior to treatment. Rats are on normal chow and water is provided ad libitum throughout the course of the experiment.

Compounds of this invention are dissolved in an aqueous vehicle such that each rat received once a day approximately 1 ml of the solution by garvage at doses 0.1, 0.3, 1 and 3 mg/kg body weight. Fenofibrate (Sigma Chemical, prepared as a suspension in the same vehicle) a known alpha-agonist given at doses of 300 mg/kg, as well as the vehicle are controls. Dosing is performed daily in the early morning for 14 days. Over the course of the experiment, body weight and food consumption are monitored.

Using this assay, compounds of this invention are identified to determine which can be associated with a significant weight reduction.

### Monkey Studies

### Efficacy Studies

Compounds of the invention may be examined in a dyslipidemic rhesus monkey model. After an oral dose-escalation study for 28 days in obese, non-diabetic rhesus monkeys a determination of HDL-c elevation is made with each dose and compared with pretreatment levels. LDL cholesterol is also determined with each dose. C-reactive protein levels are measured and compared to pretreatment levels.

Compound of Formula 1 may be shown to elevate plasma HDL-cholesterol levels in an African Green Monkey model in a manner similar to that described above in rhesus monkeys. Two groups of monkeys are placed in a dose-escalating study that consists of one week of baseline measurements, 9 weeks of treatments (vehicle, Compound of Formula I), and four weeks of washout. During baseline, monkeys in all three groups are administered vehicle once daily for seven days. Test compound of Formula I, is administered in vehicle once daily for three weeks, then at a greater concentration (double the dose may be desired) once daily for three weeks, and then a still greater concentration (double the most recent dose may be desired) once daily for three weeks. At the completion of treatment, monkeys in both groups are administered vehicle once daily and monitored for an additional six weeks.

Animals are fasted overnight and then sedated for body weight measurements and blood collection at weeks 1 (vehicle), 2, 3, 4, 6, 7, 9, 10, 12, and 14 of the study.

Parameters to Measured, for Example:

Body weight

Total plasma cholesterol

HDL

LDL

Triglycerides

Insulin

Glucose

PK parameters at week 4, 7, and 10 (plasma drug concentration at last week of each dose).

ApoAI

ApoAII

ApoB

ApoCIII

Liver enzymes (SGPT, SGOT, OGT)

Complete blood count

Additionally, other measures may be made, as appropriate, and consistent with the stated study design.

While this invention has been particularly shown and described with references to preferred embodiments thereof, it will be understood by those skilled in the art that various changes in form and details may be made therein without departing from the scope of the invention encompassed by the appended claims.

Equivalents:

While this invention has been particularly shown and described with references to preferred embodiments thereof, it will be understood by those skilled in the art that various changes in form and details may be made therein without departing from the scope of the invention encompassed by the appended claims.

1. A compound represented by the following Structural Formula: 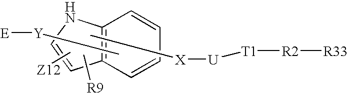
and stereoisomers, pharmaceutically acceptable salts, solvates and hydrates thereof, wherein:
(a) T1 is selected from the group consisting of 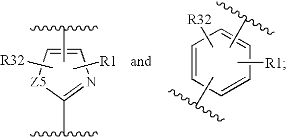
(b) R1 is selected from the group consisting of hydrogen, C₁-C₈ alkyl, C₁-C₈ alkenyl, aryl-C₀₋₄-alkyl, aryl-C₁₋₆-heteroalkyl, heteroaryl-C₀₋₄-alkyl, and C₃-C₆ cycloalkylaryl-C₀₋₂-alkyl, wherein C₁-C₈ alkyl, C₁-C₈ alkenyl, aryl-C₀₋₄-alkyl, aryl-C₁₋₆-heteroalkyl, heteroaryl-C₀₋₄-alkyl, and C₃-C₆ cycloalkylaryl-C₀₋₂-alkyl are each optionally substituted with from one to three substituents independently selected from R1′;
(c) R1′, R26, R27, R28, R31, Z14′, and Z15′ are each independently selected from the group consisting of hydrogen, hydroxy, cyano, nitro, halo, oxo, C₁-C₆ alkyl, C₁-C₆ alkyl-COOR12, C₁-C₆ alkoxy, C₁-C₆ haloalkyl, C₁-C₆ haloalkyloxy, C₃-C₇ cycloalkyl, optionally substituted aryloxy, optionally substituted aryl-C₀₋₄-alkyl, optionally substituted heteroaryl, optionally substituted heterocycloalkyl, C(O)R13, COOR14, OC(O)R15, OS(O)₂R16, N(R17)₂, NR18C(O)R19, NR20SO₂R21, SR22, S(O)R23, S(O)₂R24, and S(O)₂N(R25)₂; R12, R13, R14, R15, R16, R17, R18, R19, R20, R21, R22, R23, R24 and R25 are each independently selected from the group consisting of hydrogen, C₁-C₆ alkyl and aryl;
(d) R2 is selected from the group consisting of C₀-C₈ alkyl and C₁₋₆-heteroalkyl;
(e) X is selected from the group consisting of a bond, O, S, S(O)₂ and N;
(f) U is an aliphatic linker wherein one carbon atom of the aliphatic linker may be replaced with O, NH or S, and wherein such aliphatic linker is optionally substituted with R30;
(g) Y is selected from the group consisting of C, O, S, NH and a single bond;
(h) E is C(R3)(R4)A or A and wherein
(i) A is selected from the group consisting of C₀-C₆ alkylcarboxyl, C₀-C₆ alkyltetrazole, C₁-C₆ alkylnitrile, C₀-C₆ alkylcarboxamide, C₀-C₆ alkylsulfonamide and C₀-C₆ alkylacylsulfonamide; wherein C₀-C₆ alkylsulfonamide, C₀-C₆ alkylacylsulfonamide and C₀-C₆ alkyltetrazole are each optionally substituted with from one to two groups independently selected from R⁷;
(ii) each R⁷ is independently selected from the group consisting of hydrogen, C₁-C₆ haloalkyl, aryl-C₀-C₄ alkyl and C₁-C₆ alkyl, wherein such alkyl and arylalkyl are each optionally substituted with from one to two groups independently selected from R7′; each R7′ is independently selected from halo, C₁-C₆ alkyl, and haloC₁-C₆ alkyl;
(iii) R3 is selected from the group consisting of hydrogen, C₁-C₅ alkyl, and C₁-C₅ alkoxy; and
(iv) R4 is selected from the group consisting of hydrogen, C₁-C₅ alkyl, C₁-C₅ alkoxy, aryloxy, C₃-C₆ cycloalkyl, and aryl C₀-C₄ alkyl, and R3 and R4 are optionally combined to form a C₃-C₄ cycloalkyl, and wherein alkyl, alkoxy, cycloalkyl and aryl-alkyl are each optionally substituted with one to three each independently selected from R26;
(i) Z5 is S or O;
(j) Z12 is selected from the group consisting of hydrogen and -Z13C₀-C₃alkylZ14;
(k) Z13 is selected from the group consisting of a single bond, CO, CO₂, CONZ15, and SO₂;
(l) Z14 is selected from the group consisting of aryl and heteroaryl, wherein the aryl and heteroaryl is each optionally substituted with from one to three substituents independently selected from Z14′;
(m) Z15 is selected from the group consisting of hydrogen aryl and heteroaryl, wherein the aryl and heteroaryl is each optionally substituted with from one to three substituents independently selected from Z15′;
(n) R9 is selected from the group consisting of hydrogen, C₁-C₄ alkyl, C₁-C₄ alkylenyl, halo, aryl-C₀-C₄ alkyl, heteroaryl, C₁-C₆ allyl, and OR29, and wherein aryl-C₀-C₄ alkyl, heteroaryl are each optionally substituted with from one to three independently selected from R27; R29 is selected from the group consisting of hydrogen and C₁-C₄ alkyl;
(o) R10, R11 are each independently selected from the group consisting of hydrogen, hydroxy, cyano, nitro, halo, oxo, C₁-C₆ alkyl, C₀-C₆ alkyl-COOR12″, C₁-C₆ alkoxy, C₁-C₆ haloalkyl, C₁-C₆ haloalkyloxy, C₃-C₇ cycloalkyl, aryl-C₀₋₄-alkyl, aryl-C₁₋₆-heteroalkyl, heteroaryl-C₀₋₄-alkyl, C₃-C₆ cycloalkylaryl-C₀₋₂-alkyl, aryloxy, C(O)R13′, COOR14, OC(O)R15′, OS(O)₂R16′, N(R17′)₂, NR18′C(O)R19′, NR20′SO₂R21′, SR22′, S(O)R23′, S(O)₂R24′, and S(O)₂N(R25′)₂; and wherein aryl-C₀₋₄-alkyl, aryl-C₁₋₆-heteroalkyl, heteroaryl-C₀₋₄-alkyl, and C₃-C₆ cycloalkylaryl-C₀₋₂-alkyl are each optionally substituted with from one to three independently selected from R28;
(p) R12′, R12″, R13′, R14′, R15′, R16′, R17′, R18, R19, R20, R21′, R22′, R23′, R24′, and R25′ are each independently selected from the group consisting of hydrogen, C₁-C₆ alkyl and aryl;
(q) R30 is selected from the group consisting of C₁-C₆ alkyl, aryl-C₀₋₄-alkyl, aryl-C₁₋₆-heteroalkyl, heteroaryl-C₀₋₄-alkyl, and C₃-C₆ cycloalkylaryl-C₀₋₂-alkyl, and wherein C₁-C₆ alkyl, aryl-C₀₋₄-alkyl, aryl-C₁₋₆-heteroalkyl, heteroaryl-C₀₋₄-alkyl, and C₃-C₆ cycloalkylaryl-C₀₋₂-alkyl are each optionally substituted with from one to three substituents each independently selected from R31;
(r) R32 is selected from the group consisting of a bond, hydrogen, halo, C₁-C₆ alkyl, C₁-C₆ haloalkyl, and C₁-C₆ alkyloxo;
(s) R33 is selected from the group consisting of C2-C8 alkyl, C1-C8 alkoxy, phenyl, thiophene, pyridine, piperidine, 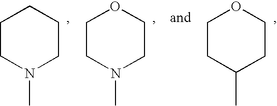
wherein the C2-C8 alkyl, C1-C8 alkoxy, phenyl, thiophene, pyridine, piperidine, 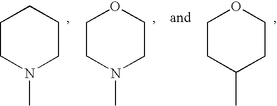
are each optionally substituted with R10 and R11; and
(t) provided that when Y is C or a bond, at least one of R1, R2, R3, and R4 is C1-C4 alkyl.

2. The compound of claim 1, wherein A is selected from the group consisting of C₀-C₆ alkylcarboxyl, C₀-C₆ alkyltetrazole, C₁-C₆ alkylnitrile, C₀-C₆ alkylsulfonamide and C₀-C₆ alkylacylsulfonamide; wherein C₀-C₆ alkylsulfonamide, C₀-C₆ alkylacylsulfonamide and C₀-C₆ alkyltetrazole are each optionally substituted with from one to two groups independently selected from R⁷.

3. The compound of claim 1, wherein the compound is represented by the following Structural Formula: 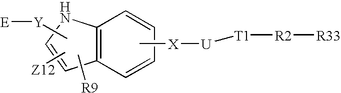
and stereoisomers, pharmaceutically acceptable salts, solvates and hydrates thereof, wherein:
(a) T1 is selected from the group consisting of 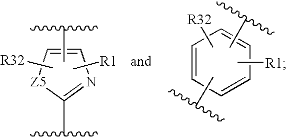
(b) R1 is selected from the group consisting of hydrogen, C₁-C₈ alkyl, C₁-C₈ alkenyl, aryl-C₀₋₄-alkyl, aryl-C₁₋₆-heteroalkyl, heteroaryl-C₀₋₄-alkyl, and C3-C6 cycloalkylaryl-C₀₋₂-alkyl, wherein C₁-C₈ alkyl, C₁-C₈ alkenyl, aryl-C₀₋₄-alkyl, aryl-C₁₋₆-heteroalkyl, heteroaryl-C₀₋₄-alkyl, and C3-C6 cycloalkylaryl-C₀₋₂-alkyl are each optionally substituted with from one to three substituents independently selected from R1′;
(c) R1′, R26, R27, R28, R31, Z14′, and Z15′ are each independently selected from the group consisting of hydrogen, hydroxy, cyano, nitro, halo, oxo, C₁-C₆ alkyl, C₁-C₆ alkyl-COOR12, C₁-C₆ alkoxy, C₁-C₆ haloalkyl, C₁-C₆ haloalkyloxy, C₃-C₇ cycloalkyl, aryloxy, aryl-C₀₋₄-alkyl, heteroaryl, heterocycloalkyl, C(O)R13, COOR14, OC(O)R15, OS(O)₂R16, N(R17)₂, NR18C(O)R19, NR20SO₂R21, SR22, S(O)R23, S(O)₂R24, and S(O)₂N(R25)₂; R12, R13, R14, R15, R16, R17, R18, R19, R20, R21, R22, R23, R24 and R25 are each independently selected from the group consisting of hydrogen, C₁-C₆ alkyl and aryl;
(d) R2 is selected from the group consisting of C₀-C₈ alkyl and C₁₋₆-heteroalkyl;
(e) X is selected from the group consisting of a bond, O, S, S(O)₂ and N;
(f) U is an aliphatic linker wherein one carbon atom of the aliphatic linker may be replaced with O, NH or S, and wherein such aliphatic linker is optionally substituted with R30;
(g) Y is selected from the group consisting of C, O, S, NH and a single bond;
(h) E is C(R3)(R4)A or A and wherein
(i) A is selected from the group consisting of carboxyl, tetrazole, C₁-C₆ alkylnitrile, carboxamide, sulfonamide and acylsulfonamide; wherein sulfonamide, acylsulfonamide and tetrazole are each optionally substituted with from one to two groups independently selected from R⁷;
(ii) each R⁷ is independently selected from the group consisting of hydrogen, C₁-C₆ haloalkyl, aryl-C₀-C₄ alkyl and C₁-C₆ alkyl, wherein such alkyl and arylalkyl are each optionally substituted with from one to two groups independently selected from R7′; each R7′ is independently selected from halo, C₁-C₆ alkyl, and haloC₁-C₆ alkyl;
(iii) R3 is selected from the group consisting of hydrogen, C₁-C₅ alkyl, and C₁-C₅ alkoxy; and
(iv) R4 is selected from the group consisting of hydrogen, C₁-C₅ alkyl, C₁-C₅ alkoxy, aryloxy, C₃-C₆ cycloalkyl, and aryl C₀-C₄ alkyl, and R3 and R4 are optionally combined to form a C₃-C₄ cycloalkyl, and wherein alkyl, alkoxy, cycloalkyl and aryl-alkyl are each optionally substituted with one to three each independently selected from R26;
(i) Z5 is S or O;
(j) Z12 is selected from the group consisting of hydrogen and -Z13C₀-C₃alkylZ14;
(k) Z13 is selected from the group consisting of a single bond, CO, CO₂, CONZ15, and SO₂;
(l) Z14 is selected from the group consisting of aryl and heteroaryl, wherein the aryl and heteroaryl is each optionally substituted with from one to three substituents independently selected from Z14′;
(m) Z15 is selected from the group consisting of hydrogen aryl and heteroaryl, wherein the aryl and heteroaryl is each optionally substituted with from one to three substituents independently selected from Z15′;
(n) R9 is selected from the group consisting of hydrogen, C₁-C₄ alkyl, C₁-C₄ alkylenyl, halo, aryl-C₀-C₄ alkyl, heteroaryl, C₀-C₆ allyl, and OR29, and wherein aryl-C₀-C₄ alkyl, heteroaryl are each optionally substituted with from one to three independently selected from R27; R29 is selected from the group consisting of hydrogen and C₁-C₄ alkyl;
(o) R10, R11 are each independently selected from the group consisting of hydrogen, hydroxy, cyano, nitro, halo, oxo, C₁-C₆ alkyl, C₀-C₆ alkyl-COOR12″, C₁-C₆ alkoxy, C₁-C₆ haloalkyl, C₁-C₆ haloalkyloxy, C₃-C₇ cycloalkyl, aryl-C₀₋₄-alkyl, aryl-C₁₋₆-heteroalkyl, heteroaryl-C₀₋₄-alkyl, C₃-C₆ cycloalkylaryl-C₀₋₂-alkyl, aryloxy, C(O)R13′, COOR14, OC(O)R15′, OS(O)₂R16′, N(R17)₂, NR18′C(O)R19′, NR20′SO₂R21′, SR22′, S(O)R23, S(O)₂R24′ and S(O)₂N(R25)₂; and wherein aryl-C₀₋₄-alkyl, aryl-C₁₋₆-heteroalkyl, heteroaryl-C₀₋₄-alkyl, and C₃-C₆ cycloalkylaryl-C₀₋₂-alkyl are each optionally substituted with from one to three independently selected from R28;
(p) R12′, R12″, R13′, R14′, R15′, R16′, R17′, R18′, R19′, R20′, R21′, R22′, R23′, R24′ and R25′ are each independently selected from the group consisting of hydrogen, C₁-C₆ alkyl and aryl;
(q) R30 is selected from the group consisting of C₁-C₆ alkyl, aryl-C₀₋₄-alkyl, aryl-C₁₋₆-heteroalkyl, heteroaryl-C₀₋₄-alkyl, and C₃-C₆ cycloalkylaryl-C₀₋₂-alkyl, and wherein C₁-C₆ alkyl, aryl-C₀₋₄-alkyl, aryl-C₁₋₆-heteroalkyl, heteroaryl-C₀₋₄-alkyl, and C₃-C₆ cycloalkylaryl-C₀₋₂-alkyl are each optionally substituted with from one to three substituents each independently selected from R31;
(r) R32 is selected from the group consisting of a bond, hydrogen, halo, C₁-C₆ alkyl, C₁-C₆ haloalkyl, and C₁-C₆ alkyloxo;
(s) R33 is selected from the group consisting of phenyl, thiophene, pyridine, piperidine, 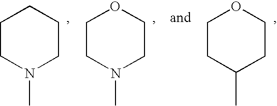
wherein the phenyl, thiophene, pyridine, piperidine, 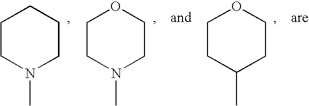
each optionally substituted with R10 and R11; and
(u) provided that when Y is C or a bond, R1, R2, R3, and R4 are each independently C₁-C₄ alkyl.

4. The compound of claim 2, wherein the compound is represented by the following Structural Formula:

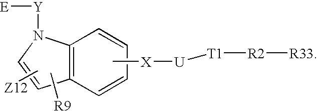

5. The compound of claim 1, wherein the compound is represented by the following Structural Formula:

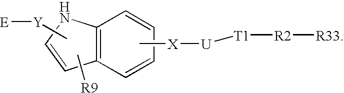

6. The compound of claim 5, wherein the compound is represented by the following Structural Formula:

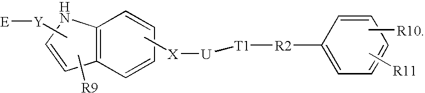

7. The compound of claim 6, wherein the compound is represented by the following Structural Formula:

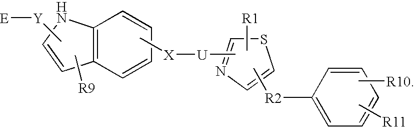

8-9. (canceled)

10. The compound of claim 7, wherein:
X is —O—;
E is —COOH, C₁-C₆ alkylcarboxyl, or C(R3)(R4)-C₁-C₆ alkyl-COOH;
R10 and R11 are each independently selected from the group consisting of hydrogen, halo, oxo, C₁-C₆ alkyl, C₁-C₆ alkyl-COOR12″, C₁-C₆ alkoxy, C₁-C₆ haloalkyl, and C₁-C₆ haloalkyloxy;
R9 is selected from the group consisting of hydrogen and C₁-C₃ alkyl;
R1, R3, and R4 are each independently selected from the group consisting of hydrogen and C₁-C₂ alkyl;
R2 is a bond; and
U is saturated C₁-C₃ alkyl optionally substituted with C₁-C₃ alkyl.

11-18. (canceled)

19. The compound of claim 6, wherein the compound is represented by the following Structural Formula:

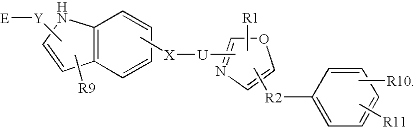

20-21. (canceled)

22. The compound of claim 19, wherein:
X is —O—;
E is —COOH, C₁-C₆ alkylcarboxyl, or C(R3)(R4)-C₁-C₆ alkyl-COOH;
R10 and R11 are each independently selected from the group consisting of hydrogen, halo, oxo, C₁-C₆ alkyl, C₁-C₆ alkyl-COOR12″, C₁-C₆ alkoxy, C₁-C₆ haloalkyl, and C₁-C₆ haloalkyloxy;
R9 is selected from the group consisting of hydrogen and C₁-C₃ alkyl;
R1, R3, and R4 are each independently selected from the group consisting of hydrogen and C₁-C₂ alkyl;
R2 is a bond; and
U is saturated C₁₋₃ alkyl optionally substituted with C₁-C₃ alkyl.

23-30. (canceled)

31. The compound of claim 6, wherein the compound is represented by the following Structural Formula:

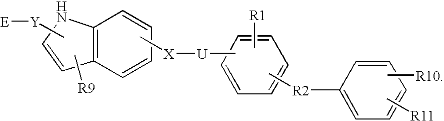

32-33. (canceled)

34. The compound of claim 31, wherein:
X is —O—;
E is —COOH, C₁-C₆ alkylcarboxyl, or C(R3)(R4)-C₁-C₆ alkyl-COOH;
R10 and R11 are each independently selected from the group consisting of hydrogen, halo, oxo, C₁-C₆ alkyl, C₁-C₆ alkyl-COOR12″, C₁-C₃ alkoxy, C₁-C₆ haloalkyl, and C₁-C₆ haloalkyloxy;
R9 is selected from the group consisting of hydrogen and C₁-C₃ alkyl;
R1, R3, and R4 are each independently selected from the group consisting of hydrogen and C₁-C₂ alkyl;
R2 is a bond: and
U is saturated C₁-C₃ alkyl optionally substituted with C₁-C₃ alkyl.

35-42. (canceled)

43. The compound of claim 1 wherein the compound is selected from the group consisting of:
Racemic-(6-{1-[4-Isopropyl-2-(4-trifluoromethyl-phenyl)-thiazol-5-yl]-ethoxy}-1H-indol-3-yl)-acetic acid;
Racemic-(6-{2-[5-Methyl-2-(4-trifluoromethyl-phenyl)-thiazol-4-yl]-propoxy}-1H-indol-3-yl)-acetic acid;
Racemic-(1-Methyl-6-{2-[5-methyl-2-(4-trifluoromethyl-phenyl)-thiazol-4-yl]-propoxy}-1H-indol-3-yl)-acetic acid;
(S)-(6-{2-[5-Methyl-2-(4-trifluoromethyl-phenyl)-thiazol-4-yl]-propoxy}-1H-indol-3-yl)-acetic acid;
(R)-(6-{2-[5-Methyl-2-(4-trifluoromethyl-phenyl)-thiazol-4-yl]-propoxy}-1H-indol-3-yl)-acetic acid;
Racemic-(6-Hydroxy-5-{1-[4-isopropyl-2-(4-trifluoromethyl-phenyl)-thiazol-5-yl]-ethyl}-1H-indol-3-yl)-acetic acid;
(6-{2-[4-Isopropyl-2-(4-trifluoromethyl-phenyl)-thiazol-5-yl]-propoxy}-1-methyl-1H-indol-3-yl)-acetic acid;
(1-Methyl-6-{2-[4-methyl-2-(4-trifluoromethyl-phenyl)-thiazol-5-yl]-propoxy}-1H-indol-3-yl)-acetic acid;
(6-{2-[5-Ethyl-2-(4-trifluoromethyl-phenyl)-thiazol-4-yl]-propoxy}-1-methyl-1H-indol-3-yl)-acetic acid;
(R)-(6-{2-[4-Isopropyl-2-(4-trifluoromethyl-phenyl)-thiazol-5-yl]-propoxy}-1-methyl-1H-indol-3-yl)-acetic acid;
(S)-(6-{2-[4-Isopropyl-2-(4-trifluoromethyl-phenyl)-thiazol-5-yl]-propoxy}-1-methyl-1H-indol-3-yl)-acetic acid;
Racemic-(1-Methyl-6-{2-[4-methyl-2-(4-trifluoromethyl-phenyl)-thiazol-5-yl]-propoxy}-1H-indol-3-yl)-acetic acid;
Racemic-(1-Ethyl-6-{2-[4-isopropyl-2-(4-trifluoromethyl-phenyl)-thiazol-5-yl]-propoxy}-1H-indol-3-yl)-acetic acid;
Racemic-(6-{2-[4-Isopropyl-2-(4-trifluoromethyl-phenyl)-thiazol-5-yl]-propoxy}-1-propyl-1H-indol-3-yl)-acetic acid;
{1-[4-Isopropyl-2-(4-trifluoromethyl-phenyl)-thiazol-5-ylmethyl]-1H-indol-5-yloxy}-acetic acid;
Racemic(6-{1-[4-Isopropyl-2-(4-trifluoromethyl-phenyl)-thiazol-5-yl]-ethoxy}-1H-indol-3-yl)-acetic acid;
Racemic(6-{1-[4-methyl-2-(4-trifluoromethyl-phenyl)-thiazol-5-yl]-ethoxy}-1H-indol-3-yl)-acetic acid;
(R)-(6-{1-[4-Isopropyl-2-(4-trifluoromethyl-phenyl)-thiazol-5-yl]-ethoxy}-1H-indol-3-yl)-acetic acid;
(S)-(6-{1-[4-Isopropyl-2-(4-trifluoromethyl-phenyl)-thiazol-5-yl]-ethoxy}-1H-indol-3-yl)-acetic acid;
(R)-(5-{2-[5-Methyl-2-(4-trifluoromethyl-phenyl)-thiazol-4-yl]-propoxy}-1H-indol-3-yl)-acetic acid;
(S)-(5-{2-[5-Methyl-2-(4-trifluoromethyl-phenyl)-thiazol-4-yl]-propoxy}-1H-indol-3-yl)-acetic acid;
(5-{2-[5-Methyl-2-(4-trifluoromethyl-phenyl)-thiazol-4-yl]-ethoxy}-1H-indol-3-yl)-acetic acid;
(R)-(1-Methyl-5-{2-[5-methyl-2-(4-trifluoromethyl-phenyl)-thiazol-4-yl]-propoxy}-1H-indol-3-yl)-acetic acid;
(S)-(1-Methyl-5-{2-[5-Methyl-2-(4-trifluoromethyl-phenyl)-thiazol-4-yl]-propoxy}-1H-indol-3-yl)-acetic acid;
(1-Methyl-5-{2-[5-methyl-2-(4-trifluoromethyl-phenyl)-thiazol-4-yl]-ethoxy}-1H-indol-3-yl)-acetic acid;
5-{2-[4-Isopropyl-2-(4-trifluoromethyl-phenyl)-oxazol-5-yl]-propoxy}-1H-indole-2-carboxylic acid; and
5-{2-[4-Isopropyl-2-(4-trifluoromethyl-phenyl)-oxazol-5-yl]-propoxy}-1-methyl-1H-indole-2-carboxylic acid.

44-46. (canceled)

47. A compound represented by the following Structural Formula: 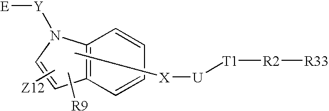
and stereoisomers, pharmaceutically acceptable salts, solvates and hydrates thereof, wherein:
(a) T1 is selected from the group consisting of 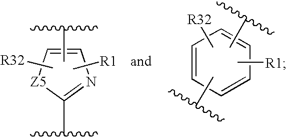
(b) R1 is selected from the group consisting of hydrogen, C₁-C₈ alkyl, C₁-C₈ alkenyl, aryl-C₀₋₄-alkyl, aryl-C₁₋₆-heteroalkyl, heteroaryl-C₀₋₄-alkyl, and C₃-C₆ cycloalkylaryl-C₀₋₂-alkyl, wherein C₁-C₈ alkyl, C₁-C₈ alkenyl, aryl-C₀₋₄-alkyl, aryl-C₁₋₆-heteroalkyl, heteroaryl-C₀₋₄-alkyl, and C₃-C₆ cycloalkylaryl-C₀₋₂-alkyl are each optionally substituted with from one to three substituents independently selected from R1′;
(c) R1′, R26, R27, R28, R31, Z14′, and Z15′ are each independently selected from the group consisting of hydrogen, hydroxy, cyano, nitro, halo, oxo, C₁-C₆ alkyl, C₁-C₆ alkyl-COOR12, C₁-C₆ alkoxy, C₁-C₆ haloalkyl, C₁-C₆ -haloalkyloxy, C₃-C₇ cycloalkyl, optionally substituted aryloxy, optionally substituted aryl-C₀₋₄-alkyl, optionally substituted heteroaryl, optionally substituted heterocycloalkyl, C(O)R13, COOR14, OC(O)R15, OS(O)₂R16, N(R17)₂, NR18C(O)R19, NR20SO₂R21, SR22, S(O)R23, S(O)₂R24, and S(O)₂N(R25)₂; R12, R13, R14, R15, R16, R17, R18, R19, R20, R21, R22, R23, R24 and R25 are each independently selected from the group consisting of hydrogen, C₁-C₆ alkyl and aryl;
(d) R2 is selected from the group consisting of C₀-C₈ alkyl and C₁₋₆-heteroalkyl;
(e) X is selected from the group consisting of a bond, O, S, S(O)₂ and N;
(f) U is an aliphatic linker wherein one carbon atom of the aliphatic linker may be replaced with O, NH or S, and wherein such aliphatic linker is optionally substituted with R30;
(g) Y is selected from the group consisting of C, O, S, NH and a single bond;
(h) E is C(R3)(R4)A or A and wherein
(i) A is selected from the group consisting of C₀-C₆ alkylcarboxyl, C₀-C₆ alkyltetrazole, C₁-C₆ alkylnitrile, C₀-C₆ alkylcarboxamide, C₀-C₆ alkylsulfonamide and C₀-C₆ alkylacylsulfonamide; wherein C₀-C₆ alkylsulfonamide, C₀-C₆ alkylacylsulfonamide and C₀-C₆ alkyltetrazole are each optionally substituted with from one to two groups independently selected from R7;
(ii) each R⁷ is independently selected from the group consisting of hydrogen, C₁-C₆ haloalkyl, aryl-C₀-C₄ alkyl and C₁-C₆ alkyl, wherein such alkyl and arylalkyl are each optionally substituted with from one to two groups independently selected from R7′; each R7′ is independently selected from halo, C₁-C₆ alkyl, and haloC₁-C₆ alkyl;
(iii) R3 is selected from the group consisting of hydrogen, C₁-C₅ alkyl, and C₁-C₅ alkoxy; and
(iv) R4 is selected from the group consisting of hydrogen, C₁-C₅ alkyl, C₁-C₅ alkoxy, aryloxy, C₃-C₆ cycloalkyl, and aryl C₀-C₄ alkyl, and R3 and R4 are optionally combined to form a C₃-C₄ cycloalkyl, and wherein alkyl, alkoxy, cycloalkyl and aryl-alkyl are each optionally substituted with one to three each independently selected from R26;
(i) Z5 is S or O;
(j) Z12 is selected from the group consisting of hydrogen and -Z13C₀-C₃alkylZ14;
(k) Z13 is selected from the group consisting of a single bond, CO, CO₂, CONZ15, and SO₂;
(l) Z14 is selected from the group consisting of aryl and heteroaryl, wherein the aryl and heteroaryl is each optionally substituted with from one to three substituents independently selected from Z14′;
(m) Z15 is selected from the group consisting of hydrogen aryl and heteroaryl, wherein the aryl and heteroaryl is each optionally substituted with from one to three substituents independently selected from Z15′;
(n) R9 is selected from the group consisting of hydrogen, C₁-C₄ alkyl, C₁-C₄ alkylenyl, halo, aryl-C₀-C₄ alkyl, heteroaryl, C₁-C₆ allyl, and OR29, and wherein aryl-C₀-C₄ alkyl, heteroaryl are each optionally substituted with from one to three independently selected from R27; R29 is selected from the group consisting of hydrogen and C₁-C₄ alkyl;
(o) R10, R11 are each independently selected from the group consisting of hydrogen, hydroxy, cyano, nitro, halo, oxo, C₁-C₆ alkyl, C₀-C₆ alkyl-COOR12″, C₁-C₆ alkoxy, C₁-C₆ haloalkyl, C₁-C₆ haloalkyloxy, C₃-C₇ cycloalkyl, aryl-C₀₋₄-alkyl, aryl-C₁₋₆-heteroalkyl, heteroaryl-C₀₋₄-alkyl, C₃-C₆ cycloalkylaryl-C₀₋₂-alkyl, aryloxy, C(O)R13′, COOR14, OC(O)R15′, OS(O)₂R16′, N(R17)₂, NR18′C(O)R19′, N20′SO₂R21′, SR22′ S(O)R23′, S(O)₂R24′, and S(O)₂N(R25′)₂; and wherein aryl-C₀₋₄-alkyl, aryl-C₁₋₆-heteroalkyl, heteroaryl-C₀₋₄-alkyl, and C₃-C₆ cycloalkylaryl-C₀₋₂-alkyl are each optionally substituted with from one to three independently selected from
(p) R12′, R12″, R13′, R14′, R15′, R16′, R17′, R18′, R19′, R20′, R21′, R22′, R23′, R24′, and R25′ are each independently selected from the group consisting of hydrogen, C₁-C₆ alkyl and aryl;
(q) R30 is selected from the group consisting of C₁-C₆ alkyl, aryl-C₀₋₄-alkyl, aryl-C₁₋₆-heteroalkyl, heteroaryl-C₀₋₄-alkyl, and C₃-C₆ cycloalkylaryl-C₀₋₂-alkyl, and wherein C₁-C₆ alkyl, aryl-C₀₋₄-alkyl, aryl-C₁₋₆-heteroalkyl, heteroaryl-C₀₋₄-alkyl, and C₃-C₆ cycloalkylaryl-C₀₋₂-alkyl are each optionally substituted with from one to three substituents each independently selected from R31;
(r) R32 is selected from the group consisting of a bond, hydrogen, halo, C₁-C₆ alkyl, C₁-C₆ haloalkyl, and C₁-C₆ alkyloxo;
(s) R33 is selected from the group consisting of C₂-C₈ alkyl, C₁-C₈ alkoxy, phenyl, thiophene, pyridine, piperidine, 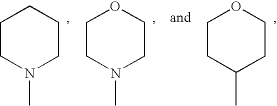
wherein the C₂-C₈ alkyl, C₁-C₈ alkoxy, phenyl, thiophene, pyridine, piperidine, 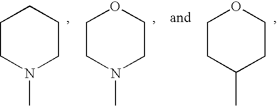
are each optionally substituted with R10 and R11.

48. The compound of claim 47, wherein A is selected from the group consisting of C₀-C₆ alkylcarboxyl, C₀-C₆ alkyltetrazole, C₁-C₆ alkylnitrile, C₀-C₆ alkylsulfonamide and C₀-C₆ alkylacylsulfonamide; wherein C₀-C₆ alkylsulfonamide, C₀-C₆ alkylacylsulfonamide and C₀-C₆ alkyltetrazole are each optionally substituted with from one to two groups independently selected from R⁷.

49. The compound of claim 47, wherein the compound is represented by the following Structural Formula: 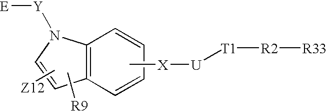
and stereoisomers, pharmaceutically acceptable salts, solvates and hydrates thereof, wherein:
(a) T1 is selected from the group consisting of 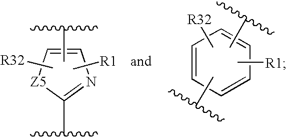
(b) R1 is selected from the group consisting of hydrogen, C₁-C₈ alkyl, C₁-C₈ alkenyl, aryl-C₀₋₄-alkyl, aryl-C₁₋₆-heteroalkyl, heteroaryl-C₀₋₄-alkyl, and C₃-C₆ cycloalkylaryl-C₀₋₂-alkyl, wherein C₁-C₈ alkyl, C₁-C₈ alkenyl, aryl-C₀₋₄-alkyl, aryl-C₁₋₆-heteroalkyl, heteroaryl-C₀₋₄-alkyl, and C₃-C₆ cycloalkylaryl-C₀₋₂-alkyl are each optionally substituted with from one to three substituents independently selected from R1′;
(c) R1′, R26, R27, R28, R31, Z14′, and Z15′ are each independently selected from the group consisting of hydrogen, hydroxy, cyano, nitro, halo, oxo, C₁-C₆ alkyl, C₁-C₆ alkyl-COOR12, C₁-C₆ alkoxy, C₁-C₆ haloalkyl, C₁-C₆ haloalkyloxy, C₃-C₇ cycloalkyl, aryloxy, aryl-C₀₋₄-alkyl, heteroaryl, heterocycloalkyl, C(O)R13, COOR14, OC(O)R15, OS(O)₂R16, N(R17)₂, NR18C(O)R19, NR20SO₂R21, SR22, S(O)R23, S(O)₂R24, and S(O)₂N(R25)₂; R12, R13, R14, R15, R16, R17, R18, R19, R20, R21, R22, R23, R24 and R25 are each independently selected from the group consisting of hydrogen, C₁-C₆ alkyl and aryl;
(d) R2 is selected from the group consisting of C₀-C₈ alkyl and C₁₋₆-heteroalkyl;
(e) X is selected from the group consisting of a bond, O, S, S(O)₂ and N;
(f) U is an aliphatic linker wherein one carbon atom of the aliphatic linker may be replaced with O, NH or S, and wherein such aliphatic linker is optionally substituted with R30;
(g) Y is selected from the group consisting of C, O, S, NH and a single bond;
(h) E is C(R3)(R4)A or A and wherein
(i) A is selected from the group consisting of carboxyl, tetrazole, C₁-C₆ alkylnitrile, carboxamide, sulfonamide and acylsulfonamide; wherein sulfonamide, acylsulfonamide and tetrazole are each optionally substituted with from one to two groups independently selected from R⁷;
(ii) each R⁷ is independently selected from the group consisting of hydrogen, C₁-C₆ haloalkyl, aryl-C₀-C₄ alkyl and C₁-C₆ alkyl, wherein such alkyl and arylalkyl are each optionally substituted with from one to two groups independently selected from R7′; each R7′ is independently selected from halo, C₁-C₆ alkyl, and haloC₁-C₆ alkyl;
(iii) R3 is selected from the group consisting of hydrogen, C₁-C₅ alkyl, and C₁-C₅ alkoxy; and
(iv) R4 is selected from the group consisting of hydrogen, C₁-C₅ alkyl, C₁-C₅ alkoxy, aryloxy, C₃-C₆ cycloalkyl, and aryl C₀-C₄ alkyl, and R3 and R4 are optionally combined to form a C₃-C₄ cycloalkyl, and wherein alkyl, alkoxy, cycloalkyl and aryl-alkyl are each optionally substituted with one to three each independently selected from R26;
(i) Z5 is S or O;
(j) Z12 is selected from the group consisting of hydrogen and -Z13C₀-C₃alkylZ14;
(k) Z13 is selected from the group consisting of a single bond, CO, CO₂, CONZ15, and SO₂;
(l) Z14 is selected from the group consisting of aryl and heteroaryl, wherein the aryl and heteroaryl is each optionally substituted with from one to three substituents independently selected from Z14′;
(m) Z15 is selected from the group consisting of hydrogen aryl and heteroaryl, wherein the aryl and heteroaryl is each optionally substituted with from one to three substituents independently selected from Z15′;
(n) R9 is selected from the group consisting of hydrogen, C₁-C₄ alkyl, C₁-C₄ alkylenyl, halo, aryl-C₀-C₄ alkyl, heteroaryl, C₁-C₆ allyl, and OR29, and wherein aryl-C₀-C₄ alkyl, heteroaryl are each optionally substituted with from one to three independently selected from R27; R29 is selected from the group consisting of hydrogen and C₁-C₄ alkyl;
(o) R10, R11 are each independently selected from the group consisting of hydrogen, hydroxy, cyano, nitro, halo, oxo, C₁-C₆ alkyl, C₀-C₆ alkyl-COOR12″, C₁-C₆ alkoxy, C₁-C₆ haloalkyl, C₁-C₆ haloalkyloxy, C₃-C₇ cycloalkyl, aryl-C₀₋₄-alkyl, aryl-C₁₋₆-heteroalkyl, heteroaryl-C₀₋₄-alkyl, C₃-C₆ cycloalkylaryl-C₀₋₂-alkyl, aryloxy, C(O)R13′, COOR14′, OC(O)R15′, OS(O)₂R16, N(R17)₂, NR18′C(O)R19, NR20′SO₂R21′, SR22′, S(O)R23′, S(O)₂R24′ and S(O)₂N(R25)₂; and wherein aryl-C₀₋₄-alkyl, aryl-C₁₋₆-heteroalkyl, heteroaryl-C₀₋₄-alkyl, and C₃-C₆ cycloalkylaryl-C₀₋₂-alkyl are each optionally substituted with from one to three independently selected from R28;
(p) R12′, R12″, R13′, R14′, R15′, R16′, R17′, R18′, R19′, R20′, R21′, R22′, R23′, R24′, and R25′ are each independently selected from the group consisting of hydrogen, C₁-C₆ alkyl and aryl;
(q) R30 is selected from the group consisting of C₁-C₆ alkyl, aryl-C₀₋₄-alkyl, aryl-C₁₋₆-heteroalkyl, heteroaryl-C₀₋₄-alkyl, and C₃-C₆ cycloalkylaryl-C₀₋₂-alkyl, and wherein C₁-C₆ alkyl, aryl-C₀₋₄-alkyl, aryl-C₁₋₆-heteroalkyl, heteroaryl-C₀₋₄-alkyl, and C₃-C₆ cycloalkylaryl-C₀₋₂-alkyl are each optionally substituted with from one to three substituents each independently selected from R31;
(r) R32 is selected from the group consisting of a bond, hydrogen, halo, C₁-C₆ alkyl, C₁-C₆ haloalkyl, and C₁-C₆ alkyloxo;
(s) R33 is selected from the group consisting of phenyl, thiophene, pyridine, piperidine, 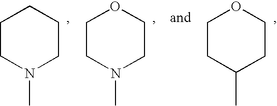
wherein the phenyl, thiophene, pyridine, piperidine, 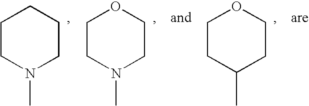
each optionally substituted with R10 and R11.

50. The compound of claim 47, wherein the compound is represented by the following Structural Formula:

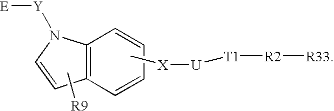

51. The compound of claim 50, wherein the compound is represented by the following Structural Formula:

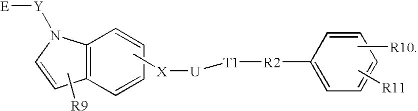

52. The compound of claim 51, wherein the compound is represented by the following Structural Formula:

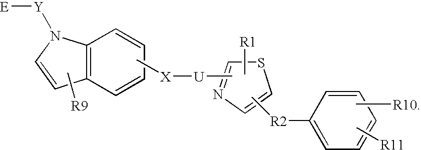

53-54. (canceled)

55. The compound of claim 52, wherein:
X is —O—;
E is —COOH, C₁-C₆ alkylcarboxyl, or C(R3)(R4)-C₁-C₆ alkyl-COOH;
R10 and R11 are each independently selected from the group consisting of hydrogen, halo, oxo, C₁-C₆ alkyl C₁-C₆ alkyl-COOR12″, C₁-C₆ alkoxy, C₁-C₆ haloalkyl, and C₁-C₆ haloalkyloxy;
R9 is selected from the group consisting of hydrogen and C₁-C₃ alkyl,
R1, R3, and R4 are each independently selected from the group consisting of hydrogen and C₁-C₂ alkyl;
R2 is a bond; and
U is saturated C₁-C₃ alkyl optionally substituted with C₁-C3 alkyl.

56-63. (canceled)

64. The compound of claim 51, wherein the compound is represented by the following Structural Formula:

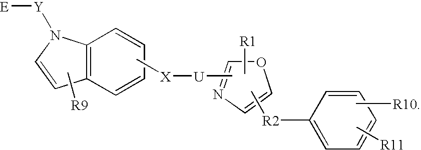

65-66. (canceled)

67. The compound of claim 64, wherein:
X is —O—,
E is —COOH, C₁-C₆ alkylcarboxyl, or C(R3)(R4)-C₁-C₆ alkyl-COOH;
R10 and R11 are each independently selected from the group consisting of hydrogen, halo, oxo, C₁-C₆ alkyl, C₁-C₆ alkyl-COOR12″, C₁-C₆ alkoxy, C₁-C₆ haloalkyl, and C₁-C₆ haloalkyloxy;
R9 is selected from the group consisting of hydrogen and C₁-C₃ alkyl;
R1, R3, and R4 are each independently selected from the group consisting of hydrogen and C₁-C₂ alkyl;
R2 is a bond: and
U is saturated C₁-C₃ alkyl optionally substituted with C₁-C₃ alkyl.

68-75. (canceled)

76. The compound of claim 51, wherein the compound is represented by the following Structural Formula:

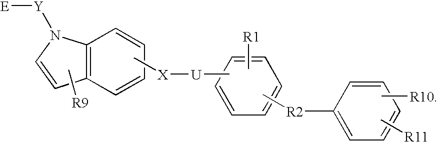

77-78. (canceled)

79. The compound of claim 76, wherein:
X is —O—;
E is —COOH, C₁-C₆ alkylcarboxyl, or C(R3)(R4)-C₁-C₆ alkyl-COOH;
R10 and R11 are each independently selected from the group consisting of hydrogen, halo, oxo, C₁-C₆ alkyl, C₁-C₆ alkyl-COOR12″, C₁-C₆ alkoxy, C₁-C₆ haloalkyl, and C—C₆ haloalkyloxy;
R9 is selected from the group consisting of hydrogen and C₁₋₃ alkyl:
R1, R3, and R4 are each independently selected from the group consisting of hydrogen and C₁-C₂ alkyl;
R2 is a bond; and
U is saturated C₁-C₃ alkyl optionally substituted with C₁-C₃ alkyl.

80-87. (canceled)

88. The compound of claim 47 wherein the compound is selected from the group consisting of:
{5-[5-Methyl-2-(4-trifluoromethyl-phenyl)-oxazol-4-ylmethoxy]-indol-1-yl}-acetic acid;
[5-(5-Methyl-2-phenyl-oxazol-4-ylmethoxy)-indol-1-yl]-acetic acid;
{5-[2-(4-Fluoro-phenyl)-5-methyl-oxazol-4-ylmethoxy]-indol-1-yl}-acetic acid;
{5-[2-(4-Benzyloxy-phenyl)-5-methyl-oxazol-4-ylmethoxy]-indol-1-yl}-acetic acid;
2-Methyl-2-(5-{2-[2-(4-trifluoromethyl-phenyl)-oxazol-4-ylmethoxy]-ethoxy}-indol-1-yl)-propionic acid;
{5-[4-Ethyl-2-(4-trifluoromethyl-phenyl)-thiazol-5-ylmethoxy]-indol-1-yl}-acetic acid;
2-Methyl-2-(5-{2-[4-methyl-2-(4-trifluoromethyl-phenyl)-thiazol-5-ylmethoxy]-ethoxy}-indol-1-yl)-propionic acid;
{5-[2-(3,5-Bis-trifluoromethyl-phenyl)-4-methyl-thiazol-5-ylmethoxy]-indol-1-yl}-acetic acid;
{5-[4-Methyl-2-(4-trifluoromethyl-phenyl)-oxazol-5-ylmethoxy]-indol-1-yl}-acetic acid;
2-Methyl-2-{5-[4-methyl-2-(4-trifluoromethyl-phenyl)-oxazol-5-ylmethoxy]-indol-1-yl}-propionic acid;
Racemic 2-{5-[4-Methyl-2-(4-trifluoromethyl-phenyl)-oxazol-5-ylmethoxy]-indol-1-yl}-propionic acid;
{5-[2-(4-Bromo-phenyl)-4-methyl-thiazol-5-ylmethoxy]-indol-1-yl}-acetic acid;
{5-[4-Butyl-2-(4-trifluoromethyl-phenyl)-thiazol-5-ylmethoxy]-indol-1-yl}-acetic acid;
2-{5-[4-Butyl-2-(4-trifluoromethyl-phenyl)-thiazol-5-ylmethoxy]-indol-1-yl}-propionic acid;
{5-[4-Phenethyl-2-(4-trifluoromethyl-phenyl)-thiazol-5-ylmethoxy]-indol-1-yl}-acetic acid;
2-{5-[4-(2-Chloro-6-fluoro-phenoxymethyl)-2-(4-trifluoromethyl-phenyl)-thiazol-5-ylmethoxy]-indol-1-yl}-propionic acid;
{5-[4-Phenoxymethyl-2-(4-trifluoromethyl-phenyl)-thiazol-5-ylmethoxy]-indol-1-yl acetic acid;
Racemic 2-Methyl-2-{5-[4-phenoxymethyl-2-(4-trifluoromethyl-phenyl)-thiazol-5-ylmethoxy]-indol-1-yl}-propionic acid;
2-Methyl-2-{5-[4-phenoxymethyl-2-(4-trifluoromethyl-phenyl)-thiazol-5-ylmethoxy]-indol-1-yl}-propionic acid;
3-{5-[4-Propyl-2-(4-trifluoromethyl-phenyl)-thiazol-5-ylmethoxy]-indol-1-yl}-propionic acid;
5-{5-[4-Methyl-2-(4-trifluoromethyl-phenyl)-thiazol-5-ylmethoxy]-indol-1-yl}-pentanoic acid;
5-{5-[4-Propyl-2-(4-trifluoromethyl-phenyl)-thiazol-5-ylmethoxy]-indol-1-yl}-pentanoic acid;
{5-[4-Isopropyl-2-(4-trifluoromethyl-phenyl)-thiazol-5-ylmethoxy]-indol-1-yl}-acetic acid;
{5-[4-(2-Chloro-6-fluoro-phenoxymethyl)-2-(4-trifluoromethyl-phenyl)-thiazol-5-ylmethoxy]-indol-1-yl}-acetic acid;
2-{5-[4-(2-Chloro-6-fluoro-phenoxymethyl)-2-(4-trifluoromethyl-phenyl)-thiazol-5-ylmethoxy]-indol-1-yl}-2-methyl-propionic acid;
5-{5-[5-Methyl-2-(4-trifluoromethyl-phenyl)-oxazol-4-ylmethoxy]-indol-1-yl}-pentanoic acid;
5-{5-[2-(4-Bromo-phenyl)-5-methyl-oxazol-4-ylmethoxy]-indol-1-yl}-pentanoic acid;
5-{5-[4-Phenethyl-2-(4-trifluoromethyl-phenyl)-thiazol-5-ylmethoxy]-indol-1}-pentanoic acid;
4-[1-(4-Carboxy-butyl)-1H-indol-5-yloxymethyl]-2-(4-trifluoromethyl-phenyl)-thiazole-5-carboxylic acid;
3-{5-[2-(4-Bromo-phenyl)-5-methyl-oxazol-4-ylmethoxy]-indol-1-yl}-propionic acid;
3-{5-[4-Phenethyl-2-(4-trifluoromethyl-phenyl)-thiazol-5-ylmethoxy]-indol-1-yl}-propionic acid;
3-{5-[4-Methyl-2-(4-trifluoromethyl-phenyl)-thiazol-5-ylmethoxy]-indol-1-yl}-propionic acid;
4-[1-(2-Carboxy-ethyl)-1H-indol-5-yloxymethyl]-2-(4-trifluoromethyl-phenyl)-thiazole-5-carboxylic acid;
{5-[4-Isopropyl-2-(4-trifluoromethyl-phenyl)-thiazol-5-ylmethylsulfanyl]-indol-1-yl}-acetic acid;
{5-[4-Ethyl-2-(4-trifluoromethyl-phenyl)-oxazol-5-ylmethoxy]-indol-1-yl}-acetic acid;
(5-{2-[5-Methyl-2-(4-trifluoromethyl-phenyl)-oxazol-4-yl]-ethoxy}-indol-1-yl)-acetic acid;
{5-[2-(5-Methyl-2-pyridin-4-yl-thiazol-4-yl)-ethoxy]-indol-1-yl}-acetic acid;
{5-[2-(5-Methyl-2-morpholin-4-yl-thiazol-4-yl)-ethoxy]-indol-1-yl}-acetic acid;
(5-{2-[5-Methyl-2-(4-trifluoromethyl-phenyl)-oxazol-4-yl]-ethoxy}-indol-1-yl)-acetic acid;
(5-{2-[5-Methyl-2-(tetrahydro-pyran-4-yl)-oxazol-4-yl]-ethoxy}-indol-1-yl)-acetic acid;
{5-[2-(2-Butoxy-5-methyl-oxazol-4-yl)-ethoxy]-indol-1-yl}-acetic acid;
{5-[2-(5-Methyl-2-pyridin-3-yl-thiazol-4-yl)-ethoxy]-indol-1-yl}-acetic acid;
{5-[2-(5-Methyl-2-pyridin-2-yl-thiazol-4-yl)-ethoxy]-indol-1-yl}-acetic acid;
(5-{2-[2-(5-Bromo-thiophen-2-yl)-5-methyl-oxazol-4-yl]-ethoxy}-indol-1-yl)-acetic acid;
{5-[3-(4-Butyl-phenoxy)-propoxy]-indol-1-yl}-acetic acid;
(5-{2-[2-(3-Bromo-phenyl)-5-methyl-oxazol-4-yl]-ethoxy}-indol-1-yl)-acetic acid;
(5-{2-[5-Ethyl-2-(4-trifluoromethyl-phenyl)-thiazol-4-yl]-ethoxy}-indol-1-yl)-acetic acid;
2-(5-{2-[5-Ethyl-2-(4-trifluoromethyl-phenyl)-thiazol-4-yl]-ethoxy}-indol-1-yl)-propionic acid;
(5-{2-[2-(2-Chloro-phenyl)-5-ethyl-thiazol-4-yl]-ethoxy}-indol-1-yl)-acetic acid;
2-(5-{2-[2-(2-Chloro-phenyl)-5-ethyl-thiazol-4-yl]-ethoxy}-indol-1-yl)-propionic acid;
(5-{2-[5-Propyl-2-(4-trifluoromethyl-phenyl)-thiazol-4-yl]-ethoxy}-indol-1-yl)-acetic acid;
2-(5-{2-[5-Propyl-2-(4-trifluoromethyl-phenyl)-thiazol-4-yl]-ethoxy}-indol-1-yl)-propionic acid;
2-(5-{2-[2-(2-Chloro-phenyl)-5-ethyl-thiazol-4-yl]-ethoxy}-indol-1-yl)-2-methyl-propionic acid;
Racemic 2-(5-{2-[5-Ethyl-2-(4-trifluoromethyl-phenyl)-oxazol-4-yl]-ethoxy}-indol-1-yl)-propionic acid;
(5-{2-[5-Ethyl-2-(4-trifluoromethyl-phenyl)-oxazol-4-yl]-ethoxy}-indol-1-yl)-acetic acid;
2-(5-{2-[5-Ethyl-2-(4-trifluoromethyl-phenyl)-oxazol-4-yl]-ethoxy}-indol-1-yl)-2-methyl-propionic acid;
3-(5-{2-[2-(2-Chloro-phenyl)-5-ethyl-thiazol-4-yl]-ethoxy}-indol-1-yl)-propionic acid;
3-(5-{2-[5-Ethyl-2-(4-trifluoromethyl-phenyl)-oxazol-4-yl]-ethoxy}-indo-1-yl)-propionic acid;
2-Methyl-2-(5-{2-[5-propyl-2-(4-trifluoromethyl-phenyl)-thiazol-4-yl]-ethoxy}-indol-1-yl)-propionic acid;
Racemic-(5-{1-[4-Methyl-2-(4-trifluoromethyl-phenyl)-thiazol-5-yl]-ethoxy}-indol-1-yl)-acetic acid;
Racemic-(5-{1-[4-Methyl-2-(4-trifluoromethyl-phenyl)-thiazol-5-yl]-ethoxy}-indol-1-yl)-acetic acid;
Racemic-(5-{1-[4-Isopropyl-2-(4-trifluoromethyl-phenyl)-thiazol-5-yl]-ethoxy}-indol-1-yl)-acetic acid;
Racemic-(5-{2-[5-Ethyl-2-(4-trifluoromethyl-phenyl)-thiazol-4-yl]-1-methyl-ethoxy}-indol-1-yl)-acetic acid;
Racemic-(5-{2-[5-Methyl-2-(4-trifluoromethyl-phenyl)-thiazol-4-yl]-propoxy}-indol-1-yl)-acetic acid;
(5-{2-[5-Methyl-2-(4-trifluoromethyl-phenyl)-oxazol-4-yl]-propoxy}-indol-1-yl)-acetic acid;
(S)-(5-{2-[5-Methyl-2-(4-trifluoromethyl-phenyl)-oxazol-4-yl]-propoxy}-indol-1-yl)-acetic acid;
(R)-(5-{2-[5-Methyl-2-(4-trifluoromethyl-phenyl)-oxazol-4-yl]-propoxy}-indol-1-yl)-acetic acid;
Racemic-(5-{1-[4-Propyl-2-(4-trifluoromethyl-phenyl)-thiazol-5-yl]-ethoxy}-indo-1-yl)-acetic acid;
Racemic-(5-{1-[4-Ethyl-2-(4-trifluoromethyl-phenyl)-oxazol-5-yl]-ethoxy}-indol-1-yl)-acetic acid;
{5-[4-Propyl-2-(4-trifluoromethyl-phenyl)-thiazol-5-ylmethoxy]-indol-1-yl}-acetic acid;
Racemic-2-{5-[4-Propyl-2-(4-trifluoromethyl-phenyl)-thiazol-5-ylmethoxy]-indol-1-yl}-propionic acid;
2-Methyl-2-{5-[4-propyl-2-(4-trifluoromethyl-phenyl)-thiazol-5-ylmethoxy]-indol-1-yl}-propionic acid;
Racemic-2-{5-[4-Phenethyl-2-(4-trifluoromethyl-phenyl)-thiazol-5-ylmethoxy]-indol-1-yl}-propionic acid;
2-Methyl-2-{5-[4-phenethyl-2-(4-trifluoromethyl-phenyl)-thiazol-5-ylmethoxy]-indol-1-yl}-propionic acid;
{5-[4-Phenethyl-2-(4-trifluoromethyl-phenyl)-thiazol-5-ylmethoxy]-indol-1-yl}-acetic acid;
{5-[4-Phenyl-2-(4-trifluoromethyl-phenyl)-thiazol-5-ylmethoxy]-indol-1-yl}-acetic acid;
{5-[4-tert-Butyl-2-(4-trifluoromethyl-phenyl)-thiazol-5-ylmethoxy]-indol-1-yl}-acetic acid;
{5-[4-[2-(2-Chloro-6-fluoro-phenyl)-ethyl]-2-(4-trifluoromethyl-phenyl)-thiazol-5-ylmethoxy]-indol-1-yl}-acetic acid;
2-Methyl-2-[5-(5-methyl-2-phenyl-oxazol-4-ylmethoxy)-indol-1-yl]-propionic acid;
2-{5-[2-(4-Trifluoromethyl-phenyl)-5-methyl-oxazol-4-ylmethoxy]-indol-1-yl}-2-methyl-propionic acid;
2-{5-[2-(4-Fluoro-phenyl)-5-methyl-oxazol-4-ylmethoxy]-indol-1-yl}-2-methyl-propionic acid;
2-{5-[2-(4-Bromo-phenyl)-5-methyl-oxazol-4-ylmethoxy]-indol-1-yl}-2-methyl-propionic acid;
2-Methyl-2-(5-{2-[5-methyl-2-(4-trifluoromethyl-phenyl)-oxazol-4-yl]-ethoxy}-indol-1-yl)-propionic acid;
2-(5-{2-[2-(4-Bromo-phenyl)-5-methyl-oxazol-4-yl]-ethoxy}-indol-1-yl)-2-methyl-propionic acid;
2-(5-{2-[2-(5-Bromo-thiophen-2-yl)-5-methyl-oxazol-4-yl]-ethoxy}-indol-1-yl)-2-methyl-propionic acid;
2-Methyl-2-{5-[2-(5-methyl-2-phenyl-thiazol-4-yl)-ethoxy]-indol-1-yl}-propionic acid;
{5-[4-Isopropyl-2-(4-trifluoromethyl-phenyl)-thiazol-5-ylmethoxy]-2-methyl-indol-1-yl}-acetic acid;
2-{5-[2-(3,5-Bis-trifluoromethyl-phenyl)-4-methyl-thiazol-5-ylmethoxy]-indol-1-yl}-2-methyl-propionic acid;
{4-[4-Propyl-2-(4-trifluoromethyl-phenyl)-thiazol-5-ylmethoxy]-indol-1-yl}-acetic acid;
Racemic-(5-{1-[4-[2-(2-Chloro-6-fluoro-phenyl)-ethyl]-2-(4-trifluoromethyl-phenyl)-thiazol-5-yl]-ethoxy}-indol-1-yl)-acetic acid;
Racemic-(5-{1-[4-Methyl-2-(4-trifluoromethyl-phenyl)-thiazol-5-yl]-2-phenyl-ethoxy}-indol-1-yl)-acetic acid;
Racemic-(5-{1-[4-Phenethyl-2-(4-trifluoromethyl-phenyl)-thiazol-5-yl]-ethoxy}-indol-1-yl)-acetic acid;
2-{5-[4-Ethyl-2-(4-trifluoromethyl-phenyl)-thiazol-5-ylmethoxy]-indol-1-yl}-2-methyl-propionic acid;
(R)-(5-{1-[4-[2-(2-Chloro-6-fluoro-phenyl)-ethyl]-2-(4-trifluoromethyl-phenyl)-thiazol-5-yl]-ethoxy}-indol-1-yl)-acetic acid;
(S)-(5-{1-[4-[2-(2-Chloro-6-fluoro-phenyl)-ethyl]-2-(4-trifluoromethyl-phenyl)-thiazol-5-yl]-ethoxy}-indol-1-yl)-acetic acid;
(S)-(5-{1-[4-[2-phenylethyl]-2-(4-trifluoromethyl-phetiyl)-thiazol-5-yl]-ethoxy}-indol-1-yl)-acetic acid;
(R)-(5-{1-[4-[2-phenylethyl]-2-(4-trifluoromethyl-phenyl)-thiazol-5-yl]-ethoxy}-indol-1-yl)-acetic acid;
(S)-(5-{1-[4-Propyl-2-(4-trifluoromethyl-phenyl)-thiazol-5-yl]-ethoxy}-indol-1-yl)-acetic acid;
(R)-(5-{1-[4-Propyl-2-(4-trifluoromethyl-phenyl)-thiazol-5-yl]-ethoxy}-indol-1-yl)-acetic acid;
(R)-(5-{1-[4-Isopropyl-2-(4-trifluoromethyl-phenyl)-thiazol-5-yl]-ethoxy}-indol-1-yl)-acetic acid;
(R)-(5-{1-[4-Methyl-2-(4-trifluoromethyl-phenyl)-thiazol-5-yl]-ethoxy}-indol-1-yl)-acetic acid;
(S)-(5-{1-[4-Methyl-2-(4-trifluoromethyl-phenyl)-thiazol-5-yl]-ethoxy}-indol-1-yl)-acetic acid;
(S)-(5-{2-[5-Ethyl-2-(4-trifluoromethyl-phenyl)-thiazol-4-yl]-1-methyl-ethoxy}-indol-1-yl)-acetic acid;
(R)-(5-{2-[5-Ethyl-2-(4-trifluoromethyl-phenyl)-thiazol-4-yl]-1-methyl-ethoxy}-indol-1-yl)-acetic acid;
N-(2-{5-[5-Methyl-2-(4-trifluoromethyl-phenyl)-oxazol-4-ylmethoxy]-indol-1-yl}-acetyl)-methanesulfonamide;
N-(2-{5-[5-Methyl-2-(4-trifluoromethyl-phenyl)-oxazol-4-ylmethoxy]-indol-1-yl}-acetyl)-benzenesulfonamide; and
N-[2-(5-{2-[5-Ethyl-2-(4-trifluoromethyl-phenyl)-thiazol-4-yl]-ethoxy}-indol-1-yl)-acetyl]-methanesulfonamide.

89-91. (canceled)

92. A method of treating a mammal in need of treatment for a disease, wherein the disease is treatable by modulating a peroxisome proliferator activated receptor, comprising the step of administering to the mammal in need thereof a therapeutically effective amount of the compound of claim 1 or 47.

93. (canceled)

94. The method of claim 92, wherein the disease is selected from the group consisting of diabetes mellitus, Syndrome X, and atherosclerosis.

95. The method of claim 94, wherein the disease is diabetes mellitus.

96. The method of claim 94, wherein the disease is Syndrome X.

97-139. (canceled)

140. The compound of claim 1, wherein the compound is selected from the group consisting of:
{5-[5-Methyl-2-(4-trifluoromethyl-phenyl)-oxazol-4-ylmethoxy]-indol-1-yl}-acetic acid;
[5-(5-Methyl-2-phenyl-oxazol-4-ylmethoxy)-indol-1-yl]-acetic acid;
{5-[2-(4-Fluoro-phenyl)-5-methyl-oxazol-4-ylmethoxy]-indol-1-yl}-acetic acid;
{5-[2-(4-Benzyloxy-phenyl)-5-methyl-oxazol-4-ylmethoxy]-indol-1-yl}-acetic acid;
2-Methyl-2-(5-{2-[2-(4-trifluoromethyl-phenyl)-oxazol-4-ylmethoxy]-ethoxy}-indol-1-yl)-propionic acid;
{5-[4-Ethyl-2-(4-trifluoromethyl-phenyl)-thiazol-5-ylmethoxy]-indol-1-yl}-acetic acid;
2-Methyl-2-(5-{2-[4-methyl-2-(4-trifluoromethyl-phenyl)-thiazol-5-ylmethoxy]-ethoxy}-indol-1-yl)-propionic acid;
{5-[2-(3,5-Bis-trifluoromethyl-phenyl)}4-methyl-thiazol-5-ylmethoxy]-indol-1-yl}-acetic acid;
{5-[4-Methyl-2-(4-trifluoromethyl-phenyl)-oxazol-5-ylmethoxy]-indol-1-yl}-acetic acid;
2-Methyl-2-{5-[4-methyl-2-(4-trifluoromethyl-phenyl)-oxazol-5-ylmethoxy]-indol-1-yl}-propionic acid;
Racemic 2-{5-[4-Methyl-2-(4-trifluoromethyl-phenyl)-oxazol-5-ylmethoxy]-indol-1-yl}-propionic acid;
{5-[2-(4-Bromo-phenyl)₄-methyl-thiazol-5-ylmethoxy]-indol-1-yl}-acetic acid;
{5-[4-Butyl-2-(4-trifluoromethyl-phenyl)-thiazol-5-ylmethoxy]-indol-1-yl}-acetic acid;
2-{5-[4-Butyl-2-(4-trifluoromethyl-phenyl)-thiazol-5-ylmethoxy]-indol-1-yl}-propionic acid;
{5-[4-Phenethyl-2-(4-trifluoromethyl-phenyl)-thiazol-5-ylmethoxy]-indol-1-yl}-acetic acid;
2-{5-[4-(2-Chloro-6-fluoro-phenoxymethyl)-2-(4-trifluoromethyl-phenyl)-thiazol-5-ylmethoxy]-indol-1-yl}-propionic acid;
Racemic 2-Methyl-2-{5-[4-phenoxymethyl-2-(4-trifluoromethyl-phenyl)-thiazol-5-ylmethoxy]-indol-1-yl}-propionic acid;
2-Methyl-2-{5-[4-phenoxymethyl-2-(4-trifluoromethyl-phenyl)-thiazol-5-ylmethoxy]-indol-1-yl}-propionic acid;
3-{5-[4-Propyl-2-(4-trifluoromethyl-phenyl)-thiazol-5-ylmethoxy]-indol-1-yl}-propionic acid;
5-{5-[4-Methyl-2-(4-trifluoromethyl-phenyl)-thiazol-5-ylmethoxy]-indol-1-yl}-pentanoic acid;
5-{5-[4-Propyl-2-(4-trifluoromethyl-phenyl)-thiazol-5-ylmethoxy]-indol-1-yl}-pentanoic acid;
{5-[4-Isopropyl-2-(4-trifluoromethyl-phenyl)-thiazol-5-ylmethoxy]-indol-1-yl}-acetic acid;
2-{5-[4-(2-Chloro-6-fluoro-phenoxymethyl)-2-(4-trifluoromethyl-phenyl)-thiazol-5-ylmethoxy]-indol-1-yl}-2-methyl-propionic acid;
5-{5-[5-Methyl-2-(4-trifluoromethyl-phenyl)-oxazol-4-ylmethoxy]-indol-1-yl}-pentanoic acid;
5-{5-[2-(4-Bromo-phenyl)-5-methyl-oxazol-4-ylmethoxy]-indol-1-yl}-pentanoic acid;
3-{5-[2-(4-Bromo-phenyl)-5-methyl-oxazol-4-ylmethoxy]-indol-1-yl}-propionic acid;
3-{5-[4-Methyl-2-(4-trifluoromethyl-phenyl)-thiazol-5-ylmethoxy]-indol-1-yl}-propionic acid;
{5-[4-Isopropyl-2-(4-trifluoromethyl-phenyl)-thiazol-5-ylmethylsulfanyl]-indol-1-yl}-acetic acid;
{5-[4-Ethyl-2-(4-trifluoromethyl-phenyl)-oxazol-5-ylmethoxy]-indol-1-yl}-acetic acid;
(5-{2-[5-Methyl-2-(4-trifluoromethyl-phenyl)-oxazol-4-yl]-ethoxy}-indol-1-yl)-acetic acid;
{5-[2-(5-Methyl-2-pyridin-4-yl-thiazol-4-yl)-ethoxy]-indol-1-yl}-acetic acid;
{5-[2-(5-Methyl-2-morpholin-4-yl-thiazol-4-yl)-ethoxy]-indol-1-yl}-acetic acid;
(5-{2-[5-Methyl-2-(4-trifluoromethyl-phenyl)-oxazol-4-yl]-ethoxy}-indol-1-yl)-acetic acid;
(5-{2-[5-Methyl-2-(tetrahydro-pyran-4-yl)-oxazol-4-yl]-ethoxy}-indol-1-yl)-acetic acid;
{5-[2-(2-Butoxy-5-methyl-oxazol-4-yl)-ethoxy]-indol-1-yl}-acetic acid;
{5-[2-(5-Methyl-2-pyridin-3-yl-thiazol-4-yl)-ethoxy]-indol-1-yl}-acetic acid;
{5-[2-(5-Methyl-2-pyridin-2-yl-thiazol-4-yl)-ethoxy]-indol-1-yl}-acetic acid;
(5-{2-[2-(5-Bromo-thiophen-2-yl)-5-methyl-oxazol-4-yl]-ethoxy}-indol-1-yl)-acetic acid;
(5-{2-[2-(3-Bromo-phenyl)-5-methyl-oxazol-4-yl]-ethoxy}-indol-1-yl)-acetic acid;
(5-{2-[5-Ethyl-2-(4-trifluoromethyl-phenyl)-thiazol-4-yl]-ethoxy}-indol-1-yl)-acetic acid;
2-(5-{2-[5-Ethyl-2-(4-trifluoromethyl-phenyl)-thiazol-4-yl]-ethoxy}-indol-1-yl)-propionic acid;
(5-{2-[2-(2-Chloro-phenyl)-5-ethyl-thiazol-4-yl]-ethoxy}-indol-1-yl)-acetic acid;
2-(5-{2-[2-(2-Chloro-phenyl)-5-ethyl-thiazol-4-yl]-ethoxy}-indol-1-yl)-propionic acid;
(5-{2-[5-Propyl-2-(4-trifluoromethyl-phenyl)-thiazol-4-yl]-ethoxy}-indol-1-yl)-acetic acid;
2-(5-{2-[5-Propyl-2-(4-trifluoromethyl-phenyl)-thiazol-4-yl]-ethoxy}-indol-1-yl)-propionic acid;
2-(5-{2-[2-(2-Chloro-phenyl)-5-ethyl-thiazol-4-yl]-ethoxy}-indol-1-yl)-2-methyl-propionic acid;
Racemic 2-(5-{2-[5-Ethyl-2-(4-trifluoromethyl-phenyl)-oxazol-4-yl]-ethoxy}-indol-1-yl)-propionic acid;
(5-{2-[5-Ethyl-2-(4-trifluoromethyl-phenyl)-oxazol-4-yl]-ethoxy}-indol-1-yl)-acetic acid;
2-(5-{2-[5-Ethyl-2-(4-trifluoromethyl-phenyl)-oxazol-4-yl]-ethoxy}-indol-1-yl)-2-methyl-propionic acid; 3-(5-{2-[2-(2-Chloro-phenyl)-5-ethyl-thiazol-4-yl]-ethoxy}-indol-1-yl)-propionic acid;
3-(5-{2-[5-Ethyl-2-(4-trifluoromethyl-phenyl)-oxazol-4-yl]-ethoxy}-indol-1-yl)-propionic acid;
2-Methyl-2-(5-{2-[5-propyl-2-(4-trifluoromethyl-phenyl)-thiazol-4-yl]-ethoxy}-indol-1-yl)-propionic acid;
Racemic-(5-{1-[4-Methyl-2-(4-trifluoromethyl-phenyl)-thiazol-5-yl]-ethoxy}-indol-1-yl)-acetic acid;
Racemic-(5-{1-[4-Methyl-2-(4-trifluoromethyl-phenyl)-thiazol-5-yl]-ethoxy}-indol-1-yl)-acetic acid;
Racemic-(5-{1-[4-Isopropyl-2-(4-trifluoromethyl-phenyl)-thiazol-5-yl]-ethoxy}-indol-1-yl)-acetic acid;
Racemic-(5-{2-[5-Ethyl-2-(4-trifluoromethyl-phenyl)-thiazol-4-yl]-1-methyl-ethoxy}-indol-1-yl)-acetic acid;
Racemic-(5-{2-[5-Methyl-2-(4-trifluoromethyl-phenyl)-thiazol-4-yl]-propoxy}-indol-1-yl)-acetic acid;
(5-{2-[5-Methyl-2-(4-trifluoromethyl-phenyl)-oxazol-4-yl]-propoxy}-indol-1-yl)-acetic acid;
(S)-(5-{2-[5-Methyl-2-(4-trifluoromethyl-phenyl)-oxazol-4-yl]-propoxy}-indol-1-yl)-acetic acid;
(R)-(5-{2-[5-Methyl-2-(4-trifluoromethyl-phenyl)-oxazol-4-yl]-propoxy}-indol-1-yl)-acetic acid;
Racemic-(5-{1-[4-Propyl-2-(4-trifluoromethyl-phenyl)-thiazol-5-yl]-ethoxy}-indol-1-yl)-acetic acid;
Racemic-(5-{1-[4-Ethyl-2-(4-trifluoromethyl-phenyl)-oxazol-5-yl]-ethoxy}-indol-1-yl)-acetic acid;
{5-[4-Propyl-2-(4-trifluoromethyl-phenyl)-thiazol-5-ylmethoxy]-indol-1-yl}-acetic acid;
Racemic-2-{5-[4-Propyl-2-(4-trifluoromethyl-phenyl)-thiazol-5-ylmethoxy]-indol-1-yl}-propionic acid;
2-Methyl-2-{5-[4-propyl-2-(4-trifluoromethyl-phenyl)-thiazol-5-ylmethoxy]-indol-1-yl}-propionic acid;
Racemic-2-{5-[4-Phenethyl-2-(4-trifluoromethyl-phenyl)-thiazol-5-ylmethoxy]-indol-1-yl}-propionic acid;
2-Methyl-2-{5-[4-phenethyl-2-(4-trifluoromethyl-phenyl)-thiazol-5-ylmethoxy]-indol-1-yl}-propionic acid;
{5-[4-tert-Butyl-2-(4-trifluoromethyl-phenyl)-thiazol-5-ylmethoxy]-indol-1-yl}-acetic acid;
2-Methyl-2-[5-(5-methyl-2-phenyl-oxazol-4-ylmethoxy)-indol-1-yl]-propionic acid;
2-{5-[2-(4-Trifluoromethyl-phenyl)-5-methyl-oxazol-4-ylmethoxy]-indol-1-yl}-2-methyl-propionic acid;
2-{5-[2-(4-Fluoro-phenyl)-5-methyl-oxazol-4-ylmethoxy]-indol-1-yl}-2-methyl-propionic acid;
2-{5-[2-(4-Bromo-phenyl)-5-methyl-oxazol-4-ylmethoxy]-indol-1-yl}-2-methyl-propionic acid;
2-Methyl-2-(5-{2-[5-methyl-2-(4-trifluoromethyl-phenyl)-oxazol-4-yl]-ethoxy}-indol-1-yl)-propionic acid;
2-(5-{2-[2-(4-Bromo-phenyl)-5-methyl-oxazol-4-yl]-ethoxy}-indol-1-yl)-2-methyl-propionic acid;
2-(5-{2-[2-(5-Bromo-thiophen-2-yl)-5-methyl-oxazol-4-yl]-ethoxy}-indol-1-yl)-2-methyl-propionic acid;
2-Methyl-2-{5-[2-(5-methyl-2-phenyl-thiazol-4-yl)-ethoxy]-indol-1-yl}-propionic acid;
{5-[4-Isopropyl-2-(4-trifluoromethyl-phenyl)-thiazol-5-ylmethoxy]-2-methyl-indol-1-yl}-acetic acid;
2-{5-[2-(3,5-Bis-trifluoromethyl-phenyl)₄-methyl-thiazol-5-ylmethoxy]-indol-1-yl}-2-methyl-propionic acid;
{4-[4-Propyl-2-(4-trifluoromethyl-phenyl)-thiazol-5-ylmethoxy]-indol-1-yl}-acetic acid;
Racemic-(5-{1-[4-[2-(2-Chloro-6-fluoro-phenyl)-ethyl]-2-(4-trifluoromethyl-phenyl)-thiazol-5-yl]-ethoxy}-indol-1-yl)-acetic acid;
Racemic-(5-{1-[4-Methyl-2-(4-trifluoromethyl-phenyl)-thiazol-5-yl]-2-phenyl-ethoxy}-indol-1-yl)-acetic acid;
Racemic-(5-{1-[4-Phenethyl-2-(4-trifluoromethyl-phenyl)-thiazol-5-yl]-ethoxy}-indol-1-yl)-acetic acid;
2-{5-[4-Ethyl-2-(4-trifluoromethyl-phenyl)-thiazol-5-ylmethoxy]-indol-1-yl}-2-methyl-propionic acid;
(R)-(5-{1-[4-[2-(2-Chloro-6-fluoro-phenyl)-ethyl]-2-(4-trifluoromethyl-phenyl)-thiazol-5-yl]-ethoxy}-indol-1-yl)-acetic acid;
(S)-(5-{1-[4-[2-(2-Chloro-6-fluoro-phenyl)-ethyl]-2-(4-trifluoromethyl-phenyl)-thiazol-5-yl]-ethoxy}-indol-1-yl)-acetic acid;
(S)-(5-{1-[4-[2-phenylethyl]-2-(4-trifluoromethyl-phenyl)-thiazol-5-yl]-ethoxy}-indol-1-yl)-acetic acid;
(R)-(5-{1-[4-[2-phenylethyl]-2-(4-trifluoromethyl-phenyl)-thiazol-5-yl]-ethoxy}-indol-1-yl)-acetic acid;
(S)-(5-{1-[4-Propyl-2-(4-trifluoromethyl-phenyl)-thiazol-5-yl]-ethoxy}-indol-1-yl)-acetic acid;
(R)-(5-{1-[4-Propyl-2-(4-trifluoromethyl-phenyl)-thiazol-5-yl]-ethoxy}-indol-1-yl)-acetic acid;
(R)-(5-{1-[4-Isopropyl-2-(4-trifluoromethyl-phenyl)-thiazol-5-yl]-ethoxy}-indol-1-yl)-acetic acid;
(R)-(5-{1-[4-Methyl-2-(4-trifluoromethyl-phenyl)-thiazol-5-yl]-ethoxy}-indol-1-yl)-acetic acid;
(S)-(5-{1-[4-Methyl-2-(4-trifluoromethyl-phenyl)-thiazol-5-yl]-ethoxy}-indol-1-yl)-acetic acid;
(S)-(5-{2-[5-Ethyl-2-(4-trifluoromethyl-phenyl)-thiazol-4-yl]-1-methyl-ethoxy}-indol-1-yl)-acetic acid;
(R)-(5-{2-[5-Ethyl-2-(4-trifluoromethyl-phenyl)-thiazol-4-yl]-1-methyl-ethoxy}-indol-1-yl)-acetic acid;
N-(2-{5-[5-Methyl-2-(4-trifluoromethyl-phenyl)-oxazol-4-ylmethoxy]-indol-1-yl}-acetyl)-methanesulfonamide;
N-(2-{5-[5-Methyl-2-(4-trifluoromethyl-phenyl)-oxazol-4-ylmethoxy]-indol-1-yl}-acetyl)-benzenesulfonamide; and
N-[2-(5-{2-[5-Ethyl-2-(4-trifluoromethyl-phenyl)-thiazol-4-yl]-ethoxy}-indol-1-yl)-acetyl]-methanesulfonamide.
